SEQUENCE LISTING (2 sequences)

SEQ ID NO: 1 - DNA, 2841 bp, Homo sapiens
  source 1..2841: mol_type = genomic DNA; note = Nucleotide sequence encoding His-TEV-hDGKzeta-(M1-V929)
atgcatcaccatcaccatcaccatcacgagaacctgtacttccagggatccatggaaccccgtgatggtagccccgaagctcgtagctccgattccgagtccgccagcgcttcctcctccggtagcgaacgtgacgctggtcccgagcccgacaaagctccccgtcgtctgaataagcgccgttttcccggtctccgtctgttcggccaccgcaaggccatcactaagtccggtctccagcatctggctcctcctcctcctacccccggtgctccttgctccgaatccgagcgccagattcgctccactgtggattggtccgaaagcgccacctatggtgagcatatctggttcgagaccaacgtctccggcgacttctgttatgtcggtgagcaatactgtgtggctcgtatgctgcagaagtccgtgtcccgccgtaaatgcgccgcttgcaaaatcgtggtccataccccttgcatcgagcaactggagaaaatcaacttccgctgcaagcccagctttcgtgagtccggttcccgcaacgtgcgcgaacctactttcgtgcgccaccactgggtgcatcgtcgtcgccaagacggcaaatgccgccactgcggcaaaggttttcagcagaaattcaccttccacagcaaggagatcgtcgccatcagctgcagctggtgcaaacaagcttaccattccaaagtgagctgcttcatgctccagcagatcgaagagccttgctctctgggtgtgcatgctgctgtcgtgattccccctacttggattctgcgtgctcgccgtccccagaacactctgaaggcctccaaaaagaagaagcgcgccagcttcaagcgtaagagctccaaaaagggtcccgaagagggccgttggcgtcccttcatcatccgccctactccttcccctctgatgaagcctctgctggtcttcgtcaaccctaagagcggcggcaaccaaggtgctaaaatcatccagtccttcctctggtatctgaaccctcgtcaagtgttcgacctcagccaaggcggtcctaaggaggctctggagatgtaccgcaaggtccacaatctgcgcatcctcgcttgtggtggcgatggcaccgtgggctggattctgtccactctggaccaactgcgtctgaaacctcccccccccgtggctattctgcctctcggtaccggcaacgatctggctcgtactctgaattggggtggtggctacaccgatgagcccgtgtccaagattctgtcccacgtcgaagaaggcaatgtcgtccaactggaccgttgggacctccacgccgaacccaaccccgaggctggccccgaggaccgtgacgagggcgctactgaccgtctgcccctcgacgtcttcaataattacttctctctgggctttgacgctcacgtgactctggaatttcatgaaagccgcgaggccaaccccgagaagttcaattcccgtttccgcaacaagatgttctacgctggcaccgccttcagcgacttcctcatgggctccagcaaggacctcgctaagcatatccgcgtggtgtgcgatggcatggatctgacccctaagatccaagatctgaagccccaatgtgtcgtgtttctgaacatcccccgctactgcgctggtactatgccttggggccatcccggtgaacaccatgacttcgaacctcagcgtcatgatgacggctatctggaggtgatcggtttcaccatgacctccctcgctgctctgcaagtgggtggccacggcgaacgtctgactcaatgccgcgaggtggtgctgaccaccagcaaagccatccccgtccaagtggatggtgagccttgcaagctggccgcctcccgtatccgtatcgctctccgcaatcaagctaccatggtccagaaggccaaacgccgcagcgctgctcctctccacagcgaccaacaacccgtccccgaacagctgcgcatccaagtgtcccgtgtcagcatgcatgactacgaggctctgcactacgacaaggaacagctgaaggaagccagcgtgcctctgggtactgtggtcgtgcccggtgacagcgatctggagctctgccgtgcccacatcgagcgtctgcagcaagagcccgacggtgctggtgccaagagccctacttgccaaaaactctcccccaagtggtgtttcctcgacgctaccaccgccagccgcttctaccgcattgatcgcgcccaagagcatctgaactatgtcaccgagatcgctcaagacgagatctacatcctcgaccccgaactcctcggtgctagcgcccgtcccgacctccccactcctacctcccctctgcccacttccccttgttcccccacccctcgtagcctccaaggtgatgctgcccctcctcaaggtgaggagctcattgaggccgctaagcgtaacgatttctgcaagctccaagagctgcatcgtgctggtggcgacctcatgcaccgcgatgagcagagccgcactctgctgcaccacgctgtgtccactggtagcaaggacgtggtgcgctatctgctggaccacgctcctcccgagatcctcgacgctgtggaagaaaacggcgagacttgcctccaccaagctgctgctctgggtcaacgtaccatctgccactacatcgtcgaagctggtgcttctctgatgaagaccgaccagcaaggtgatactccccgtcagcgcgccgagaaagcccaagacaccgaactggctgcctatctggagaaccgtcagcactaccagatgattcagcgtgaagaccaagagaccgccgtgtaa

SEQ ID NO: 2 - protein, 929 aa, Homo sapiens
  source 1..929: note = Amino acid sequence of His-TEV-hDGKzeta-(M1-V929)
MEPRDGSPEARSSDSESASASSSGSERDAGPEPDKAPRRLNKRRFPGLRLFGHRKAITKSGLQHLAPPPPTPGAPCSESERQIRSTVDWSESATYGEHIWFETNVSGDFCYVGEQYCVARMLQKSVSRRKCAACKIVVHTPCIEQLEKINFRCKPSFRESGSRNVREPTFVRHHWVHRRRQDGKCRHCGKGFQQKFTFHSKEIVAISCSWCKQAYHSKVSCFMLQQIEEPCSLGVHAAVVIPPTWILRARRPQNTLKASKKKKRASFKRKSSKKGPEEGRWRPFIIRPTPSPLMKPLLVFVNPKSGGNQGAKIIQSFLWYLNPRQVFDLSQGGPKEALEMYRKVHNLRILACGGDGTVGWILSTLDQLRLKPPPPVAILPLGTGNDLARTLNWGGGYTDEPVSKILSHVEEGNVVQLDRWDLHAEPNPEAGPEDRDEGATDRLPLDVFNNYFSLGFDAHVTLEFHESREANPEKFNSRFRNKMFYAGTAFSDFLMGSSKDLAKHIRVVCDGMDLTPKIQDLKPQCVVFLNIPRYCAGTMPWGHPGEHHDFEPQRHDDGYLEVIGFTMTSLAALQVGGHGERLTQCREVVLTTSKAIPVQVDGEPCKLAASRIRIALRNQATMVQKAKRRSAAPLHSDQQPVPEQLRIQVSRVSMHDYEALHYDKEQLKEASVPLGTVVVPGDSDLELCRAHIERLQQEPDGAGAKSPTCQKLSPKWCFLDATTASRFYRIDRAQEHLNYVTEIAQDEIYILDPELLGASARPDLPTPTSPLPTSPCSPTPRSLQGDAAPPQGEELIEAAKRNDFCKLQELHRAGGDLMHRDEQSRTLLHHAVSTGSKDVVRYLLDHAPPEILDAVEENGETCLHQAAALGQRTICHYIVEAGASLMKTDQQGDTPRQRAEKAQDTELAAYLENRQHYQMIQREDQETAV

(19) United States
(12) Patent Application Publication
(10) Pub. No.: US 2025/0353858 A1
(43) Pub. Date: Nov. 20, 2025

(54) CONDENSED HETEROCYCLIC COMPOUNDS AS INHIBITOR OF DIACYLGLYCEROL KINASES

(71) Applicant: BEONE MEDICINES I GMBH, Basel (CH)

(72) Inventors: Zhikun Ni, Beijing (CN); Guoliang Zhang, Beijing (CN); Jianzhuang Miao, Beijing (CN); Ce Wang, Beijing (CN)

(73) Assignee: BEONE MEDICINES I GMBH, Basel (CH)

(21) Appl. No.: 19/283,392

(22) Filed: Jul. 29, 2025

Related U.S. Application Data
(63) Continuation of application No. PCT/CN2024/075498, filed on Feb. 2, 2024.

(30) Foreign Application Priority Data
Feb. 2, 2023 (WO) PCT/CN2023/074238
Oct. 26, 2023 (WO) PCT/CN2023/126798

(51) Int. Cl. C07D 487/04; A61K 31/426; A61K 31/4365; A61K 31/519; C07D 495/04; C07D 513/04

(52) U.S. Cl. CPC C07D 487/04; A61K 31/426; A61K 31/4365; A61K 31/519; C07D 495/04; C07D 513/04

(57) ABSTRACT

Disclosed herein is a compound of Formula (I) for activating T cells, promoting T cell proliferation, and/or exhibiting antitumor activity, a method of using the compounds disclosed herein for treating cancer, and a pharmaceutical composition comprising the same.

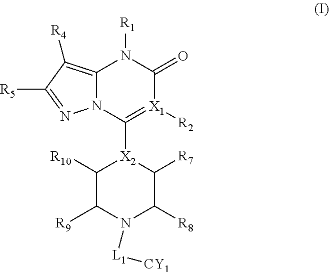

### CROSS-REFERENCE TO RELATED APPLICATIONS

This application is a continuation of International Application No. PCT/CN2024/075498, filed Feb. 2, 2024, which claims priority to International Application No. PCT/CN2023/074238, filed Feb. 2, 2023, and International Application No. PCT/CN2023/126798, filed Oct. 26, 2023, each of which is incorporated by reference herein in its entirety.

### SEQUENCE LISTING

The application contains a Sequence Listing, which has been submitted electronically in .XML format and is hereby incorporated by reference herein in its entirety. Said .XML, created on Feb. 2, 2024, is named “01368-0103-00PCT.xml,” and is 7,007 bytes in size.

### FIELD OF THE DISCLOSURE

Disclosed herein is a compound of Formula (I) for activating T cells, promoting T cell proliferation, and/or exhibiting antitumor activity, a method of using the compounds disclosed herein for treating cancer, and a pharmaceutical composition comprising the same.

### BACKGROUND OF THE DISCLOSURE

Diacylglycerol kinases (DGKs) are a family of lipid kinases that phosphorylates and converts diacylglycerol (DAG) into phosphatidic acid (PA). As the substrate of DGKs, DAG is generated from inositol phospholipids and other phospholipids at the plasma membrane by phospholipase C (PLC) hydrolysis in response to the activation of various cell-surface receptors, including G-protein coupled receptors (GPCR) and immunoreceptor tyrosine-based activation motif (ITAM)-bearing receptors (Rhee, Sue Goo. Annual review of biochemistry. 2001, 70.1: 281-312). DAG is one of the key intracellular second messengers that recruits and activates many downstream effectors including protein kinase C (PKC), protein kinase D (PKD) families, and Ras guanyl nucleotide releasing proteins (RasGRPs), which in turn activates NF-κB and extracellular regulated kinase (ERK) pathways (Mérida, Isabel, et al. Biochemical Journal. 2008, 409.1: 1-18, Joshi, Rohan P., et al. International Journal of Molecular Sciences. 2013, 14.4: 6649-6673). By consuming DAG, DGK controls and tunes the threshold and duration of DAG mediated signaling. Mammalian DGK family comprises 10 different members, in which DGKα, DGKζ and DGKδ are the three major isoforms that abundantly expressed in lymphoid tissues (Joshi, Rohan P., et al. International Journal of Molecular Sciences. 2013, 14.4: 6649-6673).

Cancer immunotherapy is a type of cancer treatment to manipulate and boost host immune system to recognize and attack cancer cells. A vast majority of studies have focused on targeting immune checkpoint inhibitors, such as CTLA-4 and PD-1/PD-L1, to reinvigorate exhausted CD8⁺ T cells within tumor sites. It was emerged that peripheral T cell tolerance, which under normal circumstances prevents detrimental autoimmune disease, can be hijacked by tumors to prevent anti-tumor immune response during carcinogenesis (Nüssing, Simone, et al. Frontiers in Immunology. 2020, 11: 2461). T cell anergy is a one of the most important mechanisms of T cell tolerance and has been reported to occur in tumor infiltrated T cells, which contributes to the immunosuppressive nature of tumor microenvironment (Abe, Brian T., and Fernando Macian. Oncoimmunology. 2013, 2.2: e22679). Anergy-associated transcription factor early growth response gene2 (Egr2) directly binds to Dgka and Dgkz promoter and increases their expression (Zheng, Yan, et al. Journal of Experimental Medicine 2012, 209.12: 2157-2163; Zheng, Yan, et al. Molecular Immunology. 2013, 55.3-4: 283-291). In anergic T cells, both DGKα and DGKζ play critical roles to negatively regulate DAG-signaling downstream of TCR and reduce the strength of TCR activation (Chen, Shelley S., et al. Frontiers in Cell and Developmental Biology. 2016, 4: 130). Thus, immune cell expressed DGKα and DGKζ were investigated as potential targets to reverse the hyporesponsiveness of the tumor infiltrated T cells. It was demonstrated that genetic deletion of DGKα or DGKζ enhanced cytokine production and proliferation of T cells (Olenchock, Benjamin A., et al. Nature immunology. 2006, 7.11: 1174-1181; Zhong, Xiao-Ping, et al. Nature immunology. 2003, 4.9: 882-890). DGKα or DGKζ single knockout in both mouse or human chimeric antigen receptor (CAR)-T cells showed superior effector function as determined by enhanced in vitro cytotoxicity and cytokine secretion when cocultured with antigen expressing titled cells (Riese, Matthew J., et al. Cancer Research. 2013, 73.12: 3566-3577; Jung, In-Young, et al. Cancer Research. 2018, 78.16: 4692-4703). MesoCAR-transduced DGKα or DGKζ deficient T cells also showed significantly elevated in vivo activity against mesotheliomas (Riese, Matthew J., et al. Cancer Research. 2013, 73.12: 3566-3577). DGKζ^{-/-} mice showed enhanced tumor suppressive efficacy with both orthotopic and subcutaneously implanted models (Wesley, Erin M., et al. Immunohorizons. 2018, 2.4: 107-118; Wee, Susan, et al. AACR; Cancer Res 2019; 79(13 Suppl): Abstract nr 936). Besides the T cell regulatory function, both DGKα and DGKζ also involve in tuning NK cell activation at tumor site (Prinz, Petra U., et al. International Journal of Cancer. 2014. 135.8: 1832-1841; Yang, Enjun, et al. The Journal of Immunology. 2016, 197.3: 934-941). In addition, DGKζ were found to play a critical role to control the activation threshold of mature B cells (Wheeler, Matthew L., et al. Science Signaling. 2013, 6.297: ra91-ra91). In summary, all these preclinical data suggest titled inhibition of DGKα and DGKζ could be therapeutic beneficial to promote immunity against cancer.

Although the existing anti-CTLA-4 and anti-PD-1 therapies have shown clear clinical benefits in a subset of patients with various tumor types, there are still unmet medical needs to develop novel immunotherapies to achieve robust and durable clinical anti-tumor efficacy. Preclinical data strongly suggests there is great potential of developing DGKα and DGKζ targeted therapies to improve antitumor immunity.

### SUMMARY OF THE DISCLOSURE

The above needs have been met by providing the compounds disclosed herein which have a novel core structure and show the desired inhibition of DGKα and DGKζ. In some embodiments, the compounds disclosed herein show the selective inhibitory activity of DGKα over DGKζ. In some embodiments, the compounds disclosed herein show the selective inhibitory activity of DGKζ over DGKα. In some embodiments, the compounds disclosed herein show the dual inhibitory activity of both DGKα and DGKζ. In some embodiments, the compounds disclosed herein show the closely inhibitory activity of DGKα and DGKζ.

Disclosed herein provides a compound of formula (I),

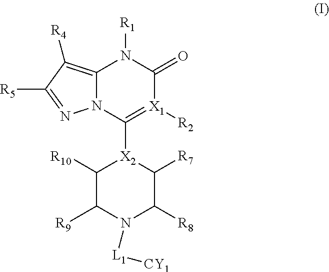

or a stereoisomer or a pharmaceutically acceptable salt thereof
wherein
X₁ is C or N;
X₂ is selected from —CH— or N;
R₁ is hydrogen, or alkyl optionally substituted with deuterium or halogen;
R₂ is hydrogen, halogen, alkyl or cyano provided R₂ is absent when X₁ is N;
R₄ is hydrogen, halogen, or alkyl, wherein the alkyl is optionally substituted with deuterium or halogen;
R₅ is hydrogen, alkyl, alkenyl, alkynyl, cycloalkyl, cyano, or heterocyclyl, wherein said alkyl or alkenyl is unsubstituted or substituted with halogen, cyano, heterocyclyl, alkoxy, hydroxy, cycloalkyl;
each of R₇, R₉, R₇, and R₁₀ is independently hydrogen, alkyl, alkoxy, wherein said alkyl is unsubstituted or substituted with halogen, provided that at least one of R₇ and R₉ is not hydrogen;
L₁ is a direct bond, or —C(R_{L1})(R_{L2})—, wherein said each of R_{L1} and R_{L2} is independently hydrogen or C₁₋₄alkyl optionally substituted with deuterium, halogen, alkyl, alkylene, alkynyl, cyano;
Cy₁ is aryl, heterocyclyl, heteroaryl, or cycloalkyl, each of which is unsubstituted or substituted with one, two or three substituents R₃ₐ, wherein each R₃ₐ is independently selected from hydroxy, alkoxy, alkyl, halogen, aminoalkyl, cycloalkyl, cyano, heterocyclyl or heterocyclyloxy,
wherein
each alkyl moiety of which is unsubstituted or substituted with deuterium, halogen, alkoxy, hydroxy, cyano or heterocyclyl; and
each of said cycloalkyl or heterocyclyl is unsubstituted or substituted with alkoxy, alkyl, halogen, or hydroxy.
   The Definitions of X¹, X²

In some embodiments, X₁ is C; In some embodiments, X₁ is N;

In some embodiments, X₂ is —CH—; In some embodiments, X₂ is N;

### The Definitions of R¹

In some embodiments, R₁ is hydrogen, or C₁₋₄alkyl optionally substituted with deuterium, halogen, hydroxy, alkoxy or cycloalkyl; In some embodiments, R₁ is hydrogen, or C₁₋₃alkyl.

In some embodiments, R₁ is hydrogen, methyl, methyl-d3, ethyl, isopropyl, n-propyl; In some embodiments, R₁ is hydrogen, methyl, or ethyl; In some embodiments, R₁ is hydrogen or methyl.

### The Definitions of R²

In some embodiments, R₂ is hydrogen, halogen, C₁₋₄alkyl or cyano; In some embodiments, R₂ is hydrogen, F, Br, Cl, CN, cyanomethyl, methyl, ethyl; In some embodiments, R₂ is hydrogen, F, Br, CN, methyl; In some embodiments, R₂ is hydrogen, F, Br or CN.

In some embodiments, R₂ is absent when X₁ is N.

### The Definitions of R⁴

In some embodiments, R₄ is hydrogen, halogen or alkyl optionally substituted with deuterium; In some embodiments, R₄ is hydrogen, methyl or methyl-d3.

### The Definitions of R⁵

In some embodiments, R₅ is hydrogen, alkyl, alkenyl, alkynyl or cyano, wherein said alkyl is unsubstituted or substituted with cyano, cycloalkyl or heterocyclyl containing one oxygen atom; in some embodiments, R₅ is C₁₋₆alkyl, C₂₋₆alkenyl or C₂₋₆alkynyl, wherein said alkyl is substituted with cyano, C₃₋₆cycloalkyl or heterocyclyl containing one oxygen atom.

In some embodiments, R₅ is hydrogen, —CN, —CH₂—CN, —CH(CH₃)CN, —CH₂—CH₂—CN, —CH₂—CH₂—CH₂—CN, —CH(CH₃)—CH₂—CN, —CH₂—CH(CH₃)—CN, —CH(CH₂CH₃)—CN, oxiran-2-ylmethyl, oxiran-2-yl, oxetane-3-ylmethyl, oxetane-2-methyl, oxetane-3-yl, oxetane-2-yl, prop-2-yn-1-yl, but-2-yn-1-yl, but-3-yn-1-yl, pent-2-yn-1-yl, pent-3-yn-1-yl, pent-4-yn-1-yl, prop-2-en-1-yl, but-2-en-1-yl, but-3-en-1-yl, pent-2-en-1-yl, pent-3-en-1-yl, pent-4-en-1-yl, methyl, ethyl, propyl, isopropyl, cyclopropyl, cyclobutyl, azetidine-2-yl, azetidine-3-yl, azetidine-3-ylmethyl, azetidine-1-yl, azetidine-1-ylmethyl, aziridine-1-yl, aziridine-1-ylmethyl, aziridine-2-yl, aziridine-2-ylmethyl, 1-cyanocyclopropyl, 2-cyanocyclopropyl or 2-cyanocyclobutyl; In some embodiments, R₅ is hydrogen, —CN, —CH₂—CN, —CH(CH₃)CN, —CH₂—CH₂—CN, CH₂—CH₂—CH₂—CN, —CH(CH₃)—CH₂—CN, —CH₂—CH(CH₃)—CN, —CH(CH₂CH₃)—CN, prop-2-yn-1-yl, but-3-yn-1-yl or pent-3-yn-1-yl; In some embodiments, R₅ is —CN, —CH₂—CN, —CH₂—CH₂—CN.

The Definitions of R⁷/R⁹, R⁸/R¹⁰

In some embodiments, each of R₇ and R₉ is independently hydrogen, or alkyl, wherein said alkyl is unsubstituted or substituted with halogen, alkoxy, amino, cycloalkyl, heterocyclyl; preferably each of R₇ and R₉ is independently C₁₋₄alkyl unsubstituted or substituted with halogen, or alkoxy; more preferably each of R₇ and R₉ is independently C₁₋₂alkyl unsubstituted or substituted with halogen, or alkoxy.

In some embodiments, R₇ and R₉ are each independently hydrogen, methyl, ethyl, isopropyl, n-propyl, methoxymethyl, 2-methoxyethyl, provided that at least one of R₇ and R₉ is not hydrogen.

In some embodiments, R₇ is methyl, and R₉ is methyl; In some embodiments, R₇ is hydrogen, and R₉ is methyl; In some embodiments, R₇ is methyl, and R₉ is hydrogen; In some embodiments, R₇ is hydrogen, and R₉ is ethyl; In some embodiments, R₇ is ethyl, and R₉ is hydrogen; In some embodiments, R₇ is ethyl, and R₉ is ethyl; In some embodiments, R₇ is methyl, and R₉ is ethyl.

In some embodiments, R₈ and R₁₀ are each hydrogen.

In some embodiments, the 5-position carbon on the piperazine ring is R-configuration, provided that R₉ is not hydrogen.

In some embodiments, the 2-position carbon on the piperazine ring is a chiral carbon; and the 5-position carbon on the piperazine ring is R-configuration, provided that R₇ is hydrogen and R₉ is not hydrogen.

In some embodiments, the 2-position carbon on the piperazine ring is S-configuration, and the 5-position carbon on the piperazine ring is R-configuration, provided that R₇ and R₉ are not both hydrogen.

In some embodiments, the 2-position carbon and the 5-position carbon on the piperazine ring are both R-configuration, provided that R₇ is methoxymethyl and R₉ is not hydrogen.

In some embodiments, the 5-position carbon on the piperazine ring is S-configuration, provided that R₉ is not hydrogen; In some embodiments, the 5-position carbon on the piperazine ring is S-configuration, provided that R₉ is methoxymethyl.

In some embodiments, the 2-position carbon on the piperazine ring is achiral carbon; and the 5-position carbon on the piperazine ring is S-configuration, provided that R₇ is hydrogen and R₉ is not hydrogen.

In some embodiments, the 2-position carbon on the piperazine ring is S-configuration, provided that R₇ is methoxymethyl.

### The Definitions of L¹

In some embodiments, L₁ is a direct bond, —C(R_{L1})(R_{L2})—, wherein said each of R_{L1}, and R_{L2} is independently hydrogen or C₁₋₄alkyl optionally substituted with halogen, deuterium, alkyl, alkylene, alkynyl or cyano.

In some embodiments, L₁ is a direct bond, —CH₂—, —CH(CH₃)—, —CH(CD₃)-, —CH(CH₂CH₃)—, —CH(C₃H₇)—, —CH(CHF₂)—, or —C(CH₃)₂—; more preferably L₁ is —CH₂—, —CH(CH₃)—, or —CH(CD₃)-.

Disclosed herein provides a compound of formula (II),

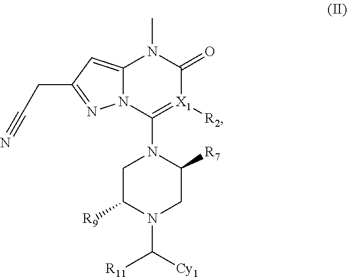

or a stereoisomer or a pharmaceutically acceptable salt thereof,

wherein R₁₁ is methyl or ethyl, and the definitions of X₁, R₂, R₇, R₉ and Cy₁ are described as above.

In some embodiments, R₁₁ is ethyl; R₇ is ethyl; and R₉ is ethyl.

In some embodiments, R₁₁ is ethyl; R₇ is methyl; and R₉ is methyl.

In some embodiments, R₁₁ is ethyl; R₇ is methyl; and R₉ is ethyl.

In some embodiments, R₁₁ is ethyl; R₇ is ethyl; and R₉ is methyl.

In some embodiments, R₁₁ is methyl; R₇ is ethyl; and R₉ is ethyl.

In some embodiments, R₁₁ is methyl; R₇ is methyl; and R₉ is ethyl.

In some embodiments, R₁₁ is methyl; R₇ is ethyl; and R₉ is methyl.

In some embodiments, R₁₁ is methyl; R₇ is methyl; and R₉ is methyl.

In some embodiments, the carbon attached to R₁₁ and Cy₁ is R-configuration.

In some embodiments, the carbon attached to R₁₁ and Cy₁ is S-configuration.

Disclosed herein provides a compound of formula (IIa),

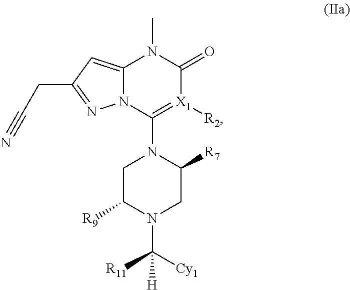

or a stereoisomer or a pharmaceutically acceptable salt thereof,

wherein R₁₁ is methyl or ethyl, and the definitions of X₁, R₂, R₇, R₉ and Cy₁ are described as above.

Disclosed herein provides a compound of formula (IIb),

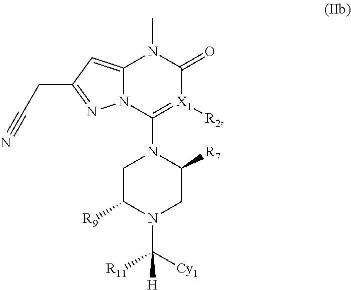

or a stereoisomer or a pharmaceutically acceptable salt thereof,
wherein R₁₁ is methyl or ethyl, and the definitions of X₁, R₂, R₇, R₉ and Cy₁ are described as above.

### The Definitions of Cy¹

In some embodiments, Cy¹ is aryl, heterocyclyl, heteroaryl, or cycloalkyl, each of which is unsubstituted or substituted with one, two or three substituents R₃ₐ, wherein each R₃ₐ is independently selected from hydroxy, alkoxy, alkyl, halogen, aminoalkyl, cycloalkyl, cycloalkyl, heterocyclyl or heterocyclyloxy, wherein each alkyl moiety of which is unsubstituted or substituted with halogen, alkoxy, hydroxy or heterocyclyl, and each of said cycloalkyl or heterocyclyl is unsubstituted or substituted with alkoxy, alkyl, halogen, or hydroxy.

In some embodiments, R₃ₐ is selected from F, Br, Cl, methyl, ethyl, isopropyl, trifluoromethyl, methoxy, ethoxy, isopropoxy, cyclopropyl, cyclobutane, difluoromethyl, 2-fluoro-2-methylethyl, oxetan-3-ylmethyloxy, difluoromethoxy, 2-methoxyethoxy, (2-methoxyethoxy)methyl, isopropoxy, cyclopropoxy.

In some embodiments, Cy₁ is heterocyclyl optionally substituted with one, two or three substituents R₃ₐ, wherein each R₃ₐ is independently selected from hydroxy, alkoxy, alkyl, halogen, aminoalkyl, cycloalkyl, cycloalkyl, heterocyclyl or heterocyclyloxy, wherein each alkyl moiety of which is unsubstituted or substituted with halogen, alkoxy, hydroxy or heterocyclyl; and each of said cycloalkyl or heterocyclyl is unsubstituted or substituted with alkoxy, alkyl, halogen, or hydroxy; In some embodiments, R₃ₐ is F, Br, Cl, methyl, ethyl, isopropyl, trifluoromethyl, methoxy, ethoxy, isopropoxy, cyclopropyl, cyclobutane, difluoromethyl, 2-fluoro-2-methylethyl, oxetan-3-ylmethyloxy, difluoromethoxy, 2-methoxyethoxy, (2-methoxyethoxy)methyl, isopropoxy, cyclopropoxy.

In some embodiments, Cy₁ is phenyl optionally substituted with one, two or three substituents R₃ₐ

In some embodiments, Cy₁ is phenyl, which is substituted with one R₃ₐ as disclosed herein at position 4 and optionally substituted with R₃ₐ on the other positions. In some embodiments, wherein Cy₁ is a monocyclic 5- to 9-membered heterocyclyl or heteroaryl, or a bicyclic 7- to 10-membered heterocyclyl or heteroaryl, each of which is unsubstituted or substituted with one, two or three R₃ₐ.

In some embodiments, wherein Cy₁ is a monocyclic 5- to 9-membered heterocyclyl or a bicyclic 7- to 10-membered heterocyclyl, each of which is unsubstituted or substituted with one, two or three R₃ₐ.

In some embodiments, wherein Cy₁ is a monocyclic 5- to 9-membered heteroaryl or a bicyclic 7- to 10-membered heteroaryl, each of which is unsubstituted or substituted with one, two or three R₃ₐ.

In some embodiments, monocyclic 5- to 9-membered heterocyclyl or heteroaryl is

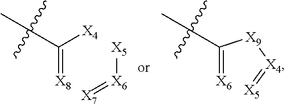

each of which is unsubstituted or substituted with one, two or three R₃ₐ, wherein each of X₄, X₅, X₆, X₇ and X₈ is independently selected from N or C, and X₉ is selected from C, N, S or O.

In some embodiments, monocyclic 5- to 9-membered heteroaryl is thiazole, isothiazole, triazole, pyridine, pyrazine, pyrimidine, each of which is unsubstituted or substituted with one, two or three R₃ₐ.

In some embodiments, monocyclic 5- to 9-membered heteroaryl is pyridin-2-yl, pyridin-3-yl, pyridin-4-yl, thiazole-2-yl, thiazole-4-yl, isothiazole-3-yl, isothiazole-4-yl, pyrazine-1-yl, pyrazine-2-yl, pyrimidin-2-yl, pyrimidin-4-yl, pyrimidin-5-yl, each of which is unsubstituted or substituted with one, two or three R₃ₐ.

In some embodiments, Cy1 is

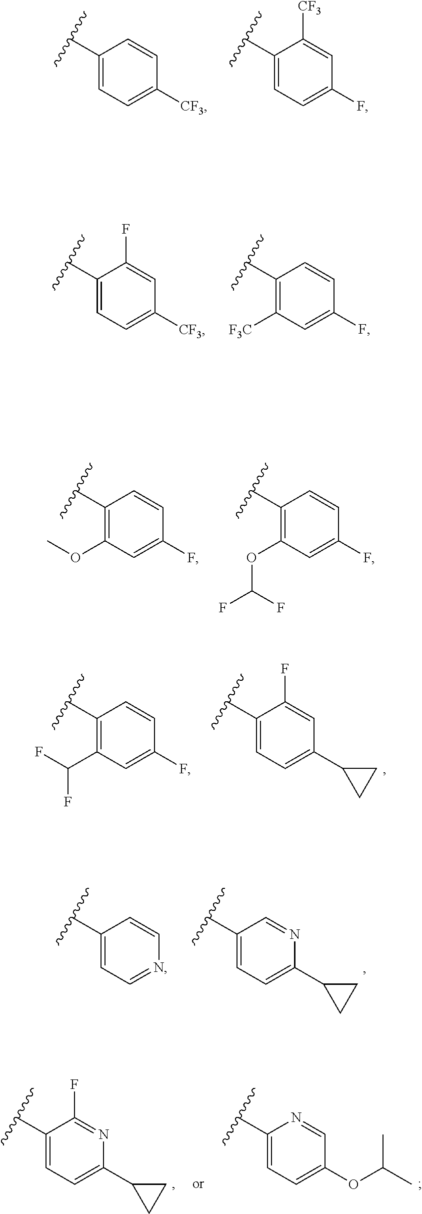

In some embodiments, Cy1 is

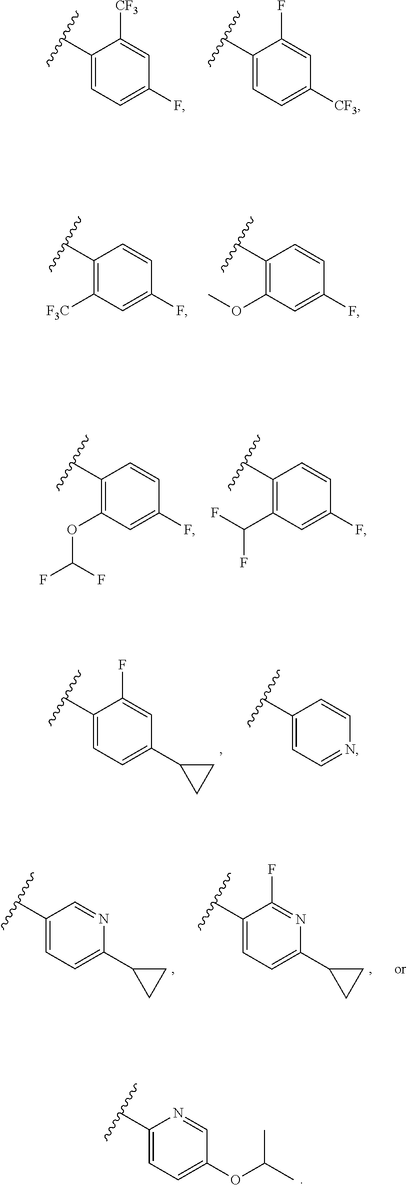

In some embodiments, bicyclic 7- to 10-membered heterocyclyl or heteroaryl is

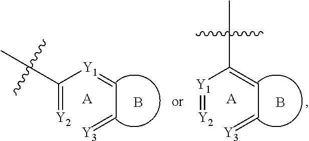

each of which is unsubstituted or substituted with one, two or three R₃ₐ, wherein ring A is a six-membered carbocycle or heterocycle; and ring B is selected from a 5- or 6-membered monocyclic carbocycle or monocyclic heterocycle fused to ring A to form an A-B bicyclic ring; and each of Y₁, Y₂, Y₃ is independently N or C.

In some embodiments, ring B is

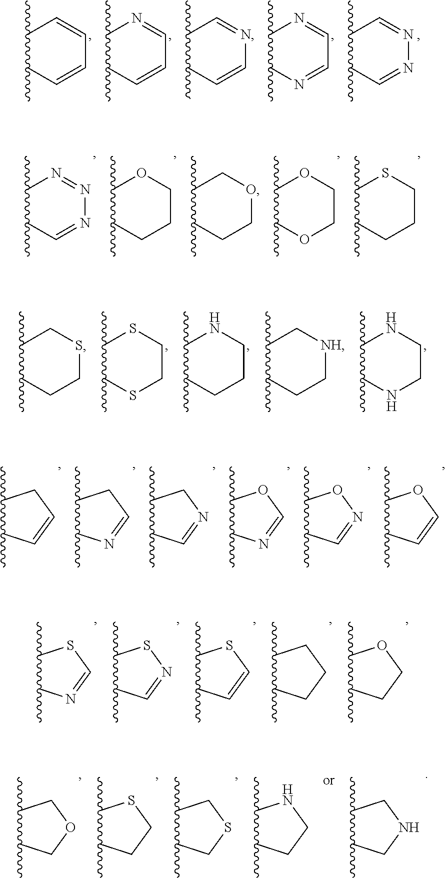

In some embodiments, the bicyclic 7- to 10-membered heterocyclyl or heteroaryl is

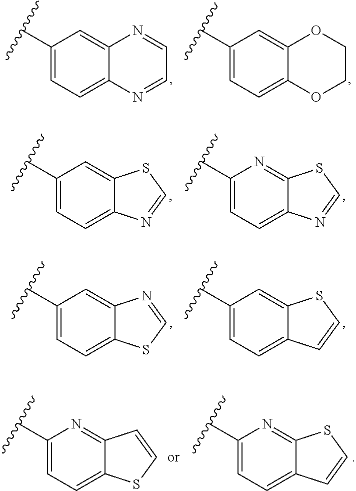

In some embodiments, the bicyclic 7- to 10-membered heterocyclyl or heteroaryl is

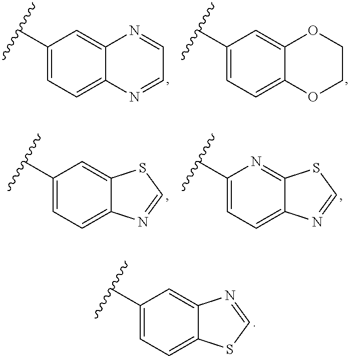

In some embodiments, the bicyclic 7- to 10-membered heterocyclyl or heteroaryl is

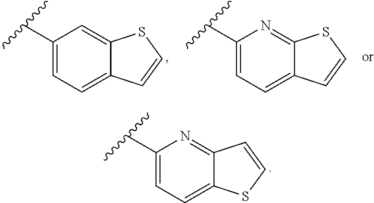 [SMILES: *c1ccc2ccsc2c1 ; *c1ccc2ccsc2n1 ; *c1ccc2sccc2n1]

In some embodiments, the bicyclic 7- to 10-membered heterocyclyl or heteroaryl is

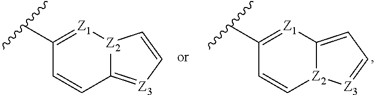

wherein each of Z₁, Z₂ and Z₃ is N or CH, provided at least two of Z₁, Z₂ and Z₃ are N; In some embodiments, the bicyclic 7- to 10-membered heterocyclyl or heteroaryl is

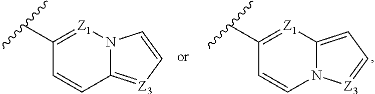

wherein each of Z₁ and Z₃ is N or CH, provided at least one of Z₁ and Z₂ is N; In some embodiments,

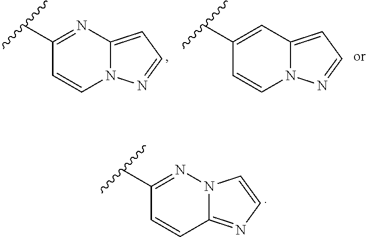 [SMILES: *c1ccn2nccc2n1 ; *c1ccn2nccc2c1 ; *c1ccc2nccn2n1]

In some embodiments, the bicyclic 7- to 10-membered heterocyclyl or heteroaryl is 6,7-dihydro-5H-cyclopenta[b]pyridine, 6,7-dihydro-5H-cyclopenta[c]pyridine, chromane, isochromane, 2,3-dihydrobenzo[b][1,4]dioxine, thiochromane, isothiochromane, 2,3-dihydrobenzo[b][1,4]dithiine, quinoxalinyl, isoquinoline, quinoxaline, 2,3-dihydro-[1,4]dioxino[2,3-b]pyridine, 2,3-dihydro-[1,4]dioxino[2,3-c]pyridine, 3,4-dihydro-2H-pyrano[3,2-b]pyridine, 3,4-dihydro-2H-thiopyrano[2,3-b]pyridine, 2,3-dihydro-[1,4]dithiino[2,3-b]pyridine, 2,3-dihydro-[1,4]dithiino[2,3-c]pyridine, 1,2,3,4-tetrahydroquinoline, 1,2,3,4-tetrahydroisoquinoline, 5,6,7,8-tetrahydro-1,7-naphthyridine, 1,2,3,4-tetrahydro-2,7-naphthyridine, 1,2,3,4-tetrahydro-1,7-naphthyridine, 3H-indole, 1H-isoindole, benzofurane, benzo[b]thiophene, 3H-pyrrolo[3,2-b]pyridine, 7H-pyrrolo[3,4-b]pyridine, furo[2,3-b]pyridine, thieno[2,3-b]pyridine, benzo[d]thiazole, benzo[d]oxazole, oxazolo[5,4-b]pyridine, thiazolo[5,4-b]pyridine, oxazolo[4,5-b]pyridine, thiazolo[4,5-b]pyridine, 2,3-dihydro-1H-indene, 2,3-dihydrobenzofurane, 1,3-dihydroisobenzofurane, 1,3-dihydrobenzo[c]thiophene, 2,3-dihydrobenzo[b]thiophene, benzo[b]thiophene, thieno[3,2-b]pyridine, limidazo[1,2-b]pyridazine, pyrazolo[1,5-a]pyrimidine, lpyrazolo[1,5-a]pyridine, pyrrolo[1,2-b]pyridazine, imidazo[1,2-a]pyridine, or pyrrolo[1,2-a]pyrimidine, each of which is unsubstituted or substituted with one, two or three R₃ₐ.

In some embodiments, the bicyclic 7- to 10-membered heteroaryl is 6,7-dihydro-5H-cyclopenta[b]pyridine, 6,7-dihydro-5H-cyclopenta[c]pyridine, chromane, isochromane, 2,3-dihydrobenzo[b][1,4]dioxine, thiochromane, isothiochromane, 2,3-dihydrobenzo[b][1,4]dithiine, quinoxalinyl, isoquinoline, quinoxaline, 2,3-dihydro-[1,4]dioxino[2,3-b]pyridine, 2,3-dihydro-[1,4]dioxino[2,3-c]pyridine, 3,4-dihydro-2H-pyrano[3,2-b]pyridine, 3,4-dihydro-2H-thiopyrano[2,3-b]pyridine, 2,3-dihydro-[1,4]dithiino[2,3-b]pyridine, 2,3-dihydro-[1,4]dithiino[2,3-c]pyridine, 1,2,3,4-tetrahydroquinoline, 1,2,3,4-tetrahydroisoquinoline, 5,6,7,8-tetrahydro-1,7-naphthyridine, 1,2,3,4-tetrahydro-2,7-naphthyridine, 1,2,3,4-tetrahydro-1,7-naphthyridine, 3H-indole, 1H-isoindole, benzofurane, benzo[b]thiophene, 3H-pyrrolo[3,2-b]pyridine, 7H-pyrrolo[3,4-b]pyridine, furo[2,3-b]pyridine, thieno[2,3-b]pyridine, benzo[d]thiazole, benzo[d]oxazole, oxazolo[5,4-b]pyridine, thiazolo[5,4-b]pyridine, oxazolo[4,5-b]pyridine, thiazolo[4,5-b]pyridine, 2,3-dihydro-1H-indene, 2,3-dihydrobenzofurane, 1,3-dihydroisobenzofurane, 1,3-dihydrobenzo[c]thiophene or 2,3-dihydrobenzo[b]thiophene, each of which is unsubstituted or substituted with one, two or three R₃a.

In some embodiments, the bicyclic 7- to 10-membered heteroaryl is quinoxaline, 2,3-dihydrobenzo[b][1,4]dioxine, benzo[d]thiazole, thiazolo[5,4-b]pyridine, benzo[b]thiophene, or thieno[3,2-b]pyridine, each of which is unsubstituted or substituted with one, two or three R₃ₐ.

In some embodiments, the bicyclic 7- to 10-membered heteroaryl is benzo[b]thiophene, thieno[3,2-b]pyridine, limidazo[1,2-b]pyridazine, pyrazolo[1,5-a]pyrimidine, lpyrazolo[1,5-a]pyridine, pyrrolo[1,2-b]pyridazine, imidazo[1,2-a]pyridine, or pyrrolo[1,2-a]pyrimidine, each of which is unsubstituted or substituted with one, two or three R₃ₐ.

In some embodiments, the bicyclic 7- to 10-membered heteroaryl is benzo[b]thiophene, thieno[3,2-b]pyridine, limidazo[1,2-b]pyridazine, pyrazolo[1,5-a]pyrimidine, or pyrazolo[1,5-a]pyridine, each of which is unsubstituted or substituted with one, two or three R₃ₐ.

In some embodiments, the bicyclic 7- to 10-membered heterocyclyl or heteroaryl is 6,7-dihydro-5H-cyclopenta[b]pyridine-2-yl, 6,7-dihydro-5H-cyclopenta[b]pyridine-2-yl, 6,7-dihydro-5H-cyclopenta[c]pyridine-3-yl, 6,7-dihydro-5H-cyclopenta[c]pyridine-3-yl, chromane-7-yl, chromane-8-yl, isochromane-7-yl, isochromane-8-yl, 2,3-dihydrobenzo[b][1,4]dioxine-7-yl, 2,3-dihydrobenzo[b][1,4]dioxine-8-yl, thiochromane-7-yl, thiochromane-8-yl, 2,3-dihydrobenzo[b][1,4]dithiine-7-yl, 2,3-dihydrobenzo[b][1,4]dithiine-8-yl, quinoxalinyl-7-yl, quinoxalinyl-8-yl, isoquinoline-7-yl, isoquinoline-8-yl, quinoxaline-7-yl, quinoxaline-8-yl, 2,3-dihydro-[1,4]dioxino[2,3-b]pyridine-6-yl, 2,3-dihydro-[1,4]dioxino[2,3-b]pyridine-7-yl, 2,3-dihydro-[1,4]dioxino[2,3-c]pyridine-5-yl, 2,3-dihydro-[1,4]dioxino[2,3-c]pyridine-7-yl, 3,4-dihydro-2H-pyrano[3,2-b]pyridine-7-yl, 3,4-dihydro-2H-pyrano[3,2-b]pyridine-8-yl, 3,4-dihydro-2H-pyrano[3,2-b]pyridine-6-yl, 3,4-dihydro-2H-pyrano[2,3-b]pyridine-7-yl, 3,4-dihydro-2H-pyrano[2,3-b]pyridine-6-yl, 3,4-dihydro-2H-pyrano[2,3-b]pyridine-5-yl, 3,4-dihydro-2H-thiopyrano[3,2-b]pyridine-7-yl, 3,4-dihydro-2H-thiopyrano[3,2-b]pyridine-8-yl, 3,4-dihydro-2H-thiopyrano [3,2-b]pyridine-6-yl, 3,4-dihydro-2H-thiopyrano [2,3-b]pyridine-7-yl, 3,4-dihydro-2H-thiopyrano [2,3-b]pyridine-6-yl, 3,4-dihydro-2H-thiopyrano [2,3-b]pyridine-5-yl, 2,3-dihydro-[1,4]dithiino[2,3-b]pyridine-6-yl, 2,3-dihydro-[1,4]dithiino[2,3-b]pyridine-7-yl, 2,3-dihydro-[1,4]dithiino[2,3-c]pyridine-5-yl, 2,3-dihydro-[1,4]dithiino[2,3-c]pyridine-7-yl, 1,2,3,4-tetrahydroquinoline-7-yl, 1,2,3,4-tetrahydroquinoline-8-yl, 1,2,3,4-tetrahydroisoquinoline-7-yl, 1,2,3,4-tetrahydroisoquinoline-8-yl, 5,6,7,8-tetrahydro-1,7-naphthyridine-7-yl, 5,6,7,8-tetrahydro-1,7-naphthyridine-6-yl, 5,6,7,8-tetrahydro-1,7-naphthyridine-5-yl, 1,2,3,4-tetrahydro-2,7-naphthyridine-8-yl, 1,2,3,4-tetrahydro-2,7-naphthyridine-6-yl, 1,2,3,4-tetrahydro-2,7-naphthyridine-5-yl, 1,2,3,4-tetrahydro-1,7-naphthyridine-7-yl, 1,2,3,4-tetrahydro-1,7-naphthyridine-8-yl, 3H-indole-4-yl, 3H-indole-5-yl, 3H-indole-6-yl, 3H-indole-7-yl, 1H-isoindole-4-yl, 1H-isoindole-5-yl, 1H-isoindole-6-yl, 1H-isoindole-7-yl, benzofuran-4-yl, benzofuran-5-yl, benzofuran-6-yl, benzofuran-7-yl, benzo[b]thiophene-4-yl, benzo[b]thiophene-5-yl, benzo[b]thiophene-6-yl, benzo[b]thiophene-7-yl, 3H-pyrrolo[3,2-b]pyridine-5-yl, 3H-pyrrolo[3,2-b]pyridine-6-yl, 3H-pyrrolo[3,2-b]pyridine-7-yl, 7H-pyrrolo[3,4-b]pyridine-2-yl, 7H-pyrrolo[3,4-b]pyridine-3-yl, 7H-pyrrolo[3,4-b]pyridine-4-yl, furo[2,3-b]pyridine-4-yl, furo[2,3-b]pyridine-5-yl, furo[2,3-b]pyridine-6-yl, thieno[2,3-b]pyridine-4-yl, thieno[2,3-b]pyridine-5-yl, thieno[2,3-b]pyridine-6-yl, benzo[d]thiazole-4-yl, benzo[d]thiazole-5-yl, benzo[d]thiazole-6-yl, benzo[d]thiazole-7-yl, benzo[d]oxazole-4-yl, benzo[d]oxazole-5-yl, benzo[d]oxazole-6-yl, benzo[d]oxazole-7yl, oxazolo[5,4-b]pyridine-5-yl, oxazolo[5,4-b]pyridine-6-yl, oxazolo[5,4-b]pyridine-7-yl, thiazolo[5,4-b]pyridine-5-yl, thiazolo[5,4-b]pyridine-6-yl, thiazolo[5,4-b]pyridine-7-yl, oxazolo[4,5-b]pyridine-5-yl, oxazolo[4,5-b]pyridine-6-yl, oxazolo[4,5-b]pyridine-7-yl, thiazolo[4,5-b]pyridine-5-yl, thiazolo[4,5-b]pyridine-6-yl, thiazolo[4,5-b]pyridine-7-yl, 2,3-dihydro-1H-indene-4-yl, 2,3-dihydro-1H-indene-5-yl, 2,3-dihydrobenzofuran-4-yl, 2,3-dihydrobenzofuran-5-yl, 2,3-dihydrobenzofuran-6-yl, 2,3-dihydrobenzofuran-7-yl, 1,3-dihydroisobenzofuran-4-yl, 1,3-dihydroisobenzofuran-5-yl, 1,3-dihydrobenzo[c]thiophene-4-yl, 1,3-dihydrobenzo[c]thiophene-5-yl, 2,3-dihydrobenzo[b]thiophene-4-yl, 2,3-dihydrobenzo[b]thiophene-5-yl, 2,3-dihydrobenzo[b]thiophene-6-yl, 2,3-dihydrobenzo[b]thiophene-7-yl, benzo[b]thiophen-6-yl, benzo[b]thiophen-5-yl, benzo[b]thiophen-7-yl, thieno[3,2-b]pyridine-5-yl, thieno[3,2-b]pyridine-6-yl, thieno[3,2-b]pyridine-7-yl, imidazo[1,2-b]pyridazine-6-yl, imidazo[1,2-b]pyridazine-7-yl, imidazo[1,2-b]pyridazine-8-yl, pyrazolo[1,5-a]pyrimidin-5-yl, pyrazolo[1,5-a]pyrimidin-6-yl, pyrazolo[1,5-a]pyrimidin-7-yl, pyrazolo[1,5-a]pyridine-4-yl, pyrazolo[1,5-a]pyridine-5-yl, pyrazolo[1,5-a]pyridine-6-yl, pyrazolo[1,5-a]pyridine-7-yl, pyrrolo[1,2-b]pyridazine-2-yl, pyrrolo[1,2-b]pyridazine-3-yl, pyrrolo[1,2-b]pyridazine-4-yl, imidazo[1,2-a]pyridine-5-yl, imidazo[1,2-a]pyridine-6-yl, imidazo[1,2-a]pyridine-7-yl, imidazo[1,2-a]pyridine-8-yl, pyrrolo[1,2-a]pyrimidin-2-yl, pyrrolo[1,2-a]pyrimidin-3-yl, or pyrrolo[1,2-a]pyrimidin-4-yl, each of which is unsubstituted or substituted with one, two or three R₃ₐ.

In some embodiments, the bicyclic 7- to 10-membered heteroaryl is 6,7-dihydro-5H-cyclopenta[b]pyridine-2-yl, 6,7-dihydro-5H-cyclopenta[b]pyridine-2-yl, 6,7-dihydro-5H-cyclopenta[c]pyridine-3-yl, 6,7-dihydro-5H-cyclopenta[c]pyridine-3-yl, chromane-7-yl, chromane-8-yl, isochromane-7-yl, isochromane-8-yl, 2,3-dihydrobenzo[b][1,4]dioxine-7-yl, 2,3-dihydrobenzo[b][1,4]dioxine-8-yl, thiochromane-7-yl, thiochromane-8-yl, 2,3-dihydrobenzo[b][1,4]dithiine-7-yl, 2,3-dihydrobenzo[b][1,4]dithiine-8-yl, quinoxalinyl-7-yl, quinoxalinyl-8-yl, isoquinoline-7-yl, isoquinoline-8-yl, quinoxaline-7-yl, quinoxaline-8-yl, 2,3-dihydro-[1,4]dioxino[2,3-b]pyridine-6-yl, 2,3-dihydro-[1,4]dioxino[2,3-b]pyridine-7-yl, 2,3-dihydro-[1,4]dioxino[2,3-c]pyridine-5-yl, 2,3-dihydro-[1,4]dioxino[2,3-c]pyridine-7-yl, 3,4-dihydro-2H-pyrano[3,2-b]pyridine-7-yl, 3,4-dihydro-2H-pyrano[3,2-b]pyridine-8-yl, 3,4-dihydro-2H-pyrano[3,2-b]pyridine-6-yl, 3,4-dihydro-2H-pyrano[2,3-b]pyridine-7-yl, 3,4-dihydro-2H-pyrano[2,3-b]pyridine-6-yl, 3,4-dihydro-2H-pyrano[2,3-b]pyridine-5-yl, 3,4-dihydro-2H-thiopyrano[3,2-b]pyridine-7-yl, 3,4-dihydro-2H-thiopyrano[3,2-b]pyridine-8-yl, 3,4-dihydro-2H-thiopyrano [3,2-b]pyridine-6-yl, 3,4-dihydro-2H-thiopyrano [2,3-b]pyridine-7-yl, 3,4-dihydro-2H-thiopyrano [2,3-b]pyridine-6-yl, 3,4-dihydro-2H-thiopyrano [2,3-b]pyridine-5-yl, 2,3-dihydro-[1,4]dithiino[2,3-b]pyridine-6-yl, 2,3-dihydro-[1,4]dithiino[2,3-b]pyridine-7-yl, 2,3-dihydro-[1,4]dithiino[2,3-c]pyridine-5-yl, 2,3-dihydro-[1,4]dithiino[2,3-c]pyridine-7-yl, 1,2,3,4-tetrahydroquinoline-7-yl, 1,2,3,4-tetrahydroquinoline-8-yl, 1,2,3,4-tetrahydroisoquinoline-7-yl, 1,2,3,4-tetrahydroisoquinoline-8-yl, 5,6,7,8-tetrahydro-1,7-naphthyridine-7-yl, 5,6,7,8-tetrahydro-1,7-naphthyridine-6-yl, 5,6,7,8-tetrahydro-1,7-naphthyridine-5-yl, 1,2,3,4-tetrahydro-2,7-naphthyridine-8-yl, 1,2,3,4-tetrahydro-2,7-naphthyridine-6-yl, 1,2,3,4-tetrahydro-2,7-naphthyridine-5-yl, 1,2,3,4-tetrahydro-1,7-naphthyridine-7-yl, 1,2,3,4-tetrahydro-1,7-naphthyridine-8-yl, 3H-indole-4-yl, 3H-indole-5-yl, 3H-indole-6-yl, 3H-indole-7-yl, 1H-isoindole-4-yl, 1H-isoindole-5-yl, 1H-isoindole-6-yl, 1H-isoindole-7-yl, benzofuran-4-yl, benzofuran-5-yl, benzofuran-6-yl, benzofuran-7-yl, benzo[b]thiophene-4-yl, benzo[b]thiophene-5-yl, benzo[b]thiophene-6-yl, benzo[b]thiophene-7-yl, 3H-pyrrolo[3,2-b]pyridine-5-yl, 3H-pyrrolo[3,2-b]pyridine-6-yl, 3H-pyrrolo[3,2-b]pyridine-7-yl, 7H-pyrrolo[3,4-b]pyridine-2-yl, 7H-pyrrolo[3,4-b]pyridine-3-yl, 7H-pyrrolo[3,4-b]pyridine-4-yl, furo[2,3-b]pyridine-4-yl, furo[2,3-b]pyridine-5-yl, furo[2,3-b]pyridine-6-yl, thieno[2,3-b]pyridine-4-yl, thieno[2,3-b]pyridine-5-yl, thieno[2,3-b]pyridine-6-yl, benzo[d]thiazole-4-yl, benzo[d]thiazole-5-yl, benzo[d]thiazole-6-yl, benzo[d]thiazole-7-yl, benzo[d]oxazole-4-yl, benzo[d]oxazole-5-yl, benzo[d]oxazole-6-yl, benzo[d]oxazole-7yl, oxazolo[5,4-b]pyridine-5-yl, oxazolo[5,4-b]pyridine-6-yl, oxazolo[5,4-b]pyridine-7-yl, thiazolo[5,4-b]pyridine-5-yl, thiazolo[5,4-b]pyridine-6-yl, thiazolo[5,4-b]pyridine-7-yl, oxazolo[4,5-b]pyridine-5-yl, oxazolo[4,5-b]pyridine-6-yl, oxazolo[4,5-b]pyridine-7-yl, thiazolo[4,5-b]pyridine-5-yl, thiazolo[4,5-b]pyridine-6-yl, thiazolo[4,5-b]pyridine-7-yl, 2,3-dihydro-1H-indene-4-yl, 2,3-dihydro-1H-indene-5-yl, 2,3-dihydrobenzofuran-4-yl, 2,3-dihydrobenzofuran-5-yl, 2,3-dihydrobenzofuran-6-yl, 2,3-dihydrobenzofuran-7-yl, 1,3-dihydroisobenzofuran-4-yl, 1,3-dihydroisobenzofuran-5-yl, 1,3-dihydrobenzo[c]thiophene-4-yl, 1,3-dihydrobenzo[c]thiophene-5-yl, 2,3-dihydrobenzo[b]thiophene-4-yl, 2,3-dihydrobenzo[b]thiophene-5-yl, 2,3-dihydrobenzo[b]thiophene-6-yl, 2,3-dihydrobenzo[b]thiophene-7-yl, each of which is unsubstituted or substituted with one, two or three R₃ₐ.

In some embodiments, the bicyclic 7- to 10-membered heteroaryl is benzo[b]thiophen-6-yl, benzo[b]thiophen-5-yl, benzo[b]thiophen-7-yl, thieno[3,2-b]pyridine-5-yl, thieno[3,2-b]pyridine-6-yl, thieno[3,2-b]pyridine-7-yl, imidazo[1,2-b]pyridazine-6-yl, imidazo[1,2-b]pyridazine-7-yl, imidazo[1,2-b]pyridazine-8-yl, pyrazolo[1,5-a]pyrimidin-5-yl, pyrazolo[1,5-a]pyrimidin-6-yl, pyrazolo[1,5-a]pyrimidin-7-yl, pyrazolo[1,5-a]pyridine-4-yl, pyrazolo[1,5-a]pyridine-5-yl, pyrazolo[1,5-a]pyridine-6-yl, pyrazolo[1,5-a]pyridine-7-yl, pyrrolo[1,2-b]pyridazine-2-yl, pyrrolo[1,2-b]pyridazine-3-yl, pyrrolo[1,2-b]pyridazine-4-yl, imidazo[1,2-a]pyridine-5-yl, imidazo[1,2-a]pyridine-6-yl, imidazo[1,2-a]pyridine-7-yl, imidazo[1,2-a]pyridine-8-yl, pyrrolo[1,2-a]pyrimidin-2-yl, pyrrolo[1,2-a]pyrimidin-3-yl, or pyrrolo[1,2-a]pyrimidin-4-yl, each of which is unsubstituted or substituted with one, two or three R₃ₐ.

In some embodiments, Cy₁ is quinoxalin-6-yl, 3-methylquinoxalin-6-yl, 3-(difluoromethyl)quinoxalin-6-yl, 3-methoxyquinoxalin-6-yl, 3-chloroquinoxalin-6-yl, 3,3-dimethyl-2,3-dihydro-[1,4]dioxino[2,3-b]pyridin-6-yl, 3,3-dimethyl-2,3-dihydrobenzo[b][1,4]dioxin-6-yl, 2,3-dihydrobenzo[b][1,4]dioxin-6-yl, 4-fluoro-2-(trifluoromethyl)phenyl, 4-fluoro-2-methoxyphenyl, 2-(difluoromethoxy)-4-fluorophenyl, 2-(difluoromethyl)-4-fluorophenyl, 4-cyclopropyl-2-fluorophenyl, 6-cyclopropylpyridin-3-yl, 5-isopropoxypyridin-2-yl, 6-cyclopropyl-2-fluoropyridin-3-yl, benzo[d]thiazol-6-yl, thiazolo[5,4-b]pyridin-5-yl, or 2-methylbenzo[d]thiazol-6-yl, benzo[d]thiazol-5-yl, 2-difluoromethyl-methylthieno[2,3-b]pyridine-6-yl, imidazo[1,2-b]pyridazine-6-yl, 2-methyl-imidazo[1,2-b]pyridazine-6-yl, 2-ethyl-imidazo[1,2-b]pyridazine-6-yl, pyrazolo[1,5-a]pyrimidin-5-yl, 2-methyl-pyrazolo[1,5-a]pyrimidin-5-yl, 2-fluoro-pyrazolo[1,5-a]pyrimidin-5-yl, 2-methyl-3-fluoro-pyrazolo[1,5-a]pyrimidin-5-yl, 3-fluoro-pyrazolo[1,5-a]pyrimidin-5-yl, 2-cyclopropyl-pyrazolo[1,5-a]pyrimidin-5-yl, 2-chloro-pyrazolo[1,5-a]pyrimidin-5-yl, 2,3-difluoro-pyrazolo[1,5-a]pyrimidin-5-yl, 2-chloro-3-fluoro-pyrazolo[1,5-a]pyrimidin-5-yl, pyrazolo[1,5-a]pyridine-5-yl, or 2-methyl-pyrazolo[1,5-a]pyridine-5-yl.

In some embodiments, Cy₁ is

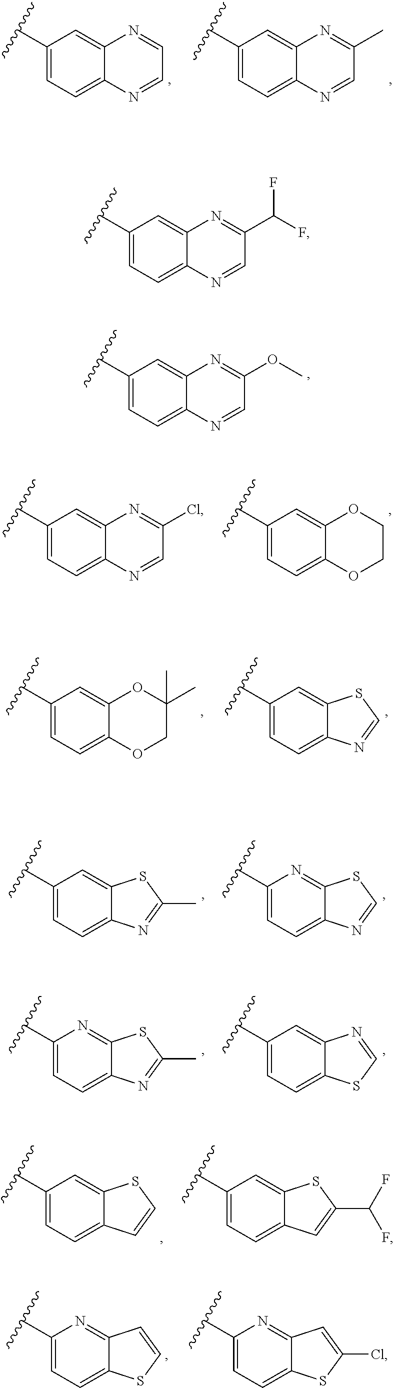

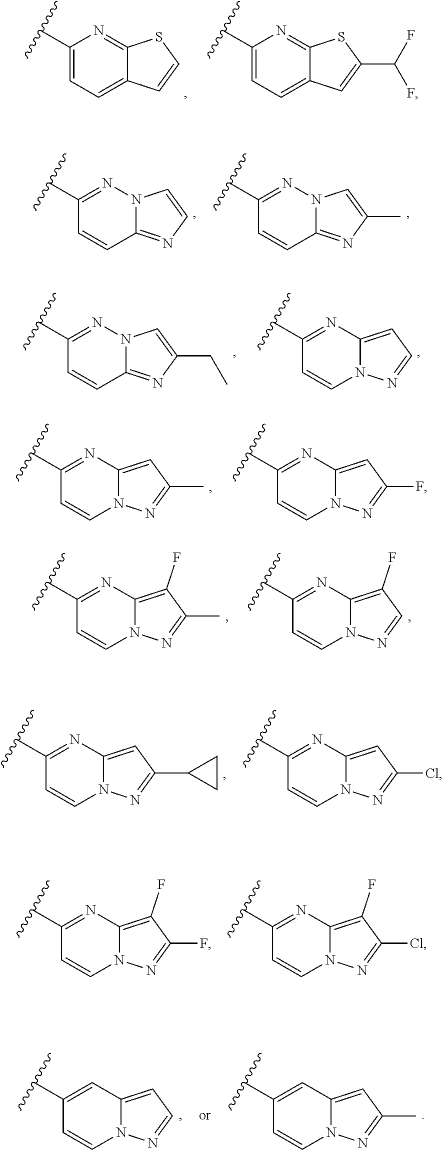

In some embodiments, Cy₁ is

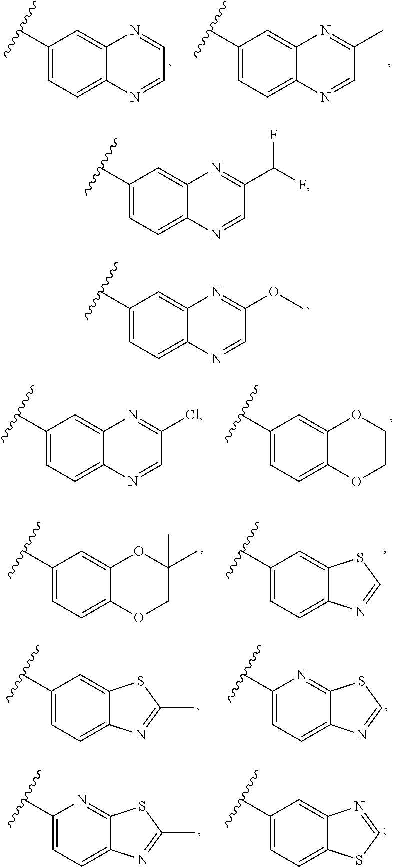

In some embodiments, Cy₁ is

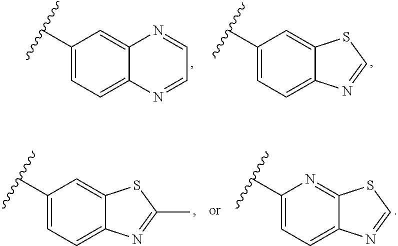

In some embodiments, Cy₁ is

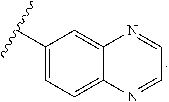 [SMILES: *c1ccc2nccnc2c1]

In some embodiments, Cy₁ is

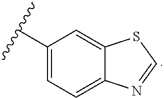 [SMILES: *c1ccc2ncsc2c1]

In some embodiments, Cy₁ is

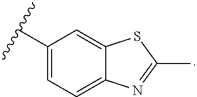 [SMILES: *c1ccc2nc(C)sc2c1]

In some embodiments, Cy₁ is

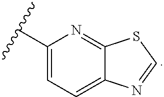 [SMILES: *c1ccc2ncsc2n1]

In some embodiments, Cy₁ is

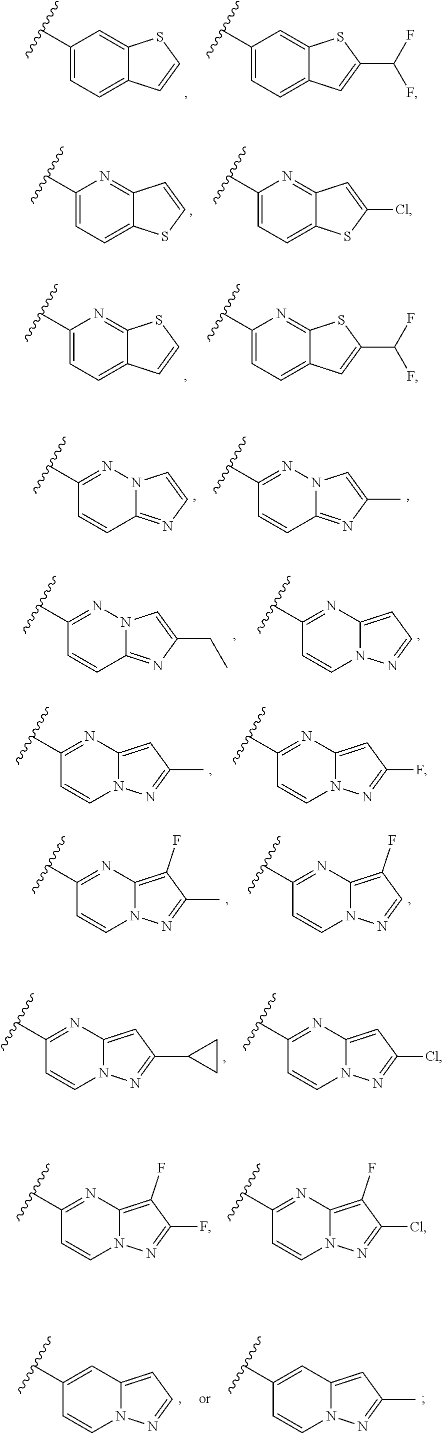

In some embodiments, Cy₁ is

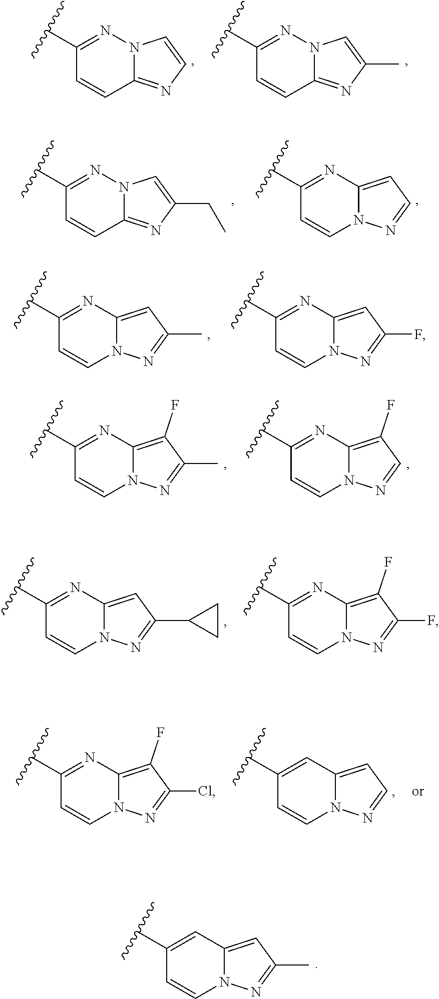

In some embodiments, Cy₁ is

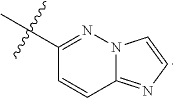 [SMILES: *c1ccc2nccn2n1]

In some embodiments, Cy₁ is

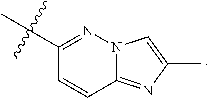 [SMILES: *c1ccc2nc(C)cn2n1]

In some embodiments, Cy₁ is

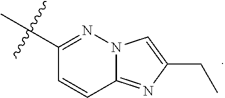 [SMILES: *c1ccc2nc(CC)cn2n1]

In some embodiments, Cy₁ is

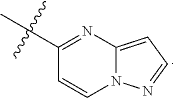 [SMILES: *c1ccn2nccc2n1]

In some embodiments, Cy₁ is

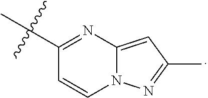 [SMILES: *c1ccn2nc(C)cc2n1]

In some embodiments, Cy₁ is

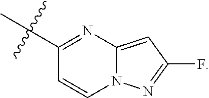 [SMILES: *c1ccn2nc(F)cc2n1]

In some embodiments, Cy₁ is

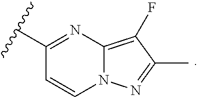 [SMILES: *c1ccn2nc(C)c(F)c2n1]

In some embodiments, Cy₁ is

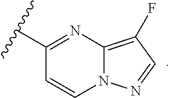 [SMILES: *c1ccn2ncc(F)c2n1]

In some embodiments, Cy₁ is

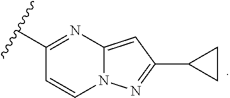 [SMILES: *c1ccn2nc(C3CC3)cc2n1]

In some embodiments, Cy₁ is

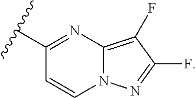 [SMILES: *c1ccn2nc(F)c(F)c2n1]

In some embodiments, Cy₁ is

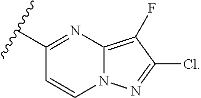 [SMILES: *c1ccn2nc(Cl)c(F)c2n1]

In some embodiments, Cy₁ is

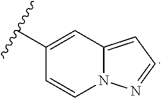 [SMILES: *c1ccn2nccc2c1]

In some embodiments, Cy₁ is

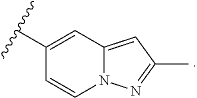 [SMILES: *c1ccn2nc(C)cc2c1]

Disclosed herein provides a compound selected from Table 1.

Disclosed herein provides a compound, or a stereoisomer, or a pharmaceutically acceptable salt thereof, wherein said compound is any one of the exemplified compounds.

Disclosed herein provides a pharmaceutical composition comprising one or more compounds herein, or a stereoisomer or a pharmaceutically acceptable salt thereof, and a pharmaceutically acceptable excipient.

Disclosed herein provides a method of treating cancer, comprising administering to a patient in need of such treatment a therapeutically effective amount of a compound herein or a pharmaceutical composition herein.

### DETAILED DESCRIPTION OF THE DISCLOSURE

### Definitions

The following terms have the indicated meanings throughout the specification:

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “alkyl” refers to a hydrocarbon group selected from linear and branched saturated hydrocarbon groups derived from an alkane by removal of one hydrogen atom from the same carbon atom, which comprises from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups. The alkyl group can be optionally enriched in deuterium, e.g., —CD₃, —CD₂CD₃ and the like. The term “alkylene” refers to a hydrocarbon group selected from linear and branched saturated hydrocarbon groups derived from an alkane by removal of two hydrogen atoms from the same carbon atom, which comprises from 1 to 6, such as from 1 to 4, carbon atoms, further such as from 1 to 3, more further such as 1, 2 or 3 carbon atoms, include, but not limited to, methylene (—CH₂—), ethylene (—CH₂CH₂—), 1-methylmethylene (—CH(CH₃)—), or trimethylene (—CH₂CH₂CH₂—).

The term “halogen” refers to fluoro (F), chloro (Cl), bromo (Br) and iodo (I).

The term “haloalkyl” refers to an alkyl group in which one or more hydrogen is/are replaced by one or more halogen atoms such as fluoro, chloro, bromo, and iodo. Examples of the haloalkyl include haloC₁₋₈alkyl, haloC₁₋₆alkyl or halo C₁₋₄alkyl, but not limited to —CF₃, —CH₂Cl, —CH₂CF₃, —CCl₂, CF₃, and the like.

The term “alkyloxy” or “alkoxy” refers to an alkyl group as defined above attached to the parent molecular moiety through an oxygen atom. Examples of an alkyloxy, e.g., C₁₋₆alkyloxy or C₁₋₄ alkyloxy include, but not limited to, methoxy, ethoxy, isopropoxy, propoxy, n-butoxy, tert-butoxy, pentoxy and hexoxy and the like.

The term “amino” refers to —NH₂.

The term “alkenyl” herein refers to a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C2-6 alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkynyl” herein refers to a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C2-6 alkynyl, include, but not limited to ethynyl, 1-propynyl, 2-propynyl (propargyl), 1-butynyl, 2-butynyl, and 3-butynyl groups.

The term “cycloalkyl” refers to a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. In particular, Examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embedment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “deuterated” is used herein to modify a chemical structure or an organic group or radical, wherein one or more carbon-bound hydrogen(s) are replaced by one or more deuterium(s), e.g., “deuterated-alkyl”, “deuterated-cycloalkyl”, “deuterated-heterocycloalkyl”, “deuterated-aryl”, “deuterated-morpholinyl”, and the like. For example, the term “deuterated-alkyl” defined above refers to an alkyl group as defined herein, wherein at least one hydrogen atom bound to carbon is replaced by a deuterium. In a deuterated alkyl group, at least one carbon atom is bound to a deuterium; and it is possible for a carbon atom to be bound to more than one deuterium; it is also possible that more than one carbon atom in the alkyl group is bound to a deuterium.

The term “aryl” used alone or in combination with other terms refers to a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

The term “heteroaryl” herein refers to a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from nitrogen, oxygen or optionally oxidized sulfur as ring member(s), with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from nitrogen, oxygen or optionally oxidized sulfur as ring member(s), with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

The term “optionally oxidized sulfur” used herein refers to —S—, SO or SO₂.

The terms “aromatic heterocyclic ring” and “heteroaryl” are used interchangeably throughout the disclosure herein. In some embodiments, a monocyclic or bicyclic aromatic heterocyclic ring has 5-, 6-, 7-, 8-, 9- or 10-ring forming members with 1, 2, 3, or 4 heteroatom ring members independently selected from nitrogen (N), sulfur (S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur (S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur (S) and oxygen (O). In some embodiments, the monocyclic or bicyclic aromatic heterocyclic ring is a 8- to 10-membered heteroaryl ring, which is bicyclic and which has 1 or 2 heteroatom ring members independently selected from nitrogen, sulfur and oxygen.

Examples of the heteroaryl group or the monocyclic or bicyclic aromatic heterocyclic ring include, but are not limited to, (as numbered from the linkage position assigned priority 1) 1H-pyrazolyl (such as 1H-pyrazol-3-yl, 1H-pyrazol-4-yl or 1H-pyrazol-5-yl), pyridyl or pyridinyl(such as 2-pyridyl, 3-pyridyl, or 4-pyridyl), cinnolinyl, pyrazinyl, pyrimidinyl (such as pyrimidin-2-yl, pyrimidin-4-yl, pyrimidin-5-yl or 2,4-pyrimidinyl, 3,5-pyrimidinyl), imidazolyl (such as 1H-imidazol-2-yl, 1H-imidazol-4-yl, 1H-imidazol-5-yl, or 2,4-imidazolyl), imidazopyridinyl, isoxazolyl, oxazolyl, thiazolyl, isothiazolyl, thiadiazolyl (such as 1,2,3-thiadiazolyl, 1,2,4-thiadiazolyl, or 1,3,4-thiadiazolyl), tetrazolyl, thienyl (such as thien-2-yl, thien-3-yl), triazinyl, benzothienyl, furyl or furanyl, benzofuryl, benzoimidazolyl, indolyl (such as 1H-indol-2-yl, 1H-indol-3-yl, 1H-indol-4-yl, 1H-indol-5-yl, 1H-indol-6-yl or 1H-indol-7-yl), isoindolyl, indolinyl, oxadiazolyl (such as 1,2,3-oxadiazolyl, 1,2,4-oxadiazolyl, or 1,3,4-oxadiazolyl), phthalazinyl, pyrazinyl (such as pyrazin-2-yl), pyridazinyl, pyrrolyl, triazolyl (such as 1,2,3-triazolyl, 1,2,4-triazolyl, or 1,3,4-triazolyl), quinolinyl (such as quinolin-2-yl, quinolin-3-yl, quinolin-4-yl, quinolin-5-yl, quinolin-6-yl, or quinolin-7-yl), isoquinolinyl (such as isoquinolin-1-yl, isoquinolin-3-yl, isoquinolin-4-yl, isoquinolin-5-yl, isoquinolin-6-yl, isoquinolin-7-yl, or isoquinolin-8-yl), pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl (such as furazan-2-yl, furazan-3-yl), benzofurazanyl, benzothiophenyl, benzothiazolyl, benzoxazolyl (such as benzo[d]oxazol-2-yl, benzo[d]oxazol-4-yl, benzo[d]oxazol-5-yl, benzo[d]oxazol-6-yl or benzo[d]oxazol-7-yl), quinazolinyl, quinoxalinyl (such as quinoxalin-2-yl, quinoxalin-3-yl, quinoxalin-4-yl, quinoxalin-5-yl, quinoxalin-6-yl, quinoxalin-7-yl or quinoxalin-8-yl), naphthyridinyl (such as 1,8-naphthyridin-2-yl, 1,8-naphthyridin-3-yl, or 1,8-naphthyridin-4-yl), 2,3-dihydro-[1,4]dioxino[2,3-b]pyridinyl (such as 2,3-dihydro-[1,4]dioxino[2,3-b]pyridin-6-yl, 2,3-dihydro-[1,4]dioxino[2,3-b]pyridin-7-yl, or 2,3-dihydro-[1,4]dioxino[2,3-b]pyridin-8-yl), furopyridinyl, benzothiazolyl (such as benzo[d]thiazol-2-yl, benzo[d]thiazol-4-yl, benzo[d]thiazol-5-yl, benzo[d]thiazol-6-yl or benzo[d]thiazol-7-yl), benzo[d]imidazolyl (such as 1H-benzo[d]imidazol-2-yl, 1H-benzo[d]imidazol-4-yl, 1H-benzo[d]imidazol-5-yl, 1H-benzo[d]imidazol-6-yl or 1H-benzo[d]imidazol-7-yl), [1,2,4]triazolo[1,5-a]pyridinyl (such as [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl, or [1,2,4]triazolo[1,5-a]pyridin-8-yl), 3H-imidazo[4,5-b]pyridinyl (such as 3H-imidazo[4,5-b]pyridin-2-yl, 3H-imidazo[4,5-b]pyridin-5-yl, 3H-imidazo[4,5-b]pyridin-6-yl or 3H-imidazo[4,5-b]pyridin-7-yl), 1H-imidazo[4,5-b]pyridinyl (such as 1H-imidazo[4,5-b]pyridin-2-yl, 1H-imidazo[4,5-b]pyridin-5-yl, 1H-imidazo[4,5-b]pyridin-6-yl, 1H-imidazo[4,5-b]pyridin-7-yl), [1,2,4]triazolo[1,5-a]pyridinyl (such as [1,2,4]triazolo[1,5-a]pyridin-2-yl, 1,2,4]triazolo[1,5-a]pyridin-5-yl, [1,2,4]triazolo[1,5-a]pyridin-6-yl, [1,2,4]triazolo[1,5-a]pyridin-7-yl or [1,2,4]triazolo[1,5-a]pyridin-8-yl), indazolyl (such as 1H-indazol-5-yl) and 5,6,7,8-tetrahydroisoquinoline.

Also, a “heteroaryl” fused with a “Heterocyclyl” is defined as “heteroaryl”.

“Heterocyclyl,” “heterocycle” or “heterocyclic” are interchangeable and refer to a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “monocyclic heterocyclyl” refers to monocyclic groups in which at least one ring member is a heteroatom selected from nitrogen, oxygen or optionally oxidized sulfur. A heterocycle may be saturated or partially saturated.

Exemplary monocyclic 4 to 9-membered heterocyclyl groups include, but not limited to, (as numbered from the linkage position assigned priority 1) pyrrolidin-1-yl, pyrrolidin-2-yl, pyrrolidin-3-yl, imidazolidin-2-yl, imidazolidin-4-yl, pyrazolidin-2-yl, pyrazolidin-3-yl, piperidin-1-yl, piperidin-2-yl, piperidin-3-yl, piperidin-4-yl, 2,5-piperazinyl, pyranyl, morpholinyl, morpholino, morpholin-2-yl, morpholin-3-yl, oxiranyl, aziridin-1-yl, aziridin-2-yl, azocan-1-yl, azocan-2-yl, azocan-3-yl, azocan-4-yl, azocan-5-yl, thiiranyl, azetidin-1-yl, azetidin-2-yl, azetidin-3-yl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepan-1-yl, azepan-2-yl, azepan-3-yl, azepan-4-yl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepanyl, 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinyl, imidazolinyl, pyrimidinonyl, or 1,1-dioxo-thiomorpholinyl.

The term “spiro heterocyclyl” refers to a 5 to 20-membered polycyclic heterocyclyl with rings connected through one common carbon atom (called a spiro atom), comprising one or more heteroatoms selected from nitrogen, oxygen or optionally oxidized sulfur as ring members, with the remaining ring members being carbon. One or more rings of a spiro heterocyclyl group may contain one or more double bonds, but none of the rings has a completely conjugated pi-electron system. Preferably a spiro heterocyclyl is 6 to 14-membered, and more preferably 7 to 12-membered. According to the number of common spiro atoms, a spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, and more preferably 4-membered/4-membered, 3-membered/5-membered, 4-membered/5-membered, 4-membered/6-membered, 5-membered/5-membered, or 5-membered/6-membered mono-spiro heterocyclyl.

The term “fused heterocyclic group” refers to a 5 to 20-membered polycyclic heterocyclyl group, wherein each ring in the system shares an adjacent pair of atoms (carbon and carbon atoms or carbon and nitrogen atoms) with another ring, comprising one or more heteroatoms selected from nitrogen, oxygen or optionally oxidized sulfur as ring members, with the remaining ring members being carbon. One or more rings of a fused heterocyclic group may contain one or more double bonds, but none of the rings has a completely conjugated pi-electron system. Preferably, a fused heterocyclyl is 6 to 14-membered, and more preferably 7 to 10-membered. According to the number of membered rings, a fused heterocyclyl is divided into bicyclic, tricyclic, tetracyclic, or polycyclic fused heterocyclyl, preferably refers to bicyclic or tricyclic fused heterocyclyl, and more preferably 5-membered/5-membered, or 5-membered/6-membered bicyclic fused heterocyclyl. Representative examples of fused heterocycles include, but not limited to, the following groups octahydrocyclopenta[c]pyrrole (e.g., octahydrocyclopenta[c]pyrrol-2-yl), octahydropyrrolo[3,4-c]pyrrolyl, octahydroisoindolyl, isoindolinyl (e.g., isoindoline-2-yl), octahydro-benzo[b][1,4]dioxin.

The term “bridged heterocyclyl” refers to a 5- to 14-membered polycyclic heterocyclic alkyl group, wherein every two rings in the system share two disconnected atoms, comprising one or more heteroatoms selected from nitrogen, oxygen or optionally oxidized sulfur as ring members, with the remaining ring members being carbon. One or more rings of a bridged heterocyclyl group may contain one or more double bonds, but none of the rings has a completely conjugated pi-electron system. Preferably, a bridged heterocyclyl is 6 to 14-membered, and more preferably 7 to 10-membered. According to the number of membered rings, a bridged heterocyclyl is divided into bicyclic, tricyclic, tetracyclic or polycyclic bridged heterocyclyl, and preferably refers to bicyclic, tricyclic or tetracyclic bridged heterocyclyl, and more preferably bicyclic or tricyclic bridged heterocyclyl. Representative examples of bridged heterocyclyls include, but not limited to, the following groups: 2-azabicyclo[2.2.1]heptyl, azabicyclo[3.1.0]hexyl, 2-azabicyclo[2.2.2]octyl and 2-azabicyclo[3.3.2]decyl.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

The term “substantially pure” as used herein means that the titled stereoisomer contains no more than 35%, such as no more than 30%, further such as no more than 25%, even further such as no more than 20%, by weight of any other stereoisomer(s). In some embodiments, the term “substantially pure” means that the titled stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

When compounds disclosed herein contain a di-substituted cyclohexyl or cyclobutyl group, substituents found on cyclohexyl or cyclobutyl ring may adopt cis and trans formations. Cis formation means that both substituents are found on the upper side of the 2 substituent placements on the carbon, while trans would mean that they were on opposing sides.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or flash column chromatography. Flash column chromatography can involve any number of methods including, for example: reverse-phase and normal phase; size exclusion; ion exchange; high, medium and low pressure liquid flash column chromatography methods and apparatus; small scale analytical; simulated moving bed (“SMB”) and preparative thin or thick layer flash column chromatography, as well as techniques of small scale thin layer and flash column flash column chromatography. One skilled in the art will apply techniques most likely to achieve the desired separation.

“Diastereomers” refers to stereoisomers of a compound with two or more chiral centers but which are not mirror images of one another. Diastereomeric mixtures can be separated into their individual diastereomers on the basis of their physical chemical differences by methods well known to those skilled in the art, such as by flash column chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents [Eliel, E. and Wilen, S. Stereochemistry of Organic Compounds. New York: John Wiley & Sons, Inc., 1994; Lochmuller, C. H., et al. “Flash column chromatographyic resolution of enantiomers: Selective review.” J. Chromatogr., 113(3) (1975): pp. 283-302]. Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993. The absolute configuration of the chiral centers in a compound can be determined using methods known to one skilled in the art, e.g., single crystal X-ray crystallography or co-crystal formation of a compound of interest with the targeted proteins, sometime coupled with a spectroscopic technique, e.g., NMR spectroscopy. In some embodiments, the absolute configuration of chiral centers in a compound can be elucidated from the X-ray single-crystal structure of the compound. In some embodiments, the absolute configuration of chiral centers elucidated by the X-ray crystal structure of a compound can be used to infer the absolute configuration of the corresponding chiral centers in another compound or an intermediate obtained from the same or similar synthetic methodologies.

“Pharmaceutically acceptable salts” refers to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

“Selective inhibitory activity” or “selectivity” refers to the difference in the degree of inhibition against DGKα and DGKζ; the greater the degree of inhibition effected for a particular isoform relative to another isoform, the greater the selectivity the inhibitor exhibits for that particular isoform. In some embodiments, “a compound showing selective inhibitory activity of DGKα over DGKζ” refers a compound which shows an IC50 against DGKα is not larger than about 2000 nM with the ratio of IC50 against DGKζ and IC50 against DGKα larger than or equal to about 20; “a compound showing selective inhibitory activity of DGKζ over DGKα” refers a compound which shows an IC50 against DGKζ is not larger than about 2000 nM with the ratio of IC50 against DGKα and IC50 against DGKζ larger than or equal to about 20; and “a compound showing dual inhibitory activity” refers to a compound which shows inhibitory activities against both DGKα and DGKζ with IC50 no larger than 500 nM and the ratio of the two IC50 values no more than 20.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” include salts of at least one compound of Formula (I), and salts of the stereoisomers of the compound of Formula (I), such as salts of enantiomers, and/or salts of diastereomers.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined above, a disease or disorder in a subject. In the case of combination therapy, the “therapeutically effective amount” refers to the total amount of the combination objects for the effective treatment of a disease, a disorder or a condition.

The pharmaceutical composition comprising the compound disclosed herein can be administrated via oral, inhalation, rectal, parenteral or topical administration to a subject in need thereof. For oral administration, the pharmaceutical composition may be a regular solid Formulation such as tablets, powder, granule, capsules and the like, a liquid Formulation such as water or oil suspension or other liquid Formulation such as syrup, solution, suspension or the like; for parenteral administration, the pharmaceutical composition may be solution, water solution, oil suspension concentrate, lyophilized powder or the like. Preferably, the Formulation of the pharmaceutical composition is selected from tablet, coated tablet, capsule, suppository, nasal spray or injection, more preferably tablet or capsule. The pharmaceutical composition can be a single unit administration with an accurate dosage. In addition, the pharmaceutical composition may further comprise additional active ingredients.

All Formulations of the pharmaceutical composition disclosed herein can be produced by the conventional methods in the pharmaceutical field. For example, the active ingredient can be mixed with one or more excipients, then to make the desired Formulation. The “pharmaceutically acceptable excipient” refers to conventional pharmaceutical carriers suitable for the desired pharmaceutical Formulation, for example: a diluent, a vehicle such as water, various organic solvents, etc., a filler such as starch, sucrose, etc. a binder such as cellulose derivatives, alginates, gelatin and polyvinylpyrrolidone (PVP); a wetting agent such as glycerol; a disintegrating agent such as agar, calcium carbonate and sodium bicarbonate; an absorption enhancer such as quaternary ammonium compound; a surfactant such as hexadecanol; an absorption carrier such as Kaolin and soap clay; a lubricant such as talc, calcium stearate, magnesium stearate, polyethylene glycol, etc. In addition, the pharmaceutical composition further comprises other pharmaceutically acceptable excipients such as a decentralized agent, a stabilizer, a thickener, a complexing agent, a buffering agent, a permeation enhancer, a polymer, aromatics, a sweetener, and a dye.

The term “disease” refers to any disease, discomfort, illness, symptoms or indications, and can be interchangeable with the term “disorder” or “condition”.

Throughout this specification and the claims which follow, unless the context requires otherwise, the term “comprise,” and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cn-m” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

### Abbreviations

Ac Acetyl
AcOH Acetic acid
Aq Aqueous
Brine Saturated aqueous sodium chloride solution
Bn Benzyl
Boc Tert-butyloxycarbonyl
Cbz Benzyloxycarbonyl
CDI N,N′-Carbonyldiimidazole
Dppf 1,1″-bis(diphenylphosphino)ferrocene
DBU 1,8-diazabicyclo[5.4.0]undec-7-ene
DCE 1,2-dichloroethane
DCM Dichloromethane
DHP 3,4-dihydro-2H-pyran
DIPEA N,N-diisopropylethylamine
DMAP 4-N,N-dimethylaminopyridine
DMF N,N-dimethylformamide
DMSO Dimethyl sulfoxide
EA Ethyl acetate
EDCI 1-ethyl-(3-dimethylaminopropyl)carbonyldiimide
eq Equivalent
g Grams
HCl Hydrochloric acid
HPLC High-performance liquid flash column chromatography
HMDS Bis(trimethylsilyl)amine
IPA Isopropyl alcohol
i-PrOH Isopropyl alcohol
mg Milligrams
mL Milliliters
mmol Millimole
MeCN Acetonitrile
MeOH Methanol
Min Minutes
MS Mass spectrum
MsCl Methanesulfonyl chloride
NMR Nuclear magnetic resonance
NCS N-Chlorosuccinimide
NBS N-Bromosuccinimide
PE Petroleum ether
Pd(dppf)₂Cl₂ 1,1′-Bis(diphenylphosphino)ferrocene-palladium(II)dichloride
RT room temperature
Selectfluor 1-(Chloromethyl)-4-fluoro-1,4-diazoniabicyclo[2.2.2]octane Bis(tetrafluoroborate)
t-BuOK Potassium tert-butoxide
tBuXPhos Bis(1,1-dimethylethyl)[2′,4′,6′-tris(1-methylethyl)[1,1′-biphenyl]-2-yl]phosphine
tBuXPhos Pd G3 Methanesulfonato(2-di-t-butylphosphino-2′,4′,6′-tri-i-propyl-1,1′-biphenyl)(2′-amino-1,1′-biphenyl-2-yl)palladium(II)
TEA Triethanolamine
TFA Trifluoroacetic acid
THF Tetrahydrofuran
THP Tetrahydropyran3,4-dihydro-2H-pyran
TLC thin layer flash column chromatography
TMSCN Trimethylsilyl cyanide
TMSOTf Trimethylsilyl trifluoromethanesulfonate
XPhos 2-Dicyclohexylphosphino-2′,4′,6′-triisopropylbiphenyl

### General Synthetic Schemes

Compounds disclosed herein, including salts thereof, can be prepared using known organic synthesis techniques and can be synthesized according to any of numerous possible synthetic routes.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from room temperature to the solvent's boiling temperature. A given reaction can be carried out in one solvent or mixture of solvents.

The selection of appropriate protecting group, can be readily determined by one skilled in the art.

Reactions can be monitored according to any suitable method known in the art, such as NMR, UV, HPLC, LC-MS and TLC. Compounds can be purified by a variety of methods, including HPLC and normal phase silica flash column chromatography.

Chiral analytic HPLC was used for the retention time analysis of different chiral examples, the conditions were divided into the methods as below according to the column, mobile phase, solvent ration used. Preparation of homochiral examples may be carried out by techniques known to one skilled in the art. The absolute stereochemistry was not assigned at the newly formed carbon-nitrogen bond.

The compounds disclosed herein can be prepared by following Scheme I to II.

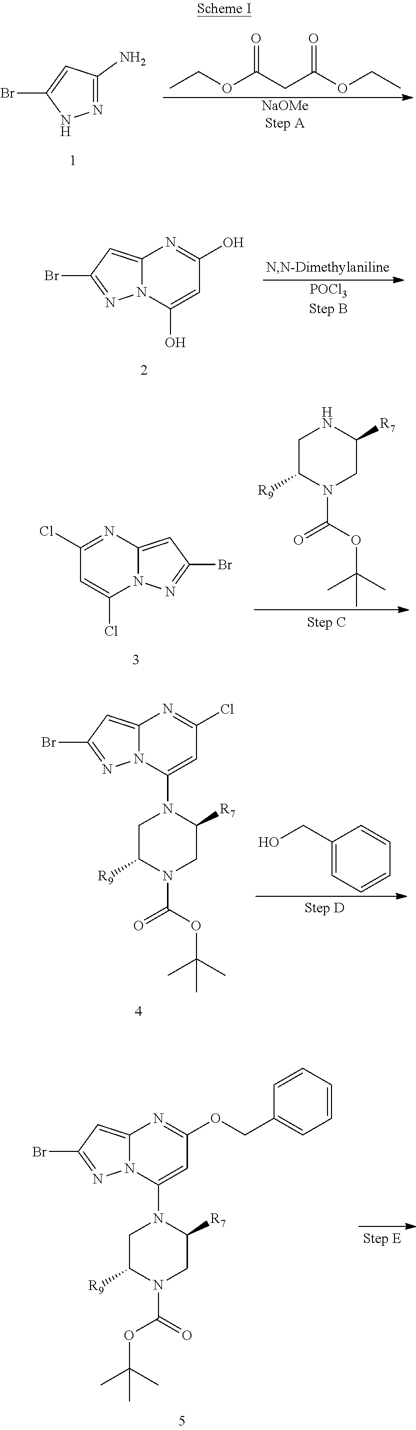

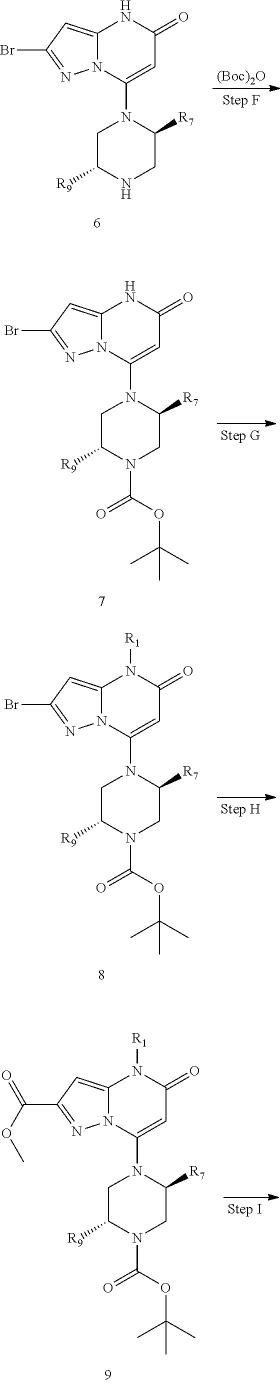

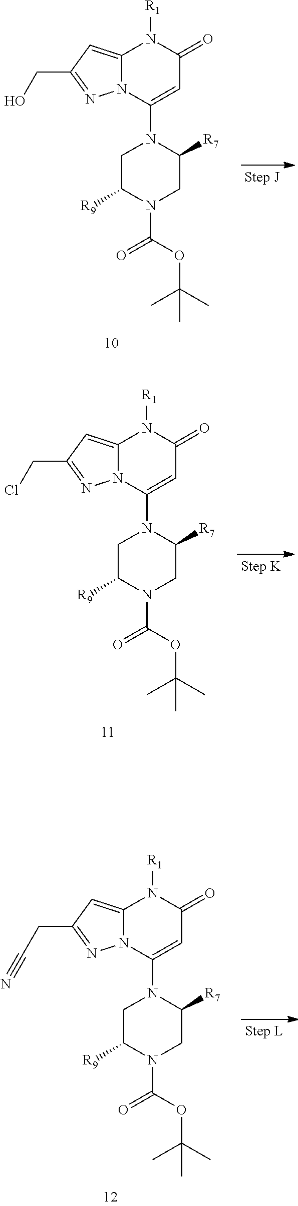

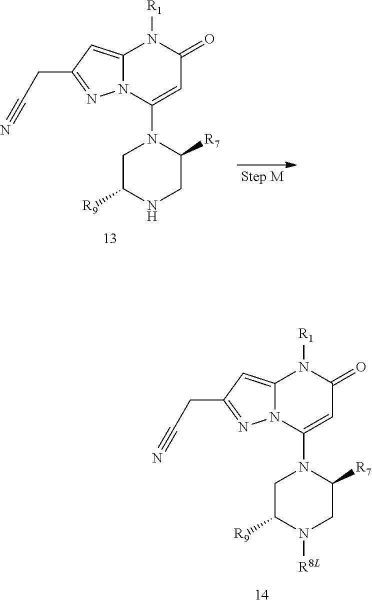

wherein the substitutions from R¹ to R⁹ and R^{8L} (corresponding to L¹-Cy¹) are as defined as mentioned in Formula (I).

In scheme I, a commercially available Compound 1 is reacted with diethyl malonate under heating condition via cyclization reaction to give Compound 2. Compound 2 is reacted with chlorination agent (such as SOCl₂ or POCl₃) to give Compound 3. Compound 3 is reacted with the appropriate chiral secondary amine by nucleophilic aromatic substitution reaction to give Compound 4. Compound 4 is reacted with the appropriate benzyl alcohol by nucleophilic aromatic substitution reaction to give Compound 5 under basic condition (such as NaH). Compound 5 is deprotected using acid/heating condition (such as TFA) to give Compound 6, following Compound 6 wherein contains a secondary amine can be protected by protecting group (such as di-tert-butyl dicarbonate) to give Compound 7. Compound 7 is reacted with appropriate R¹—X under basic condition (such as K₂CO₃, Cs₂CO₃) to give Compound 8. Compound 8 wherein contains a bromine atoms can be converted into corresponding Compound 9 by normally Pd-catalyzed methoxycarbonylation with appropriate Pd catalyst (such as Pd(dppf)Cl₂) and methoxycarbonylation reagent (such as CO/MeOH). Reduction of Compound 9 to give Compound 10 as a alcohol using reducing agent (such as NaBH₄). Compound 10 wherein contains a hydroxyl group may be chlorinated by treatment with chlorination agent such as (SOCl₂ or MsCl) to give Compound 11. Compound 11 as a benzyl halides may be converted into corresponding Compound 12 by treatment with cyanation agent (such as TMSCN) under basic condition (such as Cs₂CO₃ or nBu₄NF). Compound 12 is deprotected using acid condition (such as TFA or 4M solution of HCl in 1,4-dioxane) to give Compound 13. Tertiary amines Compound 14 is prepared by treatment with reductive alkylation with aldehydes or ketones, the most frequently used procedures via a phosphonium salt mediated alkylation of amines with corresponding alcohols (Florencio Zaragoza and Henrik Stephensen, J. Org. Chem. 2001, 66, 2518-2521).

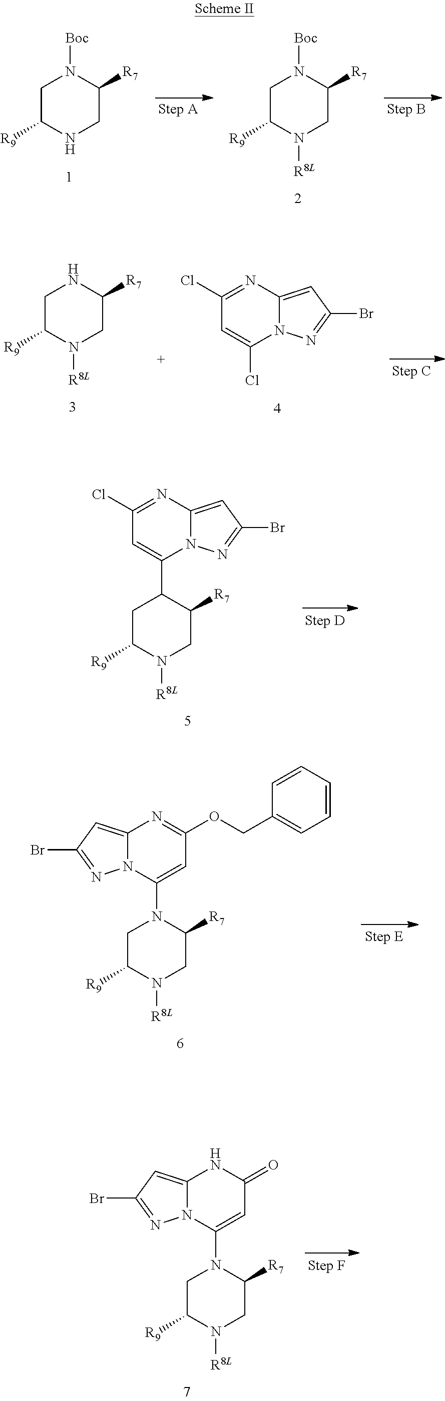

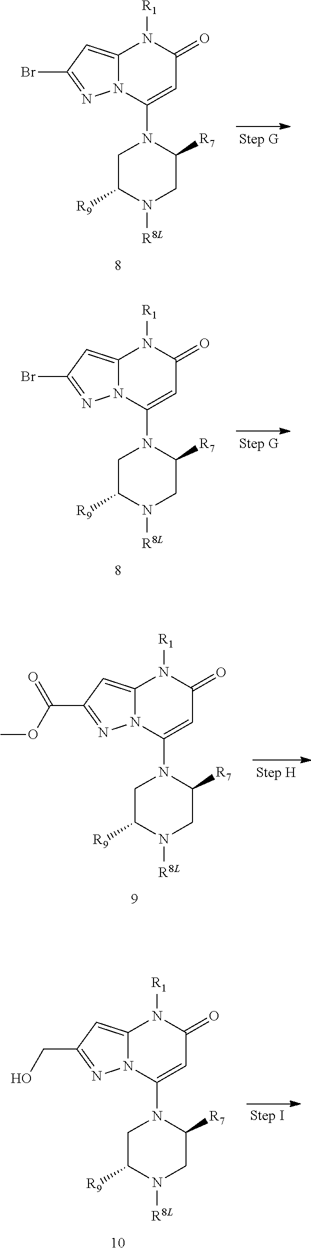

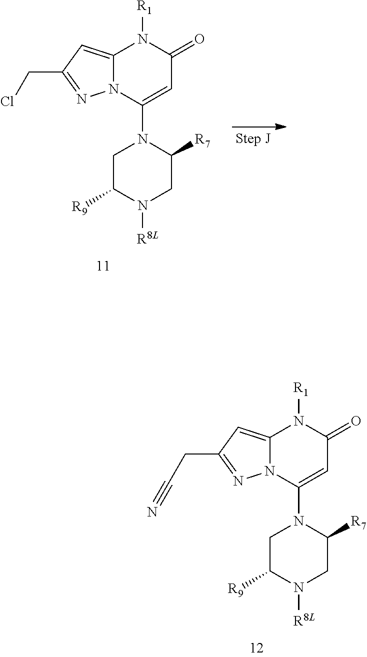

wherein the substitutions from R¹ to R⁹ and R^{8L} (corresponding to L¹-Cy¹) are as defined as mentioned in Formula (I).

In scheme H, Compound 2 is prepared by reductive amination with aldehydes or ketones, the most frequently used procedures via a phosphonium salt mediated alkylation of amines with corresponding alcohols (Florencio Zaragoza and Henrik Stephensen, J. Org. Chem. 2001, 66, 2518-2521). Compound 2 is deprotected using acid condition (such as TFA or 4M solution of HCl in 1,4-dioxane) to give Compound 3. Compound 3 is reacted with the commercially available Compound 4 by nucleophilic aromatic substitution reaction to give Compound 5. Compound 5 is reacted with the appropriate benzyl alcohol by nucleophilic aromatic substitution reaction to give Compound 6 under basic condition (such as NaH). Compound 6 is deprotected using acid/heating condition (such as TFA) to give Compound 7. Compound 7 is reacted with appropriate R¹—X under basic condition (such as K₂CO₃, Cs₂CO₃) to give Compound 8. Compound 8 wherein contains a bromine atoms can be converted into corresponding Compound 9 by normally Pd-catalyzed methoxycarbonylation with appropriate Pd catalyst (such as Pd(dppf)Cl₂) and methoxycarbonylation reagent (such as CO/MeOH). Reduction of Compound 9 to give Compound 10 as a alcohol using reducing agent (such as NaBH₄). Compound 10 wherein contains a hydroxyl group may be chlorinated by treatment with chlorination agent such as (SOCl₂ or MsCl) to give Compound 11. Compound 11 as a benzyl halides may be converted into corresponding Compound 12 by treatment with cyanation agent (such as TMSCN) under basic condition (such as Cs₂CO₃ or nBu₄NF).

### EXAMPLES

The examples below are intended to be purely exemplary and should not be considered to be limiting in any way. Unless otherwise specified, the experimental methods in the Examples described below are conventional methods. Unless otherwise specified, the reagents and materials are all commercially available. All solvents and chemicals employed are of analytical grade or chemical purity. Solvents are all redistilled before use. Anhydrous solvents are all prepared according to standard methods or reference methods. Silica gel (100-200 meshes) for flash column chromatography and silica gel (GF254) for thin-layer flash column chromatography (TLC) are commercially available from Tsingdao Haiyang Chemical Co., Ltd. or Yantai Chemical Co., Ltd. of China; all are eluted with petroleum ether (60-90° C.)/ethyl acetate (v/v), and visualized by iodine or the solution of molybdphosphoric acid in ethanol unless otherwise specified. All extraction solvents, unless otherwise specified, are dried over anhydrous Na₂SO₄. ¹H NMR spectra are recorded on Bruck-400 nuclear magnetic resonance spectrometer with TMS (tetramethylsilane) as the internal standard. LC/MS data are recorded by using Agilent1100 High Performance Liquid Flash column chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw®.

### Synthesis

### Preparative HPLC Conditions (Method A)

| | |
|---|---|
| Column | Phenomenex Gemini NX-C18, 150 × 21.2 mm, 5 μm |
| Column Temp. | R.T. |
| Detection | DAD, UV λ = 214/254 nm |
| Wavelength | |
| Run Time | 17.0 min |
| Flow Rate | 20.0 mL/min |
| Mobile Phase A | 0.1% FA—H₂O (v/v) |
| Mobile Phase B | 0.1% FA—CH₃CN (v/v) |

| | |
|---|---|
| Column | Waters XSelect CSH C18, 150 × 19 mm, 5 μm |
| Column Temp. | R.T. |
| Detection Wavelength | DAD, UV λ = 214/254 nm |
| Run Time | 17.0 min |
| Flow Rate | 17 mL/min |
| Mobile Phase A | 0.03% NH₃•H₂O—H₂O (v/v) |
| Mobile Phase B | CH₃CN |

### Intermediate 1: 7-((2S,5R)-2,5-dimethylpiperazin-1-yl)-4-methyl-5-oxo-4,5-dihydropyrazolo[1,5-a]pyrimidine-2-carbonitrile

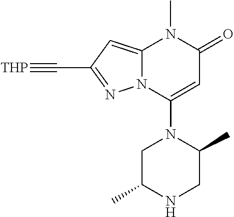

### Step A: 2-bromopyrazolo[1,5-a]pyrimidine-5,7-diol

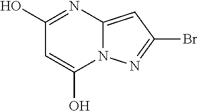 [SMILES: Oc1cc(O)n2nc(Br)cc2n1]

To a solution of 5-bromo-11H-pyrazol-3-amine (3.2 g, 20 mmol) in EtOH (50 mL) was added diethyl malonate (6.4 g, 40 mmol) and sodium methanolate (2.1 g, 40 mmol). The reaction mixture was stirred at 80° C. for 16 hours. The mixture was cooled down to RT and filtered. The filter cake was dissolved in H₂O and adjusted pH to 2-3. The resulted mixture was filtered. The filter cake was dried to give the titled compound (2 g, 44%). MS: m/e 230 (M+1)⁺.

### Step B: 2-bromo-5,7-dichloropyrazolo[1,5-a]pyrimidine

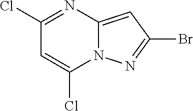 [SMILES: Clc1cc(Cl)n2nc(Br)cc2n1]

To a mixture of 2-bromopyrazolo[1,5-a]pyrimidine-5,7-diol (2 g, 0.88 mmol) in POCl₃ (10 mL) was added N,N-dimethylaniline (5 mL). The resulted mixture was stirred at 90° C. overnight. The mixture was concentrated in vacuo. The residue was added to ice-water and extracted by ethyl acetate. The organic layer was dried over Na₂SO₄, filtered, and concentrated. The resulting crude product was purified by flash column chromatography to give the titled compound (1.4 g, 61%). MS: M/e 266 (M+1)⁺.

### Step C: tert-butyl (2R,5S)-4-(2-bromo-5-chloropyrazolo[1,5-a]pyrimidin-7-yl)-2,5-dimethylpiperazine-1-carboxylate

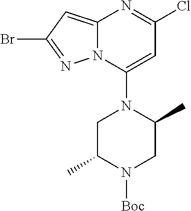 [SMILES: C[C@@H]1CN(c2cc(Cl)nc3cc(Br)nn23)[C@@H](C)CN1C(=O)OC(C)(C)C]

To a solution of 2-bromo-5,7-dichloropyrazolo[1,5-a]pyrimidine (1.1 g, 4.2 mmol) in THF (15 mL) was added tert-butyl (2R,5S)-2,5-dimethylpiperazine-1-carboxylate (1.06 g, 5 mmol) and DIPEA (1 g, 8 mmol). The resulted mixture was stirred at RT overnight. The mixture was added to water and extracted by ethyl acetate. The organic layer was dried over Na₂SO₄, filtered, and concentrated. The resulting crude product was purified by flash column chromatography to give the titled compound (1.2 g, 65%). MS: M/e 444 (M+1)⁺.

### Step D: tert-butyl (2R,5S)-4-(5-(benzyloxy)-2-bromopyrazolo[1,5-a]pyrimidin-7-yl)-2,5-dimethylpiperazine-1-carboxylate

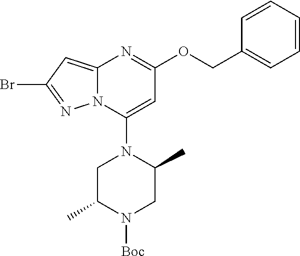 [SMILES: C[C@@H]1CN(c2cc(OCc3ccccc3)nc3cc(Br)nn23)[C@@H](C)CN1C(=O)OC(C)(C)C]

To a solution of phenylmethanol (108 mg, 1 mmol) in THF (15 mL) was added NaH (60% in oil, 80 mg. 2 mmol). The reaction was stirred at RT for 0.5 h. tert-butyl (2R,5S)-4-(2-bromo-5-chloropyrazolo[1,5-a]pyrimidin-7-yl)-2,5-dimethylpiperazine-1-carboxylate (200 mg, 0.5 mmol) was added to the mixture and the reaction was stirred at 70° C. overnight. The mixture was added to water and extracted by ethyl acetate. The organic layer was dried over Na₂SO₄, filtered, and concentrated. The resulting crude product was purified by flash column chromatography to give the titled compound (230 mg, 89%). MS: M/e 516 (M+1)⁺.

### Step E: 2-bromo-7-((2S,5R)-2,5-dimethylpiperazin-1-yl)pyrazolo[1,5-a]pyrimidin-5(4H)-one

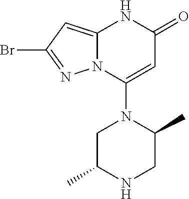 [SMILES: C[C@@H]1CN(c2cc(=O)[nH]c3cc(Br)nn23)[C@@H](C)CN1]

A mixture of tert-butyl (2R,5S)-4-(5-(benzyloxy)-2-bromopyrazolo[1,5-a]pyrimidin-7-yl)-2,5-dimethylpiperazine-1-carboxylate (180 mg, 0.35 mmol) in TFA (5 mL) was stirred at 80° C. overnight. The mixture concentrated in vacuo. The residue was added to a solution of saturated NaHCO₃ aqueous solution and extracted by DCM. The organic layer was dried over Na₂SO₄, filtered, and concentrated. The resulting crude product was used directly in next step without further purification. MS: M/e 326 (M+1).

### Step F: tert-butyl (2R,5S)-4-(2-bromo-5-oxo-4,5-dihydropyrazolo[1,5-a]pyrimidin-7-yl)-2,5-dimethylpiperazine-1-carboxylate

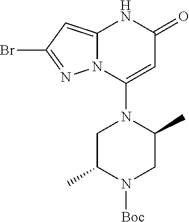 [SMILES: C[C@@H]1CN(c2cc(=O)[nH]c3cc(Br)nn23)[C@@H](C)CN1C(=O)OC(C)(C)C]

To a solution of 2-bromo-7-((2S,5R)-2,5-dimethylpiperazin-1-yl)pyrazolo[1,5-a]pyrimidin-5(4H)-one in DMF (5 mL) was added di-tert-butyl decarbonate (106 mg, 0.5 mmol) and DIPEA (129 mg, 1 mmol). The reaction mixture was stirred at RT overnight. The mixture was added H₂O and extracted by ethyl acetate. The organic layer was washed with brine, dried over Na₂SO₄, filtered, and concentrated. The resulting crude product was purified by flash column chromatography to give the titled compound (120 mg, 81% yield for two steps). MS: M/e 426 (M+1)⁺.

### Step G: tert-butyl (2R,5S)-4-(2-bromo-4-methyl-5-oxo-4,5-dihydropyrazolo[1,5-a]pyrimidin-7-yl)-2,5-dimethylpiperazine-1-carboxylate

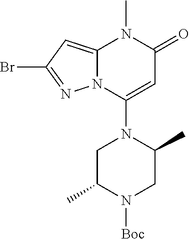 [SMILES: C[C@@H]1CN(c2cc(=O)n(C)c3cc(Br)nn23)[C@@H](C)CN1C(=O)OC(C)(C)C]

To a solution of tert-butyl (2R,5S)-4-(2-bromo-5-oxo-4,5-dihydropyrazolo[1,5-a]pyrimidin-7-yl)-2,5-dimethylpiperazine-1-carboxylate (120 mg. 0.28 mmol) in 1,4-dioxane (5 mL) was added trimethyl phosphate (197 mg, 1.4 mmol) and K₂CO₃ (58 mg, 0.42 mmol). The reaction mixture was stirred at 95° C. overnight. The mixture was added H₂O and extracted by ethyl acetate. The organic layer was washed with brine, dried over Na₂SO₄, filtered, and concentrated. The resulting crude product was purified by flash column chromatography to give the titled compound (122 mg, 100%). MS: M/e 440 (M+1)⁺.

### Step H: tert-butyl (2R,5S)-4-(2-cyano-4-methyl-5-oxo-4,5-dihydropyrazolo[1,5-a]pyrimidin-7-yl)-2,5-dimethylpiperazine-1-carboxylate

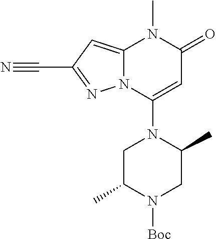 [SMILES: C[C@@H]1CN(c2cc(=O)n(C)c3cc(C#N)nn23)[C@@H](C)CN1C(=O)OC(C)(C)C]

To a solution of tert-butyl (2R,5S)-4-(2-bromo-4-methyl-5-oxo-4,5-dihydropyrazolo[1,5-a]pyrimidin-7-yl)-2,5-dimethylpiperazine-1-carboxylate (50 mg. 0.11 mmol) in 1,4-dioxane/H₂O (5 mL/2.5 ml) was added Zn(CN)₂ (38 mg, 0.33 mmol), t-BuXphos Pd G3 (39 mg, 0.05 mmol) and t-BuXPhos (47 mg, 0.11 mmol). The reaction mixture was stirred under nitrogen protection at 95° C. overnight. The mixture was added H₂O and extracted by ethyl acetate. The organic layer was washed with brine, dried over Na₂SO₄, filtered, and concentrated. The resulting crude product was purified by flash column chromatography to give the titled compound (40 mg, 95%). MS: M/e 387 (M+1)⁺.

### Step I: 7-((2S,5R)-2,5-dimethylpiperazin-1-yl)-4-methyl-5-oxo-4,5-dihydropyrazolo[1,5-a]pyrimidine-2-carbonitrile

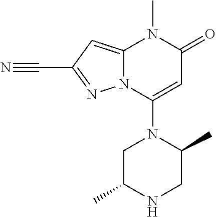 [SMILES: C[C@@H]1CN(c2cc(=O)n(C)c3cc(C#N)nn23)[C@@H](C)CN1]

A mixture of tert-butyl (2R,5S)-4-(2-cyano-4-methyl-5-oxo-4,5-dihydropyrazolo[1,5-a]pyrimidin-7-yl)-2,5-dimethylpiperazine-1-carboxylate (40 mg. 0.1 mmol) in TFA (5 mL) was stirred at RT for 2 hours. The mixture concentrated in vacuo. The residue was added to a solution of saturated NaHCO₃ aqueous solution and extracted by DCM. The organic layer was dried over Na₂SO₄, filtered, and concentrated to dryness. The resulting residue was used directly in next step without further purification. MS: M/e 287 (M+1)⁺.

### Intermediate 2: 2-(7-((2S,5R)-2,5-dimethylpiperazin-1-yl)-4-methyl-5-oxo-4,5-dihydropyrazolo[1,5-a]pyrimidin-2-yl)acetonitrile

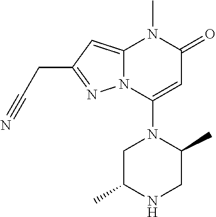 [SMILES: C[C@@H]1CN(c2cc(=O)n(C)c3cc(CC#N)nn23)[C@@H](C)CN1]

### Step A: methyl 7-((2S,5R)-4-(tert-butoxycarbonyl)-2,5-dimethylpiperazin-1-yl)-4-methyl-5-oxo-4,5-dihydropyrazolo[1,5-a]pyrimidine-2-carboxylate

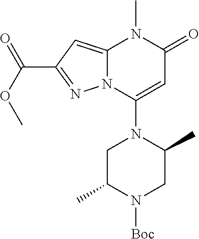 [SMILES: COC(=O)c1cc2n(C)c(=O)cc(N3C[C@@H](C)N(C(=O)OC(C)(C)C)C[C@@H]3C)n2n1]

To a solution of tert-butyl (2R,5S)-4-(2-bromo-4-methyl-5-oxo-4,5-dihydropyrazolo[1,5-a]pyrimidin-7-yl)-2,5-dimethylpiperazine-1-carboxylate (3.4 g, 7.7 mmol) in MeOH (40 mL) was added Pd(dppf)₂Cl₂ (365 mg, 0.5 mmol) and Et₃N (2 g, 20 mmol). The reaction mixture was stirred under an CO atmosphere at 90° C. for 16 h. The mixture was cooled down to rt. The mixture was added to water and extracted by ethyl acetate. The organic phase was dried over Na₂SO₄, filtered, and concentrated. The crude product was purified by column chromatography to give the titled compound (2 g, 59.8%). MS: M/e 420.2 (M+1)⁺.

### Step B: tert-butyl (2R,5S)-4-(2-(hydroxymethyl)-4-methyl-5-oxo-4,5-dihydropyrazolo[1,5-a]pyrimidin-7-yl)-2,5-dimethylpiperazine-1-carboxylate

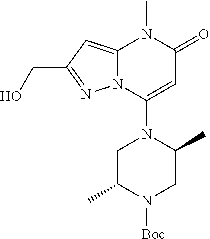 [SMILES: C[C@@H]1CN(c2cc(=O)n(C)c3cc(CO)nn23)[C@@H](C)CN1C(=O)OC(C)(C)C]

To a mixture of methyl 7-((2S,5R)-4-(tert-butoxycarbonyl)-2,5-dimethylpiperazin-1-yl)-4-methyl-5-oxo-4,5-dihydropyrazolo[1,5-a]pyrimidine-2-carboxylate (1.6 g, 3.8 mmol) in THF (10 mL) was cooled down to 0° C., The mixture was added LiAlH₄ (175 mg, 4.6 mmol). The resulted mixture was stirred at 0° C. for 30 mins. The mixture was added H₂O (0.2 ml), 15% NaOH solution (0.2 ml) and H₂O (0.6 ml). The mixture was filtered. The filtrate was concentrated in vacuo. The crude product was purified by column chromatography to give the titled compound (1.2 g, 80%). MS: M/e 392.2 (M+1)⁺.

### Step C: tert-butyl (2R,5S)-2,5-dimethyl-4-(4-methyl-2-(((methylsulfonyl)oxy)methyl)-5-oxo-4,5-dihydropyrazolo[1,5-a]pyrimidin-7-yl)piperazine-1-carboxylate

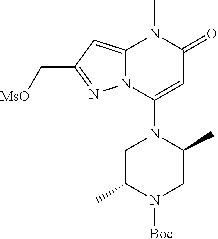 [SMILES: C[C@@H]1CN(c2cc(=O)n(C)c3cc(COS(C)(=O)=O)nn23)[C@@H](C)CN1C(=O)OC(C)(C)C]

To a mixture of tert-butyl (2R,5S)-4-(2-(hydroxymethyl)-4-methyl-5-oxo-4,5-dihydropyrazolo[1,5-a]pyrimidin-7-yl)-2,5-dimethylpiperazine-1-carboxylate (1.5 g, 3.8 mmol) in DCM (15 mL) was added methanesulfonyl chloride (877 mg, 7.7 mmol) and Et₃N (1.5 g, 15.2 mmol). The resulted mixture was stirred at rt for 30 mins. The mixture was washed with NaHCO₃ aqueous solution, dried over Na₂SO₄, filtered, and concentrated. The crude product was used directly in next step. MS: M/e 470.2 (M+1)⁺.

### Step D: tert-butyl (2R,5S)-4-(2-(cyanomethyl)-4-methyl-5-oxo-4,5-dihydropyrazolo[1,5-a]pyrimidin-7-yl)-2,5-dimethylpiperazine-1-carboxylate

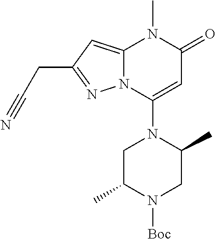 [SMILES: C[C@@H]1CN(c2cc(=O)n(C)c3cc(CC#N)nn23)[C@@H](C)CN1C(=O)OC(C)(C)C]

To a mixture of tert-butyl (2R,5S)-2,5-dimethyl-4-(4-methyl-2-(((methylsulfonyl)oxy)methyl)-5-oxo-4,5-dihydropyrazolo[1,5-a]pyrimidin-7-yl)piperazine-1-carboxylate from last step in acetonitrile (20 mL) was added TMSCN (1.1 g. 11.4 mmol) and K₂CO₃ (2.6 g, 19 mmol). The reaction was stirred at 80° C. overnight. The mixture was added to water and extracted by ethyl acetate. The organic phase was dried over Na₂SO₄, filtered, and concentrated. The crude product was purified by column chromatography to give the titled compound (700 mg, 45.7% for two steps). MS: M/e 401.2 (M+1)⁺.

### Intermediate 3: 2-(7-((2S,5R)-5-ethyl-2-methylpiperazin-1-yl)-4-methyl-5-oxo-4,5-dihydropyrazolo[1,5-a]pyrimidin-2-yl)acetonitrile

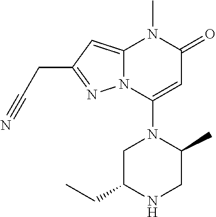 [SMILES: CC[C@@H]1CN(c2cc(=O)n(C)c3cc(CC#N)nn23)[C@@H](C)CN1]

### Step A: tert-butyl (2R,5S)-4-(2-bromo-5-chloropyrazolo[1,5-a]pyrimidin-7-yl)-2-ethyl-5-methylpiperazine-1-carboxylate

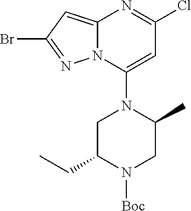 [SMILES: CC[C@@H]1CN(c2cc(Cl)nc3cc(Br)nn23)[C@@H](C)CN1C(=O)OC(C)(C)C]

To a mixture of 2-bromo-5,7-dichloropyrazolo[1,5-a]pyrimidine (5.03 g, 19 mmol) in THF (50 mL) was added tert-butyl (2R,5S)-2-ethyl-5-methylpiperazine-1-carboxylate (5.6 g, 24.7 mmol) and DIPEA (4.9 g, 38 mmol). The resulted mixture was stirred at RT overnight. The mixture was added to water and extracted by ethyl acetate. The organic phase was dried over Na₂SO₄, filtered, and concentrated. The resulting residue was purified by column chromatography to give the titled compound (7.8 g, 89.6%). MS: M/e 458 (M+1)⁺.

### Step B: tert-butyl (2R,5S)-4-(2-bromo-5-oxo-4,5-dihydropyrazolo[1,5-a]pyrimidin-7-yl)-2-ethyl-5-methylpiperazine-1-carboxylate

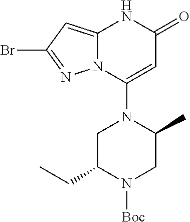 [SMILES: CC[C@@H]1CN(c2cc(=O)[nH]c3cc(Br)nn23)[C@@H](C)CN1C(=O)OC(C)(C)C]

To a mixture of 2-(methylsulfonyl)ethan-1-ol (3.6 g, 29 mmol) in THF (80 mL) was added NaH (60% in oil, 1.71 g. 42.75 mmol). The reaction was stirred at RT for 0.5 h. tert-butyl (2R,5S)-4-(2-bromo-5-chloropyrazolo[1,5-a]pyrimidin-7-yl)-2-ethyl-5-methylpiperazine-1-carboxylate (7.8 g, 17.1 mmol) was added to the mixture and the reaction was stirred at 70° C. overnight. The mixture was added to NH₄Cl solution and extracted by ethyl acetate. The organic phase was dried over Na₂SO₄, filtered, and concentrated. The crude product was purified by column chromatography to give the title product (7.83 g, 100%). MS: M/e 440 (M+1)⁺.

### Step C: tert-butyl (2R,5S)-4-(2-bromo-4-methyl-5-oxo-4,5-dihydropyrazolo[1,5-a]pyrimidin-7-yl)-2-ethyl-5-methylpiperazine-1-carboxylate

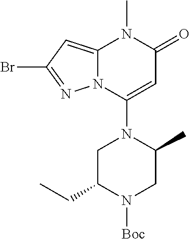 [SMILES: CC[C@@H]1CN(c2cc(=O)n(C)c3cc(Br)nn23)[C@@H](C)CN1C(=O)OC(C)(C)C]

To a mixture of tert-butyl (2R,5S)-4-(2-bromo-5-oxo-4,5-dihydropyrazolo[1,5-a]pyrimidin-7-yl)-2-ethyl-5-methylpiperazine-1-carboxylate (7.83 g, 17.8 mmol) in 1,4-dioxane (80 mL) was added Trimethyl phosphate (12.5 g, 89 mmol) and K₂CO₃ (4.95 g, 35.6 mmol). The reaction mixture was sealed and stirred at 95° C. for 4 hrs. The mixture was added H₂O and filtered. The residue was dissolved in EA and was with brine. The organic phase was dried over Na₂SO₄, filtered, and concentrated. The crude product was purified by column chromatography to give the title product (7 g, 87.5%). MS: M/e 454 (M+1)⁺.

### Step D: methyl 7-((2S,5R)-4-(tert-butoxycarbonyl)-5-ethyl-2-methylpiperazin-1-yl)-4-methyl-5-oxo-4,5-dihydropyrazolo[1,5-a]pyrimidine-2-carboxylate

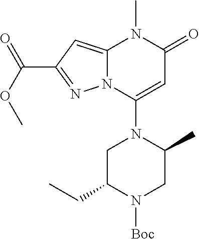 [SMILES: CC[C@@H]1CN(c2cc(=O)n(C)c3cc(C(=O)OC)nn23)[C@@H](C)CN1C(=O)OC(C)(C)C]

To a solution of tert-butyl (2R,5S)-4-(2-bromo-4-methyl-5-oxo-4,5-dihydropyrazolo[1,5-a]pyrimidin-7-yl)-2-ethyl-5-methylpiperazine-1-carboxylate (7 g, 15.5 mmol) in MeOH (200 mL) was added Pd(dppf)₂Cl₂ (543 mg, 0.78 mmol) and Et₃N (3.13 g, 31 mmol). The reaction mixture was stirred under an CO atmosphere at 90° C. for 16 hrs. The mixture was cooled down to RT. The mixture was added to water and extracted by ethyl acetate. The organic phase was dried over Na₂SO₄, filtered, and concentrated. The crude product was purified by column chromatography to give the title product (4.6 g, 68.6%). MS: M/e 434 (M+1)⁺.

### Step E: tert-butyl (2R,5S)-2-ethyl-4-(2-(hydroxymethyl)-4-methyl-5-oxo-4,5-dihydropyrazolo[1,5-a]pyrimidin-7-yl)-5-methylpiperazine-1-carboxylate

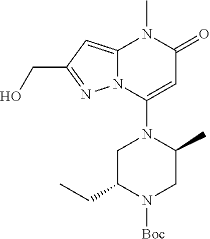 [SMILES: CC[C@@H]1CN(c2cc(=O)n(C)c3cc(CO)nn23)[C@@H](C)CN1C(=O)OC(C)(C)C]

To a mixture of methyl 7-((2S,5R)-4-(tert-butoxycarbonyl)-5-ethyl-2-methylpiperazin-1-yl)-4-methyl-5-oxo-4,5-dihydropyrazolo[1,5-a]pyrimidine-2-carboxylate (4.6 g, 10.1 mmol) in THF (25 mL) was cooled down to 0° C., The mixture was added LiAlH₄ (304 mg, 8 mmol) slowly and stirred at 0° C. for 30 mins. The mixture was added H₂O (0.3 ml), 15% NaOH solution (0.3 ml) and H₂O (0.9 ml). The mixture was filtered. The filtrate was concentrated in vacuo. The crude product was purified by column chromatography to give the title product (3.5 g, 87.5%). MS: M/e 406 (M+1)+.

### Step F: tert-butyl (2R,5S)-2-ethyl-5-methyl-4-(4-methyl-2-(((methylsulfonyl)oxy)methyl)-5-oxo-4,5-dihydropyrazolo[1,5-a]pyrimidin-7-yl)piperazine-1-carboxylate

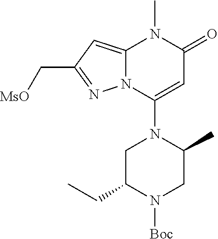 [SMILES: CC[C@@H]1CN(c2cc(=O)n(C)c3cc(COS(C)(=O)=O)nn23)[C@@H](C)CN1C(=O)OC(C)(C)C]

To a mixture of tert-butyl (2R,5S)-2-ethyl-4-(2-(hydroxymethyl)-4-methyl-5-oxo-4,5-dihydropyrazolo[1,5-a]pyrimidin-7-yl)-5-methylpiperazine-1-carboxylate (3.5 g, 9.2 mmol) in DCM (50 mL) at 0° C. was added Et₃N (1.4 g, 13.8 mmol) and methanesulfonyl chloride (1.26 g, 11 mmol). The resulted mixture was stirred at RT for 5 mins. The mixture was washed with NaHCO₃ aqueous solution, dried over Na₂SO₄, filtered, and concentrated to give the crude titled compound, which was used directly in next step. MS: M/e 484 (M+1)⁺.

### Step G: tert-butyl (2R,5S)-4-(2-(cyanomethyl)-4-methyl-5-oxo-4,5-dihydropyrazolo[1,5-a]pyrimidin-7-yl)-2-ethyl-5-methylpiperazine-1-carboxylate

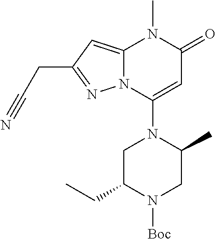 [SMILES: CC[C@@H]1CN(c2cc(=O)n(C)c3cc(CC#N)nn23)[C@@H](C)CN1C(=O)OC(C)(C)C]

To a mixture of tert-butyl (2R,5S)-2-ethyl-5-methyl-4-(4-methyl-2-(((methylsulfonyl)oxy)methyl)-5-oxo-4,5-dihydropyrazolo[1,5-a]pyrimidin-7-yl)piperazine-1-carboxylate from last step in MeCN (50 mL) was added TMSCN (4.5 g. 46 mmol) and Cs₂CO₃ (6 g, 18.4 mmol). The reaction was stirred at 80° C. for 2 hrs. The mixture was concentrated in vacuo. The residue was added to water and extracted by ethyl acetate. The organic phase was dried over Na₂SO₄, filtered, and concentrated. The crude product was purified by column chromatography to give the title compound (1.9 g, 50% for two steps). MS: M/e 415 (M+1)⁺.

### Step H: 2-(7-((2S,5R)-5-ethyl-2-methylpiperazin-1-yl)-4-methyl-5-oxo-4,5-dihydropyrazolo[1,5-a]pyrimidin-2-yl)acetonitrile

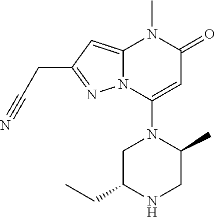 [SMILES: CC[C@@H]1CN(c2cc(=O)n(C)c3cc(CC#N)nn23)[C@@H](C)CN1]

A mixture of tert-butyl (2R,5S)-4-(2-(cyanomethyl)-4-methyl-5-oxo-4,5-dihydropyrazolo[1,5-a]pyrimidin-7-yl)-2-ethyl-5-methylpiperazine-1-carboxylate (1.9 g. 4.6 mmol) in DCM (20 ml) was added TFA (2 mL). The reaction was stirred at RT for 2 hrs. The mixture concentrated in vacuo. The residue was added to a solution of saturated NaHCO₃ aqueous solution and extracted by DCM. The organic phase was dried over Na₂SO₄, filtered, and concentrated to giver the title product (1.5 g, 100%). MS: M/e 315 (M+1)⁺.

### Intermediate 4: 2-(7-((2S,5R)-2,5-diethylpiperazin-1-yl)-4-methyl-5-oxo-4,5-dihydropyrazolo[1,5-a]pyrimidin-2-yl)acetonitrile

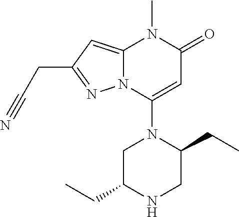 [SMILES: CC[C@@H]1CN(c2cc(=O)n(C)c3cc(CC#N)nn23)[C@@H](CC)CN1]

### Step A: tert-butyl (2R,5S)-4-(2-bromo-5-chloropyrazolo[1,5-a]pyrimidin-7-yl)-2,5-diethylpiperazine-1-carboxylate

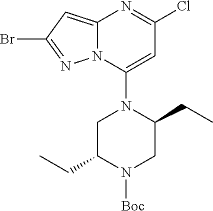 [SMILES: CC[C@@H]1CN(c2cc(Cl)nc3cc(Br)nn23)[C@@H](CC)CN1C(=O)OC(C)(C)C]

A solution of 2-bromo-5,7-dichloropyrazolo[1,5-a]pyrimidine (5 g, 0.019 mol), tert-butyl (2R,5S)-2,5-diethylpiperazine-1-carboxylate (5.93 g, 0.025 mol) and DIPEA (4.87 g, 0.038 mol) in THF (50 ml) was stirred at 80° C. overnight. The solution was diluted with EA (70 ml), washed with brine (40 ml), dried and evaporated. The residue was purified by flash column chromatography with 0-15% EA in PE to give the titled compound (8.4 g, 94%). MS: M/e 472,474 (M+1)⁺.

### Step B: tert-butyl (2R,5S)-4-(2-bromo-5-oxo-4,5-dihydropyrazolo[1,5-a]pyrimidin-7-yl)-2,5-diethylpiperazine-1-carboxylate

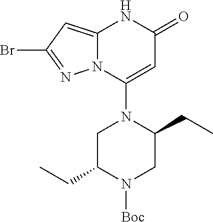 [SMILES: CC[C@@H]1CN(c2cc(=O)[nH]c3cc(Br)nn23)[C@@H](CC)CN1C(=O)OC(C)(C)C]

To a solution of 2-(methylsulfonyl)ethan-1-ol (1.43 g, 0.012 mol) in THF (20 ml), was added NaH (0.68 g, 60%, 0.017 mol) and then stirred at RT for 10 mins. Tert-butyl (2R,5S)-4-(2-bromo-5-chloropyrazolo[1,5-a]pyrimidin-7-yl)-2,5-diethylpiperazine-1-carboxylate (3.2 g, 6.79 mmol) in THF (15 ml) was added to the above solution and stirred at 70° C. for 6 hours. The reaction was quenched with H₂O (30 ml) and then extracted with EA (30 ml×2). The organic layer was washed with brine (10 ml), dried and evaporated. The residue was purified by flash column chromatography with 40-80% EA in PE to give the titled compound (2.4 g, 79%). (Another batch of titled compound (1.8 g, 22%) was obtained using similar procedure to above, totally 4.2 g titled compound was obtained). MS: M/e 454,456 (M+1)⁺.

### Step C: tert-butyl (2R,5S)-4-(2-bromo-4-methyl-5-oxo-4,5-dihydropyrazolo[1,5-a]pyrimidin-7-yl)-2,5-diethylpiperazine-1-carboxylate

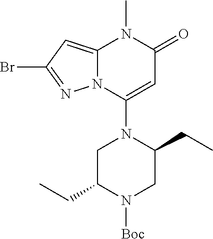 [SMILES: CC[C@@H]1CN(c2cc(=O)n(C)c3cc(Br)nn23)[C@@H](CC)CN1C(=O)OC(C)(C)C]

A solution of tert-butyl (2R,5S)-4-(2-bromo-5-oxo-4,5-dihydropyrazolo[1,5-a]pyrimidin-7-yl)-2,5-diethylpiperazine-1-carboxylate (3 g, 6.62 mmol), trimethyl phosphate (4.64 g, 33.14 mmol) and K₂CO₃ (1.83 g, 13.26 mmol) in dioxane (30 ml) was stirred at 95° C. for 4 h. The reaction was concentrated. The residue was diluted with EA (50 ml), washed with brine (20 ml×2), dried and evaporated. The residue was purified by flash column chromatography with 0-20% EA in PE to give the titled compound (2.6 g, 82%). (Another batch of titled compound (1.0 g, 78%) was obtained using similar procedure to above, totally 3.6 g titled compound was obtained). MS: M/e 468,470 (M+1)⁺.

### Step D: methyl 7-((2S,5R) 4-(tert-butoxycarbonyl)-2,5-diethylpiperazin-1-yl)-4-methyl-5-oxo-4,5-dihydropyrazolo[1,5-a]pyrimidine-2-carboxylate

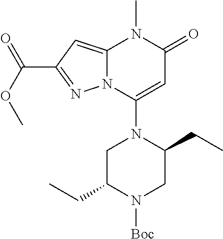 [SMILES: CC[C@@H]1CN(c2cc(=O)n(C)c3cc(C(=O)OC)nn23)[C@@H](CC)CN1C(=O)OC(C)(C)C]

A solution of tert-butyl (2R,5S)-4-(2-bromo-4-methyl-5-oxo-4,5-dihydropyrazolo[1,5-a]pyrimidin-7-yl)-2,5-diethylpiperazine-1-carboxylate (3.6 g, 7.7 mmol), Pd(dppf)Cl₂ (0.56 g, 0.77 mmol) and TEA (2.3 g, 23.2 mmol) in MeOH (40 ml) was stirred at 90° C. under CO atmosphere (2.7 MPa) overnight. The reaction was concentrated under reduced pressure. The residue was purified by flash column chromatography with 30-50% EA in PE to give the titled compound (2.4 g, 71%). MS: M/e 448 (M+1)⁺.

### Step E: tert-butyl (2R,5S)-2,5-diethyl-4-(2-(hydroxymethyl)-4-methyl-5-oxo-4,5-dihydropyrazolo[1,5-a]pyrimidin-7-yl)piperazine-1-carboxylate

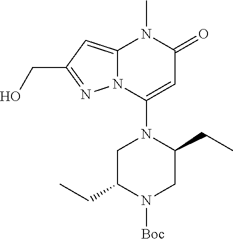 [SMILES: CC[C@@H]1CN(c2cc(=O)n(C)c3cc(CO)nn23)[C@@H](CC)CN1C(=O)OC(C)(C)C]

A mixture of methyl 7-((2S,5R)-4-(tert-butoxycarbonyl)-2,5-diethylpiperazin-1-yl)-4-methyl-5-oxo-4,5-dihydropyrazolo[1,5-a]pyrimidine-2-carboxylate (2.2 g, 5 mmol) and LiAlH₄ (0.23 g, 6 mmol) in THF (25 ml) was stirred at 0° C. for 1 h. The reaction was quenched with H₂O (25 ml) and then extracted with EA (20 ml×2). The organic layer was dried and evaporated. The resulting residue was purified by flash column chromatography with 0-5% MeOH in DCM to give the titled compound (1.4 g, 67%). MS: M/e 420 (M+1)⁺.

### Step F: tert-butyl (2R,5S)-2,5-diethyl-4-(4-methyl-2-(((methylsulfonyl)oxy)methyl)-5-oxo-4,5-dihydropyrazolo[1,5-a]pyrimidin-7-yl)piperazine-1-carboxylate

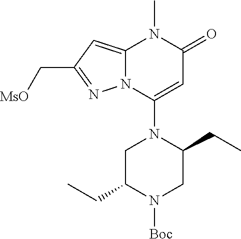 [SMILES: CC[C@@H]1CN(c2cc(=O)n(C)c3cc(COS(C)(=O)=O)nn23)[C@@H](CC)CN1C(=O)OC(C)(C)C]

To a solution of tert-butyl (2R,5S)-2,5-diethyl-4-(2-(hydroxymethyl)-4-methyl-5-oxo-4,5-dihydropyrazolo[1,5-a]pyrimidin-7-yl)piperazine-1-carboxylate (1.2 g, 2.86 mmol) and TEA (0.87 g, 8.61 mmol) in DCM (15 ml) at 0° C., was added MsCl (0.41 g, 3.57 mmol) and then stirred at 0° C. for 30 min. The solution was washed with brine (10 ml×2), dried and evaporated to dryness to give the titled compound (1.4 g, 100%), which was used directly for the next step. MS: M/e 498 (M+1)⁺.

### Step G: tert-butyl (2R,5S)-4-(2-(cyanomethyl)-4-methyl-5-oxo-4,5-dihydropyrazolo[1,5-a]pyrimidin-7-yl)-2,5-diethylpiperazine-1-carboxylate

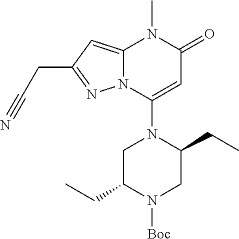 [SMILES: CC[C@@H]1CN(c2cc(=O)n(C)c3cc(CC#N)nn23)[C@@H](CC)CN1C(=O)OC(C)(C)C]

A mixture of tert-butyl (2R,5S)-2,5-diethyl-4-(4-methyl-2-(((methylsulfonyl)oxy)methyl)-5-oxo-4,5-dihydropyrazolo[1,5-a]pyrimidin-7-yl)piperazine-1-carboxylate (1.4 g, 2.9 mmol), TMSCN (0.85 g, 8.6 mmol) and K₂CO₃ (1.18 g, 8.55 mmol) in MeCN (15 ml) was stirred at 70° C. for 3.5 h. The solution was poured into water (30 ml) and then extracted with EA (20 ml×2). The organic layer was washed with brine (10 ml), dried and evaporated. The residue was purified by flash column chromatography with 40-60% EA in PE to give the titled compound (530 mg, 43%). MS: M/e 429 (M+1)⁺.

### Step H: 2-(7-((2S,5R)-2,5-diethylpiperazin-1-yl)-4-methyl-5-oxo-4,5-dihydropyrazolo[1,5-a]pyrimidin-2-yl)acetonitrile

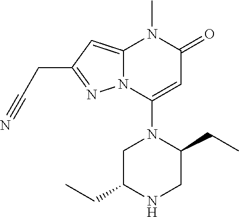 [SMILES: CC[C@@H]1CN(c2cc(=O)n(C)c3cc(CC#N)nn23)[C@@H](CC)CN1]

A solution of tert-butyl (2R,5S)-4-(2-(cyanomethyl)-4-methyl-5-oxo-4,5-dihydropyrazolo[1,5-a]pyrimidin-7-yl)-2,5-diethylpiperazine-1-carboxylate (530 mg, 1.24 mmol) and TFA (2 ml) in DCM (15 ml) was stirred at rt for 2 h. The reaction was washed with aq. NaHCO₃ (15 ml×2) and brine (10 ml), dried over Na₂SO₄ and then evaporated to dryness to give the titled compound (400 mg, 98%), which was used directly for the next step without further purification. MS: M/e 329 (M+1)⁺.

### Intermediate 5: 2-(4-((2S,5R)-2,5-diethylpiperazin-1-yl)-1-methyl-2-oxo-1,2-dihydropyrazolo[1,5-a][1,3,5]triazin-7-yl)acetonitrile

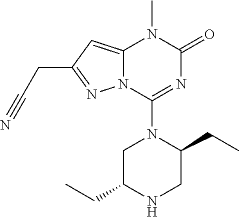 [SMILES: CC[C@@H]1CN(c2nc(=O)n(C)c3cc(CC#N)nn23)[C@@H](CC)CN1]

### Step A: ethyl 5-(3-(methoxycarbonyl)ureido)-1H-pyrazole-3-carboxylate

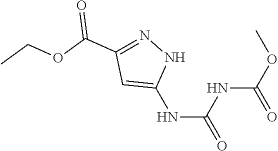 [SMILES: CCOC(=O)c1cc(NC(=O)NC(=O)OC)[nH]n1]

A solution of methyl carbamate (2.5 g, 33.33 mmol), oxalyl dichloride (4.66 g, 36.69 mmol) and HCl (4M in dioxane, 11.67 ml, 46.68 mmol) in toluene (10 mL) was stirred at 120° C. overnight. To above solution was added ethyl 5-amino-1H-pyrazole-3-carboxylate (5.17 g, 33.35 mmol) and DMF (20 mL), was stirred at RT overnight. The solution was poured into H₂O (100 mL) and then filtered. The filtration cake was dried to give the titled compound (7.8 g, 91%). MS: M/e 257 (M+1)⁺.

### Step B: ethyl 2,4-dioxo-1,2,3,4-tetrahydropyrazolo[1,5-a][1,3,5]triazine-7-carboxylate

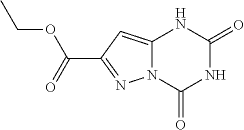 [SMILES: CCOC(=O)c1cc2[nH]c(=O)[nH]c(=O)n2n1]

A solution of ethyl 5-(3-(methoxycarbonyl)ureido)-1H-pyrazole-3-carboxylate (7.8 g, 30.47 mmol) and sodium ethoxide (21%, 19.7 g, 60.84 mmol) in EtOH (100 mL) was stirred at 80° C. for 40 min. The solution was cooled to RT and filtered. The filtration cake was dissolved in H₂O (50 mL), adjusted to pH=2, and then filtered. The filtrate was evaporated to dryness to give the titled compound (4.7 g, 68%). MS: M/e 225 (M+1)⁺.

### Step C: ethyl 2,4-dichloropyrazolo[1,5-a][1,3,5]triazine-7-carboxylate

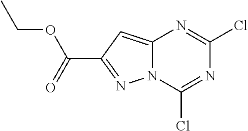 [SMILES: CCOC(=O)c1cc2nc(Cl)nc(Cl)n2n1]

A solution of ethyl 2,4-dioxo-1,2,3,4-tetrahydropyrazolo[1,5-a][1,3,5]triazine-7-carboxylate (4.7 g, 20.98 mmol) and DIPEA (5.4 g, 41.86 mmol) in POCl₃ (30 mL) was stirred at 100° C. for 4.5 hours. The solution was evaporated to dryness to give the titled compound (5.46 g, 100%, crude), which was used directly for the next step.

### Step D: ethyl 4-((2S,5R)-4-(tert-butoxycarbonyl)-2,5-diethylpiperazin-1-yl)-2-chloropyrazolo[1,5-a][1,3,5]triazine-7-carboxylate

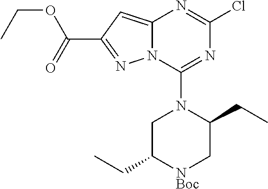 [SMILES: CCOC(=O)c1cc2nc(Cl)nc(N3C[C@@H](CC)N(C(=O)OC(C)(C)C)C[C@@H]3CC)n2n1]

A solution of ethyl 2,4-dichloropyrazolo[1,5-a][1,3,5]triazine-7-carboxylate (5.46 g, crude), tert-butyl (2R,5S)-2,5-diethylpiperazine-1-carboxylate (5.1 g, 21.07 mmol) and DIEA (13.5 g, 104.65 mmol) in THF (50 mL) was stirred at RT overnight. The solution was diluted with EtOAc (100 mL), washed with brine (30 mL×2), dried and concentrated. The residue was purified by flash with 0-20% EA in PE to give the titled compound (1.4 g). MS: M/e 467 (M+1)⁺.

### Step E: 4-((2S,5R)-4-(tert-butoxycarbonyl)-2,5-diethylpiperazin-1-yl)-2-oxo-1,2-dihydropyrazolo[1,5-a][1,3,5]triazine-7-carboxylic Acid

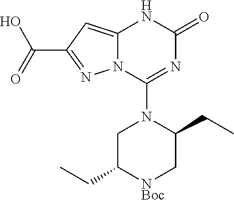 [SMILES: CC[C@@H]1CN(c2nc(=O)[nH]c3cc(C(=O)O)nn23)[C@@H](CC)CN1C(=O)OC(C)(C)C]

A solution of 2-(methylsulfonyl)ethan-1-ol (480 mg, 3.87 mmol) and NaH (60%, 290 mg, 7.25 mmol) in THF (15 mL) was stirred at RT for 30 min. ethyl 4-((2S,5R)-4-(tert-butoxycarbonyl)-2,5-diethylpiperazin-1-yl)-2-chloropyrazolo[1,5-a][1,3,5]triazine-7-carboxylate (1.4 g, 3.00 mmol) in THF (5 mL) was added to above solution and stirred at 70° C. overnight. The solution was quenched with H₂O (10 mL) and then evaporated to dryness to give product (1.26 g, crude), which was used directly for the next step without further purification. M/e 421 (M+1)⁺.

### Step F: tert-butyl (2R,5S)-2,5-diethyl-4-(7-(hydroxymethyl)-2-oxo-1,2-dihydropyrazolo[1,5-a][1,3,5]triazin-4-yl)piperazine-1-carboxylate

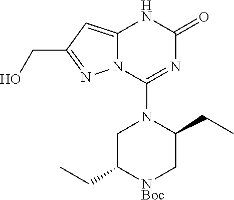 [SMILES: CC[C@@H]1CN(c2nc(=O)[nH]c3cc(CO)nn23)[C@@H](CC)CN1C(=O)OC(C)(C)C]

A solution of 4-((2S,5R)-4-(tert-butoxycarbonyl)-2,5-diethylpiperazin-1-yl)-2-oxo-1,2-dihydropyrazolo[1,5-a][1,3,5]triazine-7-carboxylic acid (1.26 g, crude) and CDI (0.61 g, 3.77 mmol) in THF (10 mL) was stirred at RT overnight. To a stirred solution of NaBH₄ (0.34 g, 8.95 mmol) in THF (5 mL) and H₂O (5 mL) at 0° C., was added above solution and stirred at RT overnight. The solution was diluted with EA (20 mL), washed with brine (10 mL×2), dried and evaporated. The residue was purified by flash with 0-10% MeOH in DCM to give the titled compound (0.4 g). M/e 407 (M+1)⁺.

### Step G: tert-butyl (2R,5S)-2,5-diethyl-4-(7-(hydroxymethyl)-1-methyl-2-oxo-1,2-dihydropyrazolo[1,5-a][1,3,5]triazin-4-yl)piperazine-1-carboxylate

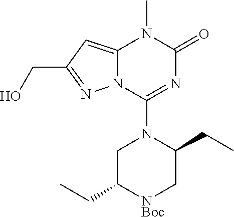 [SMILES: CC[C@@H]1CN(c2nc(=O)n(C)c3cc(CO)nn23)[C@@H](CC)CN1C(=O)OC(C)(C)C]

A solution of tert-butyl (2R,5S)-2,5-diethyl-4-(7-(hydroxymethyl)-2-oxo-1,2-dihydropyrazolo[1,5-a][1,3,5]triazin-4-yl)piperazine-1-carboxylate (400 mg, 0.99 mmol) and HMDS (206 mg, 1.28 mmol) in CH₃CN (10 mL) was stirred at 80° C. for 30 min. chloro(chloromethyl)dimethylsilane (183 mg, 1.28 mmol) was added to above solution and stirred at 80° C. for 3 hours. The solution was evaporated to dryness and then added to a solution of KF (286 mg, 4.93 mmol) in DMSO (6 mL) and H₂O (1 mL), stirred at 100° C. for 1 h. The solution was poured into H₂O (10 mL) and then extracted with EA (10 mL×2). The organic layer was washed with brine (10 mL), dried and evaporated. The residue was purified by flash with 0-10% MeOH in DCM to give the titled compound (400 mg, 96%). We 421 (M+1)⁺.

### Step H: tert-butyl (2R,5S)-2,5-diethyl-4-(1-methyl-7-(((methylsulfonyl)oxy) methyl)-2-oxo-1,2-dihydropyrazolo[1,5-a][1,3,5]triazin-4-yl)piperazine-1-carboxylate

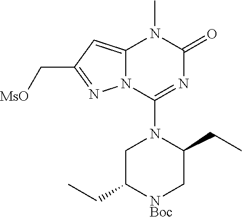 [SMILES: CC[C@@H]1CN(c2nc(=O)n(C)c3cc(COS(C)(=O)=O)nn23)[C@@H](CC)CN1C(=O)OC(C)(C)C]

A solution of tert-butyl (2R,5S)-2,5-diethyl-4-(7-(hydroxymethyl)-1-methyl-2-oxo-1,2-dihydropyrazolo[1,5-a][1,3,5]triazin-4-yl)piperazine-1-carboxylate (400 mg, 0.95 mmol), MsCl (140 mg, 1.22 mmol) and TEA (290 mg, 2.87 mmol) in DCM (10 mL) was stirred at 0° C. for 30 min. The solution was evaporated to dryness to give the titled compound (474 mg, 100%, crude), which was used directly for the next step. Me 499 (M+1)⁺.

### Step I: tert-butyl (2R,5S)-4-(7-(cyanomethyl)-1-methyl-2-oxo-1,2-dihydropyrazolo[1,5-a][1,3,5]triazin-4-yl)-2,5-diethylpiperazine-1-carboxylate

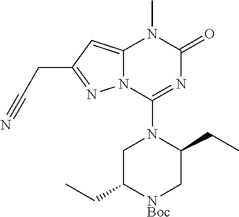 [SMILES: CC[C@@H]1CN(c2nc(=O)n(C)c3cc(CC#N)nn23)[C@@H](CC)CN1C(=O)OC(C)(C)C]

A solution of tert-butyl (2R,5S)-2,5-diethyl-4-(1-methyl-7-(((methylsulfonyl)oxy) methyl)-2-oxo-1,2-dihydropyrazolo[1,5-a][1,3,5]triazin-4-yl)piperazine-1-carboxylate (474 mg, crude), TMSCN (189 mg, 1.91 mmol) and TBAF (1M, 1.9 ml, 1.9 mmol) in DCM (10 mL) was stirred at RT overnight. The solution was washed with brine (10 mL), dried and evaporated. The residue was purified by flash with 0-5% MeOH in DCM to give the titled compound (230 mg). M/e 430 (M+1)⁺.

### Step J: 2-(4-((2S,5R)-2,5-diethylpiperazin-1-yl)-1-methyl-2-oxo-1,2-dihydro pyrazolo[1,5-a][1,3,5]triazin-7-yl)acetonitrile

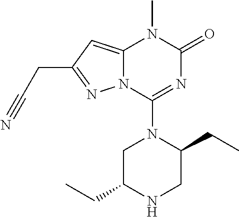 [SMILES: CC[C@@H]1CN(c2nc(=O)n(C)c3cc(CC#N)nn23)[C@@H](CC)CN1]

A solution of tert-butyl (2R,5S)-4-(7-(cyanomethyl)-1-methyl-2-oxo-1,2-dihydropyrazolo[1,5-a][1,3,5]triazin-4-yl)-2,5-diethylpiperazine-1-carboxylate (230 mg, 0.54 mmol) and TFA (2 mL) in DCM (10 mL) was stirred at RT for 3 hours. The solution was evaporated to dryness to give the titled compound (170 mg, 100%). M/e 330 (M+1)⁺.

### The Intermediate tert-butyl (2R,5S)-2,5-diethyl-4-(7-(hydroxymethyl)-1-methyl-2-oxo-1,2-dihydropyrazolo[1,5-a][1,3,5]triazin-4-yl)piperazine-1-carboxylate Also can be Synthesized by Procedure as Below

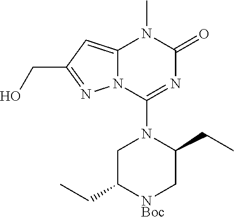 [SMILES: CC[C@@H]1CN(c2nc(=O)n(C)c3cc(CO)nn23)[C@@H](CC)CN1C(=O)OC(C)(C)C]

### Step A: ethyl 4-((2S,5R)-4-(tert-butoxycarbonyl)-2,5-diethylpiperazin-1-yl)-2-oxo-1,2-dihydropyrazolo[1,5-a][1,3,5]triazine-7-carboxylate

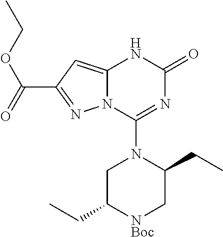 [SMILES: CCOC(=O)c1cc2[nH]c(=O)nc(N3C[C@@H](CC)N(C(=O)OC(C)(C)C)C[C@@H]3CC)n2n1]

A solution of ethyl 4-((2S,5R)-4-(tert-butoxycarbonyl)-2,5-diethylpiperazin-1-yl)-2-chloropyrazolo[1,5-a][1,3,5]triazine-7-carboxylate (13 g, 0.028 mol), Pd₂(dba)₃ (1.28 g, 1.40 mmol), tBuXPhos (1.19 g, 2.8 mmol) and K₂CO₃ (11.55 g, 0.084 mol) in dioxane (150 ml) and H₂O (30 ml) was stirred at 60° C. overnight. The reaction solution was poured into water (300 ml) and then extracted with EA (100 ml×3). The organic layer was washed with brine (50 ml), dried and concentrated. The resulting residue was purified by flash column chromatography with 30-100% EA in PE to give the titled product (11 g, 88%). MS: M/e 449 (M+1)⁺.

### Step B: ethyl 4-((2S,5R)-4-(tert-butoxycarbonyl)-2,5-diethylpiperazin-1-yl)-1-methyl-2-oxo-1,2-dihydropyrazolo[1,5-a][1,3,5]triazine-7-carboxylate

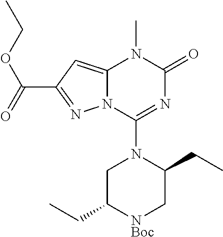 [SMILES: CCOC(=O)c1cc2n(C)c(=O)nc(N3C[C@@H](CC)N(C(=O)OC(C)(C)C)C[C@@H]3CC)n2n1]

A solution of ethyl 4-((2S,5R)-4-(tert-butoxycarbonyl)-2,5-diethylpiperazin-1-yl)-2-oxo-1,2-dihydropyrazolo[1,5-a][1,3,5]triazine-7-carboxylate (11 g, 0.025 mol) and HMDS (5.14 g, 0.032 mol) in MeCN (110 ml) was stirred at 80° C. for 30 min. chloro(chloromethyl)dimethylsilane (4.56 g, 0.032 mol) was added to above solution and stirred at 80° C. for 3 h. The reaction solution was evaporated to dryness. The resulting residue and KF (7.12 g, 0.12 mol) in DMSO (60 ml) and H₂O (15 ml) was stirred at 100° C. for 30 min. The reaction solution was poured into water (100 ml) and then extracted with EA (30 ml×3). The organic layer was washed with brine (30 ml), dried and concentrated. The residue was purified by flash column chromatography with 25-40% EA in PE to give the titled product (10 g, 88%). MS: M/e 463 (M+1)⁺.

### Step C: 4-((2S,5R)-4-(tert-butoxycarbonyl)-2,5-diethylpiperazin-1-yl)-1-methyl-2-oxo-1,2-dihydropyrazolo[1,5-a][1,3,5]triazine-7-carboxylic Acid

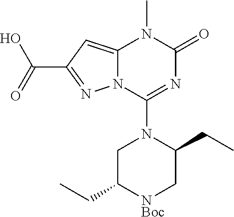 [SMILES: CC[C@@H]1CN(c2nc(=O)n(C)c3cc(C(=O)O)nn23)[C@@H](CC)CN1C(=O)OC(C)(C)C]

A solution of ethyl 4-((2S,5R)-4-(tert-butoxycarbonyl)-2,5-diethylpiperazin-1-yl)-1-methyl-2-oxo-1,2-dihydropyrazolo[1,5-a][1,3,5]triazine-7-carboxylate (10 g, 0.022 mol) and LiOH (2.1 g, 0.088 mol) in THF (100 ml) and H₂O (30 ml) was stirred at 0° C. for 40 min. The reaction solution was diluted with H₂O (150 ml), adjusted to pH=4 and then extracted with EA (30 ml×2). The organic layer was dried over Na₂SO₄, filtered and then evaporated to dryness to give the titled product (9.4 g, 100%). MS: M/e 435 (M+1)⁺.

### Step D: tert-butyl (2R,5S)-2,5-diethyl-4-(7-(hydroxymethyl)-1-methyl-2-oxo-1,2-dihydropyrazolo[1,5-a][1,3,5]triazin-4-yl)piperazine-1-carboxylate

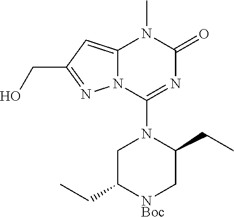 [SMILES: CC[C@@H]1CN(c2nc(=O)n(C)c3cc(CO)nn23)[C@@H](CC)CN1C(=O)OC(C)(C)C]

A solution of 4-((2S,5R)-4-(tert-butoxycarbonyl)-2,5-diethylpiperazin-1-yl)-1-methyl-2-oxo-1,2-dihydropyrazolo[1,5-a][1,3,5]triazine-7-carboxylic acid (9.4 g, 0.022 mol) and CDI (5.26 g, 0.032 mol) in THF (100 ml) was stirred at rt overnight. The solution was added to a pre-stirred solution of NaBH₄ (2.47 g, 0.065 mol) in THF (30 ml) and H₂O (30 ml) at 0° C. for 10 min and then stirred at 0° C. for 30 min. The resulting solution was poured into water (100 ml) and then extracted with EA (30 ml×3). The organic layer was dried and concentrated. The residue was purified by flash column chromatography with 0-5% MeOH in DCM to give the titled product (9 g, 98%). MS: M/e 421 (M+1)⁺.

### Intermediate 6: 2-(4-((2S,5R)-5-ethyl-2-methylpiperazin-1-yl)-1-methyl-2-oxo-1,2-dihydropyrazolo[1,5-a][1,3,5]triazin-7-yl)acetonitrile

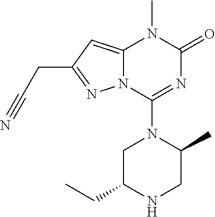 [SMILES: CC[C@@H]1CN(c2nc(=O)n(C)c3cc(CC#N)nn23)[C@@H](C)CN1]

### Step A: ethyl 4-((2S,5R)-4-(tert-butoxycarbonyl)-5-ethyl-2-methylpiperazin-1-yl)-2-hydroxypyrazolo[1,5-a][1,3,5]triazine-7-carboxylate

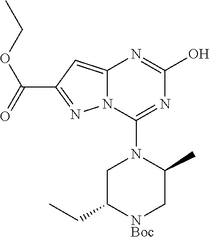 [SMILES: CCOC(=O)c1cc2nc(O)nc(N3C[C@@H](CC)N(C(=O)OC(C)(C)C)C[C@@H]3C)n2n1]

A solution of ethyl 4-((2S,5R)-4-(tert-butoxycarbonyl)-5-ethyl-2-methylpiperazin-1-yl)-2-chloropyrazolo[1,5-a][1,3,5]triazine-7-carboxylate (7 g, 15.5 mmol), Pd₂(dba)₃ (710 mg, 0.78 mmol), tBuXPhos (659 mg, 1.55 mmol) and K₂CO₃ (6.4 g, 46.5 mmol) in dioxane (70 mL) and H₂O (18 mL) was stirred at 60° C. overnight. The reaction solution was poured into water (100 mL) and then extracted with EA (50 mL×2). The aqueous layer was adjusted pH to 4-5 with HCl (1N) and extracted with EA (50 mL×2). The organic layer was washed with brine (50 mL), dried and concentrated. The resulting residue was purified by flash column chromatography with 0-10% MeOH in DCM to give the titled compound (3.7 g, 54.8%). MS: M/e 435 (M+1)⁺.

### Step B: ethyl 4-((2S,5R)-4-(tert-butoxycarbonyl)-5-ethyl-2-methylpiperazin-1-yl)-1-methyl-2-oxo-1,2-dihydropyrazolo[1,5-a][1,3,5]triazine-7-carboxylate

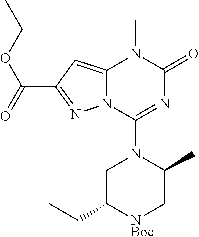 [SMILES: CCOC(=O)c1cc2n(C)c(=O)nc(N3C[C@@H](CC)N(C(=O)OC(C)(C)C)C[C@@H]3C)n2n1]

A solution of ethyl 4-((2S,5R)-4-(tert-butoxycarbonyl)-5-ethyl-2-methylpiperazin-1-yl)-2-hydroxypyrazolo[1,5-a][1,3,5]triazine-7-carboxylate (3.7 g, 8.5 mmol) and HMDS (1.8 g, 11.1 mmol) in MeCN (25 mL) was stirred at 80° C. for 1 h. chloro(chloromethyl)dimethylsilane (1.6 g, 11.1 mmol) was added to above solution and stirred at 80° C. for 4 hours. The reaction solution was evaporated to dryness. The resulting residue was added KF (2.5 g, 42.5 mmol) in DMSO (15 mL) and H₂O (15 mL) was stirred at 100° C. for 1 h. The reaction solution was poured into water (100 mL) and then extracted with EA (30 mL×3). The organic layer was washed with brine (30 mL), dried and concentrated. The residue was purified by flash column chromatography with 0-10% MeOH in DCM to give the titled compound (3.2 g, 83.5%). MS: M/e 449 (M+1)⁺.

### Step C: 4-((2S,5R)-4-(tert-butoxycarbonyl)-5-ethyl-2-methylpiperazin-1-yl)-1-methyl-2-oxo-1,2-dihydropyrazolo[1,5-a][1,3,5]triazine-7-carboxylic Acid

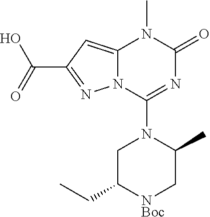 [SMILES: CC[C@@H]1CN(c2nc(=O)n(C)c3cc(C(=O)O)nn23)[C@@H](C)CN1C(=O)OC(C)(C)C]

A solution of ethyl 4-((2S,5R)-4-(tert-butoxycarbonyl)-5-ethyl-2-methylpiperazin-1-yl)-1-methyl-2-oxo-1,2-dihydropyrazolo[1,5-a][1,3,5]triazine-7-carboxylate (3.2 g, 0.007 mol) and LiOH (686 mg, 28.58 mmol) in THF (30 mL) and H₂O (8 mL) was stirred at RT for 1 h. The reaction solution was diluted with H₂O (50 mL), adjusted to pH=2-3 and then extracted with EA (30 mL×2). The organic layer was dried over Na₂SO₄, filtered and then evaporated to dryness to give the titled compound (3 g, crude). MS: M/e 421 (M+1)⁺.

### Step D: tert-butyl (2R,5S)-2-ethyl-4-(7-(hydroxymethyl)-1-methyl-2-oxo-1,2-dihydropyrazolo[1,5-a][1,3,5]triazin-4-yl)-5-methylpiperazine-1-carboxylate

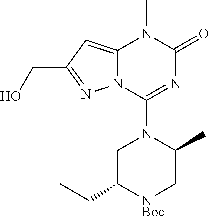 [SMILES: CC[C@@H]1CN(c2nc(=O)n(C)c3cc(CO)nn23)[C@@H](C)CN1C(=O)OC(C)(C)C]

A solution of 4-((2S,5R)-4-(tert-butoxycarbonyl)-5-ethyl-2-methylpiperazin-1-yl)-1-methyl-2-oxo-1,2-dihydropyrazolo[1,5-a][1,3,5]triazine-7-carboxylic acid (3 g, 7.14 mmol) and CDI (1.62 g, 10 mmol) in THF (15 mL) was stirred at RT for 2 hours. The solution was added to a pre-stirred solution of NaBH₄ (814 mg, 21.42 mmol) in THF (7 mL) and H₂O (7 mL) at 0° C. and then stirred at 0° C. for 1 h. The resulting solution was poured into water (50 mL) and then extracted with EA (20 mL×3). The organic layer was dried and concentrated. The residue was purified by flash column chromatography with 0-10% MeOH in DCM to give the titled compound (1.4 g, 48%). MS: M/e 407 (M+1)⁺.

### Step E: tert-butyl (2R,5S)-2-ethyl-5-methyl-4-(1-methyl-7-(((methylsulfonyl)oxy) methyl)-2-oxo-1,2-dihydropyrazolo[1,5-a][1,3,5]triazin-4-yl)piperazine-1-carboxylate

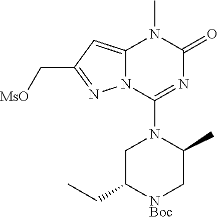 [SMILES: CC[C@@H]1CN(c2nc(=O)n(C)c3cc(COS(C)(=O)=O)nn23)[C@@H](C)CN1C(=O)OC(C)(C)C]

A solution of tert-butyl (2R,5S)-2-ethyl-4-(7-(hydroxymethyl)-1-methyl-2-oxo-1,2-dihydropyrazolo[1,5-a][1,3,5]triazin-4-yl)-5-methylpiperazine-1-carboxylate (1.4 g, 3.45 mmol), Ms₂O (660 mg, 3.79 mmol) and TEA (523 mg, 5.18 mmol) in DCM (20 mL) was stirred at 0° C. for 45 min. The reaction solution was washed with aq. NaHCO₃ (30 mL) and brine (30 mL), dried over Na₂SO₄, filtered and which was used directly for the next step. MS: M/e 485 (M+1)⁺.

### Step F: tert-butyl (2R,5S)-4-(7-(cyanomethyl)-1-methyl-2-oxo-1,2-dihydro pyrazolo[1,5-a][1,3,5]triazin-4-yl)-2-ethyl-5-methylpiperazine-1-carboxylate

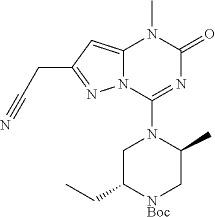 [SMILES: CC[C@@H]1CN(c2nc(=O)n(C)c3cc(CC#N)nn23)[C@@H](C)CN1C(=O)OC(C)(C)C]

A solution of tert-butyl (2R,5S)-2-ethyl-5-methyl-4-(1-methyl-7-(((methylsulfonyl)oxy)methyl)-2-oxo-1,2-dihydropyrazolo[1,5-a][1,3,5]triazin-4-yl) piperazine-1-carboxylate (1.67 g, 3.45 mmol), TMSCN (683 mg, 6.9 mmol) and TBAF (1 M in THF, 6.9 mL, 6.9 mmol) in DCM (20 mL) was stirred at RT for 1 h. The solution was washed with brine (30 mL×2), dried and concentrated. The resulting residue was purified by flash column chromatography with 0-10% MeOH in DCM to give the titled compound (1 g, 69.9%). MS: M/e 416 (M+1)⁺.

### Step G: 2-(4-((2S,5R)-5-ethyl-2-methylpiperazin-1-yl)-1-methyl-2-oxo-1,2-dihydropyrazolo[1,5-a][1,3,5]triazin-7-yl)acetonitrile

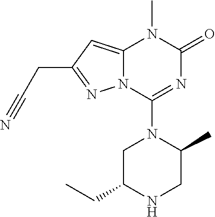 [SMILES: CC[C@@H]1CN(c2nc(=O)n(C)c3cc(CC#N)nn23)[C@@H](C)CN1]

A solution of tert-butyl (2R,5S)-4-(7-(cyanomethyl)-1-methyl-2-oxo-1,2-dihydropyrazolo[1,5-a][1,3,5]triazin-4-yl)-2-ethyl-5-methylpiperazine-1-carboxylate (1 g, 2.41 mmol) and TMSOTf (642 mg, 2.89 mmol) in DCM (10 mL) was stirred at RT for 2.5 hours. The reaction solution was concentrated under reduced pressure. The residue was diluted with DCM (50 mL), washed with aq. NaHCO₃ (30 mL) and brine (30 mL), dried over Na₂SO₄, filtered and evaporated to dryness to give the titled product (610 mg, 80%), which was used directly for the next step without further purification. MS: M/e 316 (M+1)⁺.

### Compound A1: 7-((2S,5R)-2,5-dimethyl-4-(1-(quinoxalin-6-yl)ethyl)piperazin-1-yl)-4-methyl-5-oxo-4,5-dihydropyrazolo[1,5-a]pyrimidine-2-carbonitrile

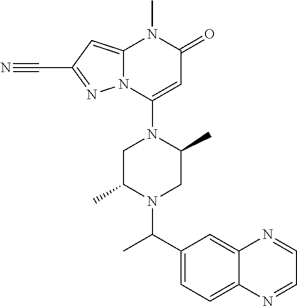 [SMILES: CC(c1ccc2nccnc2c1)N1C[C@H](C)N(c2cc(=O)n(C)c3cc(C#N)nn23)C[C@H]1C]

To a solution of 7-((2S,5R)-2,5-dimethylpiperazin-1-yl)-4-methyl-5-oxo-4,5-dihydropyrazolo[1,5-a]pyrimidine-2-carbonitrile in acetonitrile (5 mL) was added (cyanomethyl)trimethylphosphonium iodide (46 mg, 0.2 mmol), 1-(quinoxalin-6-yl)ethan-1-ol (17.4 mg, 0.2 mmol) and DIPEA (129 mg, 1 mmol). The reaction mixture was sealed and stirred under nitrogen protection at 105° C. overnight. The mixture was added H₂O and extracted by ethyl acetate. The organic layer was washed with brine, dried over Na₂SO₄, filtered, and concentrated. The resulting residue was purified by flash column chromatography to give the titled compound (6 mg, 13%). ¹H NMR (400 MHz, CD₃OD) δ 8.87 (t, J=4.9 Hz, 2H), 8.13-8.00 (m, 3H), 6.62 (d, J=3.6 Hz, 1H), 5.49 (d, J=20.0 Hz, 1H), 4.52-4.54 (m, 0.5H), 4.04 (d, J=6.3 Hz, 0.5H), 3.91 (d, J=6.6 Hz, 0.5H), 3.80 (d, J=12.2 Hz, 0.5H), 3.69 (s, 1H), 3.64-3.55 (m, 1H), 3.48 (s, 3H), 3.23-3.17 (in, 0.5H), 2.96 (in, 0.5H), 2.91 (d, J=10.4 Hz, 1H), 2.20 (d, J=10.6 Hz, 1H), 1.50-1.38 (in, 4.5H), 1.26 (d, J=6.4 Hz, 1.5H), 1.19 (d, J=6.6 Hz, 1.5H), 1.11 (d, J=6.5 Hz, 1.5H) ppm. MS: M/e 443 (M+1)⁺.

### Compound A2: 2-(7-((2S,5R)-2,5-dimethyl-4-(1-(quinoxalin-6-yl)ethyl)piperazin-1-yl)-4-methyl-5-oxo-4,5-dihydropyrazolo[1,5-a]pyrimidin-2-yl)acetonitrile

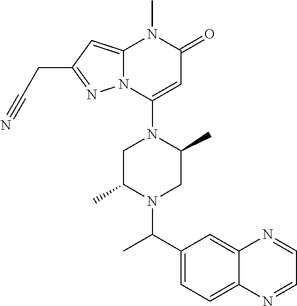 [SMILES: CC(c1ccc2nccnc2c1)N1C[C@H](C)N(c2cc(=O)n(C)c3cc(CC#N)nn23)C[C@H]1C]

To a solution of 2-(7-((2S,5R)-2,5-dimethylpiperazin-1-yl)-4-methyl-5-oxo-4,5-dihydropyrazolo[1,5-a]pyrimidin-2-yl)acetonitrile from last step in CH₃CN (5 mL) was added (cyanomethyl)trimethylphosphonium iodide (46 mg, 0.2 mmol), 1-(quinoxalin-6-yl)ethan-1-ol (17.4 mg, 0.2 mmol) and DIPEA (129 mg, 1 mmol). The reaction mixture was sealed and stirred under nitrogen protection at 105° C. overnight. The mixture was added H₂O and extracted by ethyl acetate. The organic layer was washed with brine, dried over Na₂SO₄, filtered, and concentrated. The resulting crude product was purified by column chromatography to give the titled Compound A2 (6 mg, 21% for two steps). Another batch Compound A2 (30 mg) was separated into Compound A2a (14 mg) and Compound A2b (10 mg) by chiral Prep-HPLC. The chiral separation conditions are shown below.

| | |
|---|---|
| Column | CHIRAL Cellulose SB |
| Column Size | 2 cm × 25 cm, 5 um |
| Mobile Phase A | MtBE(0.5% 2 mM NH3—MEOH) |
| Mobile Phase B | MeOH |
| Flow Rate | 20 mL/min |
| Wave Length | UV 220 nm |
| Temperature | 25° C. |
| Prep-HPLC Equipment | Prep-HPLC-Gilson |

Compound A2: ¹HNMR (400 MHz, CD₃OD) δ 8.87 (s, 2H), 8.06 (d, J=14.0 Hz, 3H), 6.10 (s, 1H), 5.30 (d, J=14.0 Hz, 1H), 4.62 (s, 0.5H), 4.02 (s, 0.5H), 3.98 (s, 2H), 3.85 (d, J=23.1 Hz, 1H), 3.73 (d, J=37.2 Hz, 1H), 3.54 (s, 1H), 3.46 (s, 3H), 3.17 (d, J=12.3 Hz, 1H), 2.91 (t, J=15.3 Hz, 2H), 1.44 (d, J=8.0 Hz, 4H), 1.27 (s, 2H), 1.16 (d, J=30.5 Hz, 3H) ppm. MS: M/e 457 (M+1)⁺.

Compound A2a (the earlier peak): ¹HNMR (400 MHz, CD₃OD) δ 8.92-8.81 (m, 2H), 8.08 (d, J=8.8 Hz, 2H), 8.04 (s, 1H), 6.10 (s, 1H), 5.32 (s, 1H), 4.62 (s, 1H), 3.98 (s, 2H), 3.88 (dd, J=13.3, 6.8 Hz, 1H), 3.86-3.73 (m, 2H), 3.67 (dd, J=13.1, 10.1 Hz, 1H), 3.46 (s, 3H), 2.94 (dd, J=12.0, 3.7 Hz, 1H), 2.16-2.21 (m, 1H), 1.46 (d, J=6.5 Hz, 3H), 1.27 (m, 3H), 1.20 (d, J=6.6 Hz, 3H) ppm. MS: M/e 457 (M+1)⁺.

Compound A2b (the later peak): ¹HNMR (400 MHz, CD₃OD) δ 8.87 (d, J=4.3 Hz, 2H), 8.15-8.07 (m, 2H), 8.04 (d, J=8.7 Hz, 1H), 6.11 (s, 1H), 5.28 (s, 1H), 5.02-4.86 (m, 1H), 4.02 (d, J=6.5 Hz, 1H), 3.99 (s, 2H), 3.54 (d, J=2.0 Hz, 2H), 3.46 (s, 3H), 3.17 (dd, J=11.8, 3.6 Hz, 1H), 2.89 (d, J=9.1 Hz, 2H), 1.43 (t, J=7.2 Hz, 6H), 1.12 (d, J=6.5 Hz, 3H) ppm. MS: M/e 457 (M+1)⁺.

### Compound A3: 2-(7-((2S,5R)-2,5-dimethyl-4-(1-(3-methylquinoxalin-6-yl)ethyl)piperazin-1-yl)-4-methyl-5-oxo-4,5-dihydropyrazolo[1,5-a]pyrimidin-2-yl)acetonitrile

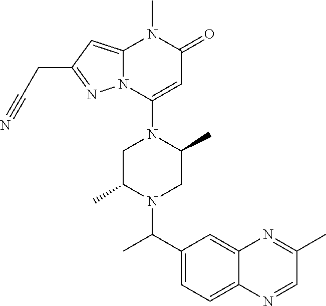 [SMILES: Cc1cnc2ccc(C(C)N3C[C@H](C)N(c4cc(=O)n(C)c5cc(CC#N)nn45)C[C@H]3C)cc2n1]

To a mixture of 2-(7-((2S,5R)-2,5-dimethylpiperazin-1-yl)-4-methyl-5-oxo-4,5-dihydropyrazolo[1,5-a]pyrimidin-2-yl)acetonitrile from last step in acetonitrile (8 mL) was added trimethyl(prop-2-yn-1-yl)phosphonium (800 mg, 3.32 mmol), 1-(3-methylquinoxalin-6-yl)ethan-1-ol (624 mg, 3.32 mmol) and DIPEA (1.29 g, 10 mmol). The reaction mixture was sealed and stirred under nitrogen protection at 105° C. overnight. The mixture was added H₂O and extracted by ethyl acetate. The organic phase was washed with brine, dried over Na₂SO₄, filtered, and concentrated. The resulting residue was purified by column chromatography to give the titled Compound A3, which was separated into Compound A3a (85 mg) and Compound A3b (105 mg) by Prep-HPLC (Method B).

Compound A3: ¹HNMR (400 MHz, CD₃OD) δ 8.78 (d, J=5.4 Hz, 1H), 8.08-7.90 (m, 3H), 6.10 (d, J=1.8 Hz, 1H), 5.30 (d, J=13.3 Hz, 1H), 4.59 (d, J=18.2 Hz, 1H), 4.04-3.94 (m, 2H), 3.87-3.80 (m, 1H), 3.76 (dd, J=12.2, 3.2 Hz, 1H), 3.71-3.62 (m, 1H), 3.46 (s, 3H), 3.17 (dd, J=11.8, 3.7 Hz, 1H), 2.91 (ddd, J=11.9, 10.7, 4.7 Hz, 2H), 2.76 (d, J=2.7 Hz, 3H), 1.43 (dd, J=7.3, 2.6 Hz, 3H), 1.30-1.25 (m, 3H), 1.20 (d, J=6.6 Hz, 1.5H), 1.11 (d, J=6.5 Hz, 1.5H) ppm. MS: M/e 471 (M+1)⁺.

Compound A3a (the earlier peak): ¹HNMR (400 MHz, CD₃OD) δ 8.79 (s, 1H), 8.05 (d, J=8.6 Hz, 1H), 7.99 (s, 1H), 7.94 (s, 1H), 6.10 (s, 1H), 5.28 (s, 1H), 4.91 (s, 1H), 3.99 (d, J=5.1 Hz, 3H), 3.54 (s, 2H), 3.45 (s, 3H), 3.16 (d, J=11.4 Hz, 1H), 2.88 (d, J=10.9 Hz, 2H), 2.77 (s, 3H), 1.46-1.39 (m, 6H), 1.11 (d, J=6.4 Hz, 3H). MS: M/e 471 (M+1)⁺.

Compound A3b (the later peak): ¹HNMR (400 MHz, CD₃OD) δ 8.77 (s, 1H), 8.02 (d, J=8.6 Hz, 1H), 7.97 (s, 1H), 7.94 (s, 1H), 6.10 (s, 11H), 5.32 (s, 1H), 4.61 (s, 1H), 3.98 (s, 2H), 3.88-3.80 (m, 2H), 3.75 (d, J=12.2 Hz, 1H), 3.66 (s, 1H), 3.46 (s, 3H), 2.97-2.89 (m, 1H), 2.76 (s, 3H), 2.17 (d, J=12.0 Hz, 1H), 1.45 (d, J=6.5 Hz, 3H), 1.29 (s, 3H), 1.20 (d, J=6.6 Hz, 3H). MS: M/e 471.2 (M+1)⁺.

### Compound A4: 2-(7-((2S,5R)-5-ethyl-2-methyl-4-(1-(quinoxalin-6-yl)ethyl)piperazin-1-yl)-5-oxo-4,5-dihydropyrazolo[1,5-a]pyrimidin-2-yl)acetonitrile

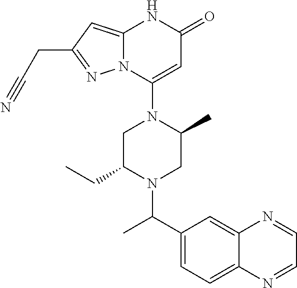 [SMILES: CC[C@@H]1CN(c2cc(=O)[nH]c3cc(CC#N)nn23)[C@@H](C)CN1C(C)c1ccc2nccnc2c1]

### Step A: tert-butyl (2R,5S)-4-(2-bromo-5-chloropyrazolo[1,5-a]pyrimidin-7-yl)-2-ethyl-5-methylpiperazine-1-carboxylate

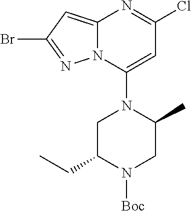 [SMILES: CC[C@@H]1CN(c2cc(Cl)nc3cc(Br)nn23)[C@@H](C)CN1C(=O)OC(C)(C)C]

A mixture of tert-butyl (2R,5S)-2-ethyl-5-methylpiperazine-1-carboxylate (640 mg, 2.8 mmol), 2-bromo-5,7-dichloropyrazolo[1,5-a]pyrimidine (500 mg, 1.9 mmol) and DIPEA (486 mg, 3.8 mmol) in THF (10 mL) was stirred at room temperature for 2 hours. The solvent was removed under vacuum. The resulting crude product was purified by silica column chromatography (PE:EtOAc=50: 1) to give the titled compound (350 mg, 41%). MS: M/e 458 (M+1)⁺.

### Step B: tert-butyl (2R,5S)-4-(2-bromo-5-(tert-butoxy)pyrazolo[1,5-a]pyrimidin-7-yl)-2-ethyl-5-methylpiperazine-1-carboxylate

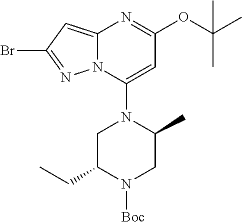 [SMILES: CC[C@@H]1CN(c2cc(OC(C)(C)C)nc3cc(Br)nn23)[C@@H](C)CN1C(=O)OC(C)(C)C]

A mixture of tert-butyl (2R,5S)-4-(2-bromo-5-chloropyrazolo[1,5-a]pyrimidin-7-yl)-2-ethyl-5-methylpiperazine-1-carboxylate (350 mg, 0.77 mmol), t-BuOK (170 mg, 1.54 mmol) and 18-crown-6 (25 mg, 0.1 mmol) in toluene (5 mL) was stirred at 60° C. for 30 mins in microwave. The solvent was removed under vacuum. The resulting crude product was purified by silica column chromatography (PE:EtOAc=50: 1) to give the titled compound (300 mg, 79%). MS: M/e 496 (M+1)⁺.

### Step C: methyl 5-(tert-butoxy)-7-((2S,5R)-4-(tert-butoxycarbonyl)-5-ethyl-2-methylpiperazin-1-yl)pyrazolo[1,5-a]pyrimidine-2-carboxylate

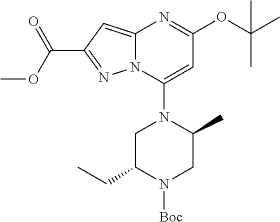 [SMILES: CC[C@@H]1CN(c2cc(OC(C)(C)C)nc3cc(C(=O)OC)nn23)[C@@H](C)CN1C(=O)OC(C)(C)C]

A mixture of tert-butyl (2R,5S)-4-(2-bromo-5-(tert-butoxy)pyrazolo[1,5-a]pyrimidin-7-yl)-2-ethyl-5-methylpiperazine-1-carboxylate (300 mg, 0.61 mmol), Pd(dppf)Cl₂ (44 mg, 0.06 mmol) and TEA (310 mg, 3.1 mmol) in MeOH (10 mL) was heated to 90° C. overnight under CO atmosphere in autoclave. The solvent was removed under vacuum. The resulting crude product was purified by silica column chromatography (PE:EtOAc=20:1) to give the target compound (240 mg, 83% yield) as a yellow oil. MS: M/e 476 (M+1)⁺.

### Step D: tert-butyl (2R,5S)-4-(5-(tert-butoxy)-2-(hydroxymethyl)pyrazolo[1,5-a]pyrimidin-7-yl)-2-ethyl-5-methylpiperazine-1-carboxylate

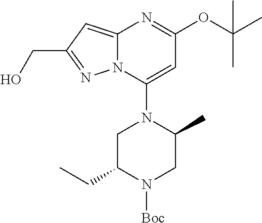 [SMILES: CC[C@@H]1CN(c2cc(OC(C)(C)C)nc3cc(CO)nn23)[C@@H](C)CN1C(=O)OC(C)(C)C]

To a solution of methyl 5-(tert-butoxy)-7-((2S,5R)-4-(tert-butoxycarbonyl)-5-ethyl-2-methylpiperazin-1-yl)pyrazolo[1,5-a]pyrimidine-2-carboxylate (220 mg, 0.46 mmol) in THF (10 mL) was added LiAlH₄ solution (0.5 mL, 0.5 mmol, 1 M in THF) at 0° C. The resulting mixture was stirred at room temperature for another 30 mins. The reaction was quenched by H₂O (5 mL) and extracted with EtOAc (50 mL×3). The organic layer were concentrated to obtained crude product which was further purified by silica column chromatography (PE:EtOAc=10:1) to give the titled compound (170 mg, 82%). MS: M/e 448 (M+1)⁺.

### Step E: tert-butyl (2R,5S)-4-(5-(tert-butoxy)-2-(((methylsulfonyl)oxy)methyl)pyrazolo[1,5-a]pyrimidin-7-yl)-2-ethyl-5-methylpiperazine-1-carboxylate

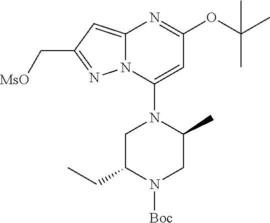 [SMILES: CC[C@@H]1CN(c2cc(OC(C)(C)C)nc3cc(COS(C)(=O)=O)nn23)[C@@H](C)CN1C(=O)OC(C)(C)C]

To a solution of tert-butyl (2R,5S)-4-(5-(tert-butoxy)-2-(hydroxymethyl)pyrazolo[1,5-a]pyrimidin-7-yl)-2-ethyl-5-methylpiperazine-1-carboxylate (130 mg, 0.29 mmol) and DIPEA (120 mg, 0.6 mmol) in DCM (5 mL) was added MsCl (35 mg, 0.3 mmol) at 0° C. The resulting mixture was stirred at room temperature for 30 mins. The reaction mixture was quenched by H₂O (5 mL) and extracted with DCM (10 mL×2). The organic layers were concentrated to give the titled compound (130 mg), which was used directly in the next step without further purification. MS: M/e 526 (M+1)⁺.

### Step F: tert-butyl (2R,5S)-4-(5-(tert-butoxy)-2-(cyanomethyl)pyrazolo[1,5-a]pyrimidin-7-yl)-2-ethyl-5-methylpiperazine-1-carboxylate

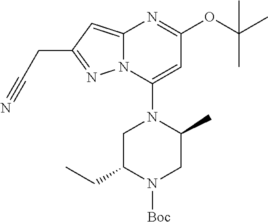 [SMILES: CC[C@@H]1CN(c2cc(OC(C)(C)C)nc3cc(CC#N)nn23)[C@@H](C)CN1C(=O)OC(C)(C)C]

To a solution of tert-butyl (2R,5S)-4-(5-(tert-butoxy)-2-(((methylsulfonyl)oxy)methyl)pyrazolo[1,5-a]pyrimidin-7-yl)-2-ethyl-5-methylpiperazine-1-carboxylate (130 mg) and TMSCN (90 mg, 0.9 mmol) in DCM (5 mL) was added TBAF THF solution (0.9 mL, 0.9 mmol, 1 M) at room temperature. The resulting mixture was stirred at room temperature for 2 hours. The reaction mixture was quenched by H₂O (5 mL) and extracted with DCM (10 mL×2). The organic layers were concentrated and purified by Prep-TLC (PE:EtOAc=1:1) to give the titled compound (60 mg, 49% yield for 2 steps). MS: M/e 457 (M+1)⁺.

### Step G: 2-(7-((2S,5R)-5-ethyl-2-methylpiperazin-1-yl)-5-oxo-4,5-dihydropyrazolo[1,5-a]pyrimidin-2-yl)acetonitrile

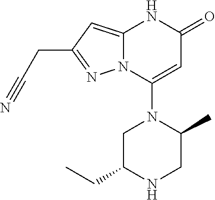 [SMILES: CC[C@@H]1CN(c2cc(=O)[nH]c3cc(CC#N)nn23)[C@@H](C)CN1]

To a solution of tert-butyl (2R,5S)-4-(5-(tert-butoxy)-2-(cyanomethyl)pyrazolo[1,5-a]pyrimidin-7-yl)-2-ethyl-5-methylpiperazine-1-carboxylate (60 mg, 0.13 mmol) in DCM (5 mL) was added TFA (2 mL). The resulting mixture was stirred at room temperature for 2 hours. The reaction mixture was neutralized with NH₃ MeOH solution to adjust pH=8. The solvent was concentrated and purified by prep-TLC to give the titled compound (50 mg, crude). MS: M/e 301 (M+1)⁺

### Step H: 2-(7-((2S,5R)-5-ethyl-2-methyl-4-(1-(quinoxalin-6-yl)ethyl)piperazin-1-yl)-5-oxo-4,5-dihydropyrazolo[1,5-a]pyrimidin-2-yl)acetonitrile

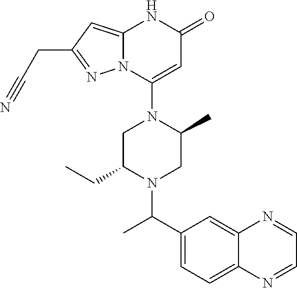 [SMILES: CC[C@@H]1CN(c2cc(=O)[nH]c3cc(CC#N)nn23)[C@@H](C)CN1C(C)c1ccc2nccnc2c1]

A mixture of 2-(7-((2S,5R)-5-ethyl-2-methylpiperazin-1-yl)-5-oxo-4,5-dihydropyrazolo[1,5-a]pyrimidin-2-yl)acetonitrile (40 mg, 0.13 mmol), 6-(1-chloroethyl)quinoxaline (128 mg, 0.65 mmol) and DIPEA (166 mg, 1.3 mmol) in DMSO (5 mL) was heated to 100° C. overnight. The reaction was diluted with EtOAc (20 mL) and washed by brine (10 mL×2). The organic layers were concentrated and purified by prep-TLC (DCM:MeOH=5:1) to give the titled Compound A4 which was further purified by Prep-HPLC (Method A) to give the Compound A4a (0.54 mg) and Compound A4b (0.63 mg).

Compound A4a (the earlier peak): ¹HNMR (400 MHz, CD₃OD) δ 8.88-8.87 (m, 2H), 8.13-8.02 (m, 3H), 5.93 (s, 1H), 5.22 (s, 1H), 4.11 (d, J=6.7 Hz, 1H), 3.94 (s, 2H), 3.83-3.81 (m, 1H), 3.44-3.42 (m, 1H), 3.13-3.12 (m, 1H), 2.94-2.92 (m, 1H), 2.45-2.44 (m, 1H), 2.03-2.01 (m 1H), 1.82-1.81 (m, 1H), 1.63-1.61 (m, 1H), 1.46-1.44 (m, 6H), 0.65 (t, J=7.4 Hz, 3H) ppm. MS: M/e 457 (M+1)⁺.

Compound A4b (the later peak): ¹HNMR (400 MHz, CD₃OD) δ 8.87-8.86 (m, 2H), 8.09-8.02 (m, 3H), 5.93 (s, 1H), 5.26 (s, 1H), 4.64-4.63 (m, 1H), 4.15-4.12 (m, 1H), 3.96-3.92 (m, 3H), 3.67-3.64 (m, 1H), 2.91-2.90 (m, 1H), 2.21-2.17 (m, 1H), 2.05-1.94 (m, 2H), 1.64-1.63 (m, 1H), 1.44 (d, J=6.5 Hz, 3H), 1.20 (d, J=6.6 Hz, 3H), 1.02 (t, J=7.4 Hz, 3H) ppm. MS: M/e 457 (M+1)⁺.

### Compound A5: 2-(7-((2S,5R)-2,5-dimethyl-4-(1-(3-methylquinoxalin-6-yl)ethyl)piperazin-1-yl)-5-oxo-4,5-dihydropyrazolo[1,5-a]pyrimidin-2-yl)acetonitrile

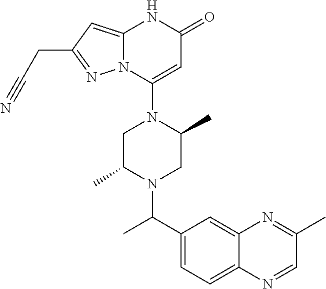 [SMILES: Cc1cnc2ccc(C(C)N3C[C@H](C)N(c4cc(=O)[nH]c5cc(CC#N)nn45)C[C@H]3C)cc2n1]

### Step A: tert-butyl (2R,5S)-4-(5-(benzyloxy)-2-bromopyrazolo[1,5-a]pyrimidin-7-yl)-2,5-dimethylpiperazine-1-carboxylate

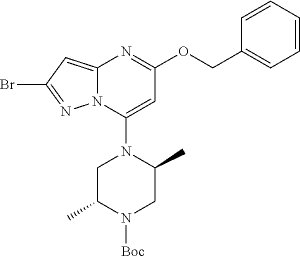 [SMILES: C[C@@H]1CN(c2cc(OCc3ccccc3)nc3cc(Br)nn23)[C@@H](C)CN1C(=O)OC(C)(C)C]

A solution of phenylmethanol (0.88 g, 8.15 mmol) and NaH (60%, 0.41 g, 10.25 mmol) in THF (20 ml) was stirred at RT for 30 min. A solution of tert-butyl (2R,5S)-4-(2-bromo-5-chloropyrazolo[1,5-a]pyrimidin-7-yl)-2,5-dimethylpiperazine-1-carboxylate (3 g, 6.77 mmol) in THF (10 ml) was added to the above solution and then stirred at 70° C. for 1 h. The solution was quenched with H₂O (30 ml) and then extracted with EA (20 ml×2). The organic layer was concentrated to dryness. The resulting residue was purified by flash column chromatography with 0-30% EA in PE to give the titled compound (3.4 g, 97%). MS: M/e 516,518 (M+1)⁺.

### Step B: methyl 5-(benzyloxyl-7-((2S,5R)-4-(tert-butoxycarbonyl)˜2,5-dimethylpiperazin-1-yl)pyrazolo[1,5-a]pyrimidine-2-carboxylate

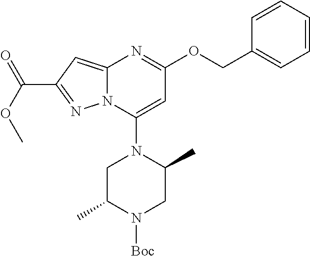 [SMILES: COC(=O)c1cc2nc(OCc3ccccc3)cc(N3C[C@@H](C)N(C(=O)OC(C)(C)C)C[C@@H]3C)n2n1]

A solution of tert-butyl (2R,5S)-4-(5-(benzyloxy)-2-bromopyrazolo[1,5-a]pyrimidin-7-yl)-2,5-dimethylpiperazine-1-carboxylate (1.5 g, 2.91 mmol), Pd(dppf)Cl₂ (0.21 g, 0.29 mmol) and TEA (0.88 g, 8.71 mmol) in MeOH was stirred at 90° C. under CO (2.7 MPa) overnight. The reaction was concentrated to dryness. The resulting residue was purified by flash column chromatography with 0-5% MeOH in DCM to give the titled compound (1.4 g, 97%). MS: M/e 496 (M+1)⁺.

### Step C: tert-butyl (2R,5S)-4-(5-(benzyloxy)-2-(hydroxymethyl)pyrazolo[1,5-a]pyrimidin-7-yl)-2,5-dimethylpiperazine-1-carboxylate

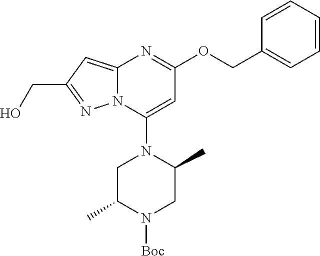 [SMILES: C[C@@H]1CN(c2cc(OCc3ccccc3)nc3cc(CO)nn23)[C@@H](C)CN1C(=O)OC(C)(C)C]

A solution of methyl 5-(benzyloxy)-7-((2S,5R)-4-(tert-butoxycarbonyl)-2,5-dimethylpiperazin-1-yl)pyrazolo[1,5-a]pyrimidine-2-carboxylate (1.2 g, 2.42 mmol) and LiAlH₄ (1M in THF, 3.64 ml, 3.64 mmol) in THF (20 ml) was stirred at 0° C. for 30 min. The solution was quenched with H₂O (30 ml) and then extracted with EA (20 ml×2). The organic layer was concentrated to dryness. The resulting residue was purified by flash column chromatography with 0-5% MeOH in DCM to give the titled compound (1.1 g, crude), which was used to the next step directly. MS: M/e 468 (M+1)⁺.

### Step D: tert-butyl (2R,5S)-4-(5-(benzyloxy)-2-(((methylsulfonyl)oxy)methyl)pyrazolo[1,5-a]pyrimidin-7-yl)-2,5-dimethylpiperazine-1-carboxylate

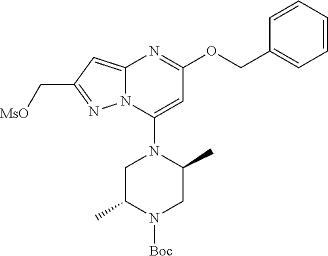 [SMILES: C[C@@H]1CN(c2cc(OCc3ccccc3)nc3cc(COS(C)(=O)=O)nn23)[C@@H](C)CN1C(=O)OC(C)(C)C]

A solution of tert-butyl (2R,5S)-4-(5-(benzyloxy)-2-(hydroxymethyl)pyrazolo[1,5-a]pyrimidin-7-yl)-2,5-dimethylpiperazine-1-carboxylate (600 mg, 1.28 mmol), MsCl (185 mg, 1.61 mmol) and TEA (389 mg, 3.85 mmol) in DCM (15 ml) was stirred at 0° C. for 30 min. The solution was washed with aq. NaHCO₃ (10 ml) and brine (10 ml), dried over Na₂SO₄ and then evaporated to give the titled compound (700 mg, 100%).

### Step E: tert-butyl (2R,5S)-4-(5-(benzyloxy)-2-(cyanomethyl)pyrazolo[1,5-a]pyrimidin-7-yl)-2,5-dimethylpiperazine-1-carboxylate

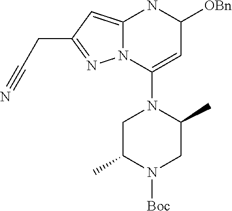 [SMILES: C[C@@H]1CN(C2=CC(OCc3ccccc3)Nc3cc(CC#N)nn32)[C@@H](C)CN1C(=O)OC(C)(C)C]

A solution of tert-butyl (2R,5S)-4-(5-(benzyloxy)-2-(((methylsulfonyl)oxy)methyl)pyrazolo[1,5-a]pyrimidin-7-yl)-2,5-dimethylpiperazine-1-carboxylate (700 mg, 1.28 mmol), TMSCN (381 mg, 3.85 mmol) and K₂CO₃ (532 mg, 3.86 mmol) in MeCN (15 ml) was stirred at 80° C. for 1 hour. The solution was poured into water (20 ml) and then extracted with EA (20 ml×2). The organic layer was washed with brine (10 ml×2), dried over Na₂SO₄ and concentrated to dryness. The resulting residue was purified by flash column chromatography with 0-30% EA in PE to give the titled compound (350 mg, 57%). MS: M/e 477 (M+1)⁺.

### Step F: 2-(5-(benzyloxy)-7-((2S,5R)-2,5-dimethylpiperazin-1-yl)pyrazolo[1,5-a]pyrimidin-2-yl)acetonitrile

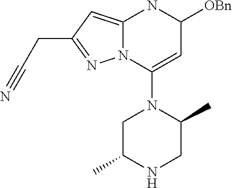 [SMILES: C[C@@H]1CN(C2=CC(OCc3ccccc3)Nc3cc(CC#N)nn32)[C@@H](C)CN1]

A solution of tert-butyl (2R,5S)-4-(5-(benzyloxy)-2-(cyanomethyl)pyrazolo[1,5-a]pyrimidin-7-yl)-2,5-dimethylpiperazine-1-carboxylate (465 mg, 0.98 mmol) and TFA (2 ml) in DCM (15 ml) was stirred at RT for 2.5 hrs. The solution was washed with aq. NaHCO₃ (10 ml) and brine (10 ml), dried over Na₂SO₄ and then evaporated to give the titled compound (360 mg, 98%). MS: M/e 377 (M+1)⁺.

### Step G: 2-(5-(benzyloxy)-7-((2S,5R)-2,5-dimethyl-4-(1-(3-methylquinoxalin-6-yl)ethyl)piperazin-1-yl)pyrazolo[1,5-a]pyrimidin-2-yl)acetonitrile

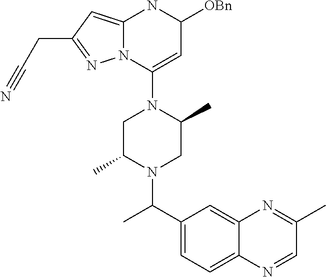 [SMILES: Cc1cnc2ccc(C(C)N3C[C@H](C)N(C4=CC(OCc5ccccc5)Nc5cc(CC#N)nn54)C[C@H]3C)cc2n1]

A solution of 2-(5-(benzyloxy)-7-((2S,5R)-2,5-dimethylpiperazin-1-yl)pyrazolo[1,5-a]pyrimidin-2-yl)acetonitrile (360 mg, 0.96 mmol), 1-(3-methylquinoxalin-6-yl)ethan-1-ol (270 mg, 1.44 mmol), (cyanomethyl)trimethylphosphonium iodide (698 mg, 2.87 mmol) and DIPEA (1.24 g, 9.61 mmol) in CH₃CN (6 ml) was stirred at 100° C. for 2 days. The reaction was diluted with EtOAc (20 ml) and washed with brine (10 ml). The organic layer was concentrated under reduced pressure. The resulting residue was purified by flash column chromatography with 10-50% EA in PE to give the titled compound (400 mg, 77%). MS: M/e 547 (M+1)⁺.

### Step H: 2-(7-((2S,5R)-2,5-dimethyl-4-(1-(3-methylquinoxalin-6-yl)ethyl)piperazin-1-yl)-5-oxo-4,5-dihydropyrazolo[1,5-a]pyrimidin-2-yl)acetonitrile

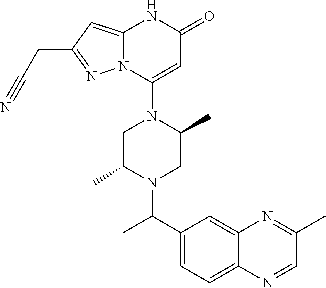 [SMILES: Cc1cnc2ccc(C(C)N3C[C@H](C)N(c4cc(=O)[nH]c5cc(CC#N)nn45)C[C@H]3C)cc2n1]

A solution of 2-(5-(benzyloxy)-7-((2S,5R)-2,5-dimethyl-4-(1-(3-methylquinoxalin-6-yl)ethyl)piperazin-1-yl)pyrazolo[1,5-a]pyrimidin-2-yl)acetonitrile (70 mg, 0.13 mmol) in TFA (2 ml) was stirred at 70° C. overnight. The solution was concentrated under reduced pressure. The residue was purified by Prep-HPLC (Method A) to give the titled compound (3.4 mg). ¹H NMR (400 MHz, DMSO-d₆) δ 11.67 (s, 1H), 8.82 (d, J=6.0 Hz, 1H), 8.03 (dd, J=13.5, 8.6 Hz, 1H), 7.94 (d, J=12.3 Hz, 1H), 7.85 (t, J=7.5 Hz, 1H), 5.77 (d, J=3.3 Hz, 1H), 5.09 (d, J=16.4 Hz, 1H), 4.82 (s, 0.5H), 4.47 (s, 0.5H), 4.07 (d, J=7.7 Hz, 2H), 3.94 (q, J=6.5 Hz, 0.5H), 3.81 (q, J=6.5 Hz, 0.5H), 3.68 (d, J=10.8 Hz, 0.5H), 3.63-3.53 (m, 1H), 3.40-3.35 (m, 0.5H), 3.32-3.28 (m, 0.5H), 3.01 (d, J=7.9 Hz, 0.5H), 2.78 (d, J=11.4 Hz, 1.5H), 2.70 (d, J=2.6 Hz, 3H), 2.06 (d, J=12.2 Hz, 05H), 1.36 (t, J=5.8 Hz, 3H), 1.28 (d, J=6.7 Hz, 15H), 1.17 (d, J=6.3 Hz, 1.5H), 1.08 (d, J=6.5 Hz, 1.5H), 1.02 (d, J=6.4 Hz, 1.5H) ppm. MS: M/e 457 (M+1)⁺.

### Compound A6: 2-(7-((2S,5R)-4-(1-(3-(difluoromethyl)quinoxalin-6-yl)ethyl)-2,5-dimethylpiperazin-1-yl)-4-methyl-5-oxo-4,5-dihydropyrazolo[1,5-a]pyrimidin-2-yl)acetonitrile

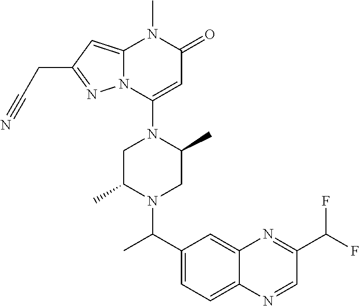 [SMILES: CC(c1ccc2ncc(C(F)F)nc2c1)N1C[C@H](C)N(c2cc(=O)n(C)c3cc(CC#N)nn23)C[C@H]1C]

A solution of 2-(7-((2S,5R)-2,5-dimethylpiperazin-1-yl)-4-methyl-5-oxo-4,5-dihydropyrazolo[1,5-a]pyrimidin-2-yl)acetonitrile (80 mg, 0.267 mmol), 1-(3-(difluoromethyl)quinoxalin-6-yl)ethan-1-ol (90 mg, 0.400 mmol), (cyanomethyl)trimethylphosphonium iodide (130 mg. 0.533 mmol) and DIPEA (103 mg, 0.801 mmol) in CH₃CN (1 ml). The mixture solution was degassed 3 times under N₂ atmosphere. Then the mixture solution was stirred at 100° C. for 24 hours. The reaction mixture was concentrated under reduced pressure. The crude product was purified by Prep-TLC (DCM:MeOH=15:1) and further purified by Prep-HPLC (Method B) to give the titled compound (25 mg, 19%). ¹H NMR (400 MHz, CD₃OD) δ 9.13 (d, J=4.9 Hz, 1H), 8.24-8.05 (m, 3H), 6.99 (td, J=54.5, 4.1 Hz, 1H), 6.10 (d, J=1.6 Hz, 1H), 5.30 (d, J=14.0 Hz, 1H), 4.96-4.92 (m, 0.5H), 4.66-4.61 (m, 0.5H), 4.05 (q, J=6.4 Hz, 0.5H), 3.98 (d, J=6.0 Hz, 2H), 3.91 (q, J=6.5 Hz, 0.5H), 3.87-3.73 (m, 1H), 3.72-3.64 (m, 0.5H), 3.58-3.49 (m, 1H), 3.46 (s, 3H), 3.18 (dd, J=11.8, 3.5 Hz, 0.5H), 2.99-2.84 (m, 1.5H), 2.16 (d, J=12.1 Hz, 0.5H), 1.52-1.37 (m, 4H), 1.28 (d, J=6.5 Hz, 2H), 1.16 (dd, J=28.1, 6.6 Hz, 3H) ppm. MS: M/e 507 (M+1)⁺.

### Compound A7: 2-(7-((2S,5R)-4-(1-(3-methoxyquinoxalin-6-yl)ethyl)-2,5-dimethylpiperazin-1-yl)-4-methyl-5-oxo-4,5-dihydropyrazolo[1,5-a]pyrimidin-2-yl)acetonitrile

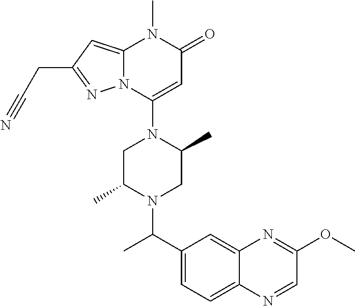 [SMILES: COc1cnc2ccc(C(C)N3C[C@H](C)N(c4cc(=O)n(C)c5cc(CC#N)nn45)C[C@H]3C)cc2n1]

A solution of 2-(7-((2S,5R)-2,5-dimethylpiperazin-1-yl)-4-methyl-5-oxo-4,5-dihydropyrazolo[1,5-a]pyrimidin-2-yl)acetonitrile (350 mg, 1.167 mmol), 1-(3-methoxyquinoxalin-6-yl)ethan-1-ol (357 mg, 1.750 mmol), (cyanomethyl)trimethylphosphonium iodide (567 mg. 2.334 mmol) and DIPEA (452 mg, 3.501 mmol) in CH₃CN (2 ml). The mixture solution was degassed 3 times under N₂ atmosphere. Then the mixture solution was stirred at 105° C. for 24 hours. The reaction mixture was concentrated under reduced pressure. The crude product was purified by flash column chromatography (DCM:MeOH=15:1) and further purified by Prep-HPLC (Method B) to give the titled compound (173 mg, 31%). ¹H NMR (400 MHz, CD₃OD) δ 8.42 (d, J=6.0 Hz, 1H), 7.95 (dd, J=12.8, 8.5 Hz, 1H), 7.84 (d, J=8.1 Hz, 1H), 7.74 (dd, J=8.5, 1.4 Hz, 1H), 6.10 (d, J=1.4 Hz, 1H), 5.29 (d, J=10.9 Hz, 1H), 4.90-4.86 (m, 0.5H), 4.62-4.56 (m, 0.5H), 4.10 (d, J=5.6 Hz, 3H), 3.99 (d, J=4.4 Hz, 2H), 3.97-3.90 (m, 0.5H), 3.85-3.78 (m, 1H), 3.76-3.70 (m, 0.5H), 3.69-3.62 (m, 0.5H), 3.53 (d, J=2.0 Hz, 1H), 3.45 (s, 3H), 3.14 (dd, J=11.8, 3.5 Hz, 0.5H), 2.95-2.85 (m, 1.5H), 2.18 (d, J=12.2 Hz, 0.5H), 1.42 (dd, J=10.7, 6.6 Hz, 4H), 1.26 (d, J=6.5 Hz, 2H), 1.19 (d, J=6.6 Hz, 2H), 1.10 (d, J=6.5 Hz, 1H) ppm. MS: M/e 487 (M+1)⁺.

### Compound A8: 2-(7-((2S,5R)-4-(1-(3-chloroquinoxalin-6-yl)ethyl)-2,5-dimethylpiperazin-1-yl)-4-methyl-5-oxo-4,5-dihydropyrazolo[1,5-a]pyrimidin-2-yl)acetonitrile

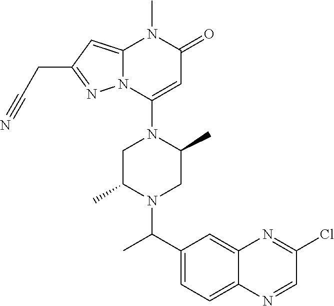 [SMILES: CC(c1ccc2ncc(Cl)nc2c1)N1C[C@H](C)N(c2cc(=O)n(C)c3cc(CC#N)nn23)C[C@H]1C]

### Step A: 2-(7-((2S,5R)-4-(1-(3-hydroxyquinoxalin-6-yl)ethyl)-2,5-dimethylpiperazin-1-yl)-4-methyl-5-oxo-4,5-dihydropyrazolo[1,5-a]pyrimidin-2-yl)acetonitrile

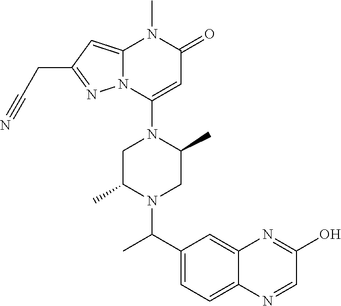 [SMILES: CC(c1ccc2ncc(O)nc2c1)N1C[C@H](C)N(c2cc(=O)n(C)c3cc(CC#N)nn23)C[C@H]1C]

To a solution of 2-(7-((2S,5R)-4-(1-(3-methoxyquinoxalin-6-yl)ethyl)-2,5-dimethylpiperazin-1-yl)-4-methyl-5-oxo-4,5-dihydropyrazolo[1,5-a]pyrimidin-2-yl)acetonitrile (160 mg, 0.329 mmol) in DCM (5 mL) was added BBr₃ (5 mL, 1M, 4.938 mmol). The mixture solution was stirred at room temperature for 12 hours. The reaction mixture was quenched with saturated NaHCO₃ aq. (20 mL), extracted with DCM (30 mL×2), combined, washed brine (50 mL×2), dried and concentrated to dryness. The resulting residue was purified by flash column chromatography (DCM:MeOH=20:1) to give the titled compound (60 mg, 39%). MS: M/e 473 (M+1)⁺.

### Step B: 2-(7-((2S,5R)-4-(1-(3-chloroquinoxalin-6-yl)ethyl-2,5-dimethylpiperazin-1-yl)-4-methyl-5-oxo-4,5-dihydropyrazolo[1,5-a]pyrimidin-2-yl)acetonitrile

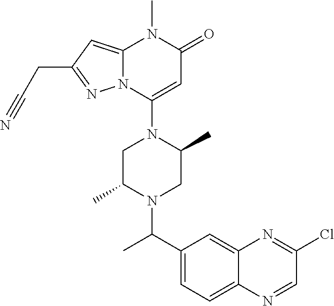 [SMILES: CC(c1ccc2ncc(Cl)nc2c1)N1C[C@H](C)N(c2cc(=O)n(C)c3cc(CC#N)nn23)C[C@H]1C]

To a solution of 2-(7-((2S,5R)-4-(1-(3-hydroxyquinoxalin-6-yl)ethyl)-2,5-dimethylpiperazin-1-yl)-4-methyl-5-oxo-4,5-dihydropyrazolo[1,5-a]pyrimidin-2-yl)acetonitrile (60 mg, 0.127 mmol) in POCl₃ (5 mL) was stirred at 80° C. for 2 hours. The reaction mixture was poured into H₂O (30 mL), extracted with DCM (30 mL×2). combined, washed brine (50 mL×2), dried over Na₂SO₄ and concentrated to dryness. The resulting residue was purified by Prep-TLC (DCM:MeOH=15:1) and Prep-HPLC (Method B) to give the titled compound (2 mg, 3%). ¹H NMR (400 MHz, CD₃OD) δ 8.83 (d, J=4.3 Hz, 1H), 8.11 (dd, J=11.6, 8.7 Hz, 1H), 8.05-7.95 (m, 2H), 7.78 (s, 1H), 5.62 (s, 1H), 4.76-4.63 (m, 1H), 4.00-3.95 (m, 0.5H), 3.93 (d, J=2.3 Hz, 2H), 3.82 (q, J=6.4 Hz, 0.5H), 3.74 (s, 3H), 3.68 (d, J=9.6 Hz, 15H), 3.49-3.42 (m, 0.5H), 3.09 (dd, J=11.9, 4.0 Hz, 0.5H), 2.95-2.66 (m, 2H), 2.20 (d, J=12.4 Hz, 0.5H), 1.48-1.38 (m, 4H), 1.22 (dd, J=12.2, 6.5 Hz, 4H), 1.06 (d, J=6.5 Hz, 1H) ppm. MS: M/e 491 (M+1)⁺.

### Compound A9: 2-(7-((2S,5R)-4-(1-(3,3-dimethyl-2,3-dihydro-[1,4]dioxino[2,3-b]pyridin-6-yl)ethyl)-2,5-dimethylpiperazin-1-yl)-4-methyl-5-oxo-4,5-dihydropyrazolo[1,5-a]pyrimidin-2-yl)acetonitrile

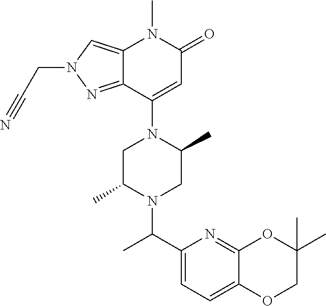 [SMILES: CC(c1ccc2c(n1)OC(C)(C)CO2)N1C[C@H](C)N(c2cc(=O)n(C)c3cn(CC#N)nc23)C[C@H]1C]

A solution of 2-(7-((2S,5R)-2,5-dimethylpiperazin-1-yl)-4-methyl-5-oxo-4,5-dihydropyrazolo[1,5-a]pyrimidin-2-yl)acetonitrile (143.5 mg, 0.48 mmol), 1-(3,3-dimethyl-2,3-dihydro-[1,4]dioxino[2,3-b]pyridin-6-yl)ethan-1-ol (100 mg, 0.48 mmol), (cyanomethyl)trimethylphosphonium iodide (349 mg, 1.44 mmol) and DIPEA (617 mg, 4.78 mmol) in CH₃CN (2 ml) was stirred at 100° C. overnight. The reaction was diluted with EtOAc (10 ml) and washed with brine (10 ml). The organic layer was concentrated under reduced pressure. The resulting residue was purified by flash column chromatography and then Prep-HPLC (Method A) to give the titled compound (38 mg). ¹H NMR (400 MHz, DMSO-d₆) δ 7.30 (dd, J=8.0, 3.4 Hz, 1H), 7.02 (t, J=7.5 Hz, 1H), 6.12 (d, J=3.6 Hz, 1H), 5.26 (d, J=6.4 Hz, 1H), 4.48 (s, 1H), 4.13 (d, J=1.6 Hz, 2H), 3.94 (d, J=7.0 Hz, 2H), 3.72 (q, J=6.6 Hz, 0.5H), 3.55 (s, 1H), 3.51-3.37 (m, 2H), 3.34 (s, 3H), 3.14 (d, J=8.6 Hz, 0.5H), 2.99 (d, J=8.4 Hz, 0.5H), 2.80-2.70 (m, 1H), 2.10 (d, J=9.8 Hz, 0.5H), 1.36-1.29 (m, 6H), 1.23 (t, J=7.6 Hz, 3H), 1.17 (d, J=6.4 Hz, 1.5H), 1.08 (dd, J=11.1, 6.5 Hz, 3H), 1.01 (d, J=6.4 Hz, 1.5H) ppm. MS: M/e 492 (M+1)⁺.

### Compound A10: 2-(7-((2S,5R)-4-(1-(3,3-dimethyl-2,3-dihydrobenzo[b][1,4]dioxin-6-yl)ethyl)-2,5-dimethylpiperazin-1-yl)-4-methyl-5-oxo-4,5-dihydropyrazolo[1,5-a]pyrimidin-2-yl)acetonitrile

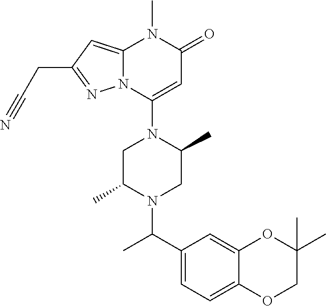 [SMILES: CC(c1ccc2c(c1)OC(C)(C)CO2)N1C[C@H](C)N(c2cc(=O)n(C)c3cc(CC#N)nn23)C[C@H]1C]

A solution of 2-(7-((2S,5R)-2,5-dimethylpiperazin-1-yl)-4-methyl-5-oxo-4,5-dihydropyrazolo[1,5-a]pyrimidin-2-yl)acetonitrile (450 mg, 1.50 mmol), 1-(3,3-dimethyl-2,3-dihydrobenzo[b][1,4]dioxin-6-yl)ethan-1-ol (468 mg, 2.25 mmol), (cyanomethyl)trimethylphosphonium iodide (1.09 g, 4.49 mmol) and DIPEA (1.94 g, 15.04 mmol) in CH₃CN (8 ml) was stirred at 100° C. overnight. The reaction was diluted with EtOAc (15 ml) and washed with brine (10 ml). The organic layer was concentrated under reduced pressure. The resulting residue was purified by flash column chromatography to give the titled Compound A10, which was further separated into Compound A10a (56 mg) and Compound A10b (61 mg) by Prep-HPLC (Method A).

Compound A10a (the earlier peak): ¹H NMR (400 MHz, DMSO-d₆) δ 6.88-6.77 (m, 3H), 6.11 (s, 1H), 5.22 (s, 1H), 4.71 (s, 1H), 4.13 (s, 2H), 3.91 (s, 2H), 3.55 (q, J=6.2 Hz, 1H), 3.39-3.34 (m, 1H), 3.33 (s, 3H), 3.25 (d, J=11.2 Hz, 1H), 2.91 (dd, J=11.8, 3.1 Hz, 1H), 2.82-2.73 (m, 1H), 2.64 (d, J=9.8 Hz, 1H), 1.28 (s, 6H), 1.21 (t, J=6.7 Hz, 6H), 0.95 (d, J=6.4 Hz, 3H) ppm. MS: M/e 491 (M+1)⁺.

Compound A10b (the later peak): ¹H NMR (400 MHz, DMSO-d₆) δ 6.82-6.77 (m, 3H), 6.10 (s, 1H), 5.23 (s, 1H), 4.48 (s, 1H), 4.12 (s, 2H), 3.89 (s, 2H), 3.62 (d, J=12.2 Hz, 1H), 3.53 (d, J=12.0 Hz, 1H), 3.46-3.39 (m, 2H), 3.33 (s, 3H), 2.65 (dd, J=11.6, 3.2 Hz, 1H), 2.09 (d, J=13.6 Hz, 1H), 1.26 (d, J=3.2 Hz, 6H), 1.21 (d, J=6.4 Hz, 3H), 1.07 (t, J=4.8 Hz, 6H) ppm. MS: M/e 491 (M+1)⁺.

### Compound A11: 2-(7-((2S,5R)-4-(1-(3,3-dimethyl-2,3-dihydrobenzo[b][1,4]dioxin-6-yl)ethyl)-2,5-dimethylpiperazin-1-yl)-6-fluoro-4-methyl-5-oxo-4,5-dihydropyrazolo[1,5-a]pyrimidin-2-yl)acetonitrile

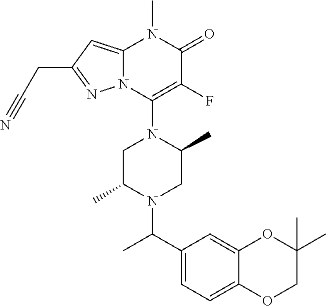 [SMILES: CC(c1ccc2c(c1)OC(C)(C)CO2)N1C[C@H](C)N(c2c(F)c(=O)n(C)c3cc(CC#N)nn23)C[C@H]1C]

### Step A: tert-butyl (2R,5S)-4-(2-(cyanomethyl)-6-fluoro-4-methyl-5-oxo-4,5-dihydropyrazolo[1,5-a]pyrimidin-7-yl)-2,5-dimethylpiperazine-1-carboxylate

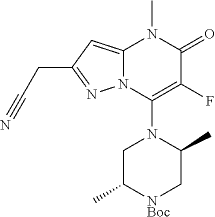 [SMILES: C[C@@H]1CN(c2c(F)c(=O)n(C)c3cc(CC#N)nn23)[C@@H](C)CN1C(=O)OC(C)(C)C]

A solution of tert-butyl (2R,5S)-4-(2-(cyanomethyl)-4-methyl-5-oxo-4,5-dihydropyrazolo[1,5-a]pyrimidin-7-yl)-2,5-dimethylpiperazine-1-carboxylate (700 mg, 1.75 mmol) and select F (774 mg, 2.19 mmol) in MeCN (10 ml) was stirred at RT overnight. The reaction was diluted with EtOAc (15 ml) and washed with brine (10 ml). The organic layer was concentrated under reduced pressure. The resulting residue was purified by flash column chromatography with 35-55% EA in PE to give the titled compound (230 mg, 31%). MS: M/e 419 (M+1)⁺.

### Step B: 2-(7-((2S,5R)-2,5-dimethylpiperazin-1-yl)-6-fluoro-4-methyl-5-oxo-4,5-dihydropyrazolo[1,5-a]pyrimidin-2-yl)acetonitrile

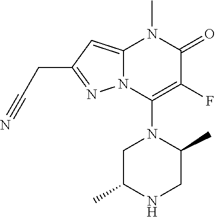 [SMILES: C[C@@H]1CN(c2c(F)c(=O)n(C)c3cc(CC#N)nn23)[C@@H](C)CN1]

A solution of tert-butyl (2R,5S)-4-(2-(cyanomethyl)-6-fluoro-4-methyl-5-oxo-4,5-dihydropyrazolo[1,5-a]pyrimidin-7-yl)-2,5-dimethylpiperazine-1-carboxylate (230 mg, 0.55 mmol) and TFA (2 ml) in DCM (10 ml) was stirred at RT for 30 min. The reaction was washed with aq. NaHCO₃ (10 ml×2) and brine (10 ml), dried over Na₂SO₄ and evaporated to dryness to give the titled compound (175 mg, 100%). MS: M/e 319 (M+1)⁺.

### Step C: 2-(7-((2S,5R)-4-(1-(3,3-dimethyl-2,3-dihydrobenzo[b][1,4]dioxin-6-yl)ethyl)-2,5-dimethylpiperazin-1-yl)-6-fluoro-4-methyl-5-oxo-4,5-dihydropyrazolo[1,5-a]pyrimidin-2-yl)acetonitrile

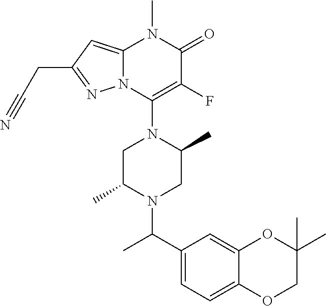 [SMILES: CC(c1ccc2c(c1)OC(C)(C)CO2)N1C[C@H](C)N(c2c(F)c(=O)n(C)c3cc(CC#N)nn23)C[C@H]1C]

A solution of 2-(7-((2S,5R)-2,5-dimethylpiperazin-1-yl)-6-fluoro-4-methyl-5-oxo-4,5-dihydropyrazolo[1,5-a]pyrimidin-2-yl)acetonitrile (175 mg, 0.55 mmol), 1-(3,3-dimethyl-2,3-dihydrobenzo[b][1,4]dioxin-6-yl)ethan-1-ol (229 mg, 1.10 mmol), (cyanomethyl)trimethylphosphonium iodide (401 mg, 1.65 mmol) and DIPEA (710 mg, 5.50 mmol) in CH₃CN (4 ml) was stirred at 100° C. for 2 days. The reaction was diluted with EtOAc (15 ml) and washed with brine (10 ml). The organic layer was concentrated under reduced pressure. The resulting residue was purified by flash column chromatography to give the titled Compound A11, which was further separated into Compound A11a (32 mg) and Compound A11b (26 mg) by Prep-HPLC (Method A).

Compound A11a (the earlier peak): ¹H NMR (400 MHz, DMSO-d₆) δ 6.89-6.79 (m, 3H), 6.22 (s, 1H), 4.13 (s, 2H), 3.90 (s, 3H), 3.76-3.63 (m, 2H), 3.42 (s, 3H), 3.19 (s, 1H), 3.06 (d, J=11.7 Hz, 1H), 2.63 (d, J=8.4 Hz, 1H), 2.08 (dd, J=10.1, 6.1 Hz, 1H), 1.27 (d, J=4.0 Hz, 6H), 1.20 (d, J=6.4 Hz, 3H), 1.03 (dd, J=12.0, 6.3 Hz, 6H) ppm. MS: M/e 509 (M+1)⁺.

Compound A11b (the later peak): ¹H NMR (400 MHz, DMSO-d₆) δ 6.87-6.82 (m, 1H), 6.80-6.75 (m, 2H), 6.22 (s, 1H), 4.13 (s, 2H), 4.01 (s, 1H), 3.91 (s, 2H), 3.81 (q, J=6.9 Hz, 1H), 3.49 (d, J=11.4 Hz, 1H), 3.41 (s, 3H), 3.02 (d, J=8.6 Hz, 1H), 2.92-2.86 (m, 1H), 2.56 (s, 1H), 2.25-2.18 (m, 1H), 1.27 (d, J=7.0 Hz, 9H), 1.12 (d, J=6.3 Hz, 3H), 1.00 (d, J=6.3 Hz, 3H) ppm. MS: M/e 509 (M+1)⁺.

### Compound A12: 2-(cyanomethyl)-7-((2S,5R)-4-(1-(3,3-dimethyl-2,3-dihydrobenzo[b][1,4]dioxin-6-yl)ethyl)-2,5-dimethylpiperazin-1-yl)-4-methyl-5-oxo-4,5-dihydropyrazolo[1,5-a]pyrimidine-6-carbonitrile

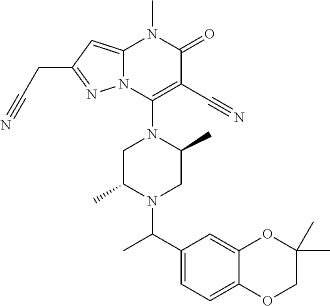 [SMILES: CC(c1ccc2c(c1)OC(C)(C)CO2)N1C[C@H](C)N(c2c(C#N)c(=O)n(C)c3cc(CC#N)nn23)C[C@H]1C]

### Step A: tert-butyl (2R,5S)-4-(6-bromo-2-(cyanomethyl)-4-methyl-5-oxo-4,5-dihydropyrazolo[1,5-a]pyrimidin-7-yl)-2,5-dimethylpiperazine-1-carboxylate

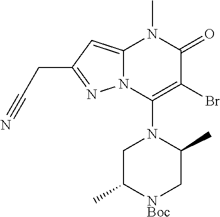 [SMILES: C[C@@H]1CN(c2c(Br)c(=O)n(C)c3cc(CC#N)nn23)[C@@H](C)CN1C(=O)OC(C)(C)C]

A solution of tert-butyl (2R,5S)-4-(2-(cyanomethyl)-4-methyl-5-oxo-4,5-dihydropyrazolo[1,5-a]pyrimidin-7-yl)-2,5-dimethylpiperazine-1-carboxylate (700 mg, 1.75 mmol) and NBS (343 mg, 1.93 mmol) in MeCN (15 ml) was stirred at RT for 3 hrs. The reaction was poured into water (20 ml) and then extracted with EA (10 ml×2). The organic layer was washed with brine (10 ml), dried and concentrated. The resulting residue was purified by flash column chromatography with 20-60/a EA in PE to give the titled compound (520 mg, 62%). MS: M/e 479,481 (M+1)⁺.

### Step B: tert-butyl (2R,5S)-4-(6-cyano-2-(cyanomethyl) 4-methyl-5-oxo-4,5-dihydropyrazolo[1,5-a]pyrimidin-7-yl)-2,5-dimethylpiperazine-1-carboxylate

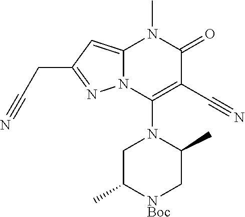 [SMILES: C[C@@H]1CN(c2c(C#N)c(=O)n(C)c3cc(CC#N)nn23)[C@@H](C)CN1C(=O)OC(C)(C)C]

A solution of tert-butyl (2R,5S)-4-(6-bromo-2-(cyanomethyl)-4-methyl-5-oxo-4,5-dihydropyrazolo[1,5-a]pyrimidin-7-yl)-2,5-dimethylpiperazine-1-carboxylate (520 mg, 1.09 mmol), ZnCN₂ (382 mg, 3.26 mmol) and Pd(PPh₃)-4 (125.6 mg, 0.11 mmol) in DMF (10 ml) was stirred at 100° C. for 2 days. The reaction was poured into water (20 ml) and then extracted with EA (10 ml×2). The organic layer was washed with brine (10 ml), dried and concentrated. The resulting residue was purified by flash column chromatography with 20-60% EA in PE to give the titled compound (200 mg, crude). MS: M/e 426 (M+1)⁺.

### Step C: 2-(cyanomethyl)-7-((2S,5R)-2,5-dimethylpiperazin-1-yl)-4-methyl-5-oxo-4,5-dihydropyrazolo[1,5-a]pyrimidine-6-carbonitrile

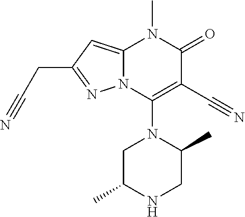 [SMILES: C[C@@H]1CN(c2c(C#N)c(=O)n(C)c3cc(CC#N)nn23)[C@@H](C)CN1]

A solution of tert-butyl (2R,5S)-4-(6-cyano-2-(cyanomethyl)-4-methyl-5-oxo-4,5-dihydropyrazolo[1,5-a]pyrimidin-7-yl)-2,5-dimethylpiperazine-1-carboxylate (200 mg, crude) and TFA (2 ml) in DCM (10 ml) was stirred at RT for 30 min. The reaction was extracted with H2O (10 ml). The aqueous solution was adjusted to pH=8-9 and then extracted with DCM (10 ml×2). The organic layer was dried over Na₂SO₄ and then evaporated to dryness to give the titled compound (130 mg). MS: M/e 326 (M+1)⁺.

### Step D: 2-(cyanomethyl)-7-((2S,5R)-4(1-(3,3-dimethyl-2,3-dihydrobenzo[b][1,4]dioxin-6-yl)ethyl)-2,5-dimethylpiperazin-1-yl)-4-methyl-5-oxo-4,5-dihydropyrazolo[1,5-a]pyrimidine-6-carbonitrile

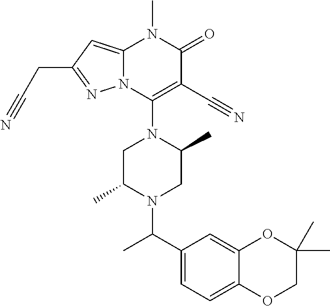 [SMILES: CC(c1ccc2c(c1)OC(C)(C)CO2)N1C[C@H](C)N(c2c(C#N)c(=O)n(C)c3cc(CC#N)nn23)C[C@H]1C]

A solution of 2-(cyanomethyl)-7-((2S,5R)-2,5-dimethylpiperazin-1-yl)-4-methyl-5-oxo-4,5-dihydropyrazolo[1,5-a]pyrimidine-6-carbonitrile (130 mg, 0.40 mmol), 1-(3,3-dimethyl-2,3-dihydrobenzo[b][1,4]dioxin-6-yl)ethan-1-ol (166 mg, 0.80 mmol), (cyanomethyl)trimethylphosphonium iodide (291 mg, 1.20 mmol) and DIPEA (514 mg, 3.98 mmol) in CH₃CN (3 ml) was stirred at 100° C. for 2 days. The reaction was diluted with EtOAc (15 ml) and washed with brine (10 ml). The organic layer was concentrated under reduced pressure. The resulting residue was purified by flash column chromatography to give the titled Compound A12, which was further separated into Compound A12a (16 mg) and Compound A12b (15 mg) by Prep-HPLC (Method A).

Compound A12a (the earlier peak): ¹H NMR (400 MHz, DMSO-d₆) δ 6.90-6.79 (m, 3H), 6.24 (s, 1H), 4.81 (s, 1H), 4.19 (s, 2H), 3.90 (s, 2H), 3.82 (d, J=10.0 Hz, 1H), 3.51 (q, J=6.4 Hz, 1H), 3.35 (s, 3H), 3.28 (d, J=10.0 Hz, 1H), 3.09 (d, J=8.6 Hz, 1H), 2.78 (s, 1H), 2.73 (d, J=11.5 Hz, 1H), 1.40 (d, J=6.6 Hz, 3H), 1.28 (d, J=3.7 Hz, 6H), 1.19 (d, J=6.4 Hz, 3H), 0.85 (d, J=6.4 Hz, 3H) ppm. MS: M/e 516 (M+1)⁺.

Compound A12b (the later peak): ¹H NMR (400 MHz, DMSO-d₆) δ 6.80 (d, J=8.6 Hz, 3H), 6.23 (s, 1H), 4.64 (s, 1H), 4.17 (s, 2H), 4.05 (d, J=10.3 Hz, 1H), 3.89 (s, 2H), 3.49 (d, J=13.8 Hz, 2H), 3.39 (q, J=6.3 Hz, 1H), 3.35 (s, 3H), 2.87 (d, J=8.5 Hz, 1H), 2.12 (d, J=11.4 Hz, 1H), 1.27 (d, J=3.7 Hz, 6H), 1.22 (d, J=6.5 Hz, 6H), 0.97 (d, J=6.2 Hz, 3H) ppm. MS: M/e 516 (M+1)⁺.

### Compound A13: 2-(7-((2S,5R)-4-(1-(2,3-dihydrobenzo[b][1,4]dioxin-6-yl)ethyl)-2,5-dimethylpiperazin-1-yl)-4-methyl-5-oxo-4,5-dihydropyrazolo[1,5-a]pyrimidin-2-yl)acetonitrile

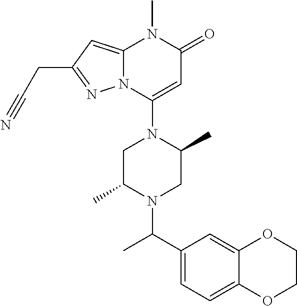 [SMILES: CC(c1ccc2c(c1)OCCO2)N1C[C@H](C)N(c2cc(=O)n(C)c3cc(CC#N)nn23)C[C@H]1C]

A solution of 2-(7-((2S,5R)-2,5-dimethylpiperazin-1-yl)-4-methyl-5-oxo-4,5-dihydropyrazolo[1,5-a]pyrimidin-2-yl)acetonitrile (140 mg, 0.47 mmol), 1-(2,3-dihydrobenzo[b][1,4]dioxin-6-yl)ethan-1-ol (168 mg, 0.93 mmol), (cyanomethyl)trimethylphosphonium iodide (340 mg, 1.40 mmol) and DIPEA (602 mg, 4.67 mmol) in CH₃CN (4 ml) was stirred at 100° C. for 3 days. The reaction was diluted with EtOAc (10 ml) and washed with brine (10 ml). The organic layer was concentrated under reduced pressure. The resulting residue was purified by flash column chromatography and then further purified by Prep-HPLC (Method A) to give the titled Compound (19 mg). ¹H NMR (400 MHz, DMSO-d₆) δ 6.86-6.76 (m, 3H), 6.11 (d, J=4.0 Hz, 1H), 5.22 (d, J=5.9 Hz, 1H), 4.74 (s, 0.5H), 4.48 (s, 0.5H), 4.22 (d, J=6.9 Hz, 4H), 4.12 (d, J=5.2 Hz, 2H), 3.64 (d, J=12.2 Hz, 0.5H), 3.56-3.50 (m, 1H), 3.47-3.38 (m, 1.5H), 3.33 (s, 3H), 3.25 (d, J=12.2 Hz, 0.5H), 2.91 (d, J=8.7 Hz, 0.5H), 2.77 (s, 0.5H), 2.66 (d, J=9.6 Hz, 1H), 2.11 (d, J=11.0 Hz, 0.5H), 1.24-1.18 (m, 4.5H), 1.10-1.05 (m, 3H), 0.95 (d, J=6.5 Hz, 1.5H) ppm. MS: M/e 463 (M+1)⁺.

### Compound A14: 2-(7-((2S,5R)-5-ethyl-4-(1-(4-fluoro-2-(trifluoromethyl)phenyl)ethyl)-2-methylpiperazin-1-yl)-4-methyl-5-oxo-4,5-dihydropyrazolo[1,5-a]pyrimidin-2-yl)acetonitrile

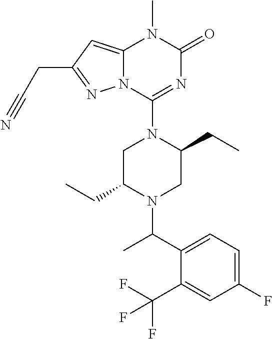 [SMILES: CC[C@H]1CN(C(C)c2ccc(F)cc2C(F)(F)F)[C@H](CC)CN1c1nc(=O)n(C)c2cc(CC#N)nn12]

To a mixture of 2-(7-((2S,5R)-2,5-dimethylpiperazin-1-yl)-4-methyl-5-oxo-4,5-dihydropyrazolo[1,5-a]pyrimidin-2-yl)acetonitrile (10 mg, 0.03 mmol) from last step in acetonitrile (5 mL) was added trimethyl(prop-2-yn-1-yl)phosphonium (46 mg, 0.2 mmol), 1-(4-fluoro-2-(trifluoromethyl)phenyl)ethan-1-ol (20 mg, 0.1 mmol) and DIPEA (129 mg, 1 mmol). The reaction mixture was sealed and stirred under nitrogen protection at 105° C. overnight. The mixture was added H₂O and extracted by ethyl acetate. The organic layer was washed with brine, dried over Na₂SO₄, filtered, and concentrated. The resulting residue was purified by Prep-TLC(EA) to give the titled compound (0.2 mg, 4%). ¹H NMR (400 MHz, CD₃OD) δ 8.05 (dd, J=18.2, 8.8 Hz, 1H), 7.40 (dd, J=12.4, 9.3 Hz, 2H), 6.12 (s, 1H), 5.33 (s, 1H), 4.68 (s, 1H), 4.03 (s, 1H), 3.98 (d, J=4.9 Hz, 2H), 3.66-3.55 (m, 1H), 3.46 (s, 4H), 3.13 (s, 1H), 2.92 (d, J=11.6 Hz, 1H), 2.19 (t, J=7.6 Hz, 1H), 2.03 (d, J=5.6 Hz, 2H), 1.13 (d, J=6.7 Hz, 2H), 0.99 (t, J=7.4 Hz, 2H), 0.90 (t, J=6.7 Hz, 3H), 0.69 (t, J=7.4 Hz, 2H) ppm. MS: M/e 505 (M+1)⁺.

### Compound A15: 2-(7-((2S,5R)-2,5-diethyl-4-(1-(4-fluoro-2-(trifluoromethyl)phenyl)ethyl)piperazin-1-yl)-4-methyl-5-oxo-4,5-dihydropyrazolo[1,5-a]pyrimidin-2-yl)acetonitrile

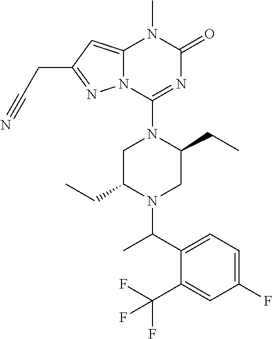 [SMILES: CC[C@H]1CN(C(C)c2ccc(F)cc2C(F)(F)F)[C@H](CC)CN1c1nc(=O)n(C)c2cc(CC#N)nn12]

### Step A: tert-butyl (2S,5R)-2,5-diethyl-4-(1-(4-fluoro-2-(trifluoromethyl)phenyl)ethyl)piperazine-1-carboxylate

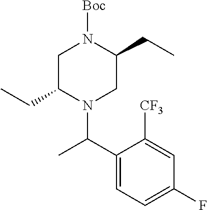 [SMILES: CC[C@H]1CN(C(C)c2ccc(F)cc2C(F)(F)F)[C@H](CC)CN1C(=O)OC(C)(C)C]

To a solution of tert-butyl (2S,5R)-2,5-diethylpiperazine-1-carboxylate (726 mg, 3 mmol) in acetonitrile (15 mL) was added 1-(4-fluoro-2-(trifluoromethyl)phenyl)ethan-1-ol (981 mg, 4.5 mmol), (cyanomethyl)trimethylphosphonium (1280 mg, 6 mmol) and DIPEA (1.9 g, 15 mmol). The resulted mixture was sealed and stirred at 100° C. for 24 hours. The mixture was added to water and extracted by ethyl acetate. The organic layer was dried over Na₂SO₄, filtered, and concentrated. The resulting crude product was purified by flash column chromatography to give the titled compound (520 mg, 40%). MS: M/e 433 (M+1)⁺.

### Step B: (2R,5S)-2,5-diethyl-1-(1-(4-fluoro-2-(trifluoromethyl)phenyl)ethyl)piperazine

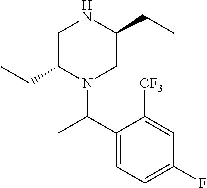 [SMILES: CC[C@H]1CN(C(C)c2ccc(F)cc2C(F)(F)F)[C@H](CC)CN1]

To a solution of tert-butyl (2S,5R)-2,5-diethyl-4-(1-(4-fluoro-2-(trifluoromethyl)phenyl)ethyl)piperazine-1-carboxylate (520 mg, 1.2 mmol) in DCM (20 mL) was added TFA (5 mL). The resulted mixture was stirred at RT overnight. The mixture was concentrated in vacuo. The residue was added to a solution of saturated NaHCO₃ aqueous solution and extracted by DCM. The organic layer was dried over Na₂SO₄, filtered, and concentrated. The resulting crude product (300 mg, 75%) was used directly in next step without further purification. MS: M/e 333 (M+1)⁺.

### Step C: 2-bromo-5-chloro-7-((2S,5R)-2,5-diethyl-4-(1-(4-fluoro-2-(trifluoromethyl)phenyl)ethyl)piperazin-1-yl)pyrazolo[1,5-a]pyrimidine

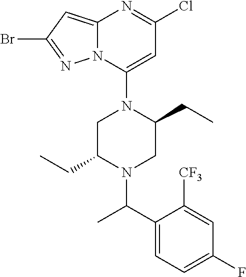 [SMILES: CC[C@H]1CN(C(C)c2ccc(F)cc2C(F)(F)F)[C@H](CC)CN1c1cc(Cl)nc2cc(Br)nn12]

To a mixture of 2-bromo-5,7-dichloropyrazolo[1,5-a]pyrimidine (212 mg, 0.8 mmol) in THF (15 mL) was added (2R,5S)-2,5-diethyl-1-(1-(4-fluoro-2-(trifluoromethyl)phenyl)ethyl)piperazine (0.4 g, 1.2 mmol) and DIPEA (258 mg, 2 mmol). The resulted mixture was stirred at rt overnight. The mixture was added to water and extracted by ethyl acetate. The organic layer was dried over Na₂SO₄, filtered, and concentrated. The resulting crude product was purified by column chromatography to give the titled compound (400 mg, 89%). MS: M/e 562 (M+1)⁺.

### Step D: 5-(benzyloxy)-2-bromo-7-((2S,5R)-2,5-diethyl-4-(1-(4-fluoro-2-(trifluoromethyl)phenyl)ethyl)piperazin-1-yl)pyrazolo[1,5-a]pyrimidine

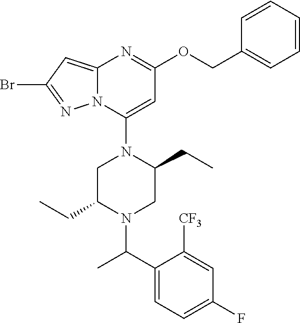 [SMILES: CC[C@H]1CN(C(C)c2ccc(F)cc2C(F)(F)F)[C@H](CC)CN1c1cc(OCc2ccccc2)nc2cc(Br)nn12]

To a mixture of phenylmethanol (216 mg, 2 mmol) in THF (15 mL) was added NaH (60% in oil, 160 mg. 4 mmol). The reaction was stirred at rt for 0.5 h. 2-bromo-5-chloro-7-((2S,5R)-2,5-diethyl-4-(1-(4-fluoro-2-(trifluoromethyl)phenyl)ethyl)piperazin-1-yl)pyrazolo[1,5-a]pyrimidine (640 mg, 1.13 mmol) was added to the mixture and the reaction was stirred at 70° C. overnight. The mixture was added to water and extracted by ethyl acetate. The organic layer was dried over Na₂SO₄, filtered, and concentrated. The resulting crude product was purified by column chromatography to give the titled compound (600 mg, 84%). MS: M/e 634 (M+1)⁺.

### Step E: 2-bromo-7-((2S,5R)-2,5-diethyl-4-(1-(4-fluoro-2-(trifluoromethyl)phenyl)ethyl)piperazin-1-yl)pyrazolo[1,5-a]pyrimidin-5(4H)-one

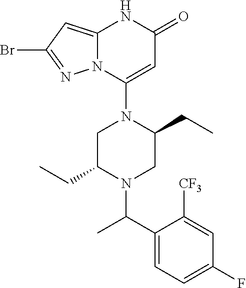 [SMILES: CC[C@H]1CN(C(C)c2ccc(F)cc2C(F)(F)F)[C@H](CC)CN1c1cc(=O)[nH]c2cc(Br)nn12]

A mixture of 5-(benzyloxy)-2-bromo-7-((2S,5R)-2,5-diethyl-4-(1-(4-fluoro-2-(trifluoromethyl)phenyl)ethyl)piperazin-1-yl)pyrazolo[1,5-a]pyrimidine (500 mg, 0.78 mmol) in TFA (10 mL) was stirred at 80° C. overnight. The mixture concentrated in vacuo. The residue was added to a solution of saturated NaHCO₃ aqueous solution and extracted by DCM. The organic layer was dried over Na₂SO₄, filtered, and concentrated. The resulting crude product was used directly in next step without further purification. MS: M/e 544 (M+1)⁺.

### Step F: 2-bromo-7-((2S,5R)-2,5-diethyl-4-(1-(4-fluoro-2-(trifluoromethyl)phenyl)ethyl)piperazin-1-yl)-4-methylpyrazolo[1,5-a]pyrimidin-5(4H)-one

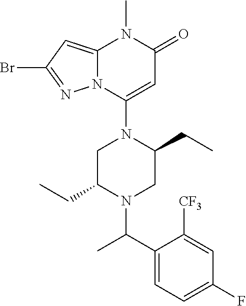 [SMILES: CC[C@H]1CN(C(C)c2ccc(F)cc2C(F)(F)F)[C@H](CC)CN1c1cc(=O)n(C)c2cc(Br)nn12]

To a mixture of 2-bromo-7-((2S,5R)-2,5-diethyl-4-(1-(4-fluoro-2-(trifluoromethyl)phenyl)ethyl)piperazin-1-yl)pyrazolo[1,5-a]pyrimidin-5(4H)-one (550 mg. 1 mmol) in 1,4-dioxane (5 mL) was added trimethyl phosphate (700 mg, 5 mmol) and K₂CO₃ (1.39 g, 10 mmol). The reaction mixture was stirred at 95° C. overnight. The mixture was added H₂O and extracted by ethyl acetate. The organic layer was washed with brine, dried over Na₂SO₄, filtered, and concentrated. The resulting crude product was purified by flash column chromatography to give the titled compound (260 mg, 47%). MS: M/e 558 (M+1)⁺.

### Step G: methyl 7-((2S,5R)-2,5-diethyl-4-(1-(4-fluoro-2-(trifluoromethyl)phenyl)ethyl)piperazin-1-yl)-4-methyl-5-oxo-4,5-dihydropyrazolo[1,5-a]pyrimidine-2-carboxylate

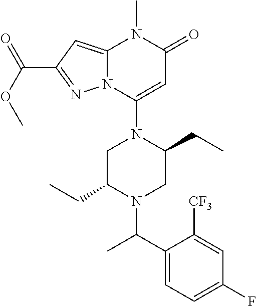 [SMILES: CC[C@H]1CN(C(C)c2ccc(F)cc2C(F)(F)F)[C@H](CC)CN1c1cc(=O)n(C)c2cc(C(=O)OC)nn12]

To a solution of 2-bromo-7-((2S,5R)-2,5-diethyl-4-(1-(4-fluoro-2-(trifluoromethyl)phenyl)ethyl)piperazin-1-yl)-4-methylpyrazolo[1,5-a]pyrimidin-5(4H)-one (130 mg, 0.23 mmol) in MeOH (20 mL) was added Pd(dppf)Cl₂ (7.3 mg, 0.01 mmol) and Et₃N (101 mg, 1 mmol). The reaction mixture was stirred under an CO atmosphere at 100° C. for 16 hours. The mixture was cooled to RT. The mixture was added to water and extracted by ethyl acetate. The organic layer was dried over Na₂SO₄, filtered, and concentrated. The resulting crude product was purified by flash column chromatography to give the titled compound (100 mg, 80%). MS: M/e 538 (M+1)⁺.

### Step H: 7-((2S,5R)-2,5-diethyl-4-(1-(4-fluoro-2-(trifluoromethyl)phenyl)ethyl)piperazin-1-yl)-2-(hydroxymethyl)-4-methylpyrazolo[1,5-a]pyrimidin-5(4H)-one

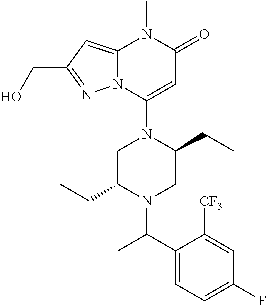 [SMILES: CC[C@H]1CN(C(C)c2ccc(F)cc2C(F)(F)F)[C@H](CC)CN1c1cc(=O)n(C)c2cc(CO)nn12]

To a mixture of methyl 7-((2S,5R)-2,5-diethyl-4-(1-(4-fluoro-2-(trifluoromethyl)phenyl)ethyl)piperazin-1-yl)-4-methyl-5-oxo-4,5-dihydropyrazolo[1,5-a]pyrimidine-2-carboxylate (80 mg, 0.15 mmol) in THF (10 mL) was added NaBH₄ (37 mg, 1 mmol). The resulted mixture was stirred at 70° C. overnight. The mixture was concentrated in vacuo. The residue was added to ice-water and extracted by ethyl acetate. The organic layer was dried over Na₂SO₄, filtered, and concentrated. The resulting residue was purified by flash column chromatography to give the titled compound (50 mg, 66%). MS: M/e 510 (M+1)⁺.

### Step I: (7-((2S,5R)-2,5-diethyl-4-(1-(4-fluoro-2-(trifluoromethyl)phenyl)ethyl)piperazin-1-yl)-4-methyl-5-oxo-4,5-dihydropyrazolo[1,5-a]pyrimidin-2-yl)methyl Methanesulfonate

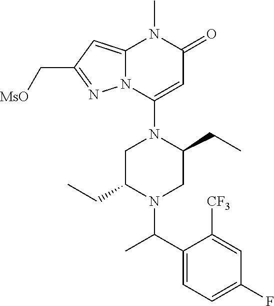 [SMILES: CC[C@H]1CN(C(C)c2ccc(F)cc2C(F)(F)F)[C@H](CC)CN1c1cc(=O)n(C)c2cc(COS(C)(=O)=O)nn12]

To a mixture of 7-((2S,5R)-2,5-diethyl-4-(1-(4-fluoro-2-(trifluoromethyl)phenyl)ethyl)piperazin-1-yl)-2-(hydroxymethyl)-4-methylpyrazolo[1,5-a]pyrimidin-5(4H)-one (50 mg, 0.1 mmol) in DCM (15 mL) was added methanesulfonyl chloride (22.6 mg, 0.2 mmol) and Et₃N (50 mg, 0.5 mmol). The resulted mixture was stirred at RT for 30 mins. The mixture was washed with NaHCO₃ aqueous solution, dried over Na₂SO₄, filtered, and concentrated. The resulting crude product was used directly in next step without further purification. MS: M/e 470 (M+1)⁺.

### Step J: 2-(7-((2S,5R)-2,5-diethyl-4-(1-(4-fluoro-2-(trifluoromethyl)phenyl)ethyl)piperazin-1-yl)-4-methyl-5-oxo-4,5-dihydropyrazolo[1,5-a]pyrimidin-2-yl)acetonitrile

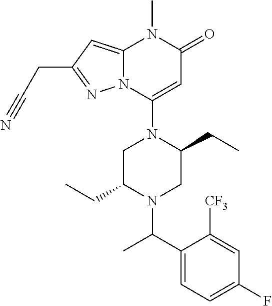 [SMILES: CC[C@H]1CN(C(C)c2ccc(F)cc2C(F)(F)F)[C@H](CC)CN1c1cc(=O)n(C)c2cc(CC#N)nn12]

To a mixture of (7-((2S,5R)-2,5-diethyl-4-(1-(4-fluoro-2-(trifluoromethyl)phenyl)ethyl)piperazin-1-yl)-4-methyl-5-oxo-4,5-dihydropyrazolo[1,5-a]pyrimidin-2-yl)methyl methanesulfonate (crude from above step I) in acetonitrile (10 mL) was added TMSCN (19.6 mg. 0.2 mmol) and K₂CO₃ (55.6 mg, 0.4 mmol). The reaction was stirred at 80° C. overnight. The mixture was added to water and extracted by ethyl acetate. The organic layer was dried over Na₂SO₄, filtered, and concentrated to dryness. The resulting residue was purified and separated into Compound A15a (8 mg) and Compound A15b (2 mg) by Prep-HPLC (Method A).

Compound A15a (the earlier peak) ¹HNMR (400 MHz, CD₃OD) δ 8.02 (dd, J=9.4, 5.8 Hz, 1H), 7.40 (d, J=8.8 Hz, 2H), 6.12 (s, 1H), 5.30 (s, 1H), 4.45 (s, 1H), 4.06 (s, 2H), 3.98 (s, 1H), 3.58 (d, J=11.5 Hz, 1H), 3.46 (s, 4H), 2.82 (d, J=9.1 Hz, 2H), 2.12 (d, J=12.0 Hz, 1H), 1.88 (dt, J=17.2, 7.8 Hz, 2H), 1.68-1.46 (m, 2H), 1.29 (d, J=6.4 Hz, 3H), 0.94 (t, J=7.4 Hz, 3H), 0.51 (t, J=7.5 Hz, 3H) ppm. MS: M/e 519 (M+1)⁺.

Compound A15b (the later peak): ¹HNMR (400 MHz, CD₃OD) δ 8.12-8.02 (m, 1H), 7.42 (t, J=8.5 Hz, 2H), 6.12 (s, 1H), 5.28 (s, 1H), 4.77 (s, 1H), 4.21 (d, J=6.2 Hz, 1H), 4.00 (s, 2H), 3.75 (d, J=10.7 Hz, 1H), 3.46 (s, 4H), 3.38 (s, 1H), 3.06-3.02 (m, 1H), 2.31 (d, J=10.1 Hz, 1H), 2.16 (dd, J=14.3, 7.0 Hz, 1H), 1.90-1.72 (m, 2H), 1.53 (d, J=7.3 Hz, 1H), 1.27 (d, J=6.3 Hz, 3H), 0.91 (t, J=7.5 Hz, 3H), 0.64 (t, J=7.2 Hz, 3H) ppm. MS: M/e 519 (M+1)⁺.

### Compound A16: 2-(7-((2S,5R)-2,5-diethyl-4-(1-(4-fluoro-2-methoxyphenyl)ethyl)piperazin-1-yl)-4-methyl-5-oxo-4,5-dihydropyrazolo[1,5-a]pyrimidin-2-yl)acetonitrile

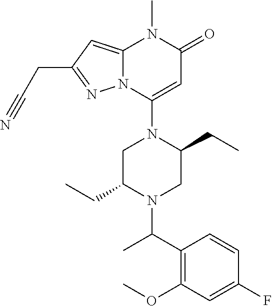 [SMILES: CC[C@H]1CN(C(C)c2ccc(F)cc2OC)[C@H](CC)CN1c1cc(=O)n(C)c2cc(CC#N)nn12]

A mixture of 1-(4-fluoro-2-methoxyphenyl)ethan-1-ol (68 mg, 0.4 mmol), 2-(7-((2S,5R)-2,5-diethylpiperazin-1-yl)-4-methyl-5-oxo-4,5-dihydropyrazolo[1,5-a]pyrimidin-2-yl)acetonitrile (65 mg, 0.2 mmol), (cyanomethyl)trimethylphosphonium iodide (97 mg, 0.4 mmol) and DIPEA (129 mg, 1 mmol) in MeCN (3 mL) was stirred at 100° C. overnight in a sealed tube. The reaction mixture was diluted with EA (20 mL), washed with brine (10 mL×3), dried over Na₂SO₄ and concentrated to dryness. The resulting residue was purified by Prep-HPLC (Method A) to give the titled compound (2 mg, 2%). ¹H NMR (400 MHz, DMSO-d₆) δ 7.45 (dt, J=23.2, 7.9 Hz, 1H), 6.87 (t, J=11.1 Hz, 1H), 6.82-6.71 (m, 1H), 6.11 (s, 1H), 5.23 (d, J=11.7 Hz, 1H), 4.70-4.28 (m, 1H), 4.23-3.98 (m, 3H), 3.85 (s, 0.5H), 3.80 (s, 3H), 3.57-3.40 (m, 1H), 3.33 (s, 3H), 3.25-2.98 (m, 1H), 2.85 (s, 1H), 2.60 (d, J=9.1 Hz, 0.5H), 2.36-2.15 (m, 1H), 2.02-1.79 (m, 1H), 1.75-1.37 (m, 3H), 1.16 (dd, J=17.3, 6.4 Hz, 3H), 0.80 (dt, J=29.2, 7.3 Hz, 3H), 0.53 (dt, J=20.1, 7.3 Hz, 3H) ppm. MS: M/e 481 (M+1)⁺.

### Compound A17: 2-(7-((2S,5R)-4-(1-(2-(difluoromethoxy)-4-fluorophenyl)ethyl)-2,5-diethylpiperazin-1-yl)-4-methyl-5-oxo-4,5-dihydropyrazolo[1,5-a]pyrimidin-2-yl)acetonitrile

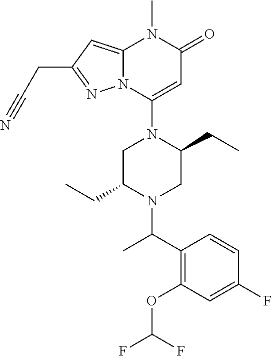 [SMILES: CC[C@H]1CN(C(C)c2ccc(F)cc2OC(F)F)[C@H](CC)CN1c1cc(=O)n(C)c2cc(CC#N)nn12]

A mixture of 1-(2-(difluoromethoxy)-4-fluorophenyl)ethan-1-ol (82 mg, 0.4 mmol), 2-(7-((2S,5R)-2,5-diethylpiperazin-1-yl)-4-methyl-5-oxo-4,5-dihydropyrazolo[1,5-a]pyrimidin-2-yl)acetonitrile (65 mg, 0.2 mmol), (cyanomethyl)trimethylphosphonium iodide (97 mg, 0.4 mmol) and DIPEA (129 mg, 1 mmol) in MeCN (3 mL) was stirred at 100° C. overnight in a sealed tube. The reaction mixture was diluted with EA (20 mL), washed with brine (10 mL×3), dried over Na₂SO₄ and concentrated to dryness. The resulting residue was purified by Prep-HPLC (Method A) to give the titled compound (3 mg, 2%). ¹H NMR (400 MHz, DMSO-d₆) δ 7.78-7.64 (m, 1H), 7.57-7.23 (m, 1H), 7.22-7.13 (m, 2H), 6.18 (s, 1H), 5.31 (d, J=15.5 Hz, 1H), 4.76-4.37 (m, 1H), 4.25-3.99 (m, 3H), 3.95-3.56 (m, 1H), 3.50 (d, J=11.4 Hz, 0.5H), 3.40 (s, 3H), 3.29 (d, J=10.5 Hz, 0.5H), 3.10 (d, J=10.4 Hz, 0.5H), 2.93 (s, 1H), 2.71 (d, J=9.4 Hz, 0.5H), 2.37-2.17 (m, 1H), 2.08-1.83 (m, 1H), 1.81-1.48 (m, 3H), 1.28 (dd, J=15.4, 6.0 Hz, 3H), 0.94-0.80 (m, 3H), 0.68-0.50 (m, 3H) ppm. MS: M/e 517 (M+1)⁺.

### Compound A18: 2-(7-((2S,5R)-4-(1-(2-(difluoromethyl)-4-fluorophenyl)ethyl)-2,5-diethylpiperazin-1-yl)-4-methyl-5-oxo-4,5-dihydropyrazolo[1,5-a]pyrimidin-2-yl)acetonitrile

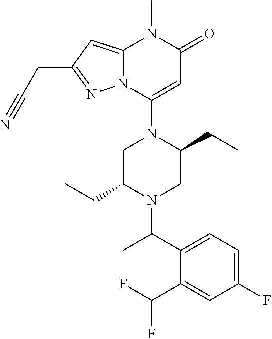 [SMILES: CC[C@H]1CN(C(C)c2ccc(F)cc2C(F)F)[C@H](CC)CN1c1cc(=O)n(C)c2cc(CC#N)nn12]

A mixture of 1-(2-(difluoromethyl)-4-fluorophenyl)ethan-1-ol (57 mg, 0.3 mmol), 2-(7-((2S,5R)-2,5-diethylpiperazin-1-yl)-4-methyl-5-oxo-4,5-dihydropyrazolo[1,5-a]pyrimidin-2-yl)acetonitrile (65 mg, 0.2 mmol), (cyanomethyl) trimethylphosphonium iodide (97 mg, 0.4 mmol) and DIPEA (103 mg, 0.8 mmol) in MeCN (2 mL) was stirred at 100° C. in a sealed tube overnight. The reaction mixture was diluted with water (20 mL), extracted with EtOAc (10 mL×3), dried over Na₂SO₄ and concentrated to dryness. The resulting residue was purified by Prep-TLC (MeOH:DCM=1:13) to give the titled compound (8 mg, 8%). ¹H NMR (400 MHz, CD₃OD) δ 7.82-7.62 (m, 1H), 7.59-7.15 (m, 3H), 6.12 (s, 1H), 5.30 (d, J=6.6 Hz, 1H), 4.81-4.38 (m, 1H), 4.16-3.74 (m, 4H), 3.61-3.35 (m, 4H), 3.21-2.80 (m, 2H), 2.40-2.18 (m, 1H), 2.11-1.79 (m, 2H), 1.77-1.49 (m, 2H), 1.33 (dd, J=13.9, 6.5 Hz, 3H), 0.91 (dt, J=22.4, 7.4 Hz, 3H), 0.62-0.48 (m, 3H) ppm. MS: M/e 501 (M+1)⁺.

### Compound A19: 2-(8-((2S,5R)-2,5-diethyl-4-(1-(pyridin-4-yl)ethyl)piperazin-1-yl)-5-methy-6-oxo-5,6-dihydroimidazo[1,2-b]pyridazin-2-yl)acetonitrile

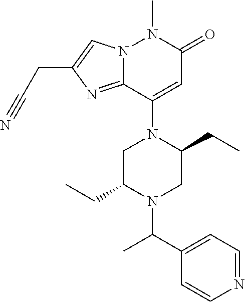 [SMILES: CC[C@H]1CN(C(C)c2ccncc2)[C@H](CC)CN1c1cc(=O)n(C)n2cc(CC#N)nc12]

### Step A: 1-(pyridin-4-yl)ethan-1-ol

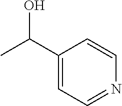

To a solution of 1-(pyridin-4-yl)ethan-1-one (1.2 g, 10 mmol) in MeOH (10 mL) was added NaBH₄ (190 mg, 5 mmol) at room temperature and the mixture was stirred at room temperature for 30 minutes. The resulting mixture was treated with water (50 ml), extracted with DCM (20 mL×2). The combined organic layers were dried over Na₂SO₄ and concentrated to dryness. The resulting residue was purified by flash column chromatography (MeOH:DCM=0-10% in 25 minutes) to give the titled compound (900 mg, 73%). MS: M/e 124 (M+1)⁺.

### Step B: 2-(8-((2S,5R)-2,5-diethyl-4-(1-(pyridin-4-yl)ethyl)piperazin-1-yl)-5-methyl-6-oxo-5,6-dihydroimidazo[1,2-b]pyridazin-2-yl)acetonitrile

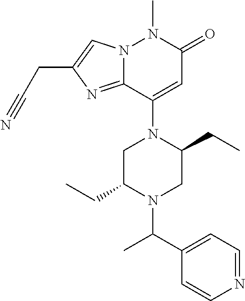 [SMILES: CC[C@H]1CN(C(C)c2ccncc2)[C@H](CC)CN1c1cc(=O)n(C)n2cc(CC#N)nc12]

A mixture of 1-(pyridin-4-yl)ethan-1-ol (37 mg, 0.3 mmol), 2-(8-((2S,5R)-2,5-diethylpiperazin-1-yl)-5-methyl-6-oxo-5,6-dihydroimidazo[1,2-b]pyridazin-2-yl)acetonitrile (65 mg, 0.2 mmol), (cyanomethyl) trimethylphosphonium iodide (97 mg, 0.4 mmol) and DIPEA (129 mg, 1 mmol) in MeCN (3 mL) was stirred at 100° C. in a sealed tube overnight. The reaction mixture was diluted with water (20 mL), extracted with EtOAc (10 mL×3), dried over Na₂SO₄ and concentrated to dryness. The resulting residue was purified by silica gel column chromatography MeOH:DCM=0-10% in 30 minutes to give the titled compound (5 mg, 6%). ¹H NMR (400 MHz, CDCl₃) δ 8.59 (s, 2H), 7.39 (s, 2H), 5.87 (s, 1H), 5.28 (d, J=11.8 Hz, 1H), 4.60-4.25 (m, 1H), 3.78 (d, J=8.5 Hz, 2H), 3.68-3.48 (m, 2H), 3.47-3.28 (m, 4H), 3.11-2.95 (m, 1H), 2.92-2.75 (m, 1H), 2.39-2.17 (m, 1H), 2.07 (s, 1H), 1.87 (s, 1H), 1.73 (s, 1H), 1.52 (d, J=6.5 Hz, 1H), 1.29 (d, J=5.7 Hz, 3H), 0.87 (dd, J=15.8, 8.2 Hz, 3H), 0.67-0.52 (m, 3H) ppm. MS: M/e 434 (M+1)⁺.

### Compound A20: 2-(7-((2S,5R)-4-(1-(4-cyclopropyl-2-fluorophenyl)ethyl)-5-ethyl-2-methylpiperazin-1-yl)-4-methyl-5-oxo-4,5-dihydropyrazolo[1,5-a]pyrimidin-2-yl)acetonitrile

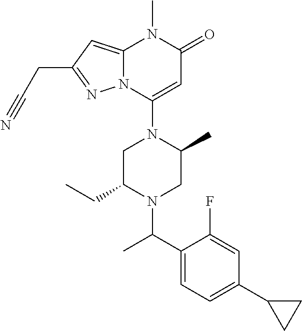 [SMILES: CC[C@@H]1CN(c2cc(=O)n(C)c3cc(CC#N)nn23)[C@@H](C)CN1C(C)c1ccc(C2CC2)cc1F]

### Step A: 1-(4-cyclopropyl-2-fluorophenyl)ethan-1-one

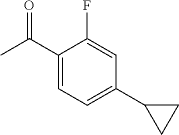

To a solution of 1-(4-bromo-2-fluorophenyl)ethan-1-one (1.95 g, 9 mol) in toluene (30 mL) was added cyclopropylboronic acid (1.16 g, 13.5 mol), (Cy₃P)₂PdCl₂, K₃PO₄ (2.86 g, 13.5 mmol) and H₂O (3 ml). The reaction mixture was protected by N₂ atmosphere and stirred at rt overnight. The mixture was added H₂O (20 ml) and extracted with EA (20 mL×3), then concentrated by using a rotary evaporator to give a residue. The resulting residue was purified by flash column chromatography to give the titled compound (1.6 g, 100%). MS: M/e 179 (M+1)⁺.

### Step B: 1-(4-cyclopropyl-2-fluorophenyl)ethan-1-ol

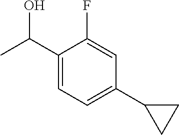

To a solution of 1-(4-cyclopropyl-2-fluorophenyl)ethan-1-one (1.6 g, 9 mol) in MeOH was added NaBH₄ (305 mg, 8 mmol). The reaction mixture was stirred at rt for 15 mins. The mixture was added H₂O (20 ml) and extracted with DCM (20 mL×3). The organic phase was dried with Na₂SO₄ and concentrated by using a rotary evaporator to give a residue. The resulting residue was purified by flash column chromatography to give the titled compound (1.2 g, 75%). MS: M/e 181 (M+1)⁺.

### Step A: 2-(7-((2S,5R)-4-(1-(4-cyclopropyl-2-fluorophenyl)ethyl)-5-ethyl-2-methylpiperazin-1-yl)-4-methyl-5-oxo-4,5-dihydropyrazolo[1,5-a]pyrimidin-2-yl)acetonitrile

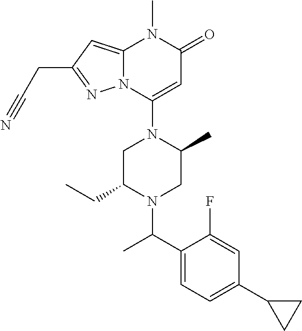 [SMILES: CC[C@@H]1CN(c2cc(=O)n(C)c3cc(CC#N)nn23)[C@@H](C)CN1C(C)c1ccc(C2CC2)cc1F]

To a solution of 2-(7-((2S,5R)-5-ethyl-2-methylpiperazin-1-yl)-4-methyl-5-oxo-4,5-dihydropyrazolo[1,5-a]pyrimidin-2-yl)acetonitrile (100 mg, 0.3 mmol), 1-(4-cyclopropyl-2-fluorophenyl)ethan-1-ol (108 mg, 0.6 mmol) and (cyanomethyl)trimethylphosphonium iodide (216 mg, 0.9 mmol) in CH₃CN (10 mL) was added DIPEA (258 mg, 5 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC (Method B) to give the Compound A20a (15 mg) and Compound A20b (17 mg).

Compound A20a (the earlier peak): ¹H NMR (400 MHz, CD₃OD)) δ 7.38 (t, J=7.8 Hz, 1H), 6.91 (d, J=8.0 Hz, 1H), 6.76 (dd, J=11.9, 1.4 Hz, 1H), 6.12 (s, 1H), 5.30 (s, 1H), 4.82 (m, 1H), 4.12 (d, J=6.6 Hz, 1H), 3.97 (d, J=5.8 Hz, 1H), 3.75 (d, J=12.0 Hz, 1H), 3.46 (s, 3H), 3.36 (m, 2H), 3.03 (dd, J=11.8, 3.7 Hz, 1H), 2.78 (dd, J=11.7, 2.3 Hz, 1H), 2.46 (d, J=9.3 Hz, 1H), 1.90 (m, 1H), 1.70 (dd, J=6.8, 3.4 Hz, 1H), 1.55 (m, 1H), 1.32 (dd, J=6.5, 1.7 Hz, 6H), 0.98 (m, 2H), 0.68 (t, J=7.1 Hz, 5H) ppm. MS: M/e 477 (M+1)⁺.

Compound A20b (the later peak): ¹H NMR (400 MHz, CD₃OD) δ 7.44 (t, J=7.9 Hz, 1H), 6.88 (d, J=8.1 Hz, 1H), 6.72 (dd, J=11.9, 1.4 Hz, 1H), 6.12 (s, 1H), 5.33 (s, 1H), 4.65 (s, 1H), 3.99 (m, 3H), 3.55 (d, J=12.6 Hz, 1H), 3.46 (s, 3H), 3.33 (s, 1H), 3.13 (d, J=9.5 Hz, 1H), 2.84 (dd, J=12.0, 3.6 Hz, 1H), 2.21 (d, J=11.3 Hz, 1H), 1.87 (m, 2H), 1.56 (dd, J=13.4, 7.3 Hz, 1H), 1.30 (t, J=6.6 Hz, 3H), 1.18 (d, J=6.6 Hz, 3H), 0.96 (m, 5H), 0.66 (m, 2H) ppm. MS: M/e 477 (M+1)⁺.

### Compound A21: 2-(7-((2S,5R)-4-(1-(4-cyclopropyl-2-fluorophenyl)ethyl)-2,5-diethylpiperazin-1-yl)-4-methyl-5-oxo-4,5-dihydropyrazolo[1,5-a]pyrimidin-2-yl)acetonitrile

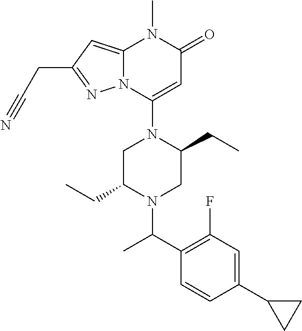 [SMILES: CC[C@H]1CN(C(C)c2ccc(C3CC3)cc2F)[C@H](CC)CN1c1cc(=O)n(C)c2cc(CC#N)nn12]

A solution of 2-(7-((2S,5R)-2,5-diethylpiperazin-1-yl)-4-methyl-5-oxo-4,5-dihydropyrazolo[1,5-a]pyrimidin-2-yl)acetonitrile (50 mg, 0.15 mmol), 1-(4-cyclopropyl-2-fluorophenyl)ethan-1-ol (54.9 mg, 0.31 mmol), (cyanomethyl)trimethylphosphonium iodide (111 mg, 0.46 mmol) and DIPEA (196.6 mg, 1.52 mmol) in CH₃CN (2 ml) was stirred at 100° C. overnight. The reaction was diluted with EtOAc (10 ml) and washed with brine (10 ml). The organic layer was concentrated under reduced pressure. The resulting residue was purified by flash column chromatography and then Prep-HPLC (Method A) to give the titled compound (21 mg). ¹H NMR (400 MHz, CD₃OD) δ 7.40 (dt, J=22.0, 7.8 Hz, 1H), 6.90 (t, J=8.1 Hz, 1H), 6.74 (t, J=11.4 Hz, 1H), 6.12 (s, 1H), 5.28 (d, J=8.1 Hz, 1H), 4.67 (s, 0.5H), 4.42 (s, 0.5H), 4.15-4.03 (m, 1H), 3.99 (d, J=3.6 Hz, 2.5H), 3.78 (d, J=12.6 Hz, 0.5H), 3.54 (d, J=13.1 Hz, 0.5H), 3.46 (s, 3H), 3.37 (d, J=13.1 Hz, 0.5H), 3.12 (d, J=9.2 Hz, 0.5H), 2.96 (s, 1H), 2.77 (d, J=9.1 Hz, 0.5H), 2.45 (d, J=8.7 Hz, 0.5H), 2.33 (d, J=12.6 Hz, 0.5H), 2.17-1.46 (m, 5H), 1.30 (dd, J=13.5, 6.5 Hz, 3H), 1.03-0.81 (m, 5H), 0.71-0.51 (m, 5H) ppm. MS: M/e 491 (M+1)⁺.

### Compound A22: 2-(7-((2S,5R)-4-(1-(6-cyclopropylpyridin-3-yl)ethyl)-5-ethyl-2-methylpiperazin-1-yl)-4-methyl-5-oxo-4,5-dihydropyrazolo[1,5-a]pyrimidin-2-yl)acetonitrile

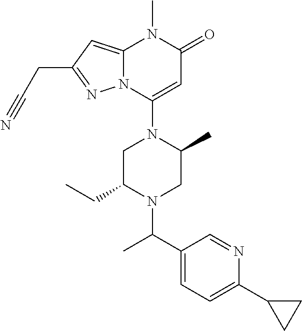 [SMILES: CC[C@@H]1CN(c2cc(=O)n(C)c3cc(CC#N)nn23)[C@@H](C)CN1C(C)c1ccc(C2CC2)nc1]

To a solution of 2-(7-((2S,5R)-5-ethyl-2-methylpiperazin-1-yl)-4-methyl-5-oxo-4,5-dihydropyrazolo[1,5-a]pyrimidin-2-yl)acetonitrile (100 mg, 0.5 mmol), 1-(6-cyclopropylpyridin-3-yl)ethan-1-ol (200 mg, 1.22 mmol) and (cyanomethyl)trimethylphosphonium iodide (240 mg, 1 mmol) in CH₃CN (10 mL) was added DIPEA (259 mg, 2 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC (Method B) to give the Compound A22a (19 mg) and Compound A22b (22 mg).

Compound A22a (the earlier peak): ¹H NMR (400 MHz, CD₃OD) δ 8.31 (d, J=1.7 Hz, 1H), 7.72 (dd, J=8.1, 2.1 Hz, 1H), 7.20 (d, J=8.1 Hz, 1H), 6.12 (s, 1H), 5.31 (s, 1H), 3.97 (d, 0.1=5.8 Hz, 1H), 3.83 (d, J=6.5 Hz, 1H), 3.73 (d, J=12.3 Hz, 1H), 3.46 (s, 3H), 3.36 (dd, J=12.7, 2.5 Hz, 3H), 3.05 (dd, J=11.7, 3.6 Hz, 1H), 2.81 (dd, J=11.8, 2.2 Hz, 1H), 2.39 (d, J=9.4 Hz, 1H), 2.09 (m, 1H), 1.76 (m, 1H), 1.56 (dd, J=13.5, 7.5 Hz, 1H), 1.34 (d, J=6.5 Hz, 6H), 1.02 (dt, J=8.1, 2.9 Hz, 2H), 0.94 (m, 2H), 0.68 (t, J=7.4 Hz, 3H) ppm. MS: M/e 460 (M+1)⁺.

Compound A22b (the later peak): ¹H NMR (400 MHz, CD₃OD) δ 8.33 (d, J=1.6 Hz, 1H), 7.72 (dd, J=8.1, 2.0 Hz, 1H), 7.17 (d, J=8.1 Hz, 1H), 6.12 (s, 1H), 5.33 (s, 1H), 4.62 (s, 1H), 3.98 (dd, J=15.8, 9.2 Hz, 2H), 3.69 (q, J=6.5 Hz, 1H), 3.57 (m, 1H), 3.46 (s, 3H), 3.32 (s, 1H), 3.16 (d, J=9.8 Hz, 1H), 2.84 (dd, J=12.0, 3.6 Hz, 1H), 2.15 (m, 1H), 2.07 (m, 1H), 1.87 (ddd, J=13.5, 10.6, 7.2 Hz, 1H), 1.58 (dd, J=13.6, 7.3 Hz, 1H), 1.32 (d, J=6.6 Hz, 3H), 1.16 (d, J=6.6 Hz, 3H), 0.97 (m, 7H) ppm. MS: M/e 460 (M+1)⁺.

### Compound A23: 2-(7-((2S,5R)-4(1-(6-cyclopropylpyridin-3-yl)ethyl)-2,5-diethylpiperazin-1-yl)-4-methyl-5-oxo-4,5-dihydropyrazolo[1,5-a]pyrimidin-2-yl)acetonitrile

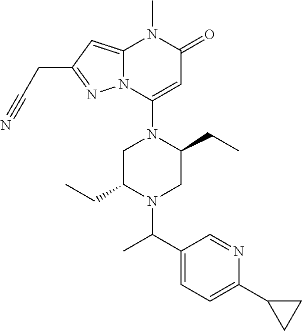 [SMILES: CC[C@H]1CN(C(C)c2ccc(C3CC3)nc2)[C@H](CC)CN1c1cc(=O)n(C)c2cc(CC#N)nn12]

A mixture of 1-(6-cyclopropylpyridin-3-yl)ethan-1-ol (33 mg, 0.2 mmol), 2-(7-((2S,5R)-2,5-diethylpiperazin-1-yl)-4-methyl-5-oxo-4,5-dihydropyrazolo[1,5-a]pyrimidin-2-yl)acetonitrile (33 mg, 0.1 mmol), (cyanomethyl) trimethylphosphonium iodide (97 mg, 0.4 mmol) and DIPEA (52 mg, 0.4 mmol) in MeCN (2 mL) was stirred at 100° C. overnight. The reaction mixture was diluted with water (20 mL), extracted with EtOAc (5 mL×3), dried over Na₂SO₄ and concentrated to dryness. The resulting residue was purified by Prep-HPLC (Method A) to give the titled compound (2 mg, 4%). ¹H NMR (400 MHz, CD₃OD) δ 8.32 (d, J=10.2 Hz, 1H), 7.71 (d, J=7.9 Hz, 1H), 7.26-7.13 (m, 1H), 6.12 (s, 1H), 5.29 (d, J=8.4 Hz, 1H), 4.75-4.35 (m, 1H), 4.14-3.90 (m, 2.5H), 3.88-3.53 (m, 2H), 3.46 (s, 3H), 3.16 (d, J=9.9 Hz, 1H), 2.98 (s, 1H), 2.80 (d, J=12.4 Hz, 0.5H), 2.42-2.22 (m, 1H), 2.14-1.76 (m, 3H), 1.73-1.49 (m, 2H), 1.33 (dd, J=11.9, 6.5 Hz, 3H), 1.08-0.81 (m, 7H), 0.67-0.49 (m, 3H) ppm. MS: M/e 474 (M+1)⁺.

### Compound A24: 2-(7-((2S,5R)-4-(1-(6-cyclopropylpyridin-3-yl)ethyl)-5-ethyl-2-methylpiperazin-1-yl)-6-fluoro-4-methyl-5-oxo-4,5-dihydropyrazolo[1,5-a]pyrimidin-2-yl)acetonitrile

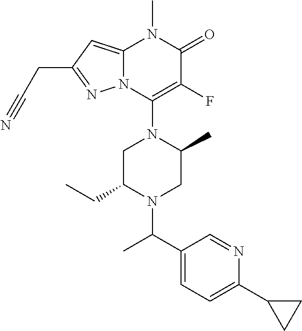 [SMILES: CC[C@@H]1CN(c2c(F)c(=O)n(C)c3cc(CC#N)nn23)[C@@H](C)CN1C(C)c1ccc(C2CC2)nc1]

To a solution of 2-(7-((2S,5R)-4-(1-(6-cyclopropylpyridin-3-yl)ethyl)-5-ethyl-2-methylpiperazin-1-yl)-4-methyl-5-oxo-4,5-dihydropyrazolo[1,5-a]pyrimidin-2-yl)acetonitrile (120 mg, 0.26 mmol) in CH₃CN (10 mL) was added Select F (101 mg, 0.28 mmol). The reaction mixture was sealed in a bottle and stirred at rt for 2 hours, The mixture was diluted with water and extracted with EA (20 mL×3). The combined organic layers were washed with brine, dried over Na₂SO₄, filtered, and concentrated to dryness. The resulting residue was purified by Prep-HPLC (Method B) to give the Compound A24a (7 mg) and Compound A24b (15 mg).

Compound A24a (the earlier peak): ¹H NMR (400 MHz, CD₃OD) δ 8.31 (d, J=1.5 Hz, 1H), 7.72 (dd, J=8.1, 2.0 Hz, 1H), 7.21 (d, J=8.1 Hz, 1H), 6.16 (s, 1H), 4.31 (s, 1H), 3.99 (q, J=6.5 Hz, 1H), 3.72 (d, J=12.1 Hz, 1H), 3.52 (s, 3H), 3.32 (s, 2H), 3.14 (m, 2H), 2.55 (dd, J=11.5, 4.5 Hz, 1H), 2.35 (d, J=5.7 Hz, 1H), 2.09 (m, 1H), 1.69 (dd, J=16.2, 8.5 Hz, 2H), 1.39 (d, J=6.7 Hz, 3H), 1.30 (d, J=6.5 Hz, 3H), 1.03 (m, 2H), 0.94 (m, 2H), 0.67 (t, J=7.4 Hz, 3H) ppm. MS: M/e 478 (M+1)⁺.

Compound A24b (the later peak): ¹H NMR (400 MHz, CD₃OD) δ 8.37 (d, J=1.6 Hz, 1H), 7.75 (dd, J=8.1, 2.0 Hz, 1H), 7.18 (d, J=8.1 Hz, 1H), 6.17 (s, 1H), 4.14 (d, J=6.0 Hz, 1H), 3.98 (d, J=5.8 Hz, 1H), 3.89 (dd, J=13.6, 9.6 Hz, 2H), 3.53 (s, 3H), 3.35 (d, J=3.3 Hz, 2H), 3.07 (m, 1H), 2.84 (dd, J=11.7, 3.5 Hz, 1H), 2.16 (dd, J=11.7, 4.1 Hz, 1H), 2.07 (m, 1H), 1.83 (m, 1H), 1.62 (dd, J=13.7, 6.9 Hz, 1H), 1.33 (d, J=6.6 Hz, 3H), 1.13 (d, J=6.5 Hz, 3H), 1.01 (m, 2H), 0.91 (m, 5H) ppm. MS: M/e 478 (M+1)⁺.

### Compound A25: 2-(7-((2S,5R)-2,5-diethyl-4-(1-(5-isopropoxypyridin-2-yl)ethyl)piperazin-1-yl)-4-methyl-5-oxo-4,5-dihydropyrazolo[1,5-a]pyrimidin-2-yl)acetonitrile

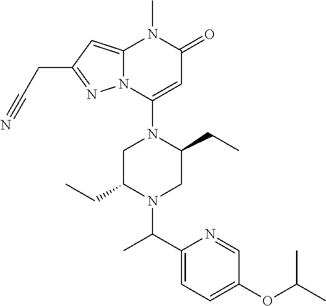 [SMILES: CC[C@H]1CN(C(C)c2ccc(OC(C)C)cn2)[C@H](CC)CN1c1cc(=O)n(C)c2cc(CC#N)nn12]

A mixture of 1-(5-isopropoxypyridin-2-yl)ethan-1-ol (33 mg, 0.2 mmol), 2-(7-((2S,5R)-2,5-diethylpiperazin-1-yl)-4-methyl-5-oxo-4,5-dihydropyrazolo[1,5-a]pyrimidin-2-yl)acetonitrile (33 mg, 0.1 mmol), (cyanomethyl) trimethylphosphonium iodide (97 mg, 0.4 mmol) and DIPEA (52 mg, 0.4 mmol) in MeCN (2 mL) was stirred at 100° C. overnight. The reaction mixture was diluted with water (20 mL), extracted with EtOAc (5 mL×3), dried over Na₂SO₄ and concentrated to dryness. The resulting residue was purified by Prep-HPLC (Method A) to give the titled compound (5 mg, 10%). ¹H NMR (400 MHz, CD₃OD) δ 8.08 (s, 1H), 7.51 (t, J=8.8 Hz, 1H), 7.43 (s, 1H), 6.12 (s, 1H), 5.30 (d, J=8.7 Hz, 1H), 4.74-4.40 (m, 2H), 4.13-3.87 (m, 3H), 3.78 (s, 1H), 3.63-3.38 (m, 4H), 3.20-2.82 (m, 2H), 2.42-2.17 (m, 1H), 2.16-1.79 (m, 2H), 1.74-1.48 (m, 2H), 1.33 (d, J=5.5 Hz, 9H), 0.98-0.81 (m, 3H), 0.67-0.52 (m, 3H) ppm. MS: M/e 492 (M+1)⁺.

### Compound A26: 2-(7-((2S,5R)-4-(1-(6-cyclopropyl-2-fluoropyridin-3-yl)ethyl)-2,5-diethylpiperazin-1-yl)-4-methyl-5-oxo-4,5-dihydropyrazolo[1,5-a]pyrimidin-2-yl)acetonitrile

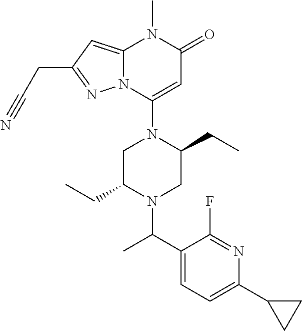 [SMILES: CC[C@H]1CN(C(C)c2ccc(C3CC3)nc2F)[C@H](CC)CN1c1cc(=O)n(C)c2cc(CC#N)nn12]

A mixture of 1-(6-cyclopropyl-2-fluoropyridin-3-yl)ethan-1-ol (36 mg, 0.2 mmol), 2-(7-((2S,5R)-2,5-diethylpiperazin-1-yl)-4-methyl-5-oxo-4,5-dihydropyrazolo[1,5-a]pyrimidin-2-yl)acetonitrile (33 mg, 0.1 mmol), (cyanomethyl) trimethylphosphonium iodide (97 mg, 0.4 mmol) and DIPEA (52 mg, 0.4 mmol) in MeCN (5 mL) was stirred at 100° C. overnight. The reaction mixture was diluted with water (40 mL), extracted with EtOAc (10 mL×3), dried over Na₂SO₄ and concentrated to dryness. The resulting residue was purified by Prep-HPLC (Method A) to give the titled compound (9 mg, 18%). ¹H NMR (400 MHz, DMSO-d₆) δ 7.85 (dd, J=18.1, 10.3 Hz, 1H), 7.26 (d, J=7.3 Hz, 1H), 6.10 (s, 1H), 5.23 (d, J=13.9 Hz, 1H), 4.67-4.26 (m, 1H), 4.10 (d, J=6.5 Hz, 2H), 4.01-3.90 (m, 0.5H), 3.87-3.74 (m, 1H), 3.58-3.38 (m, 1H), 3.32-3.28 (m, 2H), 3.27-2.97 (m, 1H), 2.90-2.73 (m, 1H), 2.70-2.51 (m, 1H), 2.30 (d, J=10.1 Hz, 0.5H), 2.18-1.86 (m, 2H), 1.83-1.35 (m, 4H), 1.24 (dd, J=18.4, 6.5 Hz, 3H), 0.94 (dd, J=7.9, 3.0 Hz, 2H), 0.89-0.69 (m, 5H), 0.61-0.43 (m, 3H) ppm. MS: M/e 492 (M+1)⁺.

### Compound A27: 2-(7-((2S,5R)-4-(1-(benzo[d]thiazol-6-yl)ethyl)-2,5-diethylpiperazin-1-yl)-4-methyl-5-oxo-4,5-dihydropyrazolo[1,5-a]pyrimidin-2-yl)acetonitrile

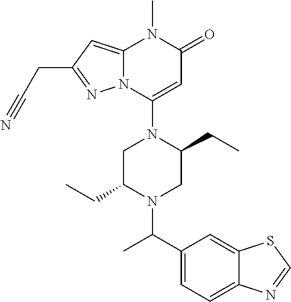 [SMILES: CC[C@H]1CN(C(C)c2ccc3ncsc3c2)[C@H](CC)CN1c1cc(=O)n(C)c2cc(CC#N)nn12]

A solution of 2-(7-((2S,5R)-2,5-diethylpiperazin-1-yl)-4-methyl-5-oxo-4,5-dihydropyrazolo[1,5-a]pyrimidin-2-yl)acetonitrile (200 mg, 0.61 mmol), 1-(benzo[d]thiazol-6-yl)ethan-1-ol (218 mg, 1.22 mmol), (cyanomethyl)trimethylphosphonium iodide (445 mg, 1.83 mmol) and DIPEA (787 mg, 6.10 mmol) in CH₃CN (6 ml) was stirred at 100° C. overnight. The reaction was diluted with EtOAc (15 ml) and washed with brine (10 ml). The organic layer was concentrated under reduced pressure. The resulting residue was purified by flash column chromatography to give the titled Compound A27, which was further separated into Compound A27a (22 mg) and Compound A27b (48 mg, crude) by Prep-HPLC (Method A).

Compound A27a (the earlier peak): ¹H NMR (400 MHz, DMSO-d₆) δ 9.33 (s, 1H), 8.13 (s, 1H), 8.05 (d, J=8.4 Hz, 1H), 7.55 (d, J=8.2 Hz, 1H), 6.10 (s, 1H), 5.20 (s, 1H), 4.66 (s, 1H), 4.10 (s, 2H), 3.89 (q, J=6.2 Hz, 1H), 3.46 (d, J=11.3 Hz, 1H), 3.24 (d, J=9.8 Hz, 1H), 2.90 (q, J=7.8 Hz, 2H), 2.55 (s, 3H), 2.31 (d, J=8.4 Hz, 1H), 2.09-1.94 (m, 1H), 1.72-1.60 (m, 1H), 1.60-1.42 (m, 2H), 1.31 (d, J=6.3 Hz, 3H), 0.78 (t, J=7.5 Hz, 3H), 0.49 (t, J=7.3 Hz, 3H) ppm. MS: M/e 490 (M+1)⁺.

Compound A27b (the later peak): 68.2% purity (214 nm), MS: M/e 490 (M+1)⁺.

### Compound A28: 2-(7-((2S,5R)-4-(1-(benzo[d]thiazol-6-yl)ethyl)-5-ethyl-2-methylpiperazin-1-yl)-4-methyl-5-oxo-4,5-dihydropyrazolo[1,5-a]pyrimidin-2-yl)acetonitrile

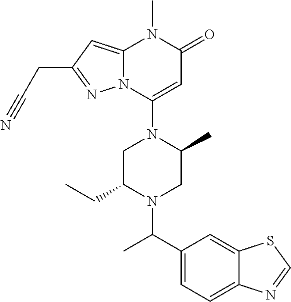 [SMILES: CC[C@@H]1CN(c2cc(=O)n(C)c3cc(CC#N)nn23)[C@@H](C)CN1C(C)c1ccc2ncsc2c1]

To a mixture of 2-(7-((2S,5R)-5-ethyl-2-methylpiperazin-1-yl)-4-methyl-5-oxo-4,5-dihydropyrazolo[1,5-a]pyrimidin-2-yl)acetonitrile (30 mg, 25.2 mmol) in acetonitrile (2 mL) was added (cyanomethyl)trimethylphosphonium iodide (120 mg, 0.5 mmol), 1-(benzo[d]thiazol-6-yl)ethan-1-ol (50 mg, 0.28 mmol) and DIPEA (129 mg, 1 mmol). The reaction mixture was sealed and stirred under nitrogen protection at 105° C. overnight. The mixture was added H₂O and extracted by ethyl acetate. The organic phase was washed with brine, dried over Na₂SO₄, filtered, and concentrated to dryness. The resulting residue was purified and separated into Compound A28a (3 mg) and Compound A28b (3 mg) by Prep-HPLC (Method B).

Compound A28a (the earlier peak): ¹H NMR (400 MHz, CD₃OD) δ 9.21 (s, 1H), 8.09 (s, 1H), 8.05 (d, J=8.5 Hz, 1H), 7.64 (d, J=7.5 Hz, 1H), 6.12 (s, 1H), 5.31 (s, 1H), 4.85 (s, 1H), 3.98 (d, J=5.0 Hz, 3H), 3.71 (d, J=12.6 Hz, 1H), 3.46 (s, 3H), 3.39 (d, J=9.9 Hz, 1H), 3.09 (dd, J=11.8, 3.6 Hz, 1H), 2.89 (d, J=9.9 Hz, 1H), 2.45 (d, J=9.0 Hz, 1H), 1.76 (m, 1H), 1.62 (dd, J=13.4, 7.2 Hz, 1H), 1.40 (t, J=6.0 Hz, 6H), 0.63 (t, J=7.4 Hz, 3H) ppm. MS: M/e 476 (M+1)⁺.

Compound A28b (the later peak): ¹H NMR (400 MHz, CD₃OD) δ 9.19 (s, 1H), 8.09 (s, 1H), 8.01 (d, J=8.5 Hz, 1H), 7.66 (d, J=8.5 Hz, 1H), 6.11 (s, 1H), 5.34 (s, 1H), 4.62 (s, 1H), 4.03 (d, J=12.6 Hz, 1H), 3.96 (s, 2H), 3.84 (d, J=6.5 Hz, 1H), 3.62 (d, J=11.4 Hz, 1H), 3.46 (s, 3H), 3.22 (d, J=10.6 Hz, 1H), 2.87 (m, 1H), 2.19 (d, J=12.1 Hz, 1H), 1.90 (d, J=4.2 Hz, 1H), 1.63 (dd, J=13.4, 7.0 Hz, 1H), 1.39 (d, J=6.4 Hz, 3H), 1.17 (d, J=6.5 Hz, 3H), 0.99 (t, J=7.3 Hz, 3H) ppm. MS: M/e 476 (M+1)⁺.

### Compound A29: 2-(7-((2S,5R)-5-ethyl-2-methyl-4-(1-(thiazolo[5,4-b]pyridin-5-yl)ethyl)piperazin-1-yl)-4-methyl-5-oxo-4,5-dihydropyrazolo[1,5-a]pyrimidin-2-yl)acetonitrile

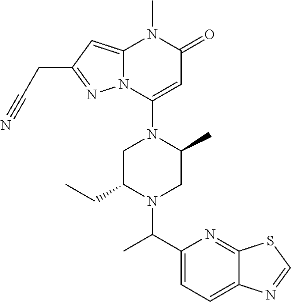 [SMILES: CC[C@@H]1CN(c2cc(=O)n(C)c3cc(CC#N)nn23)[C@@H](C)CN1C(C)c1ccc2ncsc2n1]

To a solution of 2-(7-((2S,5R)-5-ethyl-2-methylpiperazin-1-yl)-4-methyl-5-oxo-4,5-dihydropyrazolo[1,5-a]pyrimidin-2-yl)acetonitrile (150 mg, 0.5 mmol), 1-(thiazolo[5,4-b]pyridin-5-yl)ethan-1-ol (180 mg, 1 mmol) and (cyanomethyl)trimethylphosphonium iodide (361 mg, 1.5 mmol) in CH₃CN (10 mL) was added DIPEA (387 mg, 3 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified and separated into Compound A29a (34 mg) and Compound A29b (35 mg) by Prep-HPLC (Method B).

Compound A29a (the earlier peak): ¹H NMR (400 MHz, CD₃OD) δ 9.34 (s, 1H), 8.43 (d, J=8.5 Hz, 1H), 7.82 (d, J=8.5 Hz, 1H), 6.12 (s, 1H), 5.32 (s, 1H), 4.84 (s, 1H), 4.17 (t, J=6.6 Hz, 1H), 3.71 (d, J=12.3 Hz, 1H), 3.44 (m, 4H), 3.32 (s, 2H), 3.15 (dd, J=11.8, 3.6 Hz, 1H), 2.85 (dd, J=11.8, 2.6 Hz, 1H), 2.41 (d, J=8.0 Hz, 1H), 1.77 (m, 1H), 1.67 (m, 1H), 1.44 (d, J=6.7 Hz, 3H), 1.36 (d, J=6.6 Hz, 3H), 0.67 (t, J=7.4 Hz, 3H) ppm. MS: M/e 477 (M+1)⁺.

Compound A29b (the later peak): ¹H NMR (400 MHz, CD₃OD) δ 9.33 (s, 1H), 8.40 (d, J=8.6 Hz, 1H), 7.86 (d, J=8.6 Hz, 1H), 6.12 (s, 1H), 5.35 (s, 1H), 4.63 (s, 1H), 3.99 (m, 3H), 3.64 (d, J=10.4 Hz, 1H), 3.46 (s, 3H), 3.32 (s, 1H), 3.21 (d, J=10.2 Hz, 1H), 2.96 (dd, J=12.1, 3.7 Hz, 1H), 2.11 (m, 1H), 1.91 (m, 1H), 1.59 (dd, J=13.5, 7.3 Hz, 1H), 1.42 (d, J=6.7 Hz, 3H), 1.19 (d, J=6.6 Hz, 3H), 0.99 (t, J=7.4 Hz, 3H) ppm. MS: M/e 477 (M+1)⁺.

### Compound A30: 2-(7-((2S,5R)-5-ethyl-2-methyl-4-(1-(2-methylbenzo[d]thiazol-6-yl)ethyl)piperazin-1-yl)-4-methyl-5-oxo-4,5-dihydropyrazolo[1,5-a]pyrimidin-2-yl)acetonitrile

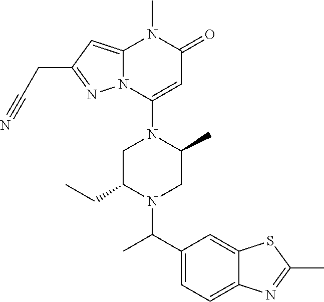 [SMILES: CC[C@@H]1CN(c2cc(=O)n(C)c3cc(CC#N)nn23)[C@@H](C)CN1C(C)c1ccc2nc(C)sc2c1]

To a solution of 2-(7-((2S,5R)-5-ethyl-2-methylpiperazin-1-yl)-4-methyl-5-oxo-4,5-dihydropyrazolo[1,5-a]pyrimidin-2-yl)acetonitrile (100 mg, 0.3 mmol), 1-(2-methylbenzo[d]thiazol-6-yl)ethan-1-ol (100 mg, 0.5 mmol) and (cyanomethyl)trimethylphosphonium iodide (241 mg, 1 mmol) in CH₃CN (5 mL) was added DIPEA (258 mg, 2 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified and separated into Compound A30a (3 mg) and Compound A30b (5 mg) by Prep-HPLC (Method B).

Compound A30a (the earlier peak): ¹H NMR (400 MHz, CD₃OD) δ 7.94 (s, 1H), 7.86 (d, J=8.4 Hz, 1H), 7.55 (m, 1H), 6.12 (s, 1H), 5.30 (s, 1H), 4.85 (s, 1H), 3.97 (s, 2H), 3.93 (d, J=6.5 Hz, 1H), 3.71 (d, J=12.5 Hz, 1H), 3.46 (d, J=6.7 Hz, 3H), 3.38 (d, J=12.2 Hz, 1H), 3.07 (dd, 1=11.8, 3.6 Hz, 1H), 2.87 (d, J=11.7 Hz, 1H), 2.83 (s, 3H), 2.45 (d, J=9.8 Hz, 1H), 1.74 (m, 1H), 1.60 (dd, J=13.1, 7.5 Hz, 1H), 1.38 (d, J=6.5 Hz, 6H), 0.63 (t, J=7.4 Hz, 3H) ppm. MS: M/e 490 (M+1)⁺.

Compound A30b (the later peak): ¹H NMR (400 MHz, CD₃OD) δ 7.94 (s, 1H), 7.82 (d, J=8.4 Hz, 1H), 7.56 (d, J=8.4 Hz, 1H), 6.11 (s, 1H), 5.34 (s, 1H), 4.62 (s, 1H), 4.02 (d, J=12.0 Hz, 1H), 3.96 (s, 2H), 3.79 (q, J=6.4 Hz, 1H), 3.61 (d, J=10.4 Hz, 1H), 3.46 (s, 3H), 3.21 (d, J=10.0 Hz, 1H), 2.86 (dd, J=12.1, 3.7 Hz, 1H), 2.81 (s, 3H), 2.18 (d, J=11.5 Hz, 1H), 1.89 (m, 1H), 1.61 (dd, J=13.5, 7.2 Hz, 1H), 1.37 (d, J=6.5 Hz, 3H), 1.16 (d, J=6.6 Hz, 3H), 0.98 (t, J=7.4 Hz, 3H) ppm. MS: M/e 490 (M+1)⁺.

### Compound A31: 2-(7-((2S,5R)-2,5-diethyl-4-(1-(2-methylbenzo[d]thiazol-6-yl)ethyl)piperazin-1-yl)-4-methyl-5-oxo-4,5-dihydropyrazolo[1,5-a]pyrimidin-2-yl)acetonitrile

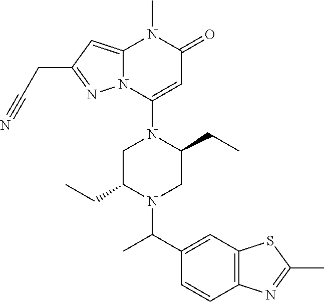 [SMILES: CC[C@H]1CN(C(C)c2ccc3nc(C)sc3c2)[C@H](CC)CN1c1cc(=O)n(C)c2cc(CC#N)nn12]

A solution of 2-(7-((2S,5R)-2,5-diethylpiperazin-1-yl)-4-methyl-5-oxo-4,5-dihydropyrazolo[1,5-a]pyrimidin-2-yl)acetonitrile (50 mg, 0.15 mmol), 1-(2-methylbenzo[d]thiazol-6-yl)ethan-1-ol (58.8 mg, 0.30 mmol), (cyanomethyl)trimethylphosphonium iodide (111.1 mg, 0.46 mmol) and DIPEA (196.6 mg, 1.52 mmol) in CH₃CN (2 ml) was stirred at 100° C. overnight. The reaction was diluted with EtOAc (10 ml) and washed with brine (10 ml). The organic layer was concentrated under reduced pressure. The resulting residue was purified by flash column chromatography and then further purified by Prep-HPLC (Method A) to give the titled compound (16 mg). ¹H NMR (400 MHz, CD₃OD) δ 7.94 (d, J=10.0 Hz, 1H), 7.89-7.80 (m, 1H), 7.55 (d, J=8.3 Hz, 1H), 6.12 (s, 1H), 5.29 (d, J=9.2 Hz, 1H), 4.72 (s, 0.5H), 4.40 (s, 0.5H), 4.07 (d, J=8.6 Hz, 0.5H), 3.98 (d, J=7.3 Hz, 2H), 3.92 (q, J=6.6 Hz, 0.5H), 3.77 (q, J=6.6 Hz, 1H), 3.60 (d, J=11.7 Hz, 0.5H), 3.46 (s, 3H), 3.39 (d, J=11.8 Hz, 1H), 3.22 (d, J=11.8 Hz, 0.5H), 3.03 (s, 0.5H), 2.82 (d, J=3.3 Hz, 3H), 2.78 (s, 0.5H), 2.43 (d, J=8.9 Hz, 0.5H), 2.31 (d, J=12.1 Hz, 0.5H), 2.20-1.92 (m, 1H), 1.90-1.78 (m, 1H), 1.70-1.55 (m, 2H), 1.37 (dd, J=10.4, 6.5 Hz, 3H), 0.92 (dt, J=23.7, 7.3 Hz, 3H), 0.53 (dt, J=35.2, 7.4 Hz, 3H) ppm. MS: M/e 504 (M+1)⁺.

### Compound A32: 2-(7-((2S,5R)-4-(1-(benzo[d]thiazol-5-yl)ethyl)-2,5-diethylpiperazin-1-yl)-4-methyl-5-oxo-4,5-dihydropyrazolo[1,5-a]pyrimidin-2-yl)acetonitrile

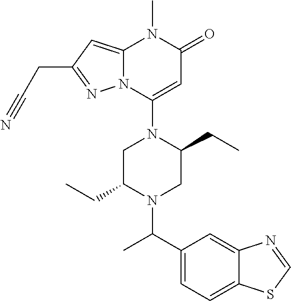 [SMILES: CC[C@H]1CN(C(C)c2ccc3scnc3c2)[C@H](CC)CN1c1cc(=O)n(C)c2cc(CC#N)nn12]

A solution of 2-(7-((2S,5R)-2,5-diethylpiperazin-1-yl)-4-methyl-5-oxo-4,5-dihydropyrazolo[1,5-a]pyrimidin-2-yl)acetonitrile (200 mg, 0.61 mmol), 1-(benzo[d]thiazol-5-yl)ethan-1-ol (218 mg, 1.22 mmol), (cyanomethyl)trimethylphosphonium iodide (445 mg, 1.83 mmol) and DIPEA (787 mg, 6.10 mmol) in CH₃CN (6 ml) was stirred at 100° C. overnight. The reaction was diluted with EtOAc (15 ml) and washed with brine (10 ml). The organic layer was concentrated under reduced pressure. The resulting residue was purified by flash column chromatography to give the titled Compound A32, which was further separated into Compound A32a (15 mg) and Compound A32b (32 mg, crude) by Prep-HPLC (Method A).

Compound A32a (the earlier peak): ¹H NMR (400 MHz, CD₃OD) δ 9.25 (s, 1H), 8.12-8.02 (m, 2H), 7.58 (d, J=8.3 Hz, 1H), 6.11 (s, 1H), 5.28 (s, 1H), 4.73 (s, 1H), 3.99 (s, 2H), 3.98-3.93 (m, 1H), 3.76 (d, J=11.8 Hz, 1H), 3.45 (s, 3H), 3.39 (d, J=11.8 Hz, 1H), 3.10-3.00 (m, 2H), 2.45 (d, J=9.1 Hz, 1H), 2.23-2.09 (m, 1H), 1.91-1.79 (m, 1H), 1.77-1.55 (m, 2H), 1.41 (d, J=6.3 Hz, 3H), 0.90 (t, J=7.4 Hz, 3H), 0.58 (t, J=7.3 Hz, 3H) ppm. MS: M/e 490 (M+1)⁺.

Compound A32b (the later peak): 70% purity (214 nm), MS: M/e 490 (M+1)⁺.

### Compound A33: 2-(4-methyl-7-((2R)-2-methyl-1-(1-(quinoxalin-6-yl)ethyl)piperidin-4-yl)-5-oxo-4,5-dihydropyrazolo[1,5-a]pyrimidin-2-yl)acetonitrile

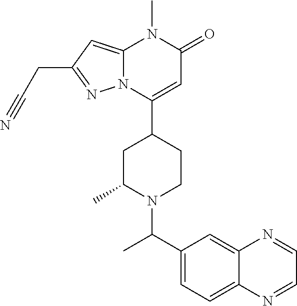 [SMILES: CC(c1ccc2nccnc2c1)N1CCC(c2cc(=O)n(C)c3cc(CC#N)nn23)C[C@H]1C]

### Step A: tert-butyl (2R) 4-cyano-2-methylpiperidine-1-carboxylate

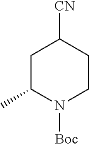 [SMILES: C[C@@H]1CC(C#N)CCN1C(=O)OC(C)(C)C]

To a solution of tert-butyl (R)-2-methyl-4-oxopiperidine-1-carboxylate (5 g, 23 mmol) in THF (12 mL) was added Tosmic (1.17 g, 6 mmol) and t-BuOK (672 mg, 6 mmol) at 0° C. The resulted mixture was and stirred at RT for 4 hrs. The mixture was added H₂O and extracted by EtOAc. The organic phase was dried over Na₂SO₄, filtered, and concentrated. The crude product was purified by column chromatography to give the title product (2.5 g, 49%). MS: M/e 225 (M+1)⁺.

### Step B: (2R)-1-(tert-butoxycarbonyl)-2-methylpiperidine-4-carboxylic Acid

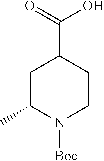 [SMILES: C[C@@H]1CC(C(=O)O)CCN1C(=O)OC(C)(C)C]

To a solution of tert-butyl (2R)-4-cyano-2-methylpiperidine-1-carboxylate (2.5 g, 10 mmol) in EtOH (80 mL) was added NaOH (1M, 80 ml). The resulted mixture was and stirred at 80° C. for 16 hrs. The mixture was concentrated in vacuo. The residue was added H₂O and adjusted pH=1-2. The mixture was extracted by DCM. The organic phase was dried over Na₂SO₄, filtered, and concentrated. The crude product was used directly in next step (1 g, 41%). MS: M/e 244 (M+1)⁺.

### Step C: tert-butyl (2R)-4-(2,2-dimethyl-4,6-dioxo-1,3-dioxane-5-carbonyl)-2-methylpiperidine-1-carboxylate

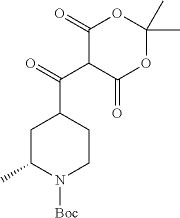

To a solution of (2R)-1-(tert-butoxycarbonyl)-2-methylpiperidine-4-carboxylic acid (1 g, 4 mmol) in DCM (20 mL) was added 2,2-dimethyl-1,3-dioxane-4,6-dione (864 mg, 6 mol), EDCI (1.3 g, 7 mmol) and DMAP (1.39 g, 10 mmol). The resulted mixture was stirred at RT overnight. The mixture was added to water and extracted by ethyl acetate. The organic phase was dried over Na₂SO₄, filtered, and concentrated. The crude product was purified by column chromatography to give the title product (700 mg, 47.6%). MS: M/e 370 (M+1)⁺.

### Step D: tert-butyl (2R)-4-(3-((3-bromo-1H-pyrazol-5-yl)amino)-3-oxopropanoyl)-2-methylpiperidine-1-carboxylate

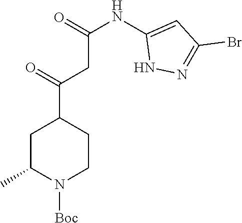 [SMILES: C[C@@H]1CC(C(=O)CC(=O)Nc2cc(Br)n[nH]2)CCN1C(=O)OC(C)(C)C]

To a mixture of tert-butyl (2R)-4-(2,2-dimethyl-4,6-dioxo-1,3-dioxane-5-carbonyl)-2-methylpiperidine-1-carboxylate (700 mg, 1.89 mmol) in THF (15 mL) was added 3-bromo-1H-pyrazol-5-amine (370 mg, 2 mmol). The resulted mixture was stirred at 70° C. overnight. The mixture was added to water and extracted by ethyl acetate. The organic phase was dried over Na₂SO₄, filtered, and concentrated. The crude product was purified by column chromatography to give the title product (660 g, 80.4%). MS: M/e 429 (M+1)⁺.

### Step E: tert-butyl (2R)-4-(2-bromo-5-oxo-4,5-dihydropyrazolo[1,5-a]pyrimidin-7-yl)-2-methylpiperidine-1-carboxylate

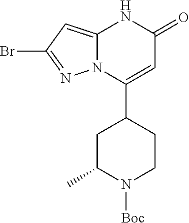 [SMILES: C[C@@H]1CC(c2cc(=O)[nH]c3cc(Br)nn23)CCN1C(=O)OC(C)(C)C]

To a mixture of tert-butyl (2R)-4-(3-((3-bromo-1H-pyrazol-5-yl)amino)-3-oxopropanoyl)-2-methylpiperidine-1-carboxylate (640 mg, 1.5 mmol) in Dioxane (5 mL) was added K₃PO₄ (424 mg. 2 mmol). The reaction was stirred sealed and stirred at 110° C. for 16 hrs. The mixture was added to water and extracted by ethyl acetate. The organic phase was dried over Na₂SO₄, filtered, and concentrated. The crude product was purified by column chromatography to give the title product (350 mg, 56.9%). MS: M/e 411 (M+1)⁺.

### Step F: tert-butyl (2R)-4-(2-bromo-4-methyl-5-oxo-4,5-dihydropyrazolo[1,5-a]pyrimidin-7-yl)-2-methylpiperidine-1-carboxylate

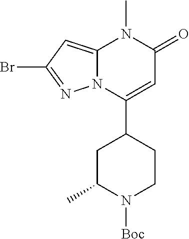 [SMILES: C[C@@H]1CC(c2cc(=O)n(C)c3cc(Br)nn23)CCN1C(=O)OC(C)(C)C]

To a mixture of tert-butyl (2R)-4-(2-bromo-5-oxo-4,5-dihydropyrazolo[1,5-a]pyrimidin-7-yl)-2-methylpiperidine-1-carboxylate (350 mg. 0.85 mmol) in Dioxane (5 mL) was added Trimethyl phosphate (595 mg, 4.25 mmol) and K₂CO₃ (1.18 g, 8.5 mmol). The reaction mixture was stirred at 95° C. overnight. The mixture was added H₂O and extracted by ethyl acetate. The organic phase was washed with brine, dried over Na₂SO₄, filtered and concentrated to dryness. The resulting residue was purified by column chromatography to give the titled compound (400 mg, 100%). MS: M/e 425 (M+1)⁺.

### Step G: methyl 7-((2R)-1-(tert-butoxycarbonyl)-2-methylpiperidin-4-v)-4-methyl-5-oxo-4,5-dihydropyrazolo[1,5-a]pyrimidine-2-carboxylate

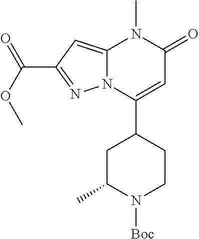 [SMILES: COC(=O)c1cc2n(C)c(=O)cc(C3CCN(C(=O)OC(C)(C)C)[C@H](C)C3)n2n1]

To a solution of tert-butyl (2R)-4-(2-bromo-4-methyl-5-oxo-4,5-dihydropyrazolo[1,5-a]pyrimidin-7-yl)-2-methylpiperidine-1-carboxylate (400 mg, 0.85 mmol) in MeOH (20 mL) was added Pd(dppf)₂Cl₂ (140 mg, 0.2 mmol) and Et₃N (404 mg, 4 mmol). The reaction mixture was stirred under an CO atmosphere at 90° C. for 16 hrs. The mixture was cooled down to RT. The mixture was added to water and extracted by ethyl acetate. The organic phase was dried over Na₂SO₄, filtered, and concentrated to dryness. The resulting residue was purified by column chromatography to give the titled compound (380 mg, 100%). MS: M/e 405 (M+1)⁺.

### Step H: tert-butyl (2R)-4-(2-(hydroxymethyl)-4-methyl-5-oxo-4,5-dihydropyrazolo[1,5-a]pyrimidin-7-yl)-2-methylpiperidine-1-carboxylate

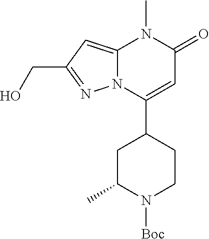 [SMILES: C[C@@H]1CC(c2cc(=O)n(C)c3cc(CO)nn23)CCN1C(=O)OC(C)(C)C]

To a mixture of methyl 7-((2R)-1-(tert-butoxycarbonyl)-2-methylpiperidin-4-yl)-4-methyl-5-oxo-4,5-dihydropyrazolo[1,5-a]pyrimidine-2-carboxylate (380 mg, 1 mmol) in THF (10 mL) was added NaBH₄ (76 mg, 2 mmol). The resulted mixture was stirred at 70° C. overnight. The mixture was concentrated in vacuo. The residue was added to ice-water and extracted by ethyl acetate. The organic phase was dried over Na₂SO₄, filtered, and concentrated to dryness. The resulting residue was purified by column chromatography to give the titled compound (200 mg, 56.1%). MS: M/e 377 (M+1)⁺.

### Step I: tert-butyl (2R)-2-methyl-4-(4-methyl-2-(((methylsulfonyl)oxy)methyl)-5-oxo-4,5-dihydropyrazolo[1,5-a]pyrimidin-7-yl)piperidine-1-carboxylate

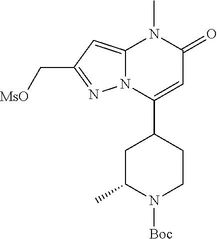 [SMILES: C[C@@H]1CC(c2cc(=O)n(C)c3cc(COS(C)(=O)=O)nn23)CCN1C(=O)OC(C)(C)C]

To a mixture of tert-butyl (2R)-4-(2-(hydroxymethyl)-4-methyl-5-oxo-4,5-dihydropyrazolo[1,5-a]pyrimidin-7-yl)-2-methylpiperidine-1-carboxylate (200 mg, 0.5 mmol) in DCM (15 mL) was added methanesulfonyl chloride (114 mg, 1 mmol) and Et₃N (202 mg, 2 mmol). The resulted mixture was stirred at RT for 30 mins. The mixture was washed with NaHCO₃ aqueous solution, dried over Na₂SO₄, filtered, and concentrated to dryness. The resulting residue was used directly in next step. MS: M/e 455 (M+1)⁺.

### Step J: tert-butyl (2R)-4-(2-(cyanomethyl)-4-methyl-5-oxo-4,5-dihydropyrazolo[1,5-a]pyrimidin-7-yl)-2-methylpiperidine-1-carboxylate

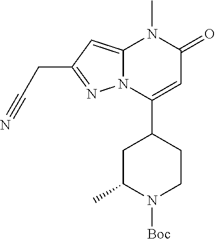 [SMILES: C[C@@H]1CC(c2cc(=O)n(C)c3cc(CC#N)nn23)CCN1C(=O)OC(C)(C)C]

To a mixture of tert-butyl (2R)-2-methyl-4-(4-methyl-2-(((methylsulfonyl)oxy)methyl)-5-oxo-4,5-dihydropyrazolo[1,5-a]pyrimidin-7-yl)piperidine-1-carboxylate from last step in acetonitrile (10 mL) was added TMSCN (160 mg. 1.6 mmol) and K₂CO₃ (359 mg, 2.6 mmol). The reaction was stirred at 80° C. overnight. The mixture was added to water and extracted by ethyl acetate. The organic phase was dried over Na₂SO₄, filtered, and concentrated to dryness. The resulting residue was purified by column chromatography to give the titled compound (60 mg). MS: M/e 386 (M+1)⁺.

### Step K: 2-(4-methyl-7-((2R)-2-methylpiperidin-4-v)-5-oxo-4,5-dihydropyrazolo[1,5-a]pyrimidin-2-yl)acetonitrile

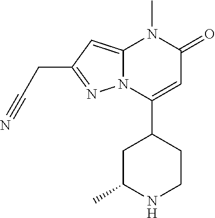 [SMILES: C[C@@H]1CC(c2cc(=O)n(C)c3cc(CC#N)nn23)CCN1]

A mixture of tert-butyl (2R)-4-(2-(cyanomethyl)-4-methyl-5-oxo-4,5-dihydropyrazolo[1,5-a]pyrimidin-7-yl)-2-methylpiperidine-1-carboxylate (60 mg. 0.16 mmol) in TFA (5 mL) was stirred at RT for 2 hrs. The mixture concentrated in vacuo. The residue was added to a solution of saturated NaHCO₃ aqueous solution and extracted by DCM. The organic phase was dried over Na₂SO₄, filtered, and concentrated to dryness. The resulting residue was used directly in next step. MS: M/e 286 (M+1)⁺.

### Step L: 2-(4-methyl-7-((2R)-2-methyl-1-(1-quinoxalin-6-yl)ethyl)piperidin-4-yl)-5-oxo-4,5-dihydropyrazolo[1,5-a]pyrimidin-2-yl)acetonitrile

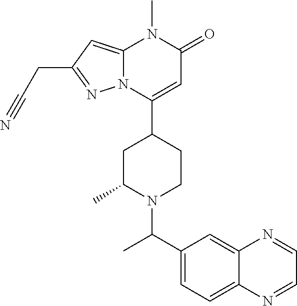 [SMILES: CC(c1ccc2nccnc2c1)N1CCC(c2cc(=O)n(C)c3cc(CC#N)nn23)C[C@H]1C]

To a solution of 2-(4-methyl-7-((2R)-2-methylpiperidin-4-yl)-5-oxo-4,5-dihydropyrazolo[1,5-a]pyrimidin-2-yl)acetonitrile from last step in acetonitrile (5 mL) was added 1-(quinoxalin-6-yl)ethan-1-ol (17.4 mg, 0.1 mmol), (cyanomethyl)trimethylphosphonium (125 mg, 0.5 mmol) and DIPEA (129 mg, 1 mmol). The reaction mixture was sealed and stirred under nitrogen protection at 105° C. for 16 hrs. The mixture was added H₂O and extracted with ethyl acetate. The combined organic extracts were dried over sodium sulfate, filtered and evaporated to dryness. The resulting residue was purified by Prep-HPLC (Method A) to give the titled Compound A33a (1.48 mg) and Compound A33b (1.75 mg).

Compound A33a (the earlier peak): ¹H NMR (400 MHz, CD₃OD) δ 8.86 (dd, J=7.1, 1.8 Hz, 2H), 8.09 (d, J=8.3 Hz, 2H), 8.02 (d, J=10.4 Hz, 1H), 6.16 (s, 1H), 5.97 (s, 1H), 4.06-3.97 (m, 2H), 3.66 (d, J=8.5 Hz, 1H), 3.49 (s, 3H), 3.13 (s, 2H), 2.80 (d, J=12.4 Hz, 2H), 2.15 (d, J=11.2 Hz, 1H), 1.87 (d, J=8.2 Hz, 2H), 1.45 (d, J=6.5 Hz, 3H), 1.34 (s, 1H), 1.16 (d, J=6.7 Hz, 3H). MS: M/e 442 (M+1)⁺.

Compound A33b (the later peak): ¹H NMR (400 MHz, CD₃OD) δ 8.86 (dd, J=7.6, 1.7 Hz, 2H), 8.08 (d, J=8.2 Hz, 2H), 8.01 (d, J=10.1 Hz, 1H), 6.16 (s, 1H), 5.97 (s, 1H), 4.00 (s, 1H), 3.94 (d, J=6.4 Hz, 1H), 3.76-3.70 (m, 1H), 3.50 (s, 3H), 2.78 (s, 2H), 2.66 (t, J=11.4 Hz, 1H), 2.53 (d, J=12.1 Hz, 1H), 2.07 (dd, J=11.0, 4.0 Hz, 2H), 1.91 (s, 1H), 1.45 (d, J=6.6 Hz, 3H), 1.34 (s, 1H), 1.29 (d, J=6.6 Hz, 3H). MS: M/e 442 (M+1)⁺.

### Compound A34: 2-(4-methyl-7-((2S)-2-methyl-1-(1-(quinoxalin-6-yl)ethyl)piperidin-4-yl)-5-oxo-4,5-dihydropyrazolo[1,5-a]pyrimidin-2-yl)acetonitrile

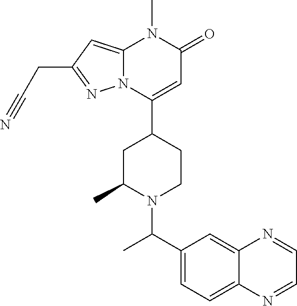 [SMILES: CC(c1ccc2nccnc2c1)N1CCC(c2cc(=O)n(C)c3cc(CC#N)nn23)C[C@@H]1C]

### Step A: tert-butyl (2S)-4-cyano-2-methylpiperidine-1-carboxylate

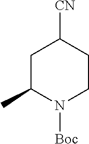 [SMILES: C[C@H]1CC(C#N)CCN1C(=O)OC(C)(C)C]

To a solution of tert-butyl (S)-2-methyl-4-oxopiperidine-1-carboxylate (4.26 g, 20 mmol) in DME (12 mL) was added EtOH (1.84 g, 40 mmol), Tosmic (5.85 g, 30 mmol) and t-BuOK (6.72 g, 60 mmol) at 0° C. The resulted mixture was and stirred at RT overnight. The mixture was added H₂O and extracted by EtOAc. The organic phase was dried over Na₂SO₄, filtered, and concentrated to dryness. The resulting residue was purified by column chromatography to give the titled compound (3 g, 66.9%). MS: M/e 225 (M+1)⁺.

### Step B: (2S)-1-(tert-butoxycarbonyl)˜2-methylpiperidine-4-carboxylic Acid

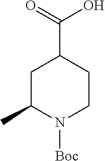 [SMILES: C[C@H]1CC(C(=O)O)CCN1C(=O)OC(C)(C)C]

To a solution of tert-butyl (2S)-4-cyano-2-methylpiperidine-1-carboxylate (3 g, 13.3 mmol) in EtOH (50 mL) was added NaOH (2M, 50 ml). The resulted mixture was and stirred at 80° C. for 16 hours. The mixture was concentrated in vacuo. The residue was added H₂O and adjusted pH=1-2. The mixture was extracted by DCM. The organic phase was dried over Na₂SO₄, filtered, and concentrated to dryness. The resulting residue was used directly in next step (3 g, 100%). MS: M/e 244 (M+1)⁺.

### Step C: tert-butyl (2S)-4-(2,2-dimethyl-4,6-dioxo-1,3-dioxane-5-carbonyl)-2-methylpiperidine-1-carboxylate

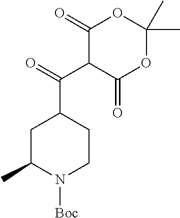

To a solution of (2S)-1-(tert-butoxycarbonyl)-2-methylpiperidine-4-carboxylic acid (10 g, 16.5 mmol) in DCM (20 mL) was added 2,2-dimethyl-1,3-dioxane-4,6-dione (2.9 g, 19.8 mol), EDCI (4.7 g, 24.8 mmol) and DMAP (3.5 g, 24.8 mmol). The resulted mixture was stirred at RT overnight. The mixture was added to water and extracted by ethyl acetate. The organic phase was dried over Na₂SO₄, filtered, and concentrated to dryness. The resulting residue was purified by column chromatography to give the titled compound (3.9 g, 65%). MS: M/e 370 (M+1)⁺.

### Step D: tert-butyl (2s)-4-(3-((3-bromo-1H-pyrazol-5-yl)amino)-3-oxopropanoyl)-2-methylpiperidine-1-carboxylate

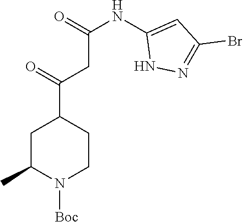 [SMILES: C[C@H]1CC(C(=O)CC(=O)Nc2cc(Br)n[nH]2)CCN1C(=O)OC(C)(C)C]

To a mixture of tert-butyl (2s)-4-(2,2-dimethyl-4,6-dioxo-1,3-dioxane-5-carbonyl)-2-methylpiperidine-1-carboxylate (3.9 g, 10.5 mmol) in THF (15 mL) was added 3-bromo-1H-pyrazol-5-amine (1.69 g, 10.5 mmol). The resulted mixture was stirred at 70° C. overnight. The mixture was added to water and extracted by ethyl acetate. The organic phase was dried over Na₂SO₄, filtered, and concentrated to dryness. The resulting residue was purified by column chromatography to give the titled compound (3 g, 66.8%). MS: M/e 429 (M+1)⁺.

### Step E: tert-butyl (2s)-4-(2-bromo-5-oxo-4,5-dihydropyrazolo[1,5-a] pyrimidin-7-yl)-2-methylpiperidine-1-carboxylate

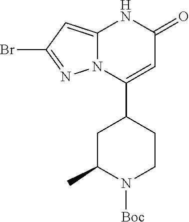 [SMILES: C[C@H]1CC(c2cc(=O)[nH]c3cc(Br)nn23)CCN1C(=O)OC(C)(C)C]

To a mixture of tert-butyl (2S)-4-(3-((3-bromo-1H-pyrazol-5-yl)amino)-3-oxopropanoyl)-2-methylpiperidine-1-carboxylate (1.5 g, 3.5 mmol) in Dioxane (5 mL) was added K₃PO₄ (3 g. 14 mmol). The reaction was stirred sealed and stirred at 110° C. for 16 hrs. The mixture was added to water and extracted by ethyl acetate. The organic phase was dried over Na₂SO₄, filtered, and concentrated to dryness. The resulting residue was purified by column chromatography to give the titled compound (900 mg, 64.2%). MS: M/e 411 (M+1)⁺.

### Step F: tert-butyl (2S)-4-(2-bromo-4-methyl-5-oxo-4,5-dihydropyrazolo [1,5-a]pyrimidin-7-yl)-2-methylpiperidine-1-carboxylate

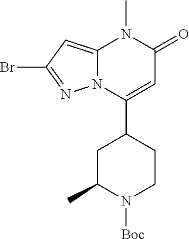 [SMILES: C[C@H]1CC(c2cc(=O)n(C)c3cc(Br)nn23)CCN1C(=O)OC(C)(C)C]

To a mixture of tert-butyl (2S)-4-(2-bromo-5-oxo-4,5-dihydropyrazolo[1,5-a]pyrimidin-7-yl)-2-methylpiperidine-1-carboxylate (430 mg. 1 mmol) in Dioxane (5 mL) was added Trimethyl phosphate (700 mg, 5 mmol) and K₂CO₃ (1.39 g, 10 mmol). The reaction mixture was stirred at 95° C. overnight. The mixture was added H₂O and extracted by ethyl acetate. The organic phase was washed with brine, dried over Na₂SO₄, filtered, and concentrated to dryness. The resulting residue was purified by column chromatography to give the title product (500 mg, 100%). MS: M/e 425 (M+1)⁺.

### Step G: methyl 74(2S)-1-(tert-butoxycarbonyl)-2-methylpiperidin-4-yl)-4-methyl-5-oxo-4,5-dihydropyrazolo[1,5-a]pyrimidine-2-carboxylate

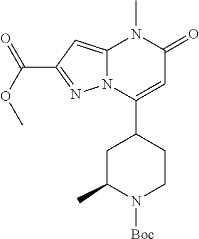 [SMILES: COC(=O)c1cc2n(C)c(=O)cc(C3CCN(C(=O)OC(C)(C)C)[C@@H](C)C3)n2n1]

To a solution of tert-butyl (2S)-4-(2-bromo-4-methyl-5-oxo-4,5-dihydropyrazolo[1,5-a]pyrimidin-7-yl)-2-methylpiperidine-1-carboxylate (500 mg, 1 mmol) in MeOH (20 mL) was added Pd(dppf)₂Cl₂ (140 mg, 0.2 mmol) and Et₃N (404 mg, 4 mmol). The reaction mixture was stirred under an CO atmosphere at 90° C. for 16 hrs. The mixture was cooled down to RT. The mixture was added to water and extracted by ethyl acetate. The organic phase was dried over Na₂SO₄, filtered, and concentrated to dryness. The resulting residue was purified by column chromatography to give the titled compound (240 mg, 59.4%). MS: M/e 405 (M+1)⁺.

### Step H: tert-butyl (2S)-4-(2-(hydroxymethyl)-4-methyl-5-oxo-4,5-dihydropyrazolo[1,5-a]pyrimidin-7-yl)-2-methylpiperidine-1-carboxylate

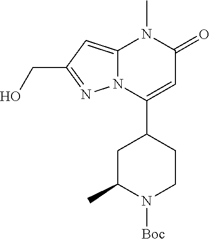 [SMILES: C[C@H]1CC(c2cc(=O)n(C)c3cc(CO)nn23)CCN1C(=O)OC(C)(C)C]

To a mixture of methyl 7-((2S)-1-(tert-butoxycarbonyl)-2-methylpiperidin-4-yl)-4-methyl-5-oxo-4,5-dihydropyrazolo[1,5-a]pyrimidine-2-carboxylate (240 mg, 0.59 mmol) in THF (10 mL) was added NaBH₄ (76 mg, 2 mmol). The resulted mixture was stirred at 70° C. overnight. The mixture was concentrated in vacuo. The residue was added to ice-water and extracted by ethyl acetate. The organic phase was dried over Na₂SO₄, filtered, and concentrated to dryness. The resulting residue was purified by column chromatography to give the titled compound (130 mg, 54.1%). MS: M/e 377 (M+1)⁺.

### Step I: tert-butyl (2S)-2-methyl-4-(4-methyl-2-(((methylsulfonyl)oxy)methyl)-5-oxo-4,5-dihydropyrazolo[1,5-a]pyrimidin-7-yl)piperidine-1-carboxylate

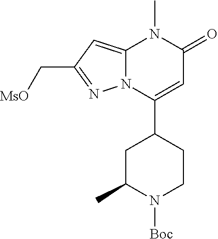 [SMILES: C[C@H]1CC(c2cc(=O)n(C)c3cc(COS(C)(=O)=O)nn23)CCN1C(=O)OC(C)(C)C]

To a mixture of tert-butyl (2S)-4-(2-(hydroxymethyl)-4-methyl-5-oxo-4,5-dihydropyrazolo[1,5-a]pyrimidin-7-yl)-2-methylpiperidine-1-carboxylate (130 mg, 0.35 mmol) in DCM (15 mL) was added methanesulfonyl chloride (114 mg, 1 mmol) and Et₃N (202 mg, 2 mmol). The resulted mixture was stirred at RT for 30 mins. The mixture was washed with NaHCO₃ aqueous solution, dried over Na₂SO₄, filtered, and concentrated to dryness. The resulting residue was used directly in next step. MS: M/e 455 (M+1)⁺.

### Step J: tert-butyl (2S)-4-(2-(cyanomethyl)-4-methyl-5-oxo-4,5-dihydropyrazolo[1,5-a]pyrimidin-7-yl)-2-methylpiperidine-1-carboxylate

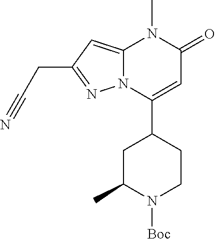 [SMILES: C[C@H]1CC(c2cc(=O)n(C)c3cc(CC#N)nn23)CCN1C(=O)OC(C)(C)C]

To a mixture of tert-butyl (2S)-2-methyl-4-(4-methyl-2-(((methylsulfonyl)oxy)methyl)-5-oxo-4,5-dihydropyrazolo[1,5-a]pyrimidin-7-yl)piperidine-1-carboxylate from last step in acetonitrile (10 mL) was added TMSCN (160 mg. 1.6 mmol) and K₂CO₃ (359 mg, 2.6 mmol). The reaction was stirred at 80° C. overnight. The mixture was added to water and extracted by ethyl acetate. The organic phase was dried over Na₂SO₄, filtered, and concentrated to dryness. The resulting residue was purified by column chromatography to give the title product (120 mg). MS: M/e 386 (M+1)⁺.

### Step K: 2-(4-methyl-7-((2S)-2-methylpiperidin-4-yl)-5-oxo-4,5-dihydropyrazolo[1,5-a]pyrimidin-2-yl)acetonitrile

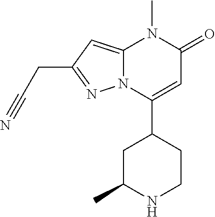 [SMILES: C[C@H]1CC(c2cc(=O)n(C)c3cc(CC#N)nn23)CCN1]

A mixture of tert-butyl (2S)-4-(2-(cyanomethyl)-4-methyl-5-oxo-4,5-dihydropyrazolo[1,5-a]pyrimidin-7-yl)-2-methylpiperidine-1-carboxylate (120 mg. 0.3 mmol) in TFA (5 mL) was stirred at RT for 2 hours. The mixture concentrated in vacuo. The residue was added to a solution of saturated NaHCO₃ aqueous solution and extracted by DCM. The organic phase was dried over Na₂SO₄, filtered, and concentrated to dryness. The resulting residue was used directly in next step. MS: M/e 286 (M+1)⁺.

### Step L: 2-(4-methyl-7-((2S)-2-methyl-1-(1-(quinoxalin-6-yl)ethyl) piperidin-4-yl)-5-oxo-4,5-dihydropyrazolo[1,5-a]pyrimidin-2-yl)acetonitrile

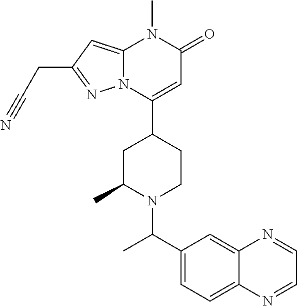 [SMILES: CC(c1ccc2nccnc2c1)N1CCC(c2cc(=O)n(C)c3cc(CC#N)nn23)C[C@@H]1C]

To a solution of 2-(4-methyl-7-((2R)-2-methylpiperidin-4-yl)-5-oxo-4,5-dihydropyrazolo[1,5-a]pyrimidin-2-yl)acetonitrile from last step in acetonitrile (5 mL) was added 1-(quinoxalin-6-yl)ethan-1-ol (174 mg, 1 mmol), (cyanomethyl)trimethylphosphonium (241 mg, 1 mmol) and DIPEA (258 mg, 2 mmol). The reaction mixture was sealed and stirred under nitrogen protection at 105° C. for 16 hrs. The mixture was added H₂O and extracted with ethyl acetate. The combined organic extracts were dried over sodium sulfate, filtered and evaporated to dryness. The resulting residue was purified by Prep-HPLC (Method A) to give the titled Compound A34a 1 (0.88 mg) and Compound A34b (1.36 mg).

Compound A34a (the earlier peak): ¹H NMR (400 MHz, CD₃OD) δ 8.86 (dd, J=7.2, 1.8 Hz, 2H), 8.09 (d, J=8.5 Hz, 2H), 8.02 (dd, J=8.8, 1.6 Hz, 1H), 6.16 (s, 1H), 5.97 (s, 1H), 4.57 (s, 1H), 4.08-3.97 (m, 2H), 3.71-3.61 (m, 1H), 3.49 (s, 3H), 3.20-3.07 (m, 1H), 2.83 (t, J=11.0 Hz, 1H), 2.18 (dd, J=17.6, 9.9 Hz, 1H), 1.88 (dd, J=9.0, 3.4 Hz, 2H), 1.46 (d, J=6.5 Hz, 3H), 1.30 (s, 2H), 1.17 (d, J=6.7 Hz, 3H) ppm. MS: M/e 442 (M+1)⁺.

Compound A34b (the later peak): ¹H NMR (400 MHz, CD₃OD) δ 8.86 (dd, J=7.7, 1.9 Hz, 2H), 8.11-8.06 (m, 2H), 8.02 (d, J=1.6 Hz, 1H), 6.16 (s, 1H), 5.97 (s, 1H), 4.57 (s, 1H), 4.01-3.91 (m, 2H), 3.77-3.70 (m, 1H), 3.50 (s, 3H), 2.65 (d, J=12.1 Hz, 1H), 2.54 (d, 0.1=12.6 Hz, 1H), 2.09 (d, J=3.7 Hz, 1H), 1.90 (d, J=10.4 Hz, 1H), 1.65 (dd, J=12.2, 4.2 Hz, 1H), 1.45 (d, J=6.6 Hz, 3H), 1.29 (d, J=6.6 Hz, 5H) ppm. MS: M/e 442 (M+1)⁺.

### Compound A35: 2-(7-((2S,5R)-2,5-diethyl-4-(1-(2-methylthiazolo[5,4-b]pyridin-5-yl)ethyl)piperazin-1-yl)-4-methyl-5-oxo-4,5-dihydropyrazolo[1,5-a]pyrimidin-2-yl)acetonitrile

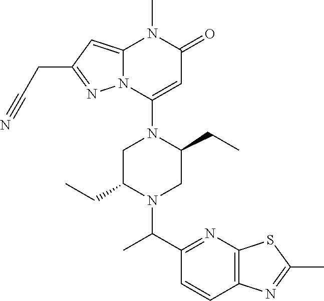 [SMILES: CC[C@H]1CN(C(C)c2ccc3nc(C)sc3n2)[C@H](CC)CN1c1cc(=O)n(C)c2cc(CC#N)nn12]

### Step A: 1-(2-methylthiazolo[5,4-b]pyridin-5-yl)ethan-1-one

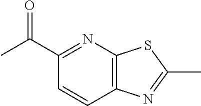

A mixture of 5-chloro-2-methylthiazolo[5,4-b]pyridine (250 mg, 1.35 mmol), tributyl(1-ethoxyvinyl)stannane (738 mg, 2.04 mmol) and Pd(PPh₃)₂Cl₂ (95 mg, 0.14 mmol) in DMF (3 mL) was stirred at 100° C. under N₂ for 16 hours. The solution was diluted with ethyl acetate and washed with water. The organic layer was purified by flash column chromatography. The product was diluted with ethyl acetate and added with HCl (3 mL, 3 M in dioxane) in drops and the mixture was stirred at RT for 2 hours. The reaction was concentrated and adjusted to pH=8˜9 with saturated NaHCO₃ solution, extracted with ethyl acetate. The combined organic layers were washed with brine, dried over Na₂SO₄. The resulting residue was purified by flash column chromatography to give the titled compound (200 mg, 77%). MS: M/e 193 (M+1)⁺

### Step B: 1-(2-methylthiazolo[5,4-b]pyridin-5-yl)ethan-1-ol

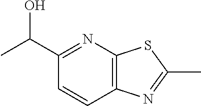

To a solution of 1-(2-methylthiazolo[5,4-b]pyridin-5-yl)ethan-1-one (200 mg, 1.04 mmol) in MeOH (3 mL) was added NaBH₄ (39 mg, 1.04 mmol) at RT and the resulting mixture was stirred at RT for 5 min. The reaction mixture was diluted with DCM and washed with water, dried over Na₂SO₄. The resulting residue was purified by flash column chromatography to give the titled compound (180 mg, 90%). MS: M/e 195 (M+1)⁺.

### Step C: 2-(7-((2S,5R)-2,5-diethyl-4-(1-(2-methylthiazolo[5,4-b]pyridin-5-yl)ethyl)piperazin-1-yl)-4-methyl-5-oxo-4,5-dihydropyrazolo[1,5-a]pyrimidin-2-yl)acetonitrile

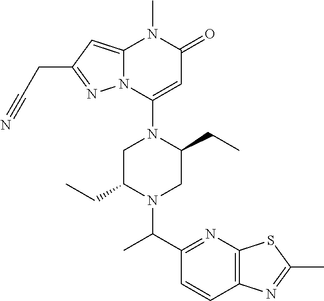 [SMILES: CC[C@H]1CN(C(C)c2ccc3nc(C)sc3n2)[C@H](CC)CN1c1cc(=O)n(C)c2cc(CC#N)nn12]

A mixture of 2-(7-((2S,5R)-2,5-diethylpiperazin-1-yl)-4-methyl-5-oxo-4,5-dihydropyrazolo[1,5-a]pyrimidin-2-yl)acetonitrile (160 mg, 0.5 mmol), 1-(2-methylthiazolo[5,4-b]pyridin-5-yl)ethan-1-ol (194 mg, 1 mmol), (cyanomethyl)trimethylphosphonium iodide (361 mg, 1.5 mmol) and DIPEA (387 mg, 3 mmol) in CH₃CN (8 mL) was stirred at 100° C. for 16 hours. The resulted mixture was diluted with EtOAc (10 mL), washed with brine (5 mL×3), dried and concentrated. The resulting residue was purified by flash column chromatography to give the titled compound (200 mg, 80%), which was further separated into Compound A35a (19 mg, 76%) and Compound A35b (20 mg, 80%) by prep-HPLC (Method A).

Compound A35a (the earlier peak)). ¹H NMR (400 MHz, CD₃OD) δ 8.22 (d, J=8.5 Hz, 1H), 7.75 (d, J=8.5 Hz, 1H), 6.12 (s, 1H), 5.31 (s, 1H), 4.45 (s, 1H), 4.08 (m, 1H), 3.93 (m, 3H), 3.62 (m, 1H), 3.46 (s, 3H), 3.20 (m, 1H), 2.89 (m, 4H), 2.21 (m, 1H), 1.90 (m, 2H), 1.68 (m, 1H), 1.57 (m, 1H), 1.39 (d, J=6.6 Hz, 3H), 0.94 (t, J=7.3 Hz, 3H), 0.53 (t, J=7.4 Hz, 3H) ppm. MS: M/e 505 (M+1)⁺.

Compound A35b (the later peak)). ¹H NMR (400 MHz, CD₃OD) δ 8.23 (d, J=8.4 Hz, 1H), 7.72 (d, J=8.5 Hz, 1H), 6.12 (s, 1H), 5.30 (d, J=9.8 Hz, 1H), 4.68 (s, 1H), 4.08 (m, 1H), 3.99 (s, 2H), 3.79 (m, 1H), 3.44 (m, 4H), 3.04 (m, 2H), 2.86 (s, 3H), 2.38 (m, 1H), 2.09 (m, 1H), 1.75 (m, 3H), 1.41 (t, J=9.8 Hz, 3H), 0.87 (t, J=7.4 Hz, 3H), 0.58 (m, 3H) ppm. MS: M/e 505 (M+1)⁺.

### Compound A36: 2-(7-((2S,5R)-2,5-diethyl-4-(1-(2-methylthiazolo[5,4-b]pyridin-5-yl)ethyl)piperazin-1-yl)-6-fluoro-4-methyl-5-oxo-4,5-dihydropyrazolo[1,5-a]pyrimidin-2-yl)acetonitrile

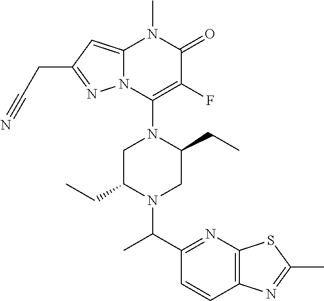 [SMILES: CC[C@H]1CN(C(C)c2ccc3nc(C)sc3n2)[C@H](CC)CN1c1c(F)c(=O)n(C)c2cc(CC#N)nn12]

To a solution of 2-(7-((2S,5R)-2,5-diethyl-4-(1-(2-methylthiazolo[5,4-b]pyridin-5-yl)ethyl)piperazin-1-yl)-4-methyl-5-oxo-4,5-dihydropyrazolo[1,5-a]pyrimidin-2-yl)acetonitrile (25 mg, 0.05 mmol) in MeCN (5 mL) was added Selectfluor (21 mg, 0.06 mmol) and the mixture was stirred at RT for 1 hour. The reaction mixture was poured into water (30 mL) and then extracted with EtOAc (30 mL×3). The organic layer was washed with brine (30 mL), dried and concentrated. The resulting residue was purified by prep-HPLC (Method A) to give the titled compound (3 mg, 11.5%). ¹H NMR (400 MHz, CD₃OD) δ 8.23 (m, 1H), 7.76 (m, 1H), 6.16 (s, 1H), 4.14-3.75 (m, 1H), 3.98 (m, 4H), 3.52 (m, 4H), 3.08 (m, 2H), 2.85 (s, 3H), 2.16 (m, 1H), 1.95 (m, 2H), 1.66 (m, 2H), 1.41 (dd, J=19.3, 6.6 Hz, 3H), 0.85 (q, J=7.3 Hz, 3H), 0.58 (dt, J=22.0, 7.4 Hz, 3H) ppm. MS: M/e 523 (M+1)⁺.

### Compound A37: 2-(7-((2S,5R)-4-(1-(2-(difluoromethyl)thieno[2,3-b]pyridin-6-yl)ethyl)-2,5-diethylpiperazin-1-yl)-4-methyl-5-oxo-4,5-dihydropyrazolo[1,5-a]pyrimidin-2-yl)acetonitrile

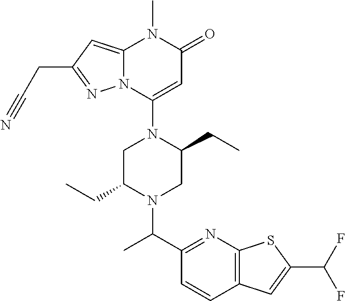 [SMILES: CC[C@H]1CN(C(C)c2ccc3cc(C(F)F)sc3n2)[C@H](CC)CN1c1cc(=O)n(C)c2cc(CC#N)nn12]

### Step A: Methyl thieno[2,3-b]pyridine-2-carboxylate

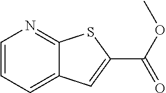

To a solution of 2-chloronicotinaldehyde (2.8 g, 20 mmol) in DMF (10 mL) was added methyl 2-mercaptoacetate (2.3 g, 22 mmol) and K₂CO₃ (4.2 g, 30 mmol) at room temperature and the mixture was stirred at 80° C. for 2 hours. The reaction mixture was poured into water (30 mL) and then extracted with EtOAc (30 mL×3). The organic layer was washed with brine (30 mL), dried and concentrated. The resulting residue was purified by flash column chromatography with 0-100% EA in PE to give the titled compound (3 g, 78.9%). MS: M/e 194 (M+1)⁺.

### Step B: thieno[2,3-b]pyridin-2-ylmethanol

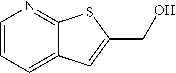 [SMILES: OCc1cc2cccnc2s1]

To a solution of methyl thieno[2,3-b]pyridine-2-carboxylate (2.6 g, 13.8 mmol) in THF (10 mL) at 0° C. was slowly added LiAlH₄ (576 mg, 15.2 mmol). The reaction mixture was stirred at 0° C. for 0.5 hour. The reaction mixture was slowly added H₂O (0.6 mL), 15% NaOH aqueous solution (0.6 mL), and H₂O (1.8 mL). The mixture was filtered. The filtrate was concentrated in vacuo. The resulting residue was purified by flash column chromatography with 0-100% EA in PE to give the titled compound (2.2 g, 100%). MS: M/e 166 (M+1)⁺.

### Step C: thieno[2,3-b]pyridine-2-carbaldehyde

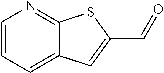 [SMILES: O=Cc1cc2cccnc2s1]

To a solution of thieno[2,3-b]pyridin-2-ylmethanol (1.8 g, 11 mmol) in DCM was added Des Martin reagent (6 g, 14.2 mmol) and the mixture was stirred at room temperature for overnight. The reaction mixture was added saturated NaHCO₃ aqueous solution (30 mL) and then extracted with EtOAc (30 mL×3). The organic layer was washed with brine (30 mL), dried and concentrated. The resulting residue was purified by flash column chromatography with 0-100% EA in PE to give the titled compound (1.45 g, 80.5%). MS: M/e 164 (M+1)⁺.

### Step D: 2-(difluoromethyl)thieno[2,3-b]pyridine

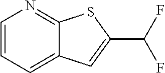 [SMILES: FC(F)c1cc2cccnc2s1]

To a solution of thieno[2,3-b]pyridine-2-carbaldehyde (1.45 g, 8.9 mmol) in DCM was added DAST (7.2 g, 44.5 mmol) and the mixture was stirred at room temperature for overnight. The reaction mixture was added saturated NaHCO₃ aqueous solution (30 mL) and then extracted with DCM (30 mL×3). The organic layer was washed with brine (30 mL), dried and concentrated. The resulting residue was purified by flash column chromatography with 0-100% EA in PE to give the titled compound (1 g, 60.9%). MS: M/e 186 (M+1)⁺.

### Step E: 2-(difluoromethyl)thieno[2,3-b]pyridine 7-oxide

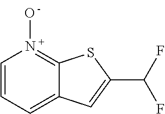

To a solution of 2-(difluoromethyl)thieno[2,3-b]pyridine (1 g, 5.3 mmol) in DCM was added m-CPBA (1.38 g, 8 mmol) and the mixture was stirred at room temperature for overnight. The reaction mixture was added saturated K₂O₃ aqueous solution (30 mL) and then extracted with DCM (30 mL×3). The organic layer was washed with brine (30 mL), dried and concentrated. The resulting residue was used directly in next step. MS: M/e 202 (M+1)⁺.

### Step F: 2-(difluoromethyl)thieno[2,3-b]pyridine-6-carbonitrile

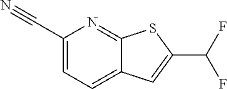 [SMILES: N#Cc1ccc2cc(C(F)F)sc2n1]

To the solution of 2-(difluoromethyl)thieno[2,3-b]pyridine 7-oxide from last step in DCM was added Et₃N (1.5 g, 15 mmol) and TMSCN (1.5 g, 15 mmol). The reaction mixture was stirred at 90° C. overnight. The reaction mixture was added H₂O (30 mL) and then extracted with EtOAc (30 mL×3). The organic layer was washed with brine (30 mL), dried and concentrated. The resulting residue was purified by flash column chromatography with 0-100/6 EA in PE to give the titled compound (700 mg, 63.6% for two steps). MS: M/e 211 (M+1)⁺.

### Step G: 2-(difluoromethyl)thieno[2,3-b]pyridine-6-carbaldehyde

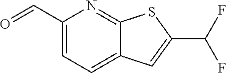 [SMILES: O=Cc1ccc2cc(C(F)F)sc2n1]

To a solution of 2-(difluoromethyl)thieno[2,3-b]pyridine-6-carbonitrile (416 mg, 1.98 mmol) in THF was added DABIL-H (1 M. 4 mmol, 4 mL) and the mixture was stirred at room temperature for 1 h. The reaction mixture was added 2N HCl solution (2 mL) and stirred at RT for 10 mins. The resulting solution was added saturated NaHCO₃ aqueous solution (30 mL) and then extracted with EtOAc (30 mL×3). The organic layer was washed with brine (30 mL), dried and concentrated. The resulting residue was purified by flash column chromatography with 0-100% EA in PE to give the titled compound (220 mg, 52.2%). MS: M/e 214 (M+1)⁺.

### Step H: 1-(2-(difluoromethyl)thieno[2,3-b]pyridin-6-yl)ethan-1-ol

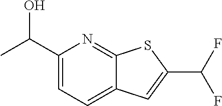

To a solution of 2-(difluoromethyl)thieno[2,3-b]pyridine-6-carbaldehyde (300 mg, 1.4 mmol) in THF was added CH₃MgBr (3 M. 1.69 mmol, 0.56 ml) at 0° C. and the mixture was stirred at 0° C. for 0.5 h. The reaction mixture was H₂O (30 mL) and then extracted with EtOAc (30 mL×3). The organic layer was washed with brine (30 mL), dried and concentrated. The resulting residue was purified by flash column chromatography with 0-100% EA in PE to give the titled compound (290 mg, 90%). MS: M/e 230 (M+1)⁺.

### Step I: 2-(7-((2S,5R)-4-(1-(2-(difluoromethyl)thieno[2,3-b]pyridin-6-yl)ethyl)-2,5-diethylpiperazin-1-yl)-4-methyl-5-oxo-4,5-dihydropyrazolo[1,5-a]pyrimidin-2-yl)acetonitrile

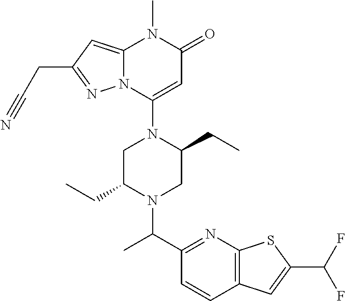 [SMILES: CC[C@H]1CN(C(C)c2ccc3cc(C(F)F)sc3n2)[C@H](CC)CN1c1cc(=O)n(C)c2cc(CC#N)nn12]

A mixture of 2-(7-((2S,5R)-2,5-diethylpiperazin-1-yl)-4-methyl-5-oxo-4,5-dihydropyrazolo[1,5-a]pyrimidin-2-yl)acetonitrile (30 mg, 0.1 mmol), 1-(2-(difluoromethyl)thieno[2,3-b]pyridin-6-yl)ethan-1-ol (34 mg, 0.15 mmol), (cyanomethyl)trimethylphosphonium iodide (120 mg, 0.5 mmol) and DIPEA (129 mg, 1 mmol) in MeCN (3 mL) was stirred at 100° C. for 16 hours. The resulted mixture was diluted with EtOAc (10 mL), washed with brine (5 mL×3), dried and concentrated. The resulting residue was purified by flash column chromatography to give the titled compound, which was further separated into Compound A37a (3 mg, 1.1%) and Compound A37b (4 mg, 1.5%) by prep-HPLC (Method A).

Compound A37a (the earlier peak)). ¹H NMR (400 MHz, CD₃OD) δ 8.29 (d, J=8.4 Hz, 1H), 7.73 (d, J=8.4 Hz, 1H), 7.66 (s, 1H), 7.14 (t, J=55.1 Hz, 1H), 6.12 (s, 1H), 5.31 (s, 1H), 4.44 (s, 1H), 4.09 (m, 1H), 3.97 (s, 2H), 3.92 (q, J=6.6 Hz, 1H), 3.63 (m, 1H), 3.46 (s, 3H), 3.20 (m, 1H), 2.92 (m, 1H), 2.21 (m, 1H), 1.90 (m, 2H), 1.64 (m, 2H), 1.40 (d, J=6.7 Hz, 3H), 0.95 (t, J=7.4 Hz, 3H), 0.54 (t, J=7.5 Hz, 3H) ppm. MS: M/e 540 (M+1)⁺.

Compound A37b (the later peak)). ¹H NMR (400 MHz, CD₃OD) δ 8.30 (d, J=8.3 Hz, 1H), 7.69 (m, 2H), 7.14 (t, J=55.1 Hz, 1H), 6.12 (s, 1H), 5.29 (s, 1H), 4.69 (s, 1H), 4.09 (q, J=6.6 Hz, 1H), 3.99 (s, 2H), 3.79 (m, 1H), 3.44 (m, 4H), 3.07 (m, 2H), 2.38 (m, 1H), 2.11 (m, 1H), 1.76 (m, 3H), 1.44 (d, J=6.6 Hz, 3H), 0.87 (t, J=7.5 Hz, 3H), 0.62 (t, J=7.4 Hz, 3H) ppm. MS: M/e 540 (M+1)⁺.

### Compound A38: 2-(7-((2S,5R)-2,5-diethyl-4-(1-(2-methylimidazo [1,2-b]pyridazin-6-yl)ethyl)piperazin-1-yl)-4-methyl-5-oxo-4,5-dihydropyrazolo[1,5-a]pyrimidin-2-yl)acetonitrile

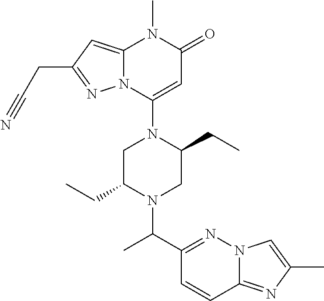 [SMILES: CC[C@H]1CN(C(C)c2ccc3nc(C)cn3n2)[C@H](CC)CN1c1cc(=O)n(C)c2cc(CC#N)nn12]

### Step A: 1-(2-methylimidazo[1,2-b]pyridazin-6-yl)ethan-1-one

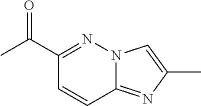

To a solution of 6-bromo-2-methylimidazo[1,2-b]pyridazine (212 mg, 1 mmol) in DMF (10 mL) was added tributyl(1-ethoxyvinyl)stannane (470 mg, 1.3 mmol) and Pd(PPh₂)₂Cl₂ (70 mg, 0.1 mmol). The reaction mixture was protected by N₂ atmosphere and stirred at 100° C. overnight. The mixture was cooled down to RT, 4M HCl in EA (2 mL) was added and the resulting mixture was stirred for one hour. The reaction was quenched with saturated NaHCO₃ solution to pH=8, diluted with H₂O, extracted with EA (60 mL×2), washed with brine, dried over Na₂SO₄, filtered, and concentrated to dryness. The residue was purified by flash column chromatography to give the titled compound (145 mg, a little triphenylphosphine oxide contained, 82%). MS: Me 176 (M+1)⁺.

### Step B: 1-(2-methylimidazo[1,2-b]pyridazin-6-yl)ethan-1-ol

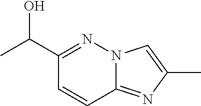

To a solution of 1-(2-methylimidazo[1,2-b]pyridazin-6-yl)ethan-1-one (140 mg, 0.8 mmol) in MeOH (5 mL) was added NaBH₄ (30 mg, 0.8 mmol). The resulting mixture was stirred at ice-bath for 30 mins. The reaction mixture was quenched with H₂O (20 mL) and extracted with EA (80 mL), washed with brine, dried over Na₂SO₄, filtered, and concentrated to dryness to give the titled compound (130 mg, 91%). MS: M/e 178 (M+1)⁺.

### Step C: 2-(7-((2S,5R)-2,5-diethyl-4-(1-(2-methylimidazo [1,2-b]pyridazin-6-yl)ethyl)piperazin-1-yl)-4-methyl-5-oxo-4,5-dihydropyrazolo[1,5-a]pyrimidin-2-yl)acetonitrile

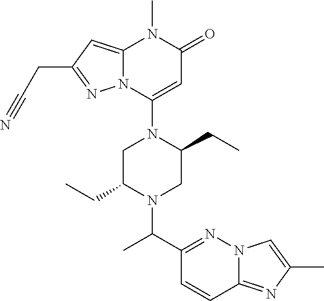 [SMILES: CC[C@H]1CN(C(C)c2ccc3nc(C)cn3n2)[C@H](CC)CN1c1cc(=O)n(C)c2cc(CC#N)nn12]

To a solution of 2-(7-((2S,5R)-2,5-diethylpiperazin-1-yl)-4-methyl-5-oxo-4,5-dihydropyrazolo[1,5-a]pyrimidin-2-yl)acetonitrile (30 mg, 0.087 mmol), 1-(2-methylimidazo[1,2-b]pyridazin-6-yl)ethan-1-ol (35 mg, 0.2 mmol) and (cyanomethyl)trimethylphosphonium iodide (120 mg, 0.5 mmol) in CH₃CN (3 mL) was added DIPEA (258 mg, 2 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified and separated into Compound A38a (5.6 mg, 23.1%) and Compound A38b (4.2 mg, 17.2%) by prep-HPLC (Method A).

Compound A38a (the earlier peak): ¹H NMR (400 MHz, CD₃OD, the first peak) δ 7.85 (m, 2H), 7.45 (d, J=9.4 Hz, 1H), 6.12 (s, 1H), 5.32 (s, 1H), 4.45 (s, 1H), 4.06 (m, 1H), 3.98 (s, 2H), 3.85 (m, 1H), 3.60 (m, 1H), 3.46 (s, 3H), 3.15 (m, 1H), 2.93 (m, 1H), 2.44 (s, 3H), 2.24 (m, 1H), 1.85 (m, 2H), 1.71 (m, 1H), 1.55 (m, 1H), 1.41 (d, J=6.7 Hz, 3H), 0.93 (t, J=7.4 Hz, 3H), 0.56 (t, J=7.5 Hz, 3H) ppm. MS: M/e 488 (M+1)⁺.

Compound A38b (the later peak): ¹H NMR (400 MHz, CD₃OD, the second peak) δ 7.86 (m, 2H), 7.41 (d, J=9.4 Hz, 1H), 6.12 (s, 1H), 5.29 (s, 1H), 4.68 (s, 1H), 4.03 (m, 3H), 3.81 (m, 1H), 3.46 (s, 4H), 3.08 (m, 1H), 2.94 (m, 1H), 2.45 (m, 4H), 2.03 (m, 1H), 1.78 (m, 2H), 1.67 (d, J=7.9 Hz, 1H), 1.43 (t, J=9.9 Hz, 3H), 0.82 (t, J=7.5 Hz, 3H), 0.68 (t, J=7.4 Hz, 3H) ppm. MS: M/e 488 (M+1)⁺.

### Compound A39: 2-(7-((2S,5R)-2,5-diethyl-4-(1-(pyrazolo[1,5-a]pyrimidin-5-yl)ethyl)piperazin-1-yl)-4-methyl-5-oxo-4,5-dihydropyrazolo[1,5-a]pyrimidin-2-yl)acetonitrile

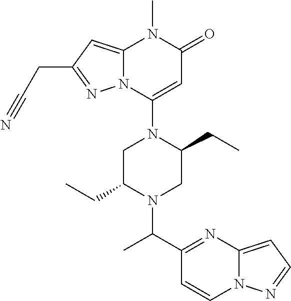 [SMILES: CC[C@H]1CN(C(C)c2ccn3nccc3n2)[C@H](CC)CN1c1cc(=O)n(C)c2cc(CC#N)nn12]

### Step A: 1-(pyrazolo[1,5-a]pyrimidin-5-yl)ethan-1-one

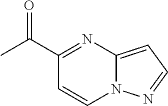

A mixture of 5-bromopyrazolo[1,5-a]pyrimidine (0.5 g, 2.5 mmol), tributyl(1-ethoxyvinyl)stannane (1.38 g, 3.8 mmol) and Pd(PPh₃)₂Cl₂ (178 mg, 0.25 mmol) in DMF (20 mL) was stirred at 100° C. under N₂ for 16 hours. The solution was diluted with ethyl acetate and washed with water. The organic layer was purified by flash column chromatography to give the 5-(1-ethoxyvinyl)pyrazolo [1,5-a]pyrimidine, then diluted with DCM and added with HCl (5 mL, 4 M in dioxane) in drops and the mixture was stirred at RT for 3 hours. The reaction was concentrated and adjusted to pH=8-9 with saturated NaHCO₃ solution, extracted with ethyl acetate. The combined organic layers were washed with brine, dried over Na₂SO₄ and the resulting residue was purified by flash column chromatography to give the titled compound (0.17 g, 41%). MS: M/e 162 (M+1)⁺

### Step B: 1-(pyrazolo[1,5-a]pyrimidin-5-yl)ethan-1-ol

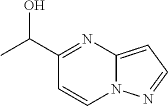

To a solution of 1-(pyrazolo[1,5-a]pyrimidin-5-yl)ethan-1-one (170 mg, 1.06 mmol) in MeOH (5 mL) was added NaBH₄ (32 mg, 0.84 mmol) at RT and the resulting mixture was stirred at RT for 5 min. The reaction mixture was diluted with DCM and washed with water, dried over Na₂SO₄ and concentrated to give the titled compound (70 mg). MS: M/e 164 (M+1)⁺.

### Step C: 2-(7-((2S,5R)-2,5-diethyl-4-(1-(pyrazolo[1,5-a]pyrimidin-5-yl)ethyl)piperazin-1-yl)-4-methyl-5-oxo-4,5-dihydropyrazolo[1,5-a]pyrimidin-2-yl)acetonitrile

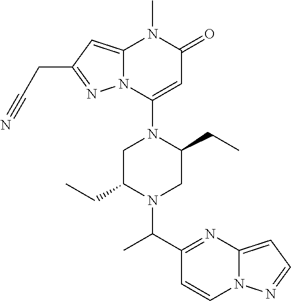 [SMILES: CC[C@H]1CN(C(C)c2ccn3nccc3n2)[C@H](CC)CN1c1cc(=O)n(C)c2cc(CC#N)nn12]

To a solution of 2-(7-((2S,5R)-2,5-diethylpiperazin-1-yl)-4-methyl-5-oxo-4,5-dihydropyrazolo[1,5-a]pyrimidin-2-yl)acetonitrile (30 mg, 0.1 mmol), 1-(pyrazolo[1,5-a]pyrimidin-5-yl)ethan-1-ol (32 mg, 0.2 mmol) and (cyanomethyl)trimethylphosphonium iodide (120 mg, 0.5 mmol) in CH₃CN (3 mL) was added DIPEA (258 mg, 2 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified and separated into Compound A39a (3.2 mg, 13.5%) and Compound A39b (2.3 mg, 9.7%) by prep-HPLC (Method A).

Compound A39a (the earlier peak): ¹H NMR (400 MHz, CD₃OD) δ 8.87 (d, J=7.3 Hz, 1H), 8.13 (d, J=2.4 Hz, 1H), 7.28 (d, J=7.3 Hz, 1H), 6.61 (m, 1H), 6.13 (s, 1H), 5.33 (s, 1H), 4.49 (s, 1H), 4.09 (m, 1H), 3.98 (s, 2H), 3.80 (q, J=6.5 Hz, 1H), 3.64 (m, 1H), 3.46 (s, 3H), 3.17 (m, 1H), 2.96 (m, 1H), 2.25 (m, 1H), 1.85 (m, 3H), 1.55 (m, 1H), 1.39 (d, J=6.7 Hz, 3H), 0.94 (t, J=7.4 Hz, 3H), 0.61 (t, J=7.5 Hz, 3H) ppm. MS: M/e 474 (M+1)⁺.

Compound A39b (the later peak): ¹H NMR (400 MHz, CD₃OD) δ 8.13 (d, J=2.3 Hz, 1H), 7.25 (d, J=7.3 Hz, 1H), 6.62 (m, 1H), 6.13 (s, 1H), 5.30 (s, 1H), 4.71 (s, 1H), 3.99 (m, 3H), 3.81 (m, 1H), 3.47 (m, 4H), 3.26 (m, 1H), 3.09 (m, 1H), 2.97 (m, 1H), 2.47 (m, 1H), 2.07 (m, 1H), 1.80 (m, 2H), 1.67 (m, 1H), 1.42 (d, J=6.6 Hz, 3H), 0.85 (t, J=7.5 Hz, 3H), 0.68 (t, J=7.4 Hz, 3H) ppm. MS: M/e 474 (M+1)⁺.

### Compound A40: 2-(7-((2S,5R)-2,5-diethyl-4-(1-(2-methylpyrazolo[1,5-a]pyrimidin-5-yl)ethyl)piperazin-1-yl)-4-methyl-5-oxo-4,5-dihydropyrazolo[1,5-a]pyrimidin-2-yl)acetonitrile

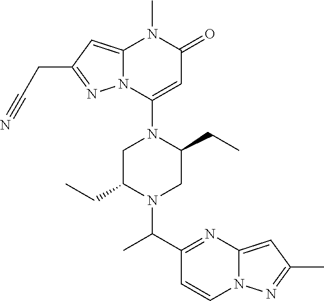 [SMILES: CC[C@H]1CN(C(C)c2ccn3nc(C)cc3n2)[C@H](CC)CN1c1cc(=O)n(C)c2cc(CC#N)nn12]

### Step A: 5-chloro-2-methylpyrazolo[1,5-a]pyrimidine

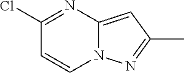

To a solution of 2-methylpyrazolo[1,5-a]pyrimidin-5(4H)-one (0.5 g, 3.36 mmol) in CH₃CN (6 mL) were added POCl₃ (1 g, 6.7 mmol). The mixture was stirred at 100° C. for 3 hours. The reaction was concentrated and poured into saturated NaHCO₃ aq., extracted with DCM. The combined organic layers were washed with brine, dried over Na₂SO₄ and concentrated to give the titled compound (0.33 g, 56%). MS: M/e 168 (M+1)⁺.

### Step B: 1-(2-methylpyrazolo[1,5-a]pyrimidin-5-yl)ethan-1-one

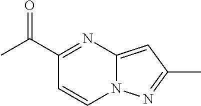

A mixture of 5-chloro-2-methylpyrazolo[1,5-a]pyrimidine (0.33 g, 1.9 mmol), tributyl(1-ethoxyvinyl)stannane (0.86 g, 2.4 mmol) and Pd(PPh₃)₂Cl₂ (139 mg, 0.19 mmol) in DMF (5 mL) was stirred at 100° C. under N₂ for 16 hours. The solution was diluted with ethyl acetate and washed with water. The organic layer was purified by flash column chromatography to give the 5-(1-ethoxyvinyl)-2-methylpyrazolo [1,5-a]pyrimidine, then diluted with DCM and added with HCl (5 mL, 4 M in dioxane) in drops and the mixture was stirred at RT for 2 hours. The reaction was concentrated and adjusted to pH=8-9 with saturated NaHCO₃ solution, extracted with ethyl acetate. The combined organic layers were washed with brine, dried over Na₂SO₄ and concentrated to dryness. The resulting residue was purified by flash column chromatography to give the titled compound (0.22 g, 76%). MS: M/e 176 (M+1)⁺

### Step C: 1-(2-methylpyrazolo[1,5-a]pyrimidin-5-yl)ethan-1-ol

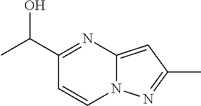

To a solution of 1-(2-methylpyrazolo[1,5-a]pyrimidin-5-yl)ethan-1-one (0.18 g, 0.73 mmol) in MeOH (5 mL) was added NaBH₄ (23 mg, 0.56 mmol) at RT and the resulting mixture was stirred at RT for 5 min. The reaction mixture was diluted with DCM and washed with water, dried over Na₂SO₄, concentrated to give the titled compound (170 mg, 85%). MS: M/e 178 (M+1)⁺.

### Step D: 2-(7-((2S,5R)-2,5-diethyl-4-(1-(2-methylpyrazolo[1,5-a]pyrimidin-5-yl)ethyl)piperazin-1-yl)-4-methyl-5-oxo-4,5-dihydropyrazolo[1,5-a]pyrimidin-2-yl)acetonitrile

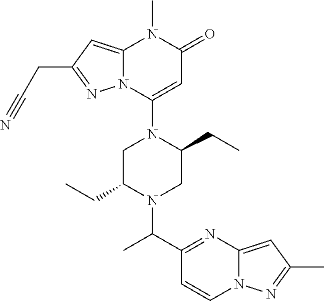 [SMILES: CC[C@H]1CN(C(C)c2ccn3nc(C)cc3n2)[C@H](CC)CN1c1cc(=O)n(C)c2cc(CC#N)nn12]

A mixture of 2-(7-((2S,5R)-2,5-diethylpiperazin-1-yl)-4-methyl-5-oxo-4,5-dihydropyrazolo[1,5-a]pyrimidin-2-yl)acetonitrile (30 mg, 0.1 mmol), 1-(2-methylpyrazolo[1,5-a]pyrimidin-5-yl)ethan-1-ol (60 mg, 0.3 mmol), (cyanomethyl)trimethylphosphonium iodide (120 mg, 0.5 mmol) and DIPEA (129 mg, 1 mmol) in MeCN (3 mL) was stirred at 100° C. for 16 hours. The resulted mixture was diluted with EtOAc (10 mL), washed with brine (5 mL×3), dried and concentrated. The resulting residue was purified by flash column chromatography to give the titled compound, which was further separated into Compound A40a (1.9 mg, 7.9%) and Compound A40b (2.2 mg, 9.1%) by prep-HPLC (Method A).

Compound A40a (the earlier peak): ¹H NMR (400 MHz, CD₃OD) δ 8.73 (d, J=7.2 Hz, 1H), 7.19 (d, J=7.2 Hz, 1H), 6.40 (s, 1H), 6.12 (s, 1H), 5.32 (s, 1H), 4.48 (s, 1H), 4.07 (m, 1H), 3.98 (s, 2H), 3.75 (m, 1H), 3.62 (m, 1H), 3.46 (s, 3H), 3.15 (m, 1H), 2.95 (m, 1H), 2.47 (s, 3H), 2.25 (m, 1H), 1.82 (m, 3H), 1.56 (m, 1H), 1.38 (d, J=6.6 Hz, 3H), 0.93 (t, J=7.3 Hz, 3H), 0.61 (t, J=7.4 Hz, 3H) ppm. MS: M/e 488 (M+1)⁺.

Compound A40b (the later peak): ¹H NMR (400 MHz, CD₃OD) δ 8.73 (d, J=7.1 Hz, 1H), 7.15 (d, J=7.2 Hz, 1H), 6.41 (s, 1H), 6.12 (s, 1H), 5.30 (s, 1H), 4.70 (s, 1H), 3.95 (m, 3H), 3.81 (m, 1H), 3.46 (m, 4H), 3.07 (m, 1H), 2.96 (m, 1H), 2.47 (m, 4H), 2.07 (m, 1H), 1.80 (m, 2H), 1.66 (d, J=7.4 Hz, 1H), 1.40 (d, J=6.6 Hz, 3H), 0.85 (t, J=7.4 Hz, 3H), 0.68 (d, J=7.3 Hz, 3H) ppm. MS: M/e 488 (M+1)⁺.

### Compound A41: 2-(7-((2S,5R)-2,5-diethyl-4-(1-(2-fluoropyrazolo[1,5-a]pyrimidin-5-yl)ethyl)piperazin-1-yl)-4-methyl-5-oxo-4,5-dihydropyrazolo[1,5-a]pyrimidin-2-yl)acetonitrile

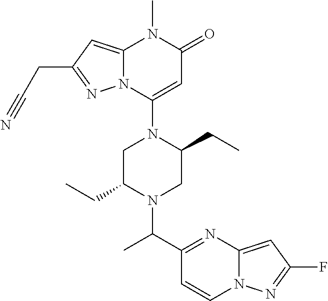 [SMILES: CC[C@H]1CN(C(C)c2ccn3nc(F)cc3n2)[C@H](CC)CN1c1cc(=O)n(C)c2cc(CC#N)nn12]

### Step A: 2-fluoropyrazolo[1,5-a]pyrimidin-5(4H)-one

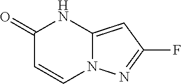 [SMILES: O=c1ccn2nc(F)cc2[nH]1]

A mixture of 3-fluoro-1H-pyrazol-5-amine (0.25 g, 2.47 mmol), ethyl (E)-3-ethoxyacrylate (0.7 g, 4.9 mmol) and Cs₂CO₃ (1.6 g, 4.9 mmol) in DMF (10 mL) was stirred at 110° C. under N₂ for 16 hours. The solution was collected by filtration, then added acetic acid (5 mL). The organic layer was concentrated to dryness. The resulting residue was purified by flash column chromatography to give the titled compound (0.46 g). MS: M/e 154 (M+1)⁺

### Step B: 5-chloro-2-fluoropyrazolo[1,5-a]pyrimidine

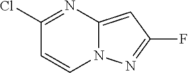 [SMILES: Fc1cc2nc(Cl)ccn2n1]

To a solution of 2-fluoropyrazolo[1,5-a]pyrimidin-5(4H)-one (0.46 g, 3 mmol) in CH₃CN (8 mL) were added POCl₃ (0.92 g, 6 mmol). The mixture was stirred at 90° C. for 3 hours. The reaction was concentrated and poured into saturated NaHCO₃ solution, extracted with DCM. The combined organic layers were washed with brine, dried over Na₂SO₄ and concentrated to dryness. The resulting residue was purified by flash column chromatography to give the titled compound (0.27 g, 52%). MS: M/e 172 (M+1)⁺.

### Step C: 1-(2-fluoropyrazolo[1,5-a]pyrimidin-5-yl)ethan-1-one

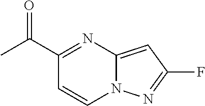

A mixture of 5-chloro-2-fluoropyrazolo[1,5-a]pyrimidine (0.27 g, 1.58 mmol), tributyl(1-ethoxyvinyl)stannane (0.68 g, 1.89 mmol) and Pd(PPh₃)₂Cl₂ (110 mg, 0.27 mmol) in DMF (5 mL) was stirred at 100° C. under N₂ for 16 hours. The solution was diluted with ethyl acetate and washed with water. The organic layer was purified by flash column chromatography to give the 5-(1-ethoxyvinyl)-2-fluoropyrazolo [1,5-a]pyrimidine, then diluted with DCM and added with HCl (5 mL, 4 M in dioxane) in drops and the mixture was stirred at RT for 2 hours. The reaction was concentrated and adjusted to pH=8-9 with saturated NaHCO₃ solution, extracted with ethyl acetate. The combined organic layers were washed with brine, dried over Na₂SO₄ and concentrated to dryness. The resulting residue was purified by flash column chromatography to give the titled compound (0.18 g, 63%). MS: M/e 180 (M+1)⁺

### Step D: 1-(2-fluoropyrazolo[1,5-a]pyrimidin-5-yl)ethan-1-ol

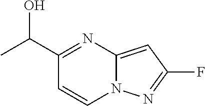

To a solution of 1-(2-fluoropyrazolo[1,5-a]pyrimidin-5-yl)ethan-1-one (0.17 g, 0.95 mmol) in MeOH (5 mL) was added NaBH₄ (22 mg, 0.57 mmol) at RT and the resulting mixture was stirred at RT for 5 mins. The reaction mixture was diluted with DCM and washed with water, dried over Na₂SO₄, concentrated to give the titled compound (150 mg). MS: M/e 182 (M+1)⁺.

### Step E: 2-(7-((2S,5R)-2,5-diethyl-4-(1-(2-fluoropyrazolo[1,5-a]pyrimidin-5-yl)ethyl)piperazin-1-yl)-4-methyl-5-oxo-4,5-dihydropyrazolo[1,5-a]pyrimidin-2-yl)acetonitrile

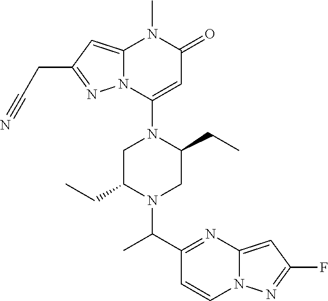 [SMILES: CC[C@H]1CN(C(C)c2ccn3nc(F)cc3n2)[C@H](CC)CN1c1cc(=O)n(C)c2cc(CC#N)nn12]

A mixture of 2-(7-((2S,5R)-2,5-diethylpiperazin-1-yl)-4-methyl-5-oxo-4,5-dihydropyrazolo[1,5-a]pyrimidin-2-yl)acetonitrile (30 mg, 0.1 mmol), 1-(2-fluoropyrazolo[1,5-a]pyrimidin-5-yl)ethan-1-ol (60 mg, 0.3 mmol), (cyanomethyl)trimethylphosphonium iodide (120 mg, 0.5 mmol) and DIPEA (129 mg, 1 mmol) in MeCN (3 mL) was stirred at 100° C. for 16 hours. The resulted mixture was diluted with EtOAc (10 mL), washed with brine (5 mL×3), dried and concentrated. The resulting residue was purified by flash column chromatography to give the titled compound, which was further separated into Compound A41a (0.69 mg, 2.8%) and Compound A41b (0.85 mg, 3.5%) by prep-HPLC (Method A).

Compound A41a (the earlier peak): ¹H NMR (400 MHz, CD₃OD) δ 8.73 (d, J=7.2 Hz, 1H), 7.29 (d, J=7.3 Hz, 1H), 6.18 (d, J=4.9 Hz, 1H), 6.12 (s, 1H), 5.33 (s, 1H), 4.48 (s, 1H), 4.07 (m, 1H), 3.98 (s, 2H), 3.78 (m, 1H), 3.64 (m, 1H), 3.46 (s, 3H), 3.13 (m, 1H), 2.95 (m, 1H), 2.27 (m, 1H), 1.81 (m, 3H), 1.55 (m, 1H), 1.38 (d, J=6.7 Hz, 3H), 0.92 (t, J=7.4 Hz, 3H), 0.63 (t, J=7.5 Hz, 3H) ppm. MS: M/e 492 (M+1)⁺.

Compound A41b (the later peak): ¹H NMR (400 MHz, CD₃OD) δ 8.72 (d, J=7.1 Hz, 1H), 7.26 (d, J=7.4 Hz, 1H), 6.18 (d, J=4.9 Hz, 1H), 6.12 (s, 1H), 5.30 (s, 1H), 4.68 (m, 1H), 3.98 (m 3H), 3.82 (m, 1H), 3.46 (m, 4H), 3.07 (m, 1H), 2.96 (m, 1H), 2.48 (m, 1H), 2.14 (m, 1H), 1.81 (m, 2H), 1.65 (m, 1H), 1.40 (d, J=6.6 Hz, 3H), 0.84 (d, J=7.5 Hz, 3H), 0.69 (m, 3H) ppm. MS: M/e 492 (M+1)⁺.

### Compound A42: 2-(7-((2S,5R)-5-ethyl-2-methyl-4-(1-(2-methylpyrazolo [1,5-a]pyrimidin-5-yl)ethyl)piperazin-1-yl)-4-methyl-5-oxo-4,5-dihydropyrazolo[1,5-a]pyrimidin-2-yl)acetonitrile

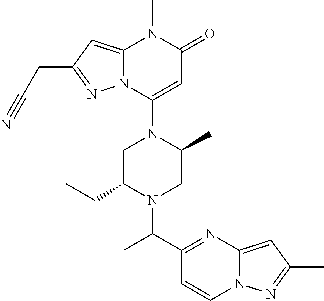 [SMILES: CC[C@@H]1CN(c2cc(=O)n(C)c3cc(CC#N)nn23)[C@@H](C)CN1C(C)c1ccn2nc(C)cc2n1]

A mixture of 2-(7-((2S,5R)-5-ethyl-2-methylpiperazin-1-yl)-4-methyl-5-oxo-4,5-dihydropyrazolo[1,5-a]pyrimidin-2-yl)acetonitrile (30 mg, 0.1 mmol), 1-(2-methylpyrazolo[1,5-a]pyrimidin-5-yl)ethan-1-ol (60 mg, 0.3 mmol), (cyanomethyl)trimethylphosphonium iodide (120 mg, 0.5 mmol) and DIPEA (129 mg, 1 mmol) in MeCN (3 mL) was stirred at 100° C. for 16 hours. The resulted mixture was diluted with EtOAc (10 mL), washed with brine (5 mL×3), dried and concentrated. The resulting residue was purified by flash column chromatography to give the titled compound, which was further separated into Compound A42a (0.69 mg, 2.76%) and Compound A42b (0.87 mg, 3.48%) by prep-HPLC (Method A).

Compound A42a (the earlier peak): ¹H NMR (400 MHz, CD₃OD) δ 8.72 (d, J=7.2 Hz, 1H), 7.21 (d, J=7.2 Hz, 1H), 6.40 (s, 1H), 6.12 (s, 1H), 5.35 (s, 1H), 4.64 (s, 1H), 3.99 (d, J=15.1 Hz, 3H), 3.78 (m, 1H), 3.64 (m, 1H), 3.46 (s, 3H), 3.16 (m, 1H), 2.97 (m, 1H), 2.46 (s, 3H), 2.15 (m, 1H), 1.91 (m, 1H), 1.56 m, 1H), 1.39 (d, J=6.6 Hz, 3H), 1.21 (d, J=6.5 Hz, 3H), 0.98 (t, J=7.3 Hz, 3H) ppm. MS: M/e 474 (M+1)⁺.

Compound A42b (the later peak): ¹H NMR (400 MHz, CD₃OD) δ 8.73 (d, J=7.1 Hz, 1H), 7.17 (d, J=7.2 Hz, 1H), 6.41 (s, 1H), 6.12 (s, 1H), 5.33 (s, 1H), 3.96 (m, 3H), 3.74 (m, 1H), 3.46 (m, 4H), 3.12 (m, 2H), 2.82 (m, 1H), 2.47 (m, 4H), 1.79 (m, 1H), 1.67 (m, 1H), 1.40 (d, J=6.6 Hz, 3H), 1.35 (d, J=6.6 Hz, 3H), 0.71 (d, J=7.3 Hz, 3H) ppm. MS: M/e 474 (M+1)⁺.

### Compound A43: 2-(7-((2S,5R)-5-ethyl-2-methyl-4-(1-(2-methylpyrazolo [1,5-a]pyrimidin-5-yl)propyl)piperazin-1-yl)-4-methyl-5-oxo-4,5-dihydropyrazolo [1,5-a]pyrimidin-2-yl)acetonitrile

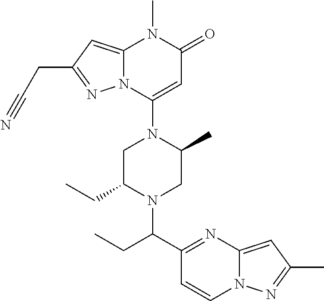 [SMILES: CCC(c1ccn2nc(C)cc2n1)N1C[C@H](C)N(c2cc(=O)n(C)c3cc(CC#N)nn23)C[C@H]1CC]

### Step A: 2-methylpyrazolo[1,5-a]pyrimidine-5-carbonitrile

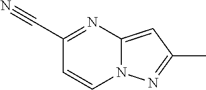

To a stirred solution of 5-chloro-2-methylpyrazolo[1,5-a]pyrimidine (1 g, 6 mmol) in DMF (15 mL) was added ZnCN₂ (2.1 g, 18 mmol), followed by Pd(PPh₃)₄ (0.7 g, 0.6 mmol). After the addition, the reaction mixture was stirred overnight at 100° C. under N₂. The reaction mixture was poured into H₂O (20 mL) and extracted with EA (30 mL×3). The combined organic layers were washed with brine, dried over Na₂SO₄, concentrated to dryness. The resulting residue was purified by flash column chromatography to give the titled compound (650 mg, 68.5%). MS: M/e 159 (M+1)⁺.

### Step B: 2-methylpyrazolo[1,5-a]pyrimidine-5-carboxylic Acid

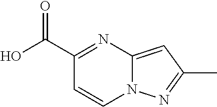

To a stirred solution of 2-methylpyrazolo[1,5-a]pyrimidine-5-carbonitrile (650 mg, 4.11 mmol) in EtOH/H₂O (20 mL/5 mL) was added NaOH (658 mg, 16.4 mmol). After the addition, the reaction mixture was stirred at 80° C. overnight. The reaction mixture was acidified to pH=5-6 with aq.HCl, then concentrated to give the titled compound (crude, 100%), which was directly used to the next step. MS: M/e 178 (M+1)⁺.

### Step C: N-methoxy-N,2-dimethylpyrazolo[1,5-a]pyrimidine-5-carboxamide

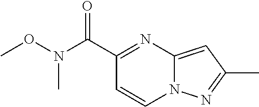

A mixture of 2-methylpyrazolo[1,5-a]pyrimidine-5-carboxylic acid (crude, 4.11 mmol), N,O-dimethylhydroxylamine hydrochloride (480 mg, 4.93 mmol), HATU (1.88 g, 4.93 mmol) and DIPEA (1.06 g, 8.22 mmol) in CH₂Cl₂ (40 mL) was stirred overnight. The reaction mixture was poured into H₂O (40 mL), then extracted with CH₂Cl₂ (40 mL×2). The combined organic layers were washed with brine, dried over Na₂SO₄, concentrated to dryness. The resulting residue was purified by flash column chromatography to give the titled compound (690 mg, 76.3%). MS: M/e 221 (M+1)⁺.

### Step D: 1-(2-methylpyrazolo[1,5-a]pyrimidin-5-yl)propan-1-one

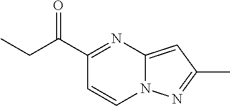

To a stirred solution of N-methoxy-N,2-dimethylpyrazolo[1,5-a]pyrimidine-5-carboxamide (690 mg, 3.14 mmol) in THF (10 mL) was added dropwise EtMgBr (3.0 M, 1.56 mL, 4.7 mL) at 0° C. After then, the mixture was stirred for 20 min. The reaction was quenched with aq.NH₄Cl, extracted with EA (10 mL×3). The combined organic layers were washed with brine, dried over Na₂SO₄, concentrated to dryness. The resulting residue was purified by flash column chromatography to give the titled compound (26 mg, 4.38%). MS: M/e 190 (M+1)⁺.

### Step E: 1-(2-methylpyrazolo[1,5-a]pyrimidin-5-yl)propan-1-ol

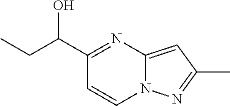

To a stirred solution of 1-(2-methylpyrazolo[1,5-a]pyrimidin-5-yl)propan-1-one (60 mg, 0.32 mol) in MeOH (5 mL) was added NaBH₄ (12 mg, 0.32 mmol). After then, the reaction mixture was stirred for 10 min. The reaction mixture was treated with H₂O (10 mL), then concentrated to remove MeOH and extracted with EA (10 mL×3). The combined organic layers were washed with brine, dried over Na₂SO₄, concentrated to give the titled compound (60 mg, 98%). MS: M/e 192 (M+1)⁺.

### Step F: 2-(7-((2S,5R)-5-ethyl-2-methyl-4-(1-(2-methylpyrazolo[1,5-a]pyrimidin-5-yl)propyl)piperazin-1-yl)-4-methyl-5-oxo-4,5-dihydropyrazolo[1,5-a]pyrimidin-2-yl)acetonitrile

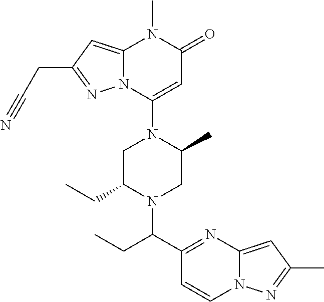 [SMILES: CCC(c1ccn2nc(C)cc2n1)N1C[C@H](C)N(c2cc(=O)n(C)c3cc(CC#N)nn23)C[C@H]1CC]

A mixture of 2-(7-((2S,5R)-5-ethyl-2-methylpiperazin-1-yl)-4-methyl-5-oxo-4,5-dihydropyrazolo[1,5-a]pyrimidin-2-yl)acetonitrile (30 mg, 0.1 mmol), 1-(2-methylpyrazolo[1,5-a]pyrimidin-5-yl)propan-1-ol (60 mg, 0.3 mmol), (cyanomethyl)trimethylphosphonium iodide (120 mg, 0.5 mmol) and DIPEA (129 mg, 1 mmol) in MeCN (3 mL) was stirred at 100° C. for 16 hours. The resulted mixture was diluted with EtOAc (10 mL), washed with brine (5 mL×3), dried and concentrated. The resulting residue was purified by flash column chromatography to give the titled compound (1.39 mg, 2.8%). ¹H NMR (400 MHz, CD₃OD) δ 8.73 (m, 1H), 7.13 (m, 1H), 6.42 (d, J=7.8 Hz, 1H), 6.11 (s, 1H), 5.33 (d, J=5.5 Hz, 1H), 4.66 (m, 1H), 3.98 (s, 2H), 3.77 (m, 1H), 3.59 (m, 1H), 3.46 (m, 4H), 3.14 (m, 1H), 3.2-2.85 (m, 1H), 2.47 (s, 3H), 2.02 (m, 1H), 1.90 (m, 1H), 1.77 (m, 2H), 1.54 (m, 1H), 1.34-1.18 (m, 3H), 0.97-0.79 (m, 3H), 0.71 (t, J=7.5 Hz, 3H) ppm. MS: M/e 488 (M+1)⁺.

### Compound A44: 2-(7-((2S,5R)-4-(1-(2-chlorothieno[3,2-b]pyridin-5-yl)ethyl)-2,5-diethylpiperazin-1-yl)-4-methyl-5-oxo-4,5-dihydropyrazolo[1,5-a]pyrimidin-2-yl)acetonitrile

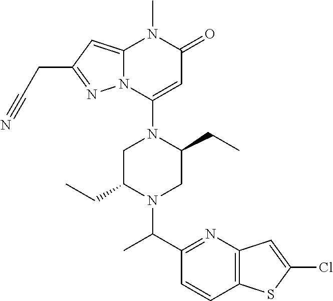 [SMILES: CC[C@H]1CN(C(C)c2ccc3sc(Cl)cc3n2)[C@H](CC)CN1c1cc(=O)n(C)c2cc(CC#N)nn12]

### Step A: 2-chlorothieno[3,2-b]pyridine

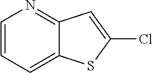

To a solution of thieno[3,2-b]pyridine (3.15 g, 23.3 mmol) in THF (30 mL), cool down to −70° C. under N₂ protection was added n-BuLi (1.6 M, 35 mmol, 21.8 mL). The reaction mixture was stirred at −70° C. for 0.5 hr. NCS (6.2 g, 46.6 mmol) was added and the reaction was slowly warmed up to RT. The mixture was added H₂O and extracted by EtOAc. The organic layer was washed with brine (30 mL), dried and concentrated. The resulting residue was purified by flash column chromatography with 0-100% EtOAc in PE to give the titled compound (3.2 g, 82.5%). MS: M/e 170 (M+1)⁺.

### Step B: 2-chlorothieno[3,2-b]pyridine 4-oxide

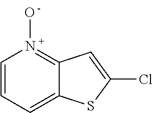

To a solution of 2-chlorothieno[3,2-b]pyridine (1.4 g, 8.3 mmol) in DCM (30 mL) was added m-CPBA (2.1 g, 12.4 mmol). The reaction mixture was stirred at RT for 16 hrs. The reaction mixture was poured into 2M K₂CO₃ solution (30 mL) and then extracted with DCM (30 mL×3). The organic layer was washed with brine (30 mL), dried and concentrated to give the titled compound which was used directly in next step. MS: M/e 186 (M+1)⁺.

### Step C: 2-chlorothieno[3,2-b]pyridine-5-carbonitrile

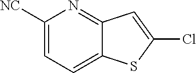 [SMILES: N#Cc1ccc2sc(Cl)cc2n1]

To a solution of 2-chlorothieno[3,2-b]pyridine 4-oxide from last step in MeCN (15 mL) was added Et₃N (2 g, 20 mmol) and TMSCN (1.9 g, 19.4 mmol). The reaction was stirred at 90° C. for 3 h. The mixture was added H₂O (30 mL) and then extracted with EtOAc (30 mL×3). The organic layer was washed with brine (30 mL), dried and concentrated. The resulting residue was purified by flash column chromatography with 0-100% EA in PE to give the titled compound (0.8 g, 50% for 2 steps). MS: M/e 195 (M+1)⁺.

### Step D: 1-(2-chlorothieno[3,2-b]pyridin-5-yl)ethan-1-one

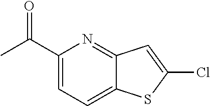

A solution of 2-chlorothieno[3,2-b]pyridine-5-carbonitrile (420 mg, 2 mmol) in THF was cooled down to 0° C. CH₃MgBr (1 M, 2 mmol, 2 mL) was slowly added to the mixture. The reaction mixture was stirred at RT for 16 hours. The reaction mixture was added H₂O (30 mL) and then extracted with EtOAc (30 mL×3). The organic layer was washed with brine (30 mL), dried and concentrated. The resulting residue was purified by flash column chromatography with 0-100% EA in PE to give the titled compound (45 mg, 10.6%). MS: M/e 212 (M+1)⁺.

### Step E: 1-(2-chlorothieno[3,2-b]pyridin-5-yl)ethan-1-ol

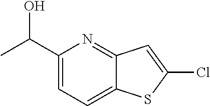

To a solution of 1-(2-chlorothieno[3,2-b]pyridin-5-yl)ethan-1-one (45 mg, 0.2 mmol) in MeOH was added NaBH₄ (9 mg, 0.25 mmol) and the reaction mixture was stirred at RT for 15 mins. The reaction mixture was added H₂O (30 mL) and then extracted with DCM (30 mL×3). The organic layer was washed with brine (30 mL), dried and concentrated. The resulting residue was purified by flash column chromatography with 0-100% EA in PE to give the titled compound (30 mg, 67%). MS: M/e 214 (M+1)⁺.

### Step F: 2-(7-((2S,5R)-4-(1-(2-chlorothieno[3,2-b]pyridin-5-yl)ethyl)-2,5-diethyl piperazin-1-yl)-4-methyl-5-oxo-4,5-dihydropyrazolo[1,5-a]pyrimidin-2-yl) acetonitrile

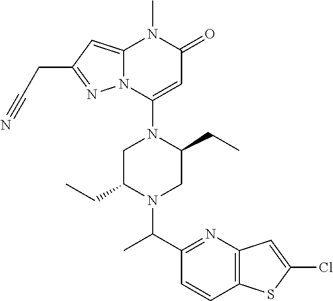 [SMILES: CC[C@H]1CN(C(C)c2ccc3sc(Cl)cc3n2)[C@H](CC)CN1c1cc(=O)n(C)c2cc(CC#N)nn12]

A mixture of 2-(7-((2S,5R)-2,5-diethylpiperazin-1-yl)-4-methyl-5-oxo-4,5-dihydropyrazolo[1,5-a]pyrimidin-2-yl)acetonitrile (30 mg, 0.1 mmol), 1-(2-chlorothieno[3,2-b]pyridin-5-yl)ethan-1-ol (30 mg, 0.15 mmol), (cyanomethyl)trimethylphosphonium iodide (120 mg, 0.5 mmol) and DIPEA (129 mg, 1 mmol) in CH₃CN (3 mL) was stirred at 100° C. for 16 hours. The resulted mixture was diluted with EtOAc (10 mL), washed with brine (5 mL×3), dried and concentrated. The resulting residue was purified by flash column chromatography to give the titled compound, which was further separated into Compound A44a (2.5 mg, 9.5%) and Compound A44b (3.5 mg, 13.4%) by prep-HPLC (Method A).

Compound A44a (the earlier peak): ¹H NMR (400 MHz, CD₃OD) δ 8.27 (d, J=8.5 Hz, 1H), 7.65 (d, J=8.5 Hz, 1H), 7.41 (s, 1H), 6.12 (s, 1H), 5.32 (s, 1H), 4.45 (s, 1H), 4.08 (m, 1H), 3.97 (s, 2H), 3.87 (m, 1H), 3.64 (m, 1H), 3.46 (s, 3H), 3.20 (m, 1H), 2.91 (m, 1H), 2.20 (m, 1H), 1.90 (m, 2H), 1.70 (m, 1H), 1.58 (m, 1H), 1.39 (d, J=6.7 Hz, 3H), 0.95 (t, J=7.4 Hz, 3H), 0.54 (t, 0.1=7.5 Hz, 3H) ppm. MS: M/e 524 (M+1)⁺.

Compound A44b (the later peak): ¹H NMR (400 MHz, CD₃OD) δ 8.28 (d, J=8.5 Hz, 1H), 7.62 (d, J=8.5 Hz, 1H), 7.42 (s, 1H), 6.12 (s, 1H), 5.29 (s, 1H), 4.71 (m, 1H), 4.05 (m, 1H), 3.99 (s, 2H), 3.79 (m, 1H), 3.45 (m, 4H), 3.06 (m, 2H), 2.38 (m, 1H), 2.12 (m, 1H), 1.77 (m, 3H), 1.41 (d, J=6.6 Hz, 3H), 0.88 (t, J=7.5 Hz, 3H), 0.60 (t, J=7.4 Hz, 3H) ppm. MS: M/e 524 (M+1)⁺.

### Compound A45: 2-(4-((2S,5R)-2,5-diethyl-4-(1-(2-methylimidazo[1,2-b]pyridazin-6-yl)ethyl)piperazin-1-yl)-1-methyl-2-oxo-1,2-dihydropyrazolo[1,5-a][1,3,5]triazin-7-yl)acetonitrile

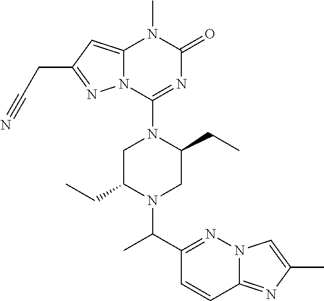 [SMILES: CC[C@H]1CN(C(C)c2ccc3nc(C)cn3n2)[C@H](CC)CN1c1nc(=O)n(C)c2cc(CC#N)nn12]

A solution of 2-(4-((2S,5R)-2,5-diethylpiperazin-1-yl)-1-methyl-2-oxo-1,2-dihydropyrazolo[1,5-a][1,3,5]triazin-7-yl)acetonitrile (50 mg, 0.15 mmol), 1-(2-methylimidazo[1,2-b]pyridazin-6-yl)ethan-1-ol (54 mg, 0.31 mmol), (cyanomethyl)trimethylphosphonium iodide (111 mg, 0.46 mmol) and DIPEA (196 mg, 1.52 mmol) in CH₃CN (2 mL) was stirred at 100° C. overnight. The reaction was diluted with EtOAc (10 mL) and washed with brine (10 mL). The organic layer was concentrated under reduced pressure. The resulting residue was purified by flash column chromatography with 0-10% MeOH in DCM and then further separated into Compound A45a (3.1 mg, 4%) and Compound A45b (3.6 mg, 5%) by prep-HPLC (Method A).

Compound A45a (the earlier peak): ¹H NMR (400 MHz, CD₃OD) δ 7.89-7.79 (m, 2H), 7.44 (d, J=9.1 Hz, 1H), 6.43-6.12 (m, 0.5H), 6.03 (s, 1H), 5.95-5.65 (m, 0.5H), 5.09-4.88 (m, 1H), 4.02-3.95 (m, 1H), 3.86-3.74 (m, 1H), 3.41 (s, 3H), 3.36-3.31 (m, 2H), 3.20-3.10 (m, 1H), 2.85-2.73 (m, 1H), 2.44 (s, 3H), 2.28 (d, J=11.9 Hz, 1H), 2.00-1.75 (m, 2H), 1.64-1.46 (m, 2H), 1.40 (d, J=5.3 Hz, 3H), 1.09-0.95 (m, 3H), 0.74-0.63 (m, 3H) ppm. MS: M/e 489 (M+1)³⁰

Compound A45b (the later peak): ¹H NMR (400 MHz, CD₃OD) δ 7.90-7.80 (m, 2H), 7.39 (d, J=9.0 Hz, 1H), 6.25-5.85 (m, 2H), 5.03-4.88 (m, 1H), 4.02-3.95 (m, 2H), 3.41 (s, 3H), 3.34-3.31 (m, 2H), 3.00-2.87 (m, 2H), 2.52-2.37 (m, 4H), 2.10-1.93 (m, 2H), 1.69-1.48 (m, 2H), 1.42 (d, J=6.3 Hz, 3H), 0.97-0.86 (m, 3H), 0.83-0.68 (m, 3H) ppm. MS: M/e 489 (M+1)⁺

### Compound A46: 2-(4-((2S,5R)-2,5-diethyl-4-(1-(2-methylpyrazolo[1,5-a]pyrimidin-5-yl)ethyl)piperazin-1-yl)-1-methyl-2-oxo-1,2-dihydropyrazolo[1,5-a][1,3,5]triazin-7-yl)acetonitrile

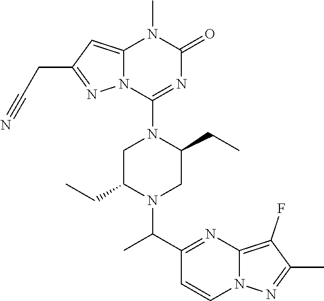 [SMILES: CC[C@H]1CN(C(C)c2ccn3nc(C)c(F)c3n2)[C@H](CC)CN1c1nc(=O)n(C)c2cc(CC#N)nn12]

A solution of 2-(4-((2S,5R)-2,5-diethylpiperazin-1-yl)-1-methyl-2-oxo-1,2-dihydropyrazolo[1,5-a][1,3,5]triazin-7-yl)acetonitrile (250 mg, 0.76 mmol), 1-(2-methylpyrazolo[1,5-a]pyrimidin-5-yl)ethan-1-ol (269 mg, 1.52 mmol), (cyanomethyl)trimethylphosphonium iodide (554 mg, 2.28 mmol) and DIPEA (980 mg, 7.60 mmol) in CH₃CN (6 mL) was stirred at 100° C. for 2 days. The reaction was diluted with EtOAc (15 mL) and washed with brine (10 mL). The organic layer was concentrated under reduced pressure. The resulting residue was purified by flash column chromatography with 0-5% MeOH in DCM to give the titled compound, which was further separated into Compound A46a (40.7 mg, 11.0%) and Compound A46b (40.0 mg, 10.8%) by prep-HPLC (Method A).

Compound A46a (the earlier peak): ¹H NMR (400 MHz, CD₃OD) δ 8.74 (d, J=7.2 Hz, 1H), 7.19 (d, J=7.2 Hz, 1H), 6.40 (s, 1H), 6.04 (s, 1H), 4.91-4.85 (m, 2H), 3.99 (s, 2H), 3.72 (q, J=6.6 Hz, 1H), 3.42 (s, 3H), 3.35-3.31 (m, 1H), 3.21-3.11 (m, 1H), 2.79 (d, J=11.7 Hz, 1H), 2.47 (s, 3H), 2.29 (d, J=11.7 Hz, 1H), 2.04-1.83 (m, 2H), 1.65-1.45 (m, 2H), 1.38 (d, J=6.6 Hz, 3H), 1.02 (t, J=6.8 Hz, 3H), 0.74 (t, J=7.3 Hz, 3H) ppm. MS: M/e 489 (M+1)⁺.

Compound A46b (the later peak): ¹H NMR (400 MHz, CD₃OD) δ 8.73 (d, J=7.2 Hz, 1H), 7.14 (d, J=7.3 Hz, 1H), 6.41 (s, 1H), 6.04 (s, 1H), 5.04-4.87 (m, 2H), 4.06-3.86 (m, 2H), 3.42 (s, 3H), 3.36-3.32 (m, 2H), 3.02-2.87 (m, 2H), 2.54-2.40 (m, 4H), 2.12-2.00 (m, 2H), 1.68-1.50 (m, 2H), 1.39 (d, J=6.5 Hz, 3H), 0.95 (t, J=6.8 Hz, 3H), 0.76 (s, 3H) ppm. MS: M/e 489 (M+1)⁺.

### Compound A47: 2-(4-((2S,5R)-2,5-diethyl-4-(1-(3-fluoro-2-methylpyrazolo[1,5-a]pyrimidin-5-yl)ethyl)piperazin-1-yl)-1-methyl-2-oxo-1,2-dihydropyrazolo[1,5-a][1,3,5]triazin-7-yl)acetonitrile

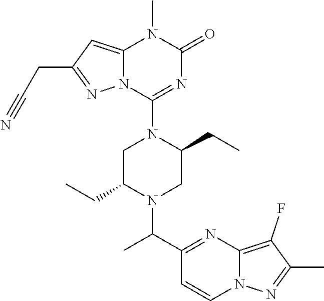 [SMILES: CC[C@H]1CN(C(C)c2ccn3nc(C)c(F)c3n2)[C@H](CC)CN1c1nc(=O)n(C)c2cc(CC#N)nn12]

### Step A: 4-fluoro-3-methyl-1H-pyrazol-5-amine

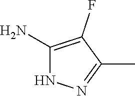

To a solution of 3-methyl-1H-pyrazol-5-amine (0.5 g, 5.1 mmol) in CH₃CN (20 mL) were added 1-Chloromethyl-4-fluoro-1,4-diazoniabicyclo[2.2.2]octane bis(tetrafluoroborate) (1.6 g, 4.6 mmol) was stirred at room temperature under N₂ for 16 hours. The solution was purified by flash column chromatography to give the titled compound (0.41 g). MS: M/e 116 (M+1)⁺

### Step B: 3-fluoro-2-methylpyrazolo[1,5-a]pyrimidin-5(4H)-one

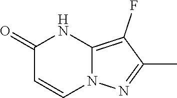

A mixture of 4-fluoro-3-methyl-1H-pyrazol-5-amine (0.41 g, 3.5 mmol), ethyl (E)-3-ethoxyacrylate (1 g, 7.1 mmol) and Cs₂CO₃ (2.3 g, 7.1 mmol) in DMF (15 mL) was stirred at 110° C. under N₂ for 16 hours. The solution was collected by filtration, then added acetic acid (5 mL). The organic layer was concentrated to dryness. The resulting residue was purified by flash column chromatography to give the titled compound (0.09 g, 15%). MS: M/e 168 (M+1)⁺

### Step C: 5-chloro-3-fluoro-2-methylpyrazolo[1,5-a]pyrimidine

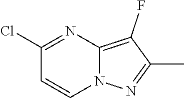

To a solution of 3-fluoro-2-methylpyrazolo[1,5-a]pyrimidin-5(4H)-one (0.09 g, 0.54 mmol) in CH₃CN (3 mL) were added POCl₃ (0.16 g, 1 mmol). The mixture was stirred at 90° C. for 3 hours. The reaction was concentrated and poured into saturated NaHCO₃ solution, extracted with DCM. The combined organic layers were washed with brine, dried over Na₂SO₄ and concentrated to dryness. The resulting residue was purified by flash column chromatography to give the titled compound (0.07 g, 70%). MS: M/e 186 (M+1)⁺.

### Step D: 1-(3-fluoro-2-methylpyrazolo[1,5-a]pyrimidin-5-yl)ethan-1-one

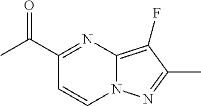

A mixture of 5-chloro-3-fluoro-2-methylpyrazolo[1,5-a]pyrimidine (0.07 g, 0.38 mmol), tributyl(1-ethoxyvinyl)stannane (0.16 g, 0.45 mmol) and Pd(PPh₃)₂Cl₂ (27 mg, 0.038 mmol) in DMF (2 mL) was stirred at 100° C. under N₂ for 16 hours. The solution was diluted with ethyl acetate and washed with water. The organic layer was concentrated to dryness. The resulting residue was purified by flash column chromatography to give the titled compound (0.06 g, 82%). MS: M/e 194 (M+1)³⁰

### Step E: 1-(3-fluoro-2-methylpyrazolo[1,5-a]pyrimidin-5-yl)ethan-1-ol

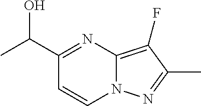

To a solution of 1-(3-fluoro-2-methylpyrazolo[1,5-a]pyrimidin-5-yl)ethan-1-one (0.06 g, 0.31 mmol) in MeOH (5 mL) was added NaBH₄ (7 mg, 0.18 mmol) at room temperature and the resulting mixture was stirred at room temperature for 5 mins. The reaction mixture was diluted with DCM and washed with water, dried over Na₂SO₄ and concentrated to give the titled compound (150 mg). MS: M/e 196 (M+1)⁺.

### Step F: 2-(4-((2S,5R)-2,5-diethyl-4-(1-(3-fluoro-2-methylpyrazolo[1,5-a]pyrimidin-5-yl)ethyl)piperazin-1-yl)-1-methyl-2-oxo-1,2-dihydropyrazolo[1,5-a][1,3,5]triazin-7-yl)acetonitrile

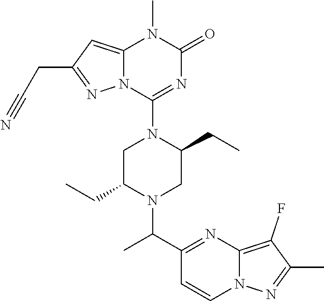 [SMILES: CC[C@H]1CN(C(C)c2ccn3nc(C)c(F)c3n2)[C@H](CC)CN1c1nc(=O)n(C)c2cc(CC#N)nn12]

A solution of 2-(4-((2S,5R)-2,5-diethylpiperazin-1-yl)-1-methyl-2-oxo-1,2-dihydropyrazolo[1,5-a][1,3,5]triazin-7-yl)acetonitrile (100 mg, 0.30 mmol), 1-(3-fluoro-2-methylpyrazolo[1,5-a]pyrimidin-5-yl)ethan-1-ol (118.5 mg, 0.61 mmol), (cyanomethyl)trimethylphosphonium iodide (222 mg, 0.91 mmol) and DIPEA (392 mg, 3.04 mmol) in CH₃CN (2 mL) was stirred at 100° C. for 2 days. The reaction was diluted with EtOAc (10 mL) and washed with brine (10 mL). The organic layer was concentrated under reduced pressure. The resulting residue was purified by flash column chromatography with 0-5% MeOH in DCM to give the titled compound, which was further separated into Compound A47a (21.9 mg, 14.2%) and Compound A47b (20.5 mg, 13.3%) by prep-HPLC (Method A).

Compound A47a (the earlier peak): ¹H NMR (400 MHz, CD₃OD) δ 8.63 (d, J=7.3 Hz, 1H), 7.16 (d, J=7.3 Hz, 1H), 6.04 (s, 1H), 4.94-4.87 (m, 2H), 3.99 (s, 2H), 3.75 (q, J=6.6 Hz, 1H), 3.42 (s, 3H), 3.35-3.32 (m, 1H), 3.21-3.10 (m, 1H), 2.86-2.76 (m, 1H), 2.45 (s, 3H), 2.32 (d, J=12.9 Hz, 1H), 2.04-1.82 (m, 2H), 1.67-1.47 (m, 2H), 1.38 (d, J=6.7 Hz, 3H), 1.02 (t, J=6.9 Hz, 3H), 0.75 (t, J=7.3 Hz, 3H) ppm. MS: M/e 507 (M+1)⁺.

Compound A47b (the later peak): ¹H NMR (400 MHz, CD₃OD) δ 8.62 (d, J=7.5 Hz, 1H), 7.12 (d, J=7.4 Hz, 1H), 6.04 (s, 1H), 4.96-4.87 (m, 2H), 4.02-3.89 (m, 2H), 3.60-3.44 (m, 1H), 3.42 (s, 3H), 3.37-3.32 (m, 1H), 3.01-2.89 (m, 2H), 2.54-2.42 (m, 4H), 2.12-1.98 (m, 2H), 1.70-1.50 (m, 2H), 1.40 (d, J=6.6 Hz, 3H), 0.94 (t, J=6.8 Hz, 3H), 0.79 (s, 3H) ppm. MS: M/e 507 (M+1)⁺.

### Compound A48: 2-(4-((2S,5R)-2,5-diethyl-4-(1-(3-fluoro-2-methylimidazo[1,2-b]pyridazin-6-yl)ethyl)piperazin-1-yl)-1-methyl-2-oxo-1,2-dihydropyrazolo[1,5-a][1,3,5]triazin-7-yl)acetonitrile

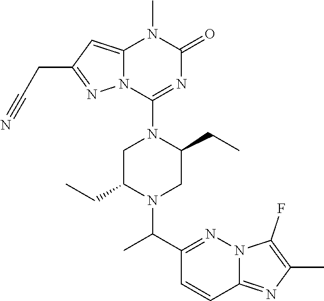 [SMILES: CC[C@H]1CN(C(C)c2ccc3nc(C)c(F)n3n2)[C@H](CC)CN1c1nc(=O)n(C)c2cc(CC#N)nn12]

### Step A: 6-chloro-3-fluoro-2-methylimidazo[1,2-b]pyridazine

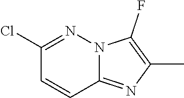

To a solution of 6-chloro-2-methylimidazo[1,2-b]pyridazine (501 mg, 3 mmol) in CH₃CN (10 mL) was added Selectfluor (1.06 g, 3 mmol) at 0° C. The mixture was stirred at 0° C. for 2 hours. The reaction mixture was diluted with H₂O (60 mL), basified with saturated NaHCO₃ solution to pH 8, extracted with EA (80 mL×2), washed with brine, dried over Na₂SO₄, filtered, and concentrated. The residue was purified by flash column chromatography to give the titled compound (50 mg, 9%). MS: M/e 186 (M+1)-.

### Step B: 1-(3-fluoro-2-methylimidazo[1,2-b]pyridazin-6-yl)ethan-1-one

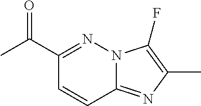

To a solution of 6-chloro-3-fluoro-2-methylimidazo[1,2-b]pyridazine (50 mg, 0.27 mmol) in DMF (5 mL) was added tributyl(1-ethoxyvinyl)stannane (117 mg, 0.32 mmol) and Pd(PPh₂)₂Cl₂ (20 mg, 0.027 mmol). The reaction mixture was protected by N₂ atmosphere and stirred at 100° C. overnight. The mixture was cooled down to rt, 4M HCl in EA (1 mL) was added and the resulting mixture was stirred for 2 hours. The reaction was quenched with saturated NaHCO₃ solution to pH 8, diluted with H₂O, extracted with EA (70 mL×2), washed with brine, dried over Na₂SO₄, filtered, and concentrated to dryness. The residue was purified by flash column chromatography to give the titled compound (20 mg, 38%). MS: M/e 194 (M+1)⁺.

### Step C: 1-(3-fluoro-2-methylimidazo[1,2-b]pyridazin-6-yl)ethan-1-ol

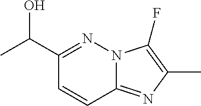

To a solution of 1-(3-fluoro-2-methylimidazo[1,2-b]pyridazin-6-yl)ethan-1-one (20 mg, 0.1 mmol) in MeOH (4 mL) was added NaBH₄ (4 mg, 0.1 mmol). The resulting mixture was stirred at ice-bath for 30 mins. The reaction mixture was quenched with H₂O (15 mL) and extracted with EA (30 mL×3), washed with brine, dried over Na₂SO₄, filtered, and concentrated to dryness to give the titled compound (20 mg, 100%). MS: M/e 196 (M+1)⁺.

### Step D: 2-(4-((2S,5R)-2,5-diethyl-4-(1-(3-fluoro-2-methylimidazo[1,2-b]pyridazin-6-yl)ethyl)piperazin-1-yl)-1-methyl-2-oxo-1,2-dihydropyrazolo[1,5-a][1,3,5]triazin-7-yl)acetonitrile

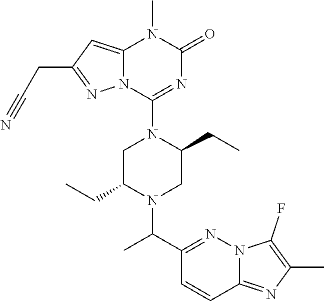 [SMILES: CC[C@H]1CN(C(C)c2ccc3nc(C)c(F)n3n2)[C@H](CC)CN1c1nc(=O)n(C)c2cc(CC#N)nn12]

A solution of 2-(4-((2S,5R)-2,5-diethylpiperazin-1-yl)-1-methyl-2-oxo-1,2-dihydropyrazolo[1,5-a][1,3,5]triazin-7-yl)acetonitrile (92.8 mg, 0.28 mmol), 1-(3-fluoro-2-methylimidazo[1,2-b]pyridazin-6-yl)ethan-1-ol (55 mg, 0.28 mmol), (cyanomethyl)trimethylphosphonium iodide (206 mg, 0.85 mmol) and DIPEA (364 mg, 2.82 mmol) in CH₃CN (2 mL) was stirred at 100° C. for 2 days. The reaction was diluted with EtOAc (10 mL) and washed with brine (10 mL). The organic layer was concentrated under reduced pressure. The resulting residue was purified by flash column chromatography with 0-5% MeOH in DCM to give the titled compound, which was further separated into Compound A48a (2.5 mg, 1.8%) and A48b (3.4 mg, 2.4%) by prep-HPLC (Method A).

Compound A48a (the earlier peak): ¹H NMR (400 MHz, CD₃OD) δ 7.84 (d, J=9.8 Hz, 1H), 7.45 (d, J=9.5 Hz, 1H), 6.45-5.73 (m, 2H), 5.10-4.87 (m, 2H), 3.99 (s, 1H), 3.87 (q, J=6.6 Hz, 1H), 3.42 (s, 3H), 3.37-3.33 (m, 1H), 3.25-3.11 (m, 1H), 2.79 (d, J=11.7 Hz, 1H), 2.41 (s, 3H), 2.29 (d, J=12.3 Hz, 1H), 2.05-1.77 (m, 2H), 1.66-1.50 (m, 2H), 1.42 (d, J=6.7 Hz, 3H), 1.03 (t, J=6.8 Hz, 3H), 0.70 (t, J=7.2 Hz, 3H) ppm. MS: M/e 507 (M+1)⁺.

Compound A48b (the later peak): ¹H NMR (400 MHz, CD₃OD) δ 7.85 (d, J=9.5 Hz, 1H), 7.40 (d, J=9.4 Hz, 1H), 6.35-5.75 (m, 2H), 5.16-4.88 (m, 1H), 4.06 (q, J=6.6 Hz, 1H), 3.98 (s, 1H), 3.42 (s, 3H), 3.36-3.33 (m, 2H), 3.00-2.90 (m, 2H), 2.53-2.37 (m, 4H), 2.08-1.95 (m, 2H), 1.70-1.54 (m, 2H), 1.44 (d, J=6.5 Hz, 3H), 0.92 (t, J=6.8 Hz, 3H), 0.82-0.77 (m, 3H) ppm. MS: M/e 507 (M+1)⁺.

### Compound A49: 2-(4-((2S,5R)-4-(1-(6-cyclopropylpyridin-3-yl)ethyl)-2,5-diethylpiperazin-1-yl)-1-methyl-2-oxo-1,2-dihydropyrazolo[1,5-a][1,3,5]triazin-7-yl)acetonitrile

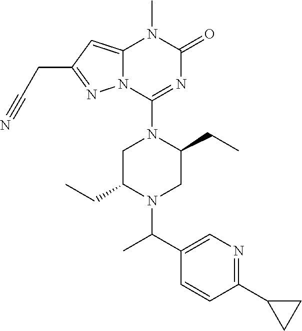 [SMILES: CC[C@H]1CN(C(C)c2ccc(C3CC3)nc2)[C@H](CC)CN1c1nc(=O)n(C)c2cc(CC#N)nn12]

### Step A: 1-(6-cyclopropylpyridin-3-yl)ethan-1-one

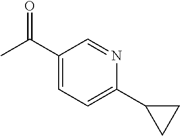

A mixture of 1-(6-bromopyridin-3-yl)ethan-1-one (1 g, 5 mmol), cyclopropylboronic acid (473 mg, 5.5 mmol), tricyclohexylphosphane (140 mg, 0.5 mmol), potassium phosphate (1.6 g, 7.5 mmol) and Pd(OAc)₂ (112 mg, 0.5 mmol) in toluene (15 mL) and water (1.5 ml) was stirred at 100° C. under N₂ overnight. The mixture was treated with water (50 ml), extracted with EtOAc (20 ml×3), washed with brine (50 mL), dried and concentrated to dryness. The resulting residue was purified by flash column chromatography (0-50% EtOAc in PE in 25 minutes) to give the titled compound (620 mg, 77%). MS: M/e 162 (M+1)⁺.

### Step B: 1-(6-cyclopropylpyridin-3-yl)ethan-1-ol

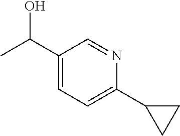

To a solution of 1-(6-cyclopropylpyridin-3-yl)ethan-1-one (620 mg, 3.8 mmol) in MeOH (10 mL) was added NaBH₄ (117 mg, 3.1 mmol) at 0° C. and the mixture was stirred at 0° C. for 30 minutes. The resulting mixture was treated with water (100 ml), extracted with DCM (20 mL×2). The combined organic layers were dried over Na₂SO₄ and concentrated to dryness. The resulting residue was purified by flash column chromatography (0-100% EtOAc in PE in 25 minutes) to give the titled compound (420 mg, 68%). MS: M/e 164 (M+1)⁺.

### Step C: 2-(4-((2S,5R)-4-(1-(6-cyclopropylpyridin-3-yl)ethyl)-2,5-diethylpiperazin-1-yl)-1-methyl-2-oxo-1,2-dihydropyrazolo[1,5-a][1,3,5]triazin-7-yl)acetonitrile

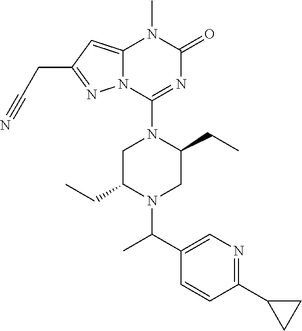 [SMILES: CC[C@H]1CN(C(C)c2ccc(C3CC3)nc2)[C@H](CC)CN1c1nc(=O)n(C)c2cc(CC#N)nn12]

A solution of 2-(4-((2S,5R)-2,5-diethylpiperazin-1-yl)-1-methyl-2-oxo-1,2-dihydropyrazolo[1,5-a][1,3,5]triazin-7-yl)acetonitrile (50 mg, 0.15 mmol), 1-(6-cyclopropylpyridin-3-yl)ethan-1-ol (49.5 mg, 0.30 mmol), (cyanomethyl)trimethylphosphonium iodide (111 mg, 0.46 mmol) and DIPEA (196 mg, 1.52 mmol) in CH₃CN (2 ml) was stirred at 100° C. for 2 days. The reaction was diluted with EtOAc (10 ml) and washed with brine (10 ml). The organic layer was concentrated under reduced pressure. The resulting residue was purified by flash column chromatography with 0-5% MeOH in DCM to give the titled compound, which was further separated into Compound A49a (1.73 mg, 2.4%) and Compound A49b (2.45 mg, 3.4%) by prep-HPLC (Method A).

Compound A49a (the earlier peak): ¹H NMR (400 MHz, CD₃OD) δ 8.29 (s, 1H), 7.70 (d, J=8.4 Hz, 1H), 7.20 (d, J=8.0 Hz, 1H), 6.19-5.89 (m, 2H), 5.00-4.88 (m, 2H), 4.02-3.94 (m, 1H), 3.77 (q, J=6.4 Hz, 1H), 3.41 (s, 3H), 3.35-3.33 (m, 1H), 2.99 (d, J=10.9 Hz, 1H), 2.91-2.80 (m, 1H), 2.44-2.31 (m, 1H), 2.15-1.97 (m, 3H), 1.58-1.45 (m, 2H), 1.34 (d, J=6.5 Hz, 3H), 1.05-1.01 (m, 2H), 0.98-0.93 (m, 5H), 0.72 (s, 3H) ppm. MS: M/e 475 (M+1)⁺.

Compound A49b (the later peak): ¹H NMR (400 MHz, CD₃OD) δ 8.32 (s, 1H), 7.71 (d, J=8.2 Hz, 1H), 7.18 (d, J=7.9 Hz, 1H), 6.35-5.73 (m, 2H), 5.00-4.88 (m, 1H), 4.02-3.95 (m, 1H), 3.61 (q, J=6.5 Hz, 1H), 3.42 (s, 3H), 3.36-3.32 (m, 2H), 3.22-3.12 (m, 1H), 2.71-2.59 (m, 1H), 2.30 (d, J=12.8 Hz, 1H), 2.09-2.04 (m, 1H), 1.93-1.83 (m, 2H), 1.59-1.53 (m, 2H), 1.31 (d, J=6.4 Hz, 3H), 1.05-0.99 (m, 5H), 0.94-0.90 (m, 2H), 0.68 (t, J=6.9 Hz, 3H) ppm. MS: M/e 475 (M+1)⁺.

### Compound A50: 2-(4-((2S,5R)-2,5-diethyl-4-(1-(3-fluoropyrazolo[1,5-a]pyrimidin-5-yl)ethyl)piperazin-1-yl)-1-methyl-2-oxo-1,2-dihydropyrazolo[1,5-a][1,3,5]triazin-7-yl)acetonitrile

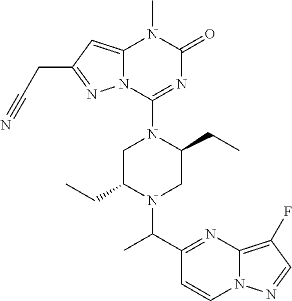 [SMILES: CC[C@H]1CN(C(C)c2ccn3ncc(F)c3n2)[C@H](CC)CN1c1nc(=O)n(C)c2cc(CC#N)nn12]

### Step A: 3-fluoropyrazolo[1,5-a]pyrimidin-5(4H)-one

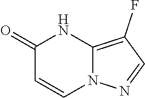 [SMILES: O=c1ccn2ncc(F)c2[nH]1]

A mixture of 4-fluoro-1H-pyrazol-5-amine (0.5 g, 5.1 mmol), ethyl (E)-3-ethoxyacrylate (1.4 g, 10 mmol) and Cs₂CO₃ (3.2 g, 10 mmol) in DMF (20 mL) was stirred at 110° C. under N₂ for 16 hours. The solution was collected by filtration, then added acetic acid (5 mL). The organic layer was concentrated to dryness. The resulting residue was purified by flash column chromatography to give the titled compound (0.7 g, 93%). MS: M/e 154 (M+1)⁺

### Step B: 5-chloro-3-fluoropyrazolo[1,5-a]pyrimidine

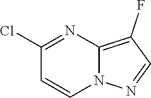 [SMILES: Fc1cnn2ccc(Cl)nc12]

To a solution of 3-fluoropyrazolo[1,5-a]pyrimidin-5(4H)-one (0.7 g, 4.5 mmol) in CH₃CN (10 mL) were added POCl₃ (1.4 g, 9.1 mmol). The mixture was stirred at 90° C. for 3 hours. The reaction was concentrated and poured into saturated NaHCO₃ solution, extracted with DCM. The combined organic layers were washed with brine, dried over Na₂SO₄ and concentrated to dryness. The resulting residue was purified by flash column chromatography to give the titled compound (0.3 g, 38%). MS: M/e 172 (M+1)⁺.

### Step C: 1-(3-fluoropyrazolo[1,5-a]pyrimidin-5-yl)ethan-1-one

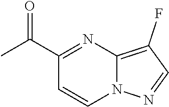

A mixture of 5-chloro-3-fluoropyrazolo[1,5-a]pyrimidine (0.3 g, 1.75 mmol), tributyl(1-ethoxyvinyl)stannane (0.76 g, 2.1 mmol) and Pd(PPh₃)₂Cl₂ (123 mg, 0.17 mmol) in DMF (5 mL) was stirred at 100° C. under N₂ for 16 hours. The solution was diluted with EA and washed with water. The organic layer was concentrated to dryness. The resulting residue was purified by flash column chromatography to give the titled compound (0.21 g, 66%). MS: M/e 180 (M+1)⁺

### Step D: 1-(3-fluoropyrazolo[1,5-a]pyrimidin-5-yl)ethan-1-ol

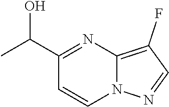

To a solution of 1-(3-fluoropyrazolo[1,5-a]pyrimidin-5-yl)ethan-1-one (0.21 g, 1.17 mmol) in MeOH (5 mL) was added NaBH₄ (27 mg, 0.7 mmol) at room temperature and the resulting mixture was stirred at room temperature for 5 mins. The reaction mixture was diluted with DCM and washed with water, dried over Na₂SO₄ and concentrated to give the titled compound (150 mg). MS: M/e 182 (M+1)⁺.

### Step E: 2-(4-((2S,5R)-2,5-diethyl-4-(1-(3-fluoropyrazolo[1,5-a]pyrimidin-5-yl)ethyl)piperazin-1-yl)-1-methyl-2-oxo-1,2-dihydropyrazolo[1,5-a][1,3,5]triazin-7-yl)acetonitrile

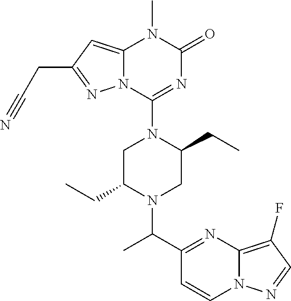 [SMILES: CC[C@H]1CN(C(C)c2ccn3ncc(F)c3n2)[C@H](CC)CN1c1nc(=O)n(C)c2cc(CC#N)nn12]

A solution of 2-(4-((2S,5R)-2,5-diethylpiperazin-1-yl)-1-methyl-2-oxo-1,2-dihydropyrazolo[1,5-a][1,3,5]triazin-7-yl)acetonitrile (50 mg, 0.15 mmol), 1-(3-fluoropyrazolo[1,5-a]pyrimidin-5-yl)ethan-1-ol (55 mg, 0.30 mmol), (cyanomethyl)trimethylphosphonium iodide (111 mg, 0.46 mmol) and DIPEA (196 mg, 1.52 mmol) in CH₃CN (2 mL) was stirred at 100° C. for 2 days. The reaction was diluted with EtOAc (10 mL) and washed with brine (10 mL). The organic layer was concentrated under reduced pressure. The resulting residue was purified by flash column chromatography with 0-5% MeOH in DCM and then further purification by prep-HPLC (Method A) to give the titled compound (4.5 mg, 6.0%). ¹H NMR (400 MHz, CD₃OD) δ 8.76-8.69 (m, 1H), 8.09 (d, J=3.1 Hz, 1H), 7.22 (dd, J=19.7, 7.3 Hz, 1H), 6.35-5.75 (m, 2H), 5.10-4.88 (m, 1H), 4.84-4.65 (m, 1H), 3.98 (q, J=6.6 Hz, 1H), 3.79 (q, J=6.6 Hz, 0.5H), 3.42 (s, 3H), 3.36-3.32 (m, 1.5H), 3.23-3.12 (m, 0.5H), 3.01-2.90 (m, 1H), 2.88-2.77 (m, 0.5H), 2.55-2.43 (m, 0.5H), 2.31 (d, J=13.2 Hz, 0.5H), 2.13-1.83 (m, 2H), 1.69-1.51 (m, 2H), 1.43-1.37 (m, 3H), 1.06-1.00 (m, 1.5H), 0.96-0.92 (m, 1.5H), 0.78-0.71 (m, 3H) ppm. MS: M/e 493 (M+1)⁺.

### Compound A51: 2-(4-((2S,5R)-2,5-diethyl-4-(1-(2-ethylimidazo[1,2-b]pyridazin-6-yl)ethyl)piperazin-1-yl)-1-methyl-2-oxo-1,2-dihydropyrazolo[1,5-a][1,3,5]triazin-7-yl)acetonitrile

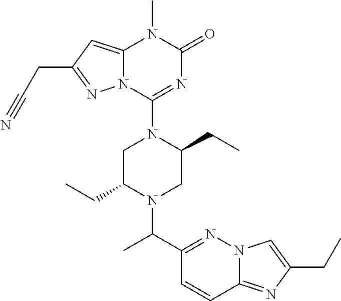 [SMILES: CCc1cn2nc(C(C)N3C[C@H](CC)N(c4nc(=O)n(C)c5cc(CC#N)nn45)C[C@H]3CC)ccc2n1]

### Step A: 6-chloro-2-ethylimidazo[1,2-b]pyridazine

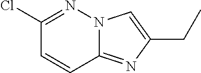

To a solution of 6-chloropyridazin-3-amine (0.65 g, 5 mmol) in EtOH (10 mL) was added 1-bromobutan-2-one (0.83 g, 5.5 mmol). The mixture was stirred at 85° C. for 16 hours. The reaction mixture was concentrated to dryness under reduced pressure. The residue was diluted with H₂O (50 ml), basified with saturated NaHCO₃ solution to pH=8, extracted with EtOAc (70 mL×2), washed with brine, dried over Na₂SO₄, filtered, and concentrated. The residue was purified by flash column chromatography to give the titled compound (605 mg, 66%). MS: M/e 182 (M+1)⁺.

### Step B: 1-(2-ethylimidazo[1,2-b]pyridazin-6-yl)ethan-1-one

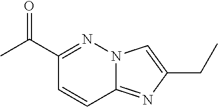

To a solution of 6-chloro-2-ethylimidazo[1,2-b]pyridazine (605 mg, 3.34 mmol) in DMF (15 mL) was added tributyl(1-ethoxyvinyl)stannane (1.33 g, 3.67 mmol) and Pd(PPh₂)₂Cl₂ (233 mg, 0.33 mmol). The reaction mixture was protected by N₂ atmosphere and stirred at 100° C. overnight. The mixture was cooled down to RT, 4M HCl in EA (10 mL) was added and the resulting mixture was stirred for 5 hours. The reaction was diluted with water (80 mL), extracted with EtOAc (70 mL×2). The aqueous layer was basified with saturated NaHCO₃ solution to pH=8, extracted with EtOAc (80 mL×2), washed with brine, dried over Na₂SO₄, filtered, and concentrated to dryness. The residue was purified by flash column chromatography to give the titled compound (560 mg, 88%). MS: M/e 190 (M+1)⁺.

### Step C: 1-(2-ethylimidazo[1,2-b]pyridazin-6-yl)ethan-1-ol

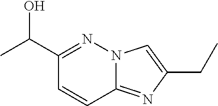

To a solution of 1-(2-ethylimidazo[1,2-b]pyridazin-6-yl)ethan-1-one (560 mg, 2.97 mmol) in MeOH (10 mL) was added NaBH₄ (112 mg, 2.97 mmol). The resulting mixture was stirred at ice-bath for 30 mins. The reaction mixture was quenched with H₂O (60 mL) and extracted with EtOAc (70 mL×2), washed with brine, dried over Na₂SO₄, filtered, and concentrated to dryness to give the desired product (500 mg, 87%). MS: M/e 192 (M+1)⁺.

### Step D: 2-(4-((2S,5R)-2,5-diethyl-4-(1-(2-ethylimidazo[1,2-b]pyridazin-6-yl)ethyl)piperazin-1-yl)-1-methyl-2-oxo-1,2-dihydropyrazolo[1,5-a][1,3,5]triazin-7-yl)acetonitrile

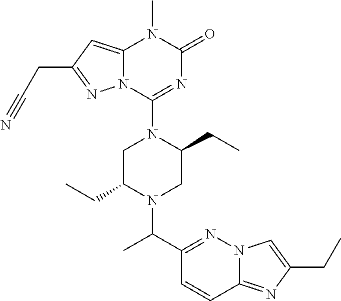 [SMILES: CCc1cn2nc(C(C)N3C[C@H](CC)N(c4nc(=O)n(C)c5cc(CC#N)nn45)C[C@H]3CC)ccc2n1]

A solution of 2-(4-((2S,5R)-2,5-diethylpiperazin-1-yl)-1-methyl-2-oxo-1,2-dihydropyrazolo[1,5-a][1,3,5]triazin-7-yl)acetonitrile (66 mg, 0.20 mmol), 1-(2-ethylimidazo[1,2-b]pyridazin-6-yl)ethan-1-ol (76.6 mg, 0.40 mmol), (cyanomethyl)trimethylphosphonium iodide (146.2 mg, 0.60 mmol) and DIPEA (258.8 mg, 2.01 mmol) in CH₃CN (2 mL) was stirred at 100° C. for 2 days. The reaction was diluted with EtOAc (10 mL) and washed with brine (10 mL). The organic layer was concentrated under reduced pressure. The resulting residue was purified by flash column chromatography with 0-5% MeOH in DCM to give the titled compound, which was further separated into Compound A51a (12.6 mg, 12.5%) and Compound A51b (15.2 mg, 15.1%) by prep-HPLC (Method A).

Compound A51a (the earlier peak): ¹H NMR (400 MHz, CD₃OD) δ 7.87 (d, J=9.4 Hz, 1H), 7.83 (s, 1H), 7.45 (d, J=9.4 Hz, 1H), 6.40-5.70 (m, 2H), 5.11-4.88 (m, 1H), 4.85-4.62 (m, 1H), 4.05-3.93 (m, 1H), 3.81 (q, J=6.6 Hz, 1H), 3.42 (s, 3H), 3.37-3.32 (m, 1H), 3.22-3.10 (m, 1H), 2.87-2.73 (m, 3H), 2.29 (d, J=12.1 Hz, 1H), 2.00-1.78 (m, 2H), 1.70-1.48 (m, 2H), 1.41 (d, J=6.5 Hz, 3H), 1.34 (t, J=7.5 Hz, 3H), 1.02 (t, J=7.1 Hz, 3H), 0.69 (t, J=7.1 Hz, 3H) ppm. MS: M/e 503 (M+1)⁺.

Compound A51b (the later peak): ¹H NMR (400 MHz, CD₃OD) δ 7.88 (d, J=9.5 Hz, 1H), 7.85 (s, 1H), 7.40 (d, J=9.4 Hz, 1H), 6.22-5.85 (m, 2H), 5.13-4.89 (m, 1H), 4.06-3.92 (m, 2H), 3.46-3.34 (m, 5H), 3.00-2.88 (m, 2H), 2.82 (q, J=7.4 Hz, 2H), 2.52-2.38 (m, 1H), 2.10-1.94 (m, 2H), 1.70-1.53 (m, 2H), 1.43 (d, J=6.4 Hz, 3H), 1.34 (t, J=7.5 Hz, 3H), 0.92 (t, J=7.0 Hz, 3H), 0.77 (s, 3H) ppm. MS: M/e 503 (M+1)⁺.

### Compound A52: 2-(4-((2S,5R)-2,5-diethyl-4-(1-(2-methylthiazolo[5,4-b]pyridin-5-yl)ethyl)piperazin-1-yl)-1-methyl-2-oxo-1,2-dihydropyrazolo[1,5-a][1,3,5]triazin-7-yl)acetonitrile

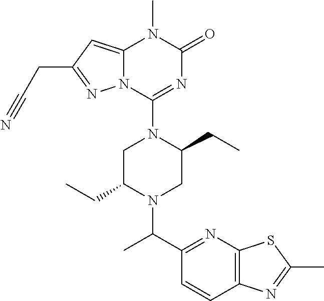 [SMILES: CC[C@H]1CN(C(C)c2ccc3nc(C)sc3n2)[C@H](CC)CN1c1nc(=O)n(C)c2cc(CC#N)nn12]

A solution of 2-(4-((2S,5R)-2,5-diethylpiperazin-1-yl)-1-methyl-2-oxo-1,2-dihydropyrazolo[1,5-a][1,3,5]triazin-7-yl)acetonitrile (66 mg, 0.20 mmol), 1-(2-methylthiazolo[5,4-b]pyridin-5-yl)ethan-1-ol (77.8 mg, 0.40 mmol), (cyanomethyl)trimethylphosphonium iodide (146.2 mg, 0.60 mmol) and DIPEA (258.8 mg, 2.01 mmol) in CH₃CN (2 mL) was stirred at 100° C. for 2 days. The reaction was diluted with EtOAc (10 mL) and washed with brine (10 mL). The organic layer was concentrated under reduced pressure. The resulting residue was purified by flash column chromatography with 0-5% MeOH in DCM to give the titled compound, which was further separated into Compound A52a (10.2 mg, 10%) and Compound A52b (17.5 mg, 17%) by prep-HPLC (Method A).

Compound A52a (the earlier peak): ¹H NMR (400 MHz, CD₃OD) δ 8.22 (d, J=8.3 Hz, 1H), 7.75 (d, J=8.5 Hz, 1H), 6.40-5.70 (m, 2H), 5.14-4.88 (m, 1H), 4.86-4.62 (m, 1H), 3.98 (s, 1H), 3.86 (q, J=6.5 Hz, 1H), 3.42 (s, 3H), 3.38-3.32 (m, 1H), 3.26-3.11 (m, 1H), 2.85 (s, 3H), 2.74 (d, J=6.5 Hz, 1H), 2.25 (d, J=12.5 Hz, 1H), 2.00-1.85 (m, 2H), 1.64-1.50 (m, 2H), 1.39 (d, J=6.4 Hz, 3H), 1.03 (t, J=6.8 Hz, 3H), 0.67 (t, J=6.8 Hz, 3H) ppm. MS: M/e 506 (M+1)⁺.

Compound A52b (the later peak): ¹H NMR (400 MHz, CD₃OD) δ 8.23 (d, J=8.2 Hz, 1H), 7.70 (d, J=8.4 Hz, 1H), 6.20-5.80 (m, 2H), 5.07-4.88 (m, 1H), 4.05 (q, J=6.7 Hz, 1H), 3.98 (s, 1H), 3.45-3.32 (m, 5H), 3.06-2.89 (m, 2H), 2.86 (s, 3H), 2.44-2.33 (m, 1H), 2.14-1.98 (m, 2H), 1.69-1.46 (m, 2H), 1.42 (d, J=6.4 Hz, 3H), 0.96 (t, J=7.2 Hz, 3H), 0.70 (t, J=7.2 Hz, 3H) ppm. MS: M/e 506 (M+1)⁺.

### Compound A53: 2-(4-((2S,5R)-2,5-diethyl-4-(1-(2-methylbenzo[d]thiazol-6-yl)ethyl)piperazin-1-yl)-1-methyl-2-oxo-1,2-dihydropyrazolo[1,5-a][1,3,5]triazin-7-yl)acetonitrile

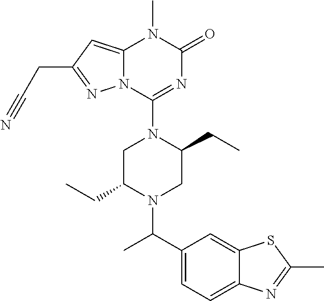 [SMILES: CC[C@H]1CN(C(C)c2ccc3nc(C)sc3c2)[C@H](CC)CN1c1nc(=O)n(C)c2cc(CC#N)nn12]

A solution of 2-(4-((2S,5R)-2,5-diethylpiperazin-1-yl)-1-methyl-2-oxo-1,2-dihydropyrazolo[1,5-a][1,3,5]triazin-7-yl)acetonitrile (66 mg, 0.20 mmol), 1-(2-methylbenzo[d]thiazol-6-yl)ethan-1-ol (77.4 mg, 0.40 mmol), (cyanomethyl)trimethylphosphonium iodide (146.2 mg, 0.60 mmol) and DIPEA (258.8 mg, 2.01 mmol) in CH₃CN (2 mL) was stirred at 100° C. for 2 days. The reaction was diluted with EtOAc (10 mL) and washed with brine (10 mL). The organic layer was concentrated under reduced pressure. The resulting residue was purified by flash column chromatography with 0-5% MeOH in DCM to give the titled compound, which was further separated into Compound A53a (8.1 mg, 8.0%) and Compound A53b (10.3 mg, 10.2%) by prep-HPLC (Method A).

Compound A53a (the earlier peak): ¹H NMR (400 MHz, CD₃OD) δ 7.91 (s, 1H), 7.85 (d, J=8.3 Hz, 1H), 7.52 (d, J=8.4 Hz, 1H), 6.16-5.90 (m, 2H), 5.05-4.88 (m, 1H), 4.85-4.61 (m, 1H), 4.04-3.92 (m, 1H), 3.87 (q, J=6.0 Hz, 1H), 3.50-3.32 (m, 4H), 3.05 (d, J=12.2 Hz, 1H), 2.93-2.78 (m, 4H), 2.50-2.34 (m, 1H), 2.18-1.99 (m, 2H), 1.62-1.41 (m, 2H), 1.38 (d, J=6.1 Hz, 3H), 0.98 (t, J=7.1 Hz, 3H), 0.66 (s, 3H) ppm. MS: M/e 505 (M+1)⁺.

Compound A53b (the later peak): ¹H NMR (400 MHz, CD₃OD) δ 7.94 (s, 1H), 7.83 (d, J=8.4 Hz, 1H), 7.54 (d, J=8.3 Hz, 1H), 6.40-5.67 (m, 2H), 5.05-4.88 (m, 1H), 4.85-4.62 (m, 1H), 4.02-3.90 (m, 1H), 3.72 (q, J=6.6 Hz, 1H), 3.41 (s, 3H), 3.25-3.15 (m, 1H), 2.82 (s, 3H), 2.70-2.58 (m, 1H), 2.36 (d, J=12.0 Hz, 1H), 1.98-1.83 (m, 3H), 1.64-1.53 (m, 2H), 1.35 (d, J=6.4 Hz, 3H), 1.03 (s, 3H), 0.62 (t, J=7.1 Hz, 3H) ppm. MS: M/e 505 (M+1)⁺.

### Compound A54: 2-(4-((2S,5R)-4-(1-(2-cyclopropylpyrazolo[1,5-a]pyrimidin-5-yl)ethyl)-2,5-diethylpiperazin-1-yl)-1-methyl-2-oxo-1,2-dihydropyrazolo[1,5-a][1,3,5]triazin-7-yl)acetonitrile

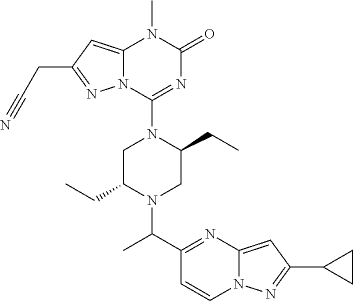 [SMILES: CC[C@H]1CN(C(C)c2ccn3nc(C4CC4)cc3n2)[C@H](CC)CN1c1nc(=O)n(C)c2cc(CC#N)nn12]

### Step A: 2-cyclopropylpyrazolo[1,5-a]pyrimidin-5(4H)-one

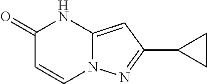 [SMILES: O=c1ccn2nc(C3CC3)cc2[nH]1]

A mixture of 3-cyclopropyl-1H-pyrazol-5-amine (0.5 g, 4.07 mmol), ethyl (E)-3-ethoxyacrylate (1.17 g, 8.13 mmol) and Cs₂CO₃ (2.65 g, 8.13 mmol) in DMF (15 mL) was stirred at 110° C. under N₂ for 16 hours. The solution was collected by filtration, then added acetic acid (5 mL). The organic layer was concentrated to dryness. The resulting residue was purified by flash column chromatography to give the titled compound (0.38 g, 53%). MS: M/e 176 (M+1)⁺

### Step B: 5-chloro-2-cyclopropylpyrazolo[1,5-a]pyrimidine

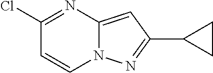 [SMILES: Clc1ccn2nc(C3CC3)cc2n1]

To a solution of 2-cyclopropylpyrazolo[1,5-a]pyrimidin-5(4H)-one (0.38 g, 2.1 mmol) in CH₃CN (5 mL) were added POCl₃ (0.66 g, 4.3 mmol). The mixture was stirred at 100° C. for 3 hours. The reaction was concentrated and poured into saturated NaHCO₃ solution, extracted with DCM. The combined organic layers were washed with brine, dried over Na₂SO₄ and the resulting residue was purified by flash column chromatography to give the titled compound (0.36 g, 85%). MS: M/e 194 (M+1)⁺.

### Step C: 1-(2-cyclopropylpyrazolo[1,5-a]pyrimidin-5-yl)ethan-1-one

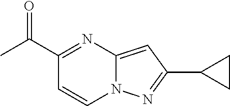

A mixture of 5-chloro-2-cyclopropylpyrazolo[1,5-a]pyrimidine (0.35 g, 1.8 mmol), tributyl(1-ethoxyvinyl)stannane (0.78 g, 2.1 mmol) and Pd(PPh₃)₂Cl₂ (127 mg, 0.18 mmol) in DMF (5 mL) was stirred at 100° C. under N₂ for 16 hours. The solution was diluted with ethyl acetate and washed with water. The organic layer was purified by flash column chromatography to give the 2-cyclopropyl-5-(1-ethoxyvinyl)pyrazolo [1,5-a]pyrimidine, then diluted with DCM and added with HCl (5 mL, 4 M in dioxane) in drops and the mixture was stirred at RT for 2 hours. The reaction was concentrated and adjusted to pH=8˜9 with saturated NaHCO₃ solution, extracted with ethyl acetate. The combined organic layers were washed with brine, dried over Na₂SO₄ and the resulting residue was purified by flash column chromatography to give the titled compound (0.09 g, 24%). MS: M/e 202 (M+1)⁺

### Step D: 2-(4-((2S,5R)-4-(1-(2-cyclopropylpyrazolo[1,5-a]pyrimidin-5-yl)ethyl)-2,5-diethylpiperazin-1-yl)-1-methyl-2-oxo-1,2-dihydropyrazolo[1,5-a][1,3,5]triazin-7-yl)acetonitrile

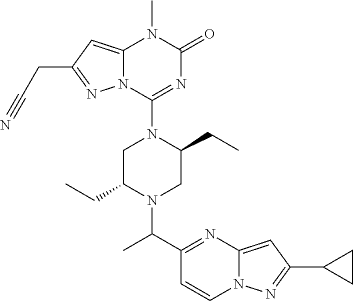 [SMILES: CC[C@H]1CN(C(C)c2ccn3nc(C4CC4)cc3n2)[C@H](CC)CN1c1nc(=O)n(C)c2cc(CC#N)nn12]

A solution of 2-(4-((2S,5R)-2,5-diethylpiperazin-1-yl)-1-methyl-2-oxo-1,2-dihydropyrazolo[1,5-a][1,3,5]triazin-7-yl)acetonitrile (66 mg, 0.20 mmol), 1-(2-cyclopropylpyrazolo[1,5-a]pyrimidin-5-yl)ethan-1-ol (81.4 mg, 0.40 mmol), (cyanomethyl)trimethylphosphonium iodide (146.2 mg, 0.60 mmol) and DIPEA (258.8 mg, 2.01 mmol) in CH₃CN (2 mL) was stirred at 100° C. overnight. The reaction was diluted with EtOAc (10 mL) and washed with brine (10 mL). The organic layer was concentrated under reduced pressure. The resulting residue was purified by flash column chromatography with 0-5% MeOH in DCM to give the titled compound, which was further separated into Compound A54a (11.8 mg, 11.4%) and Compound A54b (12.8 mg, 12.4%) by prep-HPLC (Method A).

Compound A54a (the earlier peak): ¹H NMR (400 MHz, CD₃OD) δ 8.69 (d, J=7.2 Hz, 1H), 7.16 (d, J=7.2 Hz, 1H), 6.40-5.80 (m, 3H), 5.10-4.89 (m, 1H), 4.85-4.65 (m, 1H), 3.99 (s, 1H), 3.70 (q, J=6.1 Hz, 1H), 3.42 (s, 3H), 3.37-3.32 (m, 1H), 3.21-3.09 (m, 1H), 2.85-2.73 (m, 1H), 2.29 (d, J=12.2 Hz, 1H), 2.14-2.05 (m, 1H), 2.04-1.81 (m, 2H), 1.68-1.46 (m, 2H), 1.37 (d, J=6.5 Hz, 3H), 1.09-0.99 (m, 5H), 0.92-0.88 (m, 2H), 0.74 (t, J=7.0 Hz, 3H) ppm. MS: M/e 515 (M+1)⁺.

Compound A54b (the later peak): ¹H NMR (400 MHz, CD₃OD) δ 8.69 (d, J=7.1 Hz, 1H), 7.11 (d, J=7.2 Hz, 1H), 6.29 (s, 1H), 6.24-5.90 (m, 2H), 5.20-4.89 (m, 1H), 3.98 (s, 1H), 3.90 (q, J=6.2 Hz, 1H), 3.60-3.35 (m, 5H), 3.05-2.85 (m, 2H), 2.55-2.38 (m, 1H), 2.20-1.95 (m, 3H), 1.68-1.46 (m, 2H), 1.38 (d, J=6.4 Hz, 3H), 1.07 (d, J=7.8 Hz, 2H), 0.99-0.87 (m, 5H), 0.76 (s, 3H) ppm. MS: M/e 515 (M+1)⁺.

### Compound A55: 2-(4-((2S,5R)-2,5-diethyl-4-(1-(2-methylpyrazolo[1,5-a]pyridin-5-yl)ethyl)piperazin-1-yl)-1-methyl-2-oxo-1,2-dihydropyrazolo[1,5-a][1,3,5]triazin-7-yl)acetonitrile

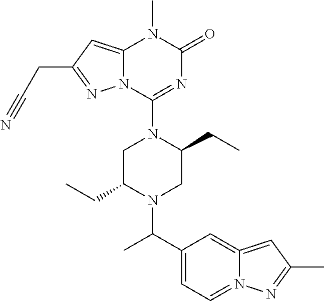

### Step A: 4-bromo-2-(prop-1-yn-1-yl)pyridine

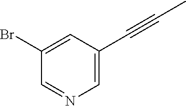

To a stirred solution of 4-bromo-2-iodopyridine (800 mg, 2.8 mmol) in THF (10 mL) was added Pd(PPh₃)₂Cl₂ (196 mg, 0.28 mmol), CuI (106 mg, 0.56 mmol) and Et₃N (566 mg, 5.6 mmol), followed by prop-1-yne (1.0 M, 3.38 mL, 3.38 mmol). After the addition, the reaction mixture was stirred for 2 hours under N₂ at room temperature. The reaction mixture was poured into H₂O (50 mL), then extracted with EA (15 mL×3). The combined organic layers were washed with brine, dried over Na₂SO₄ and concentrated to dryness. The resulting residue was purified by flash column chromatography to give the titled compound (381 mg, 69.4%). MS: M/e 196/198 (M+1)⁺.

### Step B: 5-bromo-2-methylpyrazolo[1,5-a]pyridine

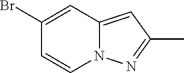

4-bromo-2-(prop-1-yn-1-yl)pyridine (571 mg, 2.91 mmol) was added to a stirred solution of O-(mesitylsulfonyl)hydroxylamine (1.25 g, 2.82 mmol) in CH₂Cl₂ (10 mL). Then the mixture was stirred overnight. The reaction mixture was concentrated to give the residue, which was dissolved in DMF (10 mL) and K₂CO₃ (1.6 g, 11.64 mmol) was added and stirred for 3 hours. The reaction mixture was poured into H₂O (20 mL) and extracted with EA (20 mL×3). The combined organic layers were washed with brine, dried over Na₂SO₄, concentrated to dryness. The resulting residue was purified by flash column chromatography to give the titled compound (140 mg, 23%). MS: M/e 211/213 (M+1)⁺.

### Ste C: 1-(2-methylpyrazolo[1,5-a]pyridin-5-yl)ethan-1-one

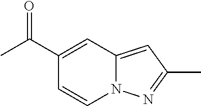

A mixture of 5-bromo-2-methylpyrazolo[1,5-a]pyridine (140 mg, 0.66 mmol), tributyl(1-ethoxyvinyl)stannane (359 mg, 0.99 mmol) and Pd(PPh₃)₂Cl₂ (46.2 mg, 0.066 mmol) in DMF (8 mL) was stirred at 100° C. overnight under N₂. The reaction mixture was treated with EA/HCl (g) (4.0 M, 5 mL) and stirred for an hour, then treated with H₂O (30 mL) and extracted with EA (30 mL×2). The aqueous layer was basified to pH=8-9 with aq.NaHCO₃, then extracted with EA (30 mL×3). The combined organic layers were washed with brine, dried over Na₂SO₄ and concentrated to dryness. The resulting residue was purified by flash column chromatography to give the titled compound (100 mg, 87%). MS: M/e 175 (M+1)⁺.

### Step D: 1-(2-methylpyrazolo[1,5-a]pyridin-5-yl)ethan-1-ol

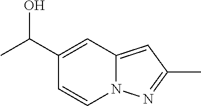

To a stirred solution of 1-(2-methylpyrazolo[1,5-a]pyridin-5-yl)ethan-1-one (100 mg, 0.57 mmol) in MeOH (10 mL) was added NaBH₄ (21.8 mg, 0.57 mmol). After then, the mixture was stirred for 10 min. The reaction mixture was poured into H₂O (10 mL) and most MeOH was removed to give the aqueous layer, then extracted with EA (20 mL×3). The combined organic layers were washed with brine, dried over Na₂SO₄, concentrated to give the titled compound (100 mg, 99%). MS: M/e 177 (M+1)⁺.

### Step E: 2-(4-((2S,5R)-2,5-diethyl-4-(1-(2-methylpyrazolo[1,5-a]pyridin-5-yl)ethyl)piperazin-1-yl)-1-methyl-2-oxo-1,2-dihydropyrazolo[1,5-a][1,3,5]triazin-7-yl)acetonitrile

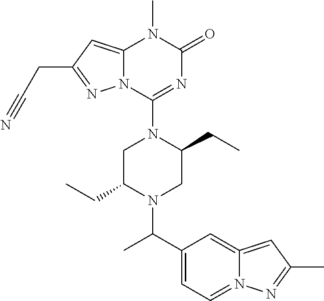

A solution of 2-(4-((2S,5R)-2,5-diethylpiperazin-1-yl)-1-methyl-2-oxo-1,2-dihydropyrazolo[1,5-a][1,3,5]triazin-7-yl)acetonitrile (66 mg, 0.20 mmol), 1-(2-methylpyrazolo[1,5-a]pyridin-5-yl)ethan-1-ol (70.6 mg, 0.40 mmol), (cyanomethyl)trimethylphosphonium iodide (146.2 mg, 0.60 mmol) and DIPEA (258.8 mg, 2.01 mmol) in CH₃CN (2 mL) was stirred at 100° C. for 2 days. The reaction was diluted with EtOAc (10 mL) and washed with brine (10 mL). The organic layer was concentrated under reduced pressure. The resulting residue was purified by flash column chromatography with 0-5% MeOH in DCM and then further purified to give the titled compound (10.3 mg, 10.5%) by prep-HPLC (Method A). ¹H NMR (400 MHz, CD₃OD) δ 8.35 (t, J=7.9 Hz, 1H), 7.43 (d, J=10.9 Hz, 1H), 6.92 (t, J=7.2 Hz, 1H), 6.30 (d, J=7.8 Hz, 1H), 6.20-5.75 (m, 2H), 5.05-4.89 (m, 1H), 4.86-4.65 (m, 1H), 3.78 (q, J=6.1 Hz, 0.5H), 3.63 (q, J=6.5 Hz, 0.5H), 3.42 (s, 3H), 3.37-3.31 (m, 2H), 3.21-3.11 (m, 0.5H), 3.00 (d, J=11.9 Hz, 0.5H), 2.91-2.80 (m, 0.5H), 2.74-2.63 (m, 0.5H), 2.55-2.38 (m, 4H), 2.15-2.00 (m, 1H), 1.99-1.85 (m, 1H), 1.64-1.40 (m, 2H), 1.34 (t, J=7.2 Hz, 3H), 1.05-0.93 (m, 3H), 0.71 (t, J=6.8 Hz, 3H) ppm. MS: M/e 488 (M+1)⁺.

### Compound A56: 2-(4-((2S,5R)-2,5-diethyl-4-(1-(2-fluoropyrazolo[1,5-a]pyrimidin-5-yl)ethyl)piperazin-1-yl)-1-methyl-2-oxo-1,2-dihydropyrazolo[1,5-a][1,3,5]triazin-7-yl)acetonitrile

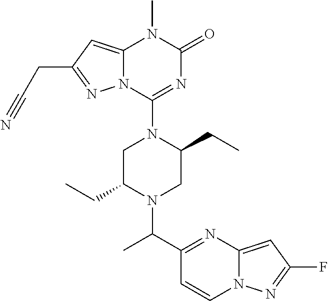 [SMILES: CC[C@H]1CN(C(C)c2ccn3nc(F)cc3n2)[C@H](CC)CN1c1nc(=O)n(C)c2cc(CC#N)nn12]

To a solution of 2-(4-((2S,5R)-2,5-diethylpiperazin-1-yl)-1-methyl-2-oxo-1,2-dihydropyrazolo[1,5-a][1,3,5]triazin-7-yl)acetonitrile (30 mg, 0.09 mmol) in CH₃CN (4 mL) and was added 1-(2-fluoropyrazolo[1,5-a]pyrimidin-5-yl)ethan-1-ol (49 mg, 0.27 mmol), (cyanomethyl)trimethylphosphonium iodide (89 mg, 0.36 mmol) and DIPEA (117 mg, 0.9 mmol). The resulting mixture was stirred at 105° C. overnight. The reaction solvent was removed under reduce pressure. The resulting residue was purified by flash column chromatography to give the titled compound (crude), which was further purified to give the titled compound (1.9 mg, 4%) by prep-HPLC (Method B). ¹H NMR (400 MHz, DMSO-d₆) δ 9.01-8.91 (m, 1H), 7.22 (d, J=7.0 Hz, 1H), 6.45-6.37 (m, 1H), 6.04 (s, 1H), 5.91-5.44 (m, 1H), 4.81-4.27 (m, 1H), 4.12 (s, 2H), 3.89-3.64 (m, 1H), 3.27 (s, 3H), 3.03 (s, 1H), 2.81 (s, 1H), 2.65 (s, 1H), 2.22 (d, J=11.6 Hz, 1H), 1.97-1.75 (m, 2H), 1.55-1.36 (m, 2H), 1.34-1.24 (m, 3H), 0.95-0.80 (m, 3H), 0.74-0.61 (m, 3H) ppm. MS: M/e 493 (M+1)⁺.

### Compound A57: 2-(4-((2S,5R)-4-(1-(6-cyclopropylpyridin-3-yl) propyl)-2,5-diethylpiperazin-1-yl)-1-methyl-2-oxo-1,2-dihydropyrazolo[1,5-a][1,3,5]triazin-7-yl)acetonitrile

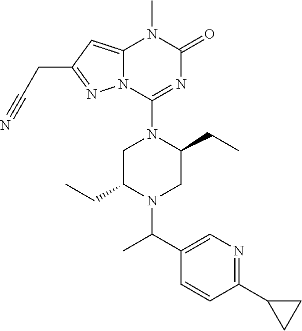 [SMILES: CC[C@H]1CN(C(C)c2ccc(C3CC3)nc2)[C@H](CC)CN1c1nc(=O)n(C)c2cc(CC#N)nn12]

### Step A: 6-cyclopropylnicotinaldehyde

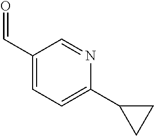 [SMILES: O=Cc1ccc(C2CC2)nc1]

To a solution of 6-bromonicotinaldehyde (1.86 g, 10 mmol) and cyclopropylboronic acid (1.72 g, 20 mmol) in toluene/H₂O (15 mL/1.5 mL) were added dichlorobis(tricyclohexylphosphine)palladium(II) (74 mg, 0.1 mmol) and K₃PO₄ (3.18 g, 15 mmol). The reaction mixture was stirred at 100° C. under N₂ overnight. The mixture was cooled to room temperature, diluted with water (40 mL), extracted with EA (80 mL×2), washed with brine, dried over Na₂SO₄, filtered, and concentrated to dryness. The residue was purified by flash column chromatography to give the titled compound (1.2 g, 81%). MS: M/e 148 (M+1)⁺.

### Step B: 1-(6-cyclopropylpyridin-3-yl)propan-1-ol

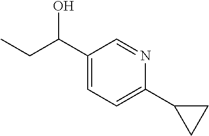

To a solution of 6-cyclopropylnicotinaldehyde (200 mg, 1.36 mmol) in THF (5 mL) was added a solution of EtMgBr in THF (0.5 mL, 3 mol/L). The reaction mixture was stirred at room temperature for 2 hrs. The mixture was diluted with saturated NH₄Cl (20 mL), extracted with EA (70 mL), washed with brine, dried over Na₂SO₄, filtered, and concentrated to dryness. The residue was purified by flash column chromatography to give the titled compound (200 mg, 83%). MS: M/e 178 (M+1)⁺.

### Step C: 2-(4-((2S,5R)-4-(1-(6-cyclopropylpyridin-3-yl) propyl)-2,5-diethylpiperazin-1-yl)-1-methyl-2-oxo-1,2-dihydropyrazolo[1,5-a][1,3,5]triazin-7-yl)acetonitrile

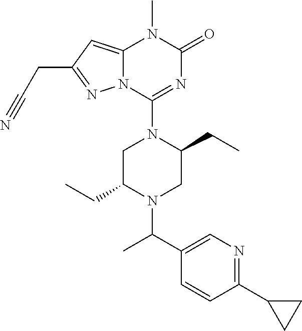 [SMILES: CC[C@H]1CN(C(C)c2ccc(C3CC3)nc2)[C@H](CC)CN1c1nc(=O)n(C)c2cc(CC#N)nn12]

To a solution of 2-(4-((2S,5R)-2,5-diethylpiperazin-1-yl)-1-methyl-2-oxo-1,2-dihydropyrazolo[1,5-a][1,3,5]triazin-7-yl)acetonitrile (66 mg, 0.2 mmol), 1-(6-cyclopropylpyridin-3-yl)propan-1-ol (71 mg, 0.4 mmol) and (cyanomethyl) trimethylphosphonium iodide (193 mg, 0.8 mmol) in CH₃CN (2 mL) was added DIPEA (103 mg, 0.8 mmol). The reaction mixture was sealed in a bottle and heated at 100° C. for 16 hours, and then cooled to room temperature, added 1-(6-cyclopropylpyridin-3-yl)propan-1-ol (71 mg, 0.4 mmol) and stirred at 100° C. for 2 days. The reaction was diluted with water (20 mL), extracted with EtOAc (5 mL×2). The combined organic layers were washed with brine, dried over Na₂SO₄, filtered, and concentrated to dryness. The resulting residue was purified and separated into Compound A57a (1.8 mg, 1.8%) and Compound A57b (2.8 mg, 2.8%) by prep-HPLC (Method A).

Compound A57a (the earlier peak): ¹H NMR (400 MHz, CDCl₃) δ 8.36 (s, 1H), 7.27 (s, 1H), 7.15 (s, 1H), 5.80 (s, 1H), 5.75 (s, 1H), 5.20-4.80 (m, 1H), 3.74 (s, 3H), 3.43 (s, 4H), 2.88 (s, 2H), 2.37-2.26 (m, 1H), 1.91 (s, 5H), 1.41 (s, 4H), 1.05 (s, 2H), 0.93 (s, 3H), 0.68 (d, J=6.3 Hz, 6H) ppm. MS: M/e 489 (M+1)⁺.

Compound A57b (the later peak): ¹H NMR (400 MHz, CDCl₃) δ 8.38 (s, 1H), 7.27 (s, 1H), 7.15 (s, 1H), 5.76 (s, 2H), 4.88 (s, 1H), 3.74 (s, 3H), 3.43 (s, 3H), 3.39-3.25 (m, 2H), 3.14-3.03 (m, 1H), 2.85 (s, 1H), 2.65 (s, 1H), 2.22-2.09 (m, 2H), 1.89 (s, 3H), 1.68 (s, 2H), 1.44-1.41 (m, 1H), 0.99 (s, 4H), 0.66 (d, J=7.0 Hz, 7H) ppm. MS: M/e 489 (M+1)⁺.

### Compound A58: 2-(4-((2S,5R)-4-(l-(2-chloropyrazolo[1,5-a]pyrimidin-5-yl)ethyl)-2,5-diethylpiperazin-1-yl)-1-methyl-2-oxo-1,2-dihydropyrazolo[1,5-a][1,3,5]triazin-7-yl)acetonitrile

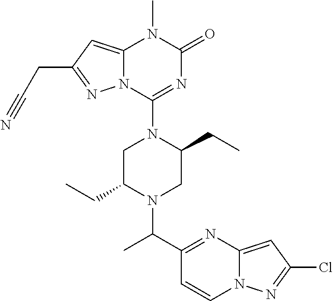 [SMILES: CC[C@H]1CN(C(C)c2ccn3nc(Cl)cc3n2)[C@H](CC)CN1c1nc(=O)n(C)c2cc(CC#N)nn12]

### Step A: 2-chloropyrazolo[1,5-a]pyrimidin-5-ol

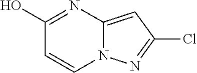 [SMILES: Oc1ccn2nc(Cl)cc2n1]

To a solution of 3-chloro-1H-pyrazol-5-amine (2.34 g, 20 mmol) in DMF (10 mL) was added ethyl (E)-3-ethoxyacrylate (4.3 g, 30 mmol) and Cs₂CO₃ (13 g, 40 mmol). The reaction mixture was stirred at 100° C. for 16 hrs. The mixture was cooled down to RT added filtered. The filtrate was added CH₃COOH (10 mL) and concentrated in vacuo. The resulting residue was purified by flash column chromatography with 0-100% EtOAc in PE to give the titled compound (2.1 g, 60%). MS: M/e 170 (M+1)⁺.

### Step B: 5-bromo-2-chloropyrazolo[1,5-a]pyrimidine

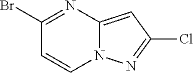 [SMILES: Clc1cc2nc(Br)ccn2n1]

To a solution of 2-chloropyrazolo[1,5-a]pyrimidin-5-ol (2.1 g, 12.4 mmol) in MeCN (20 mL) was added POBr₃ (6.6 g, 24.8 mmol). The reaction mixture was stirred at 90° C. for 2 hrs. The mixture was cooled down to RT and concentrated in vacuo. The residue was added to saturated NaHCO₃ aqueous solution (30 mL) and then extracted with EtOAc (30 mL×3). The organic layer was washed with brine (30 mL), dried and concentrated. The resulting residue was purified by flash column chromatography with 0-100% EA in PE to give the titled compound (1 g, 35.1%). MS: M/e 232 (M+1)⁺.

### Step C: 1-(2-chloropyrazolo[1,5-a]pyrimidin-5-yl)ethan-1-one

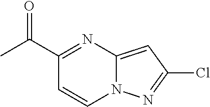

To a solution of 5-bromo-2-chloropyrazolo[1,5-a]pyrimidine (231 mg, 1 mmol) in toluene was added Pd(PPh₃)₂Cl₂ (70 mg, 0.1 mmol) and tributyl(1-ethoxyvinyl)stannane (547 mg, 1.5 mmol). The reaction mixture was stirred at 100° C. under N₂ for 8 hours. The reaction mixture was added 4N HCl in 1,4-dioxane (2 mL) and stirred at RT for 15 mins. The mixture was added saturated NaHCO₃ aqueous solution (30 mL) and then extracted with EtOAc (30 mL×3). The organic layer was washed with brine (30 mL), dried and concentrated. The resulting residue was purified by flash column chromatography with 0-100% EA in PE to give the titled compound (110 mg, 51.2%). MS: M/e 196 (M+1)⁺.

### Step D: 1-(2-chloropyrazolo[1,5-a]pyrimidin-5-yl)ethan-1-ol

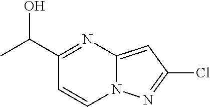

To a solution of 1-(2-chloropyrazolo[1,5-a]pyrimidin-5-yl)ethan-1-one (110 mg, 0.56 mmol) in MeOH was added NaBH₄ (19 mg, 0.5 mmol) and the reaction mixture was stirred at RT for 15 mins. The reaction mixture was added H₂O (30 mL) and then extracted with DCM (30 mL×3). The organic layer was washed with brine (30 mL), dried and concentrated. The resulting residue was purified by flash column chromatography with 0-100% EA in PE to give the titled compound (70 mg, 63.6%). MS: M/e 198 (M+1)⁺.

### Step E: 2-(4-((2S,5R)-4-(1-(2-chloropyrazolo[1,5-a]pyrimidin-5-yl)ethyl)-2,5-diethylpiperazin-1-yl)-1-methyl-2-oxo-1,2-dihydropyrazolo[1,5-a][1,3,5]triazin-7-yl)acetonitrile

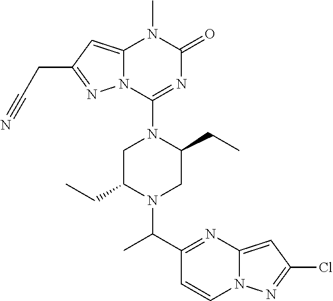 [SMILES: CC[C@H]1CN(C(C)c2ccn3nc(Cl)cc3n2)[C@H](CC)CN1c1nc(=O)n(C)c2cc(CC#N)nn12]

A mixture of 2-(4-((2S,5R)-2,5-diethylpiperazin-1-yl)-1-methyl-2-oxo-1,2-dihydropyrazolo[1,5-a][1,3,5]triazin-7-yl)acetonitrile (30 mg, 0.1 mmol), 1-(2-chloropyrazolo[1,5-a]pyrimidin-5-yl)ethan-1-ol (50 mg, 0.25 mmol), (cyanomethyl)trimethylphosphonium iodide (120 mg, 0.5 mmol) and DIPEA (129 mg, 1 mmol) in MeCN (3 mL) was stirred at 100° C. for 16 hours. The resulted mixture was diluted with EtOAc (10 mL), washed with brine (5 mL×3), dried and concentrated. The resulting residue was purified by flash column chromatography and further purified to give the titled compound (1.3 mg, 0.25%) by prep-HPLC (Method A). ¹H NMR (400 MHz, CD₃OD) δ 8.78 (d, J=6.2 Hz, 1H), 7.28 (m, 1H), 6.58 (d, J=4.6 Hz, 1H), 6.04 (s, 1H), 3.96 (m, 2H), 3.75-3.47 (m, 1H), 3.42 (m, 5H), 3.13-2.79 (m, 1H), 2.95 (m, 1H), 2.49 (m, 1H), 2.05 (m, 1H), 1.88 (m, 1H), 1.57 (m, 3H), 1.39 (t, J=7.7 Hz, 3H), 0.94 (s, 3H), 0.76 (d, J=7.4 Hz, 3H) ppm. MS: M/e 509 (M+1)⁺.

### Compound A59: 2-(4-((2S,5R)-4-(1-(2-chloro-3-fluoropyrazolo[1,5-a]pyrimidin-5-yl)ethyl)-2,5-diethylpiperazin-1-yl)-1-methyl-2-oxo-1,2-dihydro pyrazolo[1,5-a][1,3,5]triazin-7-yl)acetonitrile

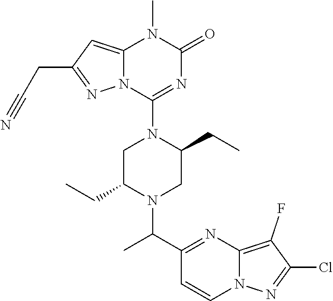 [SMILES: CC[C@H]1CN(C(C)c2ccn3nc(Cl)c(F)c3n2)[C@H](CC)CN1c1nc(=O)n(C)c2cc(CC#N)nn12]

### Step A: 5-bromo-2-chloro-3-fluoropyrazolo[1,5-a]pyrimidine

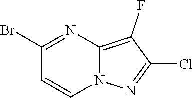 [SMILES: Fc1c(Cl)nn2ccc(Br)nc12]

To a solution of 5-bromo-2-chloropyrazolo[1,5-a]pyrimidine (115 mg, 0.5 mmol) in MeCN (10 mL) was added Select F (265 mg, 0.75 mmol). The reaction mixture was stirred at RT for 16 hrs. The mixture was cooled down to RT and concentrated in vacuo. The resulting residue was purified by flash column chromatography with 0-100% EtOAc in PE to give the titled compound (80 mg, 64.5%). MS: M/e 250 (M+1)⁺.

### Step B: 1-(2-chloro-3-fluoropyrazolo[1,5-a]pyrimidin-5-yl)ethan-1-one

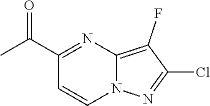

To a solution of 5-bromo-2-chloro-3-fluoropyrazolo[1,5-a]pyrimidine (80 mg, 0.32 mmol) in toluene was added Pd(PPh₃)₂Cl₂ (35 mg, 0.05 mmol) and tributyl(1-ethoxyvinyl)stannane (180 mg, 0.5 mmol). The reaction mixture was stirred at 100° C. under N₂ for 8 hours. The reaction mixture was added 4N HCl/Dioxane (1 mL) and stirred at RT for 15 mins. The mixture was added saturated NaHCO₃ aqueous solution (30 mL) and then extracted with EtOAc (30 mL×3). The organic layer was washed with brine (30 mL), dried and concentrated. The resulting residue was purified by flash column chromatography with 0-100% EA in PE to give the titled compound (40 mg, 58.8%). MS: M/e 214 (M+1)⁺.

### Step C: 1-(2-chloro-3-fluoropyrazolo[1,5-a]pyrimidin-5-yl)ethan-1-ol

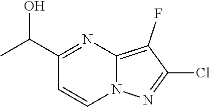

To a solution of 1-(2-chloro-3-fluoropyrazolo[1,5-a]pyrimidin-5-yl)ethan-1-one (40 mg, 0.18 mmol) in MeOH was added NaBH₄ (19 mg, 0.5 mmol) and the reaction mixture was stirred at RT for 15 mins. The reaction mixture was added H₂O (30 mL) and then extracted with DCM (30 mL×3). The organic layer was washed with brine (30 mL), dried and concentrated. The resulting residue was purified by flash column chromatography with 0-100% EA in PE to give the titled compound (30 mg, 75%). MS: M/e: 216 (M+1)⁺.

### Step D: 2-(4-((2S,5R)-4-(1-(2-chloro-3-fluoropyrazolo[1,5-a] pyrimidin-5-yl)ethyl)-2,5-diethylpiperazin-1-yl)-1-methyl-2-oxo-1,2-dihydro pyrazolo[1,5-a][1,3,5]triazin-7-yl)acetonitrile

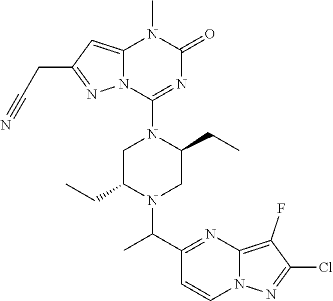 [SMILES: CC[C@H]1CN(C(C)c2ccn3nc(Cl)c(F)c3n2)[C@H](CC)CN1c1nc(=O)n(C)c2cc(CC#N)nn12]

A mixture of 2-(4-((2S,5R)-2,5-diethylpiperazin-1-yl)-1-methyl-2-oxo-1,2-dihydropyrazolo[1,5-a][1,3,5]triazin-7-yl)acetonitrile (30 mg, 0.1 mmol), 1-(2-chloro-3-fluoropyrazolo[1,5-a]pyrimidin-5-yl)ethan-1-ol (50 mg, 0.25 mmol), (cyanomethyl)trimethylphosphonium iodide (120 mg, 0.5 mmol) and DIPEA (129 mg, 1 mmol) in MeCN (3 mL) was stirred at 100° C. for 16 hours. The resulted mixture was diluted with EtOAc (10 mL), washed with brine (5 mL×3), dried and concentrated. The resulting residue was purified by flash column chromatography and further purified to give the titled compound (0.5 mg) by prep-HPLC (Method A). ¹H NMR (400 MHz, CDCl₃) δ 8.39 (s, 1H), 7.10 (s, 1H), 5.78 (s, 1H), 3.75 (m, 3H), 3.44 (s, 3H), 3.08 (m, 1H), 2.88 (m, 1H), 2.39 (m, 1H), 2.22 (m, 1H), 2.01 (m, 2H), 1.59 (m, 4H), 1.26 (m, 6H), 0.76 (m, 3H) ppm. MS: M/e 527 (M+1)⁺.

### Compound A60: 2-(4-((2S,5R)-4-(1-(2,3-difluoropyrazolo[1,5-a]pyrimidin-5-yl)ethyl)-2,5-diethylpiperazin-1-yl)-1-methyl-2-oxo-1,2-dihydropyrazolo[1,5-a][1,3,5]triazin-7-yl)acetonitrile

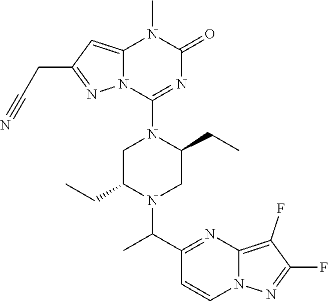 [SMILES: CC[C@H]1CN(C(C)c2ccn3nc(F)c(F)c3n2)[C@H](CC)CN1c1nc(=O)n(C)c2cc(CC#N)nn12]

### Step A: 1-(2,3-difluoropyrazolo[1,5-a]pyrimidin-5-yl)ethan-1-one

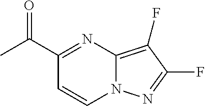

To a solution of 1-(2-fluoropyrazolo[1,5-a]pyrimidin-5-yl)ethan-1-one (0.41 g, 2.2 mmol) in CH₃CN (15 mL) were added 1-Chloromethyl-4-fluoro-1,4-diazoniabicyclo[2.2.2]octane bis(tetrafluoroborate) (2.3 g, 6.6 mmol) was stirred at 50° C. for 5 hours. The solution was concentrated and purified by flash column chromatography to give the titled compound (0.12 g, 26%). MS: M/e 198 (M+1)⁺

### Step B: 1-(2,3-difluoropyrazolo[1,5-a]pyrimidin-5-yl)ethan-1-ol

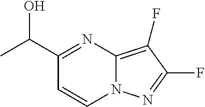

To a solution of 1-(2,3-difluoropyrazolo[1,5-a]pyrimidin-5-yl)ethan-1-one (0.12 g, 0.6 mmol) in MeOH (3 mL) was added NaBH₄ (11 mg, 0.3 mmol) at room temperature and the resulting mixture was stirred at room temperature for 5 mins. The reaction mixture was diluted with DCM and washed with water, dried over Na₂SO₄, concentrated to give the titled compound (55 mg). MS: M/e 200 (M+1)⁺.

### Step C: 2-(4-((2S,5R)-4-(1-(2,3-difluoropyrazolo[1,5-a]pyrimidin-5-yl)ethyl)-2,5-diethylpiperazin-1-yl)-1-methyl-2-oxo-1,2-dihydropyrazolo[1,5-a][1,3,5]triazin-7-yl)acetonitrile

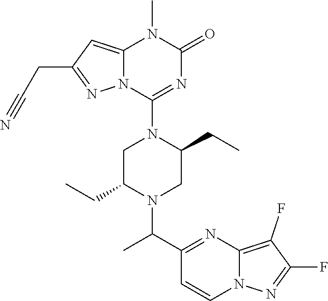 [SMILES: CC[C@H]1CN(C(C)c2ccn3nc(F)c(F)c3n2)[C@H](CC)CN1c1nc(=O)n(C)c2cc(CC#N)nn12]

To a solution of 2-(4-((2S,5R)-2,5-diethylpiperazin-1-yl)-1-methyl-2-oxo-1,2-dihydropyrazolo[1,5-a][1,3,5]triazin-7-yl)acetonitrile (30 mg, 0.09 mmol) in CH₃CN (4 mL) and was added 1-(2,3-difluoropyrazolo[1,5-a]pyrimidin-5-yl)ethan-1-ol (54 mg, 0.27 mmol), (cyanomethyl)trimethylphosphonium iodide (89 mg, 0.36 mmol) and DIPEA (117 mg, 0.9 mmol). The resulting mixture was stirred at 105° C. overnight. The reaction solvent was removed under reduce pressure. The resulting residue was purified by flash column chromatography to give the titled compound (crude), which was further purified to give the titled compound (0.9 mg, 2%) by prep-HPLC (Method A). ¹H NMR (400 MHz, DMSO-d₆) δ 8.94 (dd, J=18.9, 7.4 Hz, 1H), 7.26 (d, J=7.2 Hz, 1H), 6.06 (s, 1H), 5.95-5.27 (m, 1H), 4.91-4.26 (m, 1H), 4.14 (s, 2H), 3.98-3.69 (m, 1H), 3.55-3.37 (m, 3H), 3.15-2.67 (m, 3H), 2.34-2.12 (m, 1H), 2.02-1.41 (m, 4H), 1.32 (dd, J=20.1, 6.6 Hz, 3H), 1.02-0.57 (m, 6H) ppm. MS: M/e 511 (M+1)⁺.

### Compound A61: 2-(4-((2S,5R)-5-ethyl-2-methyl-4-(1-(2-methylpyrazolo[1,5-a]pyrimidin-5-yl)ethyl)piperazin-1-yl)-1-methyl-2-oxo-1,2-dihydropyrazolo[1,5-a][1,3,5]triazin-7-yl)acetonitrile

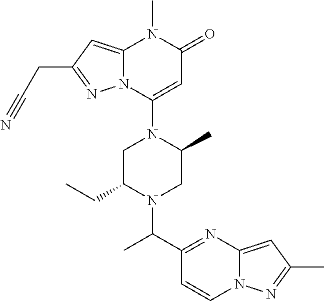 [SMILES: CC[C@@H]1CN(c2cc(=O)n(C)c3cc(CC#N)nn23)[C@@H](C)CN1C(C)c1ccn2nc(C)cc2n1]

To a solution of 2-(4-((2S,5R)-5-ethyl-2-methylpiperazin-1-yl)-1-methyl-2-oxo-1,2-dihydropyrazolo[1,5-a][1,3,5]triazin-7-yl)acetonitrile (63 mg, 0.2 mmol), 1-(2-methylpyrazolo[1,5-a]pyrimidin-5-yl)ethan-1-ol (53 mg, 0.3 mmol) and (cyanomethyl)trimethylphosphonium iodide (97 mg, 0.4 mmol) in CH₃CN (2 mL) was added DIPEA (103 mg, 0.8 mmol). The reaction mixture was sealed in a bottle and heated at 100° C. overnight. The reaction was diluted with water (20 mL), extracted with EtOAc (5 mL×2). The combined organic layers were washed with brine, dried over Na₂SO₄, filtered, and concentrated to dryness. The resulting residue was purified to give the titled compound (20 mg) by prep-HPLC (Method A), which was further separated into Compound A61a (6.8 mg, 7.1%) and Compound A61b (6.3 mg, 6.7%) by chiral Prep-SFC. The chiral separation conditions are shown below.

| | |
|---|---|
| Column | Lux ®Cellulose-3 |
| Column Size | 30 × 250 mm, 5 um |
| Solvent | CO₂ |
| Co-Solvent | 20% MEOH |
| Flow Rate | 90.0 mL/min |
| Wave Length | UV 220 nm and 230 nm |
| Temperature | 35° C. |
| Back Pressure | 100 bar |
| SFC Equipment | Waters Prep SFC 150 AP |

Compound A61: ¹H NMR (400 MHz, CDCl₃) δ 8.53 (s, 1H), 7.00 (s, 1H), 6.40 (s, 1H), 5.77 (s, 1H), 3.75 (s, 3H), 3.44 (s, 3H), 3.15-2.70 (m, 2H), 2.52 (s, 3H), 2.00 (s, 2H), 1.57 (s, 3H), 1.53-1.42 (m, 3H), 1.36 (s, 4H), 1.09-0.72 (m, 3H) ppm. MS: M/e 475 (M+1)⁺.

Compound A61a (the earlier peak): ¹H NMR (400 MHz, CDCl₃) δ 8.54 (s, 1H), 7.27 (s, 1H), 6.44 (s, 1H), 5.79 (s, 1H), 4.48-4.15 (m, 1H), 3.73 (s, 3H), 3.44 (s, 3H), 3.15-2.71 (m, 2H), 2.53 (s, 3H), 1.99 (s, 5H), 1.49-1.21 (m, 6H), 0.75 (s, 3H) ppm. MS: M/e 475 (M+1)⁺.

Compound A61b (the later peak): ¹H NMR (400 MHz, CDCl₃) δ 8.76-8.35 (m, 1H), 7.04 (s, 1H), 6.39 (s, 1H), 5.79 (s, 1H), 4.34-3.93 (m, 1H), 3.76 (s, 3H), 3.44 (s, 3H), 3.27-3.02 (m, 1H), 2.83 (s, 1H), 2.53 (s, 3H), 2.00 (s, 2H), 1.75 (s, 3H), 1.45-1.16 (m, 6H), 1.03 (s, 3H) ppm. MS: M/e 475 (M+1)⁺.

### Compound A62: 2-(4-((2S,5R)-5-ethyl-2-methyl-4-(1-(2-methylpyrazolo[1,5-a]pyrimidin-5-yl)propyl)piperazin-1-yl)-1-methyl-2-oxo-1,2-dihydropyrazolo[1,5-a][1,3,5]triazin-7-yl)acetonitrile

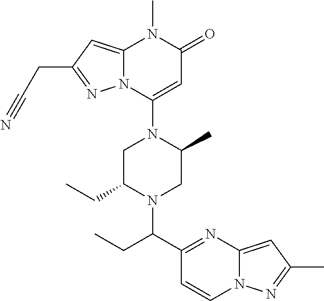 [SMILES: CCC(c1ccn2nc(C)cc2n1)N1C[C@H](C)N(c2cc(=O)n(C)c3cc(CC#N)nn23)C[C@H]1CC]

To a solution of 2-(4-((2S,5R)-5-ethyl-2-methylpiperazin-1-yl)-1-methyl-2-oxo-1,2-dihydropyrazolo[1,5-a][1,3,5]triazin-7-yl)acetonitrile (110 mg, 0.35 mmol), 1-(2-methylpyrazolo[1,5-a]pyrimidin-5-yl)propan-1-ol (100 mg, 0.52 mmol) and (cyanomethyl)trimethylphosphonium iodide (169 mg, 0.7 mmol) in CH₃CN (3 mL) was added DIPEA (180 mg, 1.4 mmol). The reaction mixture was sealed in a bottle and heated at 100° C. for 16 hours. The reaction was diluted with water (20 mL), extracted with EtOAc (5 mL×2). The combined organic layers were washed with brine, dried over Na₂SO₄, filtered, and concentrated to dryness. The resulting residue was purified and separated into Compound A62a (13.6 mg, 7.9%) and Compound A62b (8.7 mg, 5.1%) by prep-HPLC (Method B).

Compound A62a (the earlier peak): ¹H NMR (400 MHz, CDCl₃) δ 8.53 (s, 1H), 6.89 (s, 1H), 6.42 (s, 1H), 5.76 (s, 1H), 3.73 (s, 3H), 3.43 (s, 4H), 2.97 (s, 1H), 2.79 (s, 1H), 2.60-2.38 (m, 4H), 1.97 (s, 2H), 1.57 (s, 3H), 1.46 (s, 4H), 0.78 (s, 6H) ppm. MS: M/e 489 (M+1)⁺.

Compound A62b (the later peak): ¹H NMR (400 MHz, CDCl₃) δ 8.50 (s, 1H), 6.97 (s, 1H), 6.40 (s, 1H), 5.76 (s, 1H), 5.01-3.87 (m, 1H), 3.74 (s, 2H), 3.55 (s, 1H), 3.43 (s, 3H), 3.10 (s, 1H), 2.84 (s, 1H), 2.51 (s, 3H), 2.12 (d, J=11.5 Hz, 1H), 1.95 (s, 1H), 1.58 (s, 3H), 1.52-1.41 (m, 2H), 1.32 (s, 3H), 1.01 (s, 3H), 0.75 (s, 3H) ppm. MS: M/e 489 (M+1)⁺.

### Compound A63: 2-(4-((2S,5R)-2,5-diethyl-4-(1-(2-fluoro-4-(trifluoromethyl)phenyl)ethyl)piperazin-1-yl)-1-methyl-2-oxo-1,2-dihydropyrazolo[1,5-a][1,3,5]triazin-7-yl)acetonitrile

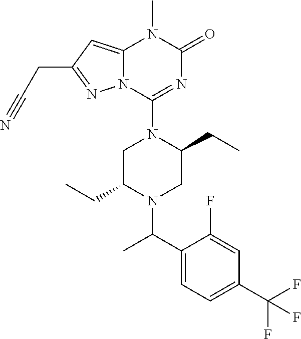 [SMILES: CC[C@H]1CN(C(C)c2ccc(C(F)(F)F)cc2F)[C@H](CC)CN1c1nc(=O)n(C)c2cc(CC#N)nn12]

A solution of 2-(4-((2S,5R)-2,5-diethylpiperazin-1-yl)-1-methyl-2-oxo-1,2-dihydropyrazolo[1,5-a][1,3,5]triazin-7-yl)acetonitrile (66 mg, 0.20 mmol), 1-(2-fluoro-4-(trifluoromethyl)phenyl)ethan-1-ol (83.5 mg, 0.40 mmol), (cyanomethyl)trimethylphosphonium iodide (146.2 mg, 0.60 mmol) and DIPEA (258.8 mg, 2.01 mmol) in CH₃CN (2 mL) was stirred at 100° C. overnight. The reaction was diluted with EtOAc (10 mL) and washed with brine (10 mL). The organic layer was concentrated under reduced pressure. The resulting residue was purified by flash column chromatography with 0-5% MeOH in DCM to give the titled compound, which was further separated into Compound A63a (4.6 mg, 4.4%) and Compound A63b (8.1 mg, 7.8%) by prep-HPLC (Method A).

Compound A63a (the earlier peak): ¹H NMR (400 MHz, CD₃OD) δ 7.77 (t, J=7.1 Hz, 1H), 7.53 (d, J=8.3 Hz, 1H), 7.43 (d, J=10.2 Hz, 1H), 6.15-5.90 (m, 2H), 5.06-4.86 (m, 2H), 4.24 (q, J=6.6 Hz, 1H), 3.98 (s, 1H), 3.41 (s, 3H), 3.37-3.33 (m, 1H), 3.02-2.85 (m, 2H), 2.48-2.34 (m, 1H), 2.12-1.96 (m, 2H), 1.58-1.46 (m, 2H), 1.37 (d, J=6.4 Hz, 3H), 0.96 (t, J=7.1 Hz, 3H), 0.75 (s, 3H) ppm. MS: M/e 520 (M+1)⁺.

Compound A63b (the later peak): ¹H NMR (400 MHz, CD₃OD) δ 7.85 (t, J=7.4 Hz, 1H), 7.50 (d, J=7.9 Hz, 1H), 7.40 (d, J=10.2 Hz, 1H), 6.38-5.74 (m, 2H), 5.05-4.85 (m, 2H), 4.12-3.92 (m, 2H), 3.42 (s, 3H), 3.37-3.32 (m, 1H), 3.25-3.10 (m, 1H), 2.78-2.66 (m, 1H), 2.29 (d, J=12.0 Hz, 1H), 1.99-1.83 (m, 2H), 1.69-1.47 (m, 2H), 1.34 (d, J=6.2 Hz, 3H), 1.02 (s, 3H), 0.70 (t, J=7.1 Hz, 3H) ppm. MS: M/e 520 (M+1)⁺.

### Compound A64: 2-(4-((2S,5R)-2,5-diethyl-4-(1-(4-(trifluoromethyl)phenyl) propyl)piperazin-1-yl)-1-methyl-2-oxo-1,2-dihydropyrazolo[1,5-a][1,3,5]triazin-7-yl)acetonitrile

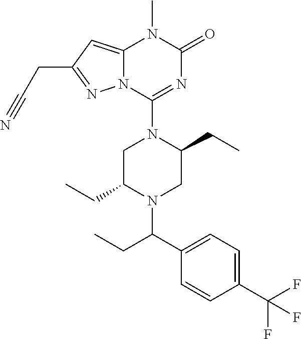 [SMILES: CCC(c1ccc(C(F)(F)F)cc1)N1C[C@H](CC)N(c2nc(=O)n(C)c3cc(CC#N)nn23)C[C@H]1CC]

A mixture of 2-(4-((2S,5R)-2,5-diethylpiperazin-1-yl)-1-methyl-2-oxo-1,2-dihydropyrazolo[1,5-a][1,3,5]triazin-7-yl)acetonitrile (32.9 mg, 0.1 mmol), 1-(4-(trifluoromethyl)phenyl)propan-1-ol (60 mg, 0.3 mmol), (cyanomethyl) trimethylphosphonium iodide (72.9 mg, 0.3 mmol) and DIPEA (64.5 mg, 0.5 mmol) in CH₃CN (4 mL) was stirred at 100° C. overnight in a sealed tube. The reaction mixture was diluted with EtOAc (10 mL), washed with brine, dried over Na₂SO₄, concentrated and purified by Pre-TLC (EtOAc) to give the titled compound, which was further separated into Compound A64a (7.3 mg, 14.2%) and Compound A64b (8.1 mg, 15.7%) by Prep-HPLC (Method A).

Compound A64a (the earlier peak). ¹H NMR (400 MHz, CD₃OD) δ 7.65 (d, J=8.0 Hz, 2H), 7.53 (d, J=8.0 Hz, 2H), 6.17-5.82 (m, 1H), 6.03 (s, 1H), 4.82-4.56 (m, 1H), 3.98 (s, 2H), 3.68-3.59 (m, 1H), 3.41 (s, 3H), 3.51-3.38 (m, 1H), 3.05-2.86 (m, 2H), 2.45-2.32 (m, 1H), 2.13-1.92 (m, 3H), 1.71-1.38 (m, 3H), 0.97 (t, J=7.2 Hz, 3H), 0.67 (t, J=7.2 Hz, 6H) ppm. MS: M/e 516 (M+1)⁺.

Compound 64b (the later peak). ¹H NMR (400 MHz, CD₃OD) δ 7.63 (d, J=8.0 Hz, 2H), 7.55 (d, J=8.0 Hz, 2H), 6.03 (s, 1H), 6.34-5.75 (m, 1H), 4.82-4.59 (m, 1H), 3.99 (d, J=15.2 Hz, 2H), 3.72-3.45 (m, 2H), 3.41 (s, 3H), 3.25-3.09 (m, 1H), 2.75-2.61 (m, 1H), 2.32-2.20 (m, 1H), 1.99-1.79 (m, 3H), 1.69-1.46 (m, 3H), 1.12-0.92 (m, 3H), 0.71-0.58 (m, 6H) ppm. MS: M/e 516 (M+1)⁺.

### Assay

### Biochemical DGK IC₅₀ Assays

Enzymatic reactions of DGKζ, DGKα and DGKδ were performed using ADP-Glo assay with lipid micelle substrate. Full length DGKζ in-house protein M1-V929 with SEQ ID No: 2) was expressed in baculovirus expression system. Full length DGKα (D21-10BG, SignalChem) and full length DGKδ (D23-10G, SignalChem) were purchased. Lipid micelle was prepared by dissolving DAG (Sigma, 317505-10MG) and PS (Sigma, P7769-100MG) with chloroform which was furtherly removed by rotary evaporation. The resulted product was resuspended in buffer containing 25 mM HEPES pH 7.0, 0.5 mM EDTA and 160 mM Octyl β-D glucopyranoside by vigorous mixing and ultrasonic (IID, Scientz).

Final concentration in reaction

| Enzyme | Enzyme (nM) | ATP (μM) | DAG (μM) |
|---|---|---|---|
| DGKα | 7.5 | 140 | 80 |
| DGKδ | 10 | 140 | 80 |
| DGKζ | 0.5 | 140 | 80 |

The inhibition activities testing for the compound disclosed herein were carried out at room temperature in assay buffer containing 50 mM HEPES, 10 mM MgCl₂, 0.01% BSA, 0.1 mM Na₃VO₄, 0.005% Tween-20 and 0.01 mM CaCl₂. Compounds in DMSO were dispensed into wells of a black 384 well plate (Corning 4514) using D300e digital dispenser (Tecan). The ranges of compounds final concentration were 1.55-10000 nM or 23.3-150000 nM. 3 μL 2× enzyme solution was added to wells. After incubation for 1 hour, 3 μL 2× substates solution containing 160 μM DAG and 280 μM ATP was added to the wells to initiate reaction. After 1 hour reaction, 5 μL ADP-Glo reagent (Promga V9101) was added and incubated for 40 minutes. 10 μL Kinase Detection reagent was added and incubated for 30 minutes. Luminescence was measured on a microplate reader (PHERAstar FSX, BMG labtech). The IC₅₀s are calculated based on inhibition of enzyme activity in the presence of increasing concentrations of compounds. Selected compounds had no inhibitory activity on DGKδ. IC₅₀s of the compounds disclosed herein for DGKζ and DGKα are shown in Table 1.

### Baculovirus Expression of Human DGKζ

Human His-TEV-DGK-zeta-pFastBac1 and human baculovirus samples was generated using the Bac-to-Bac baculovirus expression system (Invitrogen) according to the manufacturer's protocol. The DNA used for expression of DGK-zeta, have SEQ ID Nos: 1. Baculovirus amplification was achieved using infected SF9 cells at 1:2000 virus/cells ratios, and grown for 96 hours at 27° C. post-transfected.

The expression scaled up for each protein was carried out in the flask 3 L from CORNING. 4 L of 3×10⁶ cells/mL Sf9 cells (Expression System, Invitrogen) grown in SF900™ II SFM insect medium (Expression System) was infected with virus stock at 1:200 virus/cells ratio, and grown for 48 hours at 27° C. post-transfection. The infected cell culture was harvested by centrifugation at 6000 rpm for 15 minutes 4° C. in a SORVALL LYNX6000 centrifuge. The cell pellets were stored at −80° C.

### Purification of Human DGK-Zeta

Full length human DGKζ produced as described above, was purified from Sf9 baculovirus-infected insect cell paste. The cells were lysed using sonication method, and the lysates were clarified by centrifugation. The clarified lysates were purified to ˜90% homogeneity, using two successive column chromatography steps on an AKTA Purifier system. The two steps column chromatography included nickel affinity resin capture (i.e. Ni-NTA Agarose, Qiagen), followed by size exclusion chromatography (i.e. Hiload16/60 Superdex200 prep grade, GE Healthcare. The protein was delivered and stored at −80° C. The formulation buffers were identical for the protein: 25 mM Tris, 150 mmol/L NaCl, 2 mM DTT, pH8.0.

SEQ ID No: 1 is Nucleotide sequence encoding His-TEV-hDGKζ-(M1-V929):
ATGCATCACCATCACCATCACCATCACGAGAACCTGTACTTCCAGGGATCC
ATGGAACCCCGTGATGGTAGCCCCGAAGCTCGTAGCTCCGATTCCGAGTCCGCCAGCGC
TTCCTCCTCCGGTAGCGAACGTGACGCTGGTCCCGAGCCCGACAAAGCTCCCCGTCGTC
TGAATAAGCGCCGTTTTCCCGGTCTCCGTCTGTTCGGCCACCGCAAGGCCATCACTAAG
TCCGGTCTCCAGCATCTGGCTCCTCCTCCTCCTACCCCCGGTGCTCCTTGCTCCGAATCC
GAGCGCCAGATTCGCTCCACTGTGGATTGGTCCGAAAGCGCCACCTATGGTGAGCATAT
CTGGTTCGAGACCAACGTCTCCGGCGACTTCTGTTATGTCGGTGAGCAATACTGTGTGG
CTCGTATGCTGCAGAAGTCCGTGTCCCGCCGTAAATGCGCCGCTTGCAAAATCGTGGTC
CATACCCCTTGCATCGAGCAACTGGAGAAAATCAACTTCCGCTGCAAGCCCAGCTTTCG
TGAGTCCGGTTCCCGCAACGTGCGCGAACCTACTTTCGTGCGCCACCACTGGGTGCATC
GTCGTCGCCAAGACGGCAAATGCCGCCACTGCGGCAAAGGTTTTCAGCAGAAATTCAC
CTTCCACAGCAAGGAGATCGTCGCCATCAGCTGCAGCTGGTGCAAACAAGCTTACCATT
CCAAAGTGAGCTGCTTCATGCTCCAGCAGATCGAAGAGCCTTGCTCTCTGGGTGTGCAT
GCTGCTGTCGTGATTCCCCCTACTTGGATTCTGCGTGCTCGCCGTCCCCAGAACACTCTG
AAGGCCTCCAAAAAGAAGAAGCGCGCCAGCTTCAAGCGTAAGAGCTCCAAAAAGGGT
CCCGAAGAGGGCCGTTGGCGTCCCTTCATCATCCGCCCTACTCCTTCCCCTCTGATGAAG
CCTCTGCTGGTCTTCGTCAACCCTAAGAGCGGCGGCAACCAAGGTGCTAAAATCATCCA
GTCCTTCCTCTGGTATCTGAACCCTCGTCAAGTGTTCGACCTCAGCCAAGGCGGTCCTA
AGGAGGCTCTGGAGATGTACCGCAAGGTCCACAATCTGCGCATCCTCGCTTGTGGTGGC
GATGGCACCGTGGGCTGGATTCTGTCCACTCTGGACCAACTGCGTCTGAAACCTCCCCC
CCCCGTGGCTATTCTGCCTCTCGGTACCGGCAACGATCTGGCTCGTACTCTGAATTGGGG
TGGTGGCTACACCGATGAGCCCGTGTCCAAGATTCTGTCCCACGTCGAAGAAGGCAATG
TCGTCCAACTGGACCGTTGGGACCTCCACGCCGAACCCAACCCCGAGGCTGGCCCCGA
GGACCGTGACGAGGGCGCTACTGACCGTCTGCCCCTCGACGTCTTCAATAATTACTTCTC
TCTGGGCTTTGACGCTCACGTGACTCTGGAATTTCATGAAAGCCGCGAGGCCAACCCCG
AGAAGTTCAATTCCCGTTTCCGCAACAAGATGTTCTACGCTGGCACCGCCTTCAGCGAC
TTCCTCATGGGCTCCAGCAAGGACCTCGCTAAGCATATCCGCGTGGTGTGCGATGGCATG
GATCTGACCCCTAAGATCCAAGATCTGAAGCCCCAATGTGTCGTGTTTCTGAACATCCCC
CGCTACTGCGCTGGTACTATGCCTTGGGGCCATCCCGGTGAACACCATGACTTCGAACCT
CAGCGTCATGATGACGGCTATCTGGAGGTGATCGGTTTCACCATGACCTCCCTCGCTGCT
CTGCAAGTGGGTGGCCACGGCGAACGTCTGACTCAATGCCGCGAGGTGGTGCTGACCA
CCAGCAAAGCCATCCCCGTCCAAGTGGATGGTGAGCCTTGCAAGCTGGCCGCCTCCCGT
ATCCGTATCGCTCTCCGCAATCAAGCTACCATGGTCCAGAAGGCCAAACGCCGCAGCGC
TGCTCCTCTCCACAGCGACCAACAACCCGTCCCCGAACAGCTGCGCATCCAAGTGTCCC
GTGTCAGCATGCATGACTACGAGGCTCTGCACTACGACAAGGAACAGCTGAAGGAAGC
CAGCGTGCCTCTGGGTACTGTGGTCGTGCCCGGTGACAGCGATCTGGAGCTCTGCCGTG
CCCACATCGAGCGTCTGCAGCAAGAGCCCGACGGTGCTGGTGCCAAGAGCCCTACTTG
CCAAAAACTCTCCCCCAAGTGGTGTTTCCTCGACGCTACCACCGCCAGCCGCTTCTACC
GCATTGATCGCGCCCAAGAGCATCTGAACTATGTCACCGAGATCGCTCAAGACGAGATC
TACATCCTCGACCCCGAACTCCTCGGTGCTAGCGCCCGTCCCGACCTCCCCACTCCTACC
TCCCCTCTGCCCACTTCCCCTTGTTCCCCCACCCCTCGTAGCCTCCAAGGTGATGCTGCC
CCTCCTCAAGGTGAGGAGCTCATTGAGGCCGCTAAGCGTAACGATTTCTGCAAGCTCCA
AGAGCTGCATCGTGCTGGTGGCGACCTCATGCACCGCGATGAGCAGAGCCGCACTCTGC
TGCACCACGCTGTGTCCACTGGTAGCAAGGACGTGGTGCGCTATCTGCTGGACCACGCT
CCTCCCGAGATCCTCGACGCTGTGGAAGAAAACGGCGAGACTTGCCTCCACCAAGCTG
CTGCTCTGGGTCAACGTACCATCTGCCACTACATCGTCGAAGCTGGTGCTTCTCTGATGA
AGACCGACCAGCAAGGTGATACTCCCCGTCAGCGCGCCGAGAAAGCCCAAGACACCGA
ACTGGCTGCCTATCTGGAGAACCGTCAGCACTACCAGATGATTCAGCGTGAAGACCAAG
AGACCGCCGTGTAA
SEQ ID No: 2 is Amino acid sequence of His-TEV-hDGKζ-(M1-V929):
MEPRDGSPEARSSDSESASASSSGSERDAGPEPDKAPRRLNKRRFPGLRLFGH
RKAITKSGLQHLAPPPPTPGAPCSESERQIRSTVDWSESATYGEHIWFETNVSGDFCYVGEQ
YCVARMLQKSVSRRKCAACKIVVHTPCIEQLEKINFRCKPSFRESGSRNVREPTFVRHHWV
HRRRQDGKCRHCGKGFQQKFTFHSKEIVAISCSWCKQAYHSKVSCFMLQQIEEPCSLGVHA
AVVIPPTWILRARRPQNTLKASKKKKRASFKRKSSKKGPEEGRWRPFIIRPTPSPLMKPLLVF
VNPKSGGNQGAKIIQSFLWYLNPRQVFDLSQGGPKEALEMYRKVHNLRILACGGDGTVGW
ILSTLDQLRLKPPPPVAILPLGTGNDLARTLNWGGGYTDEPVSKILSHVEEGNVVQLDRWDL
HAEPNPEAGPEDRDEGATDRLPLDVFNNYFSLGFDAHVTLEFHESREANPEKFNSRFRNKM
FYAGTAFSDFLMGSSKDLAKHIRVVCDGMDLTPKIQDLKPQCVVFLNIPRYCAGTMPWGHP
GEHHDFEPQRHDDGYLEVIGFTMTSLAALQVGGHGERLTQCREVVLTTSKAIPVQVDGEPC
KLAASRIRIALRNQATMVQKAKRRSAAPLHSDQQPVPEQLRIQVSRVSMHDYEALHYDKE
QLKEASVPLGTVVVPGDSDLELCRAHIERLQQEPDGAGAKSPTCQKLSPKWCFLDATTASR
FYRIDRAQEHLNYVTEIAQDEIYILDPELLGASARPDLPTPTSPLPTSPCSPTPRSLQGDAAPP
QGEELIEAAKRNDFCKLQELHRAGGDLMHRDEQSRTLLHHAVSTGSKDVVRYLLDHAPPEI
LDAVEENGETCLHQAAALGQRTICHYIVEAGASLMKTDQQGDTPRQRAEKAQDTELAAYL
ENRQHYQMIQREDQETAV*

TABLE 1

| | DGKζ ADP-Glo | DGKα ADP-Glo |
|---|---|---|
| | assay | assay |
| Compound | IC₅₀(nM) | IC₅₀(nM) |
| A1 | 1,000 | 810 |
| A2 | 50 | 3,120 |
| A2a | 1,710 | >10,000 |
| A2b | 32 | 6,050 |
| A3 | 20.6 | 25,700 |
| A3a | 8.2 | 29,000 |
| A3b | 2,170 | 13,900 |
| A4a | 153 | 64,900 |
| A4b | 7,390 | >10,000 |
| A5 | 100 | 116,000 |
| A6 | 58 | 4,640 |
| A7 | 23 | 16,900 |
| A8 | 42 | 13,300 |
| A9 | 75 | 61,500 |
| A10 | 5 | 20,200 |
| A10a | 3 | 28,900 |
| A10b | 864 | 12,800 |
| A11 | 57 | 10,900 |
| A11a | 949 | 3,340 |
| A11b | 14 | 103,000 |
| A12 | 29 | 6,520 |
| A12a | 11 | 12,500 |
| A12b | 1,150 | 10,400 |
| A13 | 53 | 10,200 |
| A14 | 227 | >10,000 |
| A15a | 5,460 | >10,000 |
| A15b | 36 | >10,000 |
| A16 | 100 | 27,800 |
| A17 | 136 | 56,700 |
| A18 | 28 | 15,700 |
| A19 | 7,900 | 161 |
| A20a | 55 | 1,100 |
| A20b | 3,310 | 3,480 |
| A21 | 717 | 2,940 |
| A22a | 206 | 939 |
| A22b | 3,570 | 2,940 |
| A23 | 105 | 913 |
| A24a | 3,870 | 2,670 |
| A24b | 2,650 | 4,480 |
| A25 | 175 | 148 |
| A26 | 61 | 1,200 |
| A27a | 6 | 36 |
| A27b | 1,300 | 2,090 |
| A28a | 21 | 233 |
| A28b | 2,220 | 1,470 |
| A29a | 42 | 212 |
| A29b | 1,540 | 1,590 |
| A30a | 24 | 1,030 |
| A30b | 2,230 | 7,720 |
| A31 | 80 | 617 |
| A32a | 51 | 335 |
| A32b | 2,550 | 5,610 |
| A33a | >10,000 | >10,000 |
| A33b | >10,000 | >10,000 |
| A34a | 5,710 | >150,000 |
| A34b | >10,000 | >150,000 |
| A35a | 4,690 | 2,260 |
| A35b | 31 | 72 |
| A36 | 629 | 1,580 |
| A37a | 1,580 | 200 |
| A37b | 83 | 24 |
| A38a | >10,000 | 3,880 |
| A38b | 469 | 205 |
| A39a | >10,000 | 585 |
| A39b | 104 | 52 |
| A40a | 5,769 | 1,538 |
| A40b | 53 | 61 |
| A41a | 80 | 25 |
| A41b | 4,560 | 1,592 |
| A42a | 7,839 | 2,532 |
| A42b | 207 | 199 |
| A43 | 266 | 267 |
| A44a | 691 | 34 |
| A44b | 11 | 70 |
| A45a | >10,000 | 1,880 |
| A45b | 116 | 66 |
| A46a | 8,954 | 969 |
| A46b | 50 | 47 |
| A47a | 4,691 | 3,749 |
| A47b | 11 | 22 |
| A48a | >10,000 | 5,325 |
| A48b | 24 | 63 |
| A49a | 57 | 207 |
| A49b | 922 | 618 |
| A50 | 38 | 55 |
| A51a | 4,582 | 753 |
| A51b | 50 | 39 |
| A52a | 3,315 | 849 |
| A52b | 8.7 | 49 |
| A53a | 20 | 114 |
| A53b | 552 | 947 |
| A54a | 4,903 | 485 |
| A54b | 19 | 69 |
| A55 | 53 | 150 |
| A56 | 73 | 17 |
| A57a | 21 | 61 |
| A57b | 338 | 576 |
| A58 | 42 | 15 |
| A59 | 38 | 111 |
| A60 | 40 | 63 |
| A61 | 130 | 456 |
| A61a | 89 | 167 |
| A61b | >10,000 | 2,347 |
| A62a | 58 | 57 |
| A62b | 3,939 | 632 |
| A63a | 72 | 206 |
| A63b | 1,101 | 546 |
| A64a | 129 | 59 |
| A64b | 383 | 60 |

### Cell Culture and DGKα/ζ Knockout Jurkat Cell Line Construction

Jurkat cells and human PBMC were maintained in RPMI 1640 medium (Gibco) supplemented with 10% fetal bovine serum (FBS, Thermo Scientific), 100 units/mL penicillin and 0.1 mg/mL streptomycin (Gibco) in a humidified 37° C. environment with 5% CO₂. HepG2-OS8 cells, which express the single chain variable fragment (scFv) of an anti-human CD₃ mAb OKT3 fused to the C-terminal domain (113-220) of mouse CD8α which includes hinge, transmembrane and cytoplasmic domains, were maintained in MEM medium (Gibco) supplemented with 10% fetal bovine serum (FBS, Thermo Scientific), 100 units/mL penicillin and 0.1 mg/mL streptomycin (Gibco) in a humidified 37° C. environment with 5% CO₂.

Jurkat cells were infected with the lentivirus expressing spCas9 and sgRNA targeting human DGKα or DGKζ. Cell clones that stably knockout with DGKα/ζ were established and maintained in the RPMI 1640 complete medium. Knockout efficiency of eSPCas9-Lenticrispr DGKα or DGKζ sgRNA in single cell clone was determined using genomic sequencing and immunoblotting method. Selected compounds did not induce DGKα or DGKζ independent IL-2 production in DGKα/ζ KO jurkat cells.

### Non-Stimulated Phosphorylated ERK Detection Assay

Cellular non-stimulated phospho-ERK were measured using a AlphaLISA-based method (Beaudet, Lucille, et al. Nature Methods. 2008, 5.12: an8-an9). Jurkat cells were subcultured in T75 flasks. The next day, growth medium was replaced to serum free RPMI 1640 for 4 hours or overnight. The cells were then seeded into 96-well plates and treated with compounds. After 2 hours compound treatment, lysis buffer (PerkinElmer) was added to each well. Plates were then incubated at room temperature with shaking for 30 minutes. A total of 10 μL of cell lysate from each well of a 96-well plate was transferred to a 384-well white assay plate. Phosphor-ERK was quantitated using the AlphaLISA kit (Cat #ALSU-PERK-A10K) as described by the manufacturer manual (PerkinElmer). AlphaLISA signals were measured using a PHERAstar FSX reader (BMG Labtech). Selected compounds did not elevate ERK phosphorylation in Jurkat cells without TCR activation.

### IL-2 Production Assay in Human PBMC

Frozen human PBMC were thawed in RPMI 1640 medium and incubated at 37° C. overnight. OS8 overexpressing HepG2 cells were seeded into 384-well plates overnight. The next day, PBMC were added into the 384-well plates and then treated with compounds. PBMC and HepG2-OS8 cells were co-cultured for 48 hours at 37° C. Culture supernatant was collected for subsequent measurement of IL-2 concentration by a TR-FRET-based method (Degorce, François, et al. Current chemical genomics. 2009, 3: 22) as described by the manufacturer manual (Cisbio). FRET signals were measured using a PHERAstar FSX reader (BMG Labtech). Selected compounds showed good potency in Human PBMC assay.

### Metabolic Stability Assay

Microsomes were incubated at a concentration of 0.5 mg/mL in 96-well microplates in the presence of 1 uM of the compound at 37° C. The reaction was initiated by the addition of NADPH. The final volume was of 100 uL and the incubations were performed in duplicate. The reactions were stopped by the addition of 200 uL acetonitrile (containing internal standards), after which the samples were mixed and centrifuged and the supernatant thus obtained, and analyzed for parent loss by LC-MS/MS. Selected compounds showed good stability in metabolic stability assay.

### CYP Inhibition Assay

The incubation will be carried out in 96-well plates. 1 μL of the compound working solution or vehicle will be added into 179 μL of substrate fortified human liver microsomes. The incubation plate will be pre-warmed at 37° C. for 5 min in water bath before the reactions are started by the addition of 20 μL of 10 mM NADPH solution. The reaction will be carried out in the 37° C. water bath. At the predetermined time points, the reaction will be stopped by adding 300 μL of quenching solution (acetonitrile with internal standards) to each well. The sample plate will be vortexed for 1 min and centrifuged at 3000 g for 10 min. Transfer 100 μL of the supernatant to a new 96-well plate then mixed with 100 μL water for analysis by LC-MS/MS, followed by data processing (i.e., percent inhibition at 10 uM or IC50 determination). Selected compounds showed low CYP inhibition potential in CYP inhibition assay.

### Time-Dependent Inhibition (TDI) Assay

The TDI assay involves pre-incubation (“inactivation incubation”) of 0.1 mg·mL⁻¹ HLMs with 10 uM test compound and Positive Control in the presence or absence of 1 mM NADPH at 37° C. for 30 min. Following the pre-incubation period, remaining CYP activity was determined by subsequently adding substrates (1A2, 40 M phenacetin; CYP2B6, 50 μM bupropion; CYP2C8, 5 μM paclitaxel; CYP2C9, 6 μM diclofenac; CYP2C19, 50 μM (S)-mephenytoin; CYP2D6, 10 μM dextromethorphan, CYP3A, 1 μM Midazolam or 50 μM Testosterone) and NADPH to the pre-incubation mixtures and an “activity incubation” was done for another 20 min for CYP1A2, 2B6, 2C19, 2D6, 10 min for CYP2C8, CYP3A (testosterone), 6 min for CYP2C9 and 5 min for 3A (midazolam). All reactions are terminated by the addition of ice-cold acetonitrile with internal standard and then centrifuge for LC-MS/MS analysis.

### Percentage Time-Dependent Inhibition (% TDI)

$$Percent inhibition = 100 * ( 1 - ( ( \frac{{R}_{Test cmpd + NAPDH}}{{R}_{Vehicle + NAPDH}} ) / ( \frac{{R}_{Test cmpd + NAPDH}}{{R}_{Vehicle + NAPDH}} ) ) )$$

R is the response of the metabolite measured in the sample from the incubation. Selected compounds showed no TDI concern in Time-dependent Inhibition (TDI) assay.

### TDI IC50 Shift Assay

The TDI IC50 shift assay determines the IC50 value of test compound under three different experimental conditions: 0 min pre-incubation, 30 min pre-incubation minus NADPH and 30 min pre-incubation plus NADPH. Following the pre-incubation with/without NADPH, isoform-specific substrates are added to measure the residual enzyme activity without a dilution step, The formation of metabolites was analyzed by using LC-MS/MS, A decrease in the formation of the metabolites to vehicle control was used to calculate IC50 valves. If the compound is a time-dependent inhibitor, a shift to the left (increase in potency) will occur between 0 min pre-incubation and 30 min pre-incubation plus NADPH. Selected compounds showed no TDI concern in TDI IC50 shift assay.

### CYP Induction Assay

Cryopreserved hepatocytes are seeded at a cell density of 55,000 cells per well in a collagen I coated 96-well plate and allowed to adhere for 4-6 h. The plating medium is then replaced with an incubation medium to allow cells to acclimate for 18 h; Then the cultures are ready for induction studies. Remove the Hepatocyte plate from the incubator. Replace the medium in the appropriate wells with toxicity control, DMSO controls, inducers, or test article solutions, each in triplicate. Final concentration of DMSO in the treatment group will be 0.1%. After 24 hours treatment, remove the Hepatocyte plate from the incubator and observe cell morphology under microscope. Renew the medium with test articles that freshly diluted from DMSO stocks. Return plate to the incubator for another 24 hours treatment. After 48 hours of treatment, mRNA levels for CYP1A2, CYP2B6, and CYP3A4 in hepatocytes are quantified using the Cells-to-Ct kit purchased from Life Technologies. Cell morphology is assessed throughout the study with images taken each day. This along with the lactate dehydrogenase leakage and CellTiter-Fluor™ Cell Viability Assay, provide an overall representation of cell health. Fold of induction=mRNA_{(induced)}/mRNA_{(vehicle)}. Selected compounds showed no CYP induction at 1 uM or 5 uM in CYP induction assay.

### Mouse and Rat PK

### Dosing and Sampling

Test compound was administered intravenously at 1 mg/kg and orally at 10 mg/kg to male CD-1 mice or SD rats (n=3). Blood samples was collected at 0.083, 0.25, 0.5, 1, 2, 4, 8 and 24 h post dosing. The blood samples were centrifuged at 3000 g for 5 min at 4° C. to obtain plasma samples.

Plasma concentrations were carried out under the following sample processing method and measurement conditions:

### Sample Processing Method:

An aliquot of 10 μL sample was added with 200 μL IS (Terfenadine, 5 ng/mL) in ACN. The mixture was vortexed for 1 min, and centrifuged at 4000 rpm for 10 min at 4° C. An aliquot of 80 μL supernatant was diluted with 80 μL water, and the mixed sample was injected to liquid chromatography-tandem mass spectrometry (LC-MS/MS) for analysis.

### Bioanalysis:

Instrument: LC-MS/MS (Triple Quad 5500)
Monitor: MRM
Column: Advanced Materials Technology, HALO AQ-C18 2.7 μm 90 Å, 50*2.1 mm
Column temperature: 40° C.
Mobile phase A: H₂O—0.1% FA
Mobile phase B: ACN—0.1% FA
Gradient program: 15% B-15% B (0 min-0.3 min), 15% B-90% B (0.3 min-1.0 min), 90% B-90% B (1.0 min-1.8 min), 90% B-30% B (1.8 min-2.0 min), 30% B-30% B (2.0 min-2.5 min).
Injected sample amount: 2 μL Selected compounds showed acceptable PK profile in mouse and/or rat

### Explorative Acute Toxicity Study in BALB/c Mice

The test article was dissolved in vehicle formulation (DMA:30% solutol HS-15 (w/v):saline=20:20:60) and injected through tail vein into BALB/c mice at the doses of 2 and/or 10 mg/kg. Continuous clinical observation within 2 hours post injection was performed. Selected compounds were well tolerant at the doses of 2 and/or 10 mg/kg.

It is to be understood that, if any prior art publication is referred to herein, such reference does not constitute an admission that the publication forms a part of the common general knowledge in the art in any country.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

The disclosures of all publications, patents, patent applications and published patent applications referred to herein by an identifying citation are hereby incorporated herein by reference in their entirety.

1. A compound of formula (I), 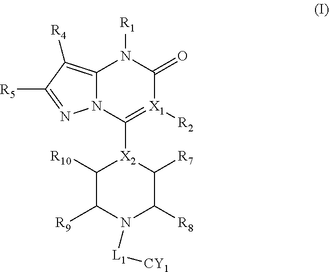
or a pharmaceutically acceptable salt, tautomer, stereoisomer, isotopologue, or enantiomer thereof,
wherein
X₁ is C or N;
X₂ is selected from —CH— or N;
R₁ is hydrogen, or alkyl optionally substituted with deuterium or halogen;
R₂ is hydrogen, halogen, alkyl or cyano, provided R₂ is absent when X₁ is N;
R₄ is hydrogen, halogen, or alkyl, wherein the alkyl is optionally substituted with deuterium or halogen;
R₅ is hydrogen, alkyl, alkenyl, alkynyl, cycloalkyl, cyano, or heterocyclyl, wherein said alkyl or alkenyl is unsubstituted or substituted with halogen, cyano, heterocyclyl, alkoxy, hydroxy, cycloalkyl;
each of R₇, R₉, R₈, and R₁₀ is independently hydrogen, alkyl, alkoxy, wherein said alkyl is unsubstituted or substituted with halogen, provided that at least one of R₇ and R₉ is not hydrogen;
L₁ is a direct bond, —C(R_{L1})(R_{L2})—, wherein said each of R_{L1} and R_{L2} is independently hydrogen or C₁₋₄alkyl optionally substituted with deuterium, halogen, alkyl, alkylene, alkynyl, or cyano;
Cy₁ is aryl, heterocyclyl, heteroaryl, or cycloalkyl, each of which is unsubstituted or substituted with one, two or three substituents R₃ₐ, wherein each R₃ₐ is independently selected from hydroxy, alkoxy, alkyl, halogen, aminoalkyl, cycloalkyl, cyano, heterocyclyl or heterocyclyloxy,
wherein
each alkyl moiety of which is unsubstituted or substituted with deuterium, halogen, alkoxy, hydroxy, cyano or heterocyclyl; and
each of said cycloalkyl or heterocyclyl is unsubstituted or substituted with alkoxy, alkyl, halogen, or hydroxy.

2. The compound of claim 1, wherein R₁ is hydrogen, or C₁₋₄alkyl optionally substituted with deuterium, halogen, hydroxy, alkoxy or cycloalkyl; preferably R₁ is hydrogen, or C₁₋₃alkyl.

3. The compound of any one of claim 1-2, wherein R₁ is hydrogen, methyl, ethyl, isopropyl, n-propyl; preferably R₁ is hydrogen, methyl, methyl-d3, or ethyl; more preferably R₁ is hydrogen or methyl.

4. The compound of any one of claim 1-3, wherein X₁ is C.

5. The compound of any one of claims 1-5, wherein R₂ is hydrogen, halogen, C₁₋₄alkyl or cyano; preferably R₂ is hydrogen, F, Br, Cl, CN, cyanomethyl, methyl, ethyl; more preferably R₂ is hydrogen, F, Br, CN, methyl.

6. The compound of any one of claim 1-3, wherein X₁ is N.

7. The compound of any one of claims 1-6, wherein R₄ is hydrogen, halogen or alkyl optionally substituted with deuterium; preferably R₄ is hydrogen, methyl or methyl-d3; more preferably hydrogen.

8. The compound of any one of claims 1-7, wherein R₅ is hydrogen, alkyl, alkenyl, alkynyl or cyano, wherein said alkyl is unsubstituted or substituted with cyano, cycloalkyl or heterocyclyl containing one oxygen atom; preferably R₅ is C₁₋₆alkyl, C₂₋₆alkenyl or C₂₋₆alkynyl, wherein said alkyl is substituted with cyano, C₃₋₆cycloalkyl or heterocyclyl containing one oxygen atom; more preferably R₅ is C₁₋₆alkyl, C₂₋₆alkenyl or C₂₋₆alkynyl, wherein said alkyl is substituted with cyano, C₃₋₆cycloalkyl or heterocyclyl containing one oxygen atom.

9. The compound of any one of claims 1-8, wherein R₅ is hydrogen, —CN, —CH₂—CN, —CH(CH₃)CN, —CH₂—CH₂—CN, —CH₂—CH₂—CH₂—CN, —CH(CH₃)—CH₂—CN, —CH₂—CH(CH₃)—CN, —CH(CH₂CH₃)—CN, oxiran-2-ylmethyl, oxiran-2-yl, oxetane-3-ylmethyl, oxetane-2-methyl, oxetane-3-yl, oxetane-2-yl, prop-2-yn-1-yl, but-2-yn-1-yl, but-3-yn-1-yl, pent-2-yn-1-yl, pent-3-yn-1-yl, pent-4-yn-1-yl, prop-2-en-1-yl, but-2-en-1-yl, but-3-en-1-yl, pent-2-en-1-yl, pent-3-en-1-yl, pent-4-en-1-yl, methyl, ethyl, propyl, isopropyl, cyclopropyl, cyclobutyl, azetidine-2-yl, azetidine-3-yl, azetidine-3-ylmethyl, azetidine-1-yl, azetidine-1-ylmethyl, aziridine-1-yl, aziridine-1-ylmethyl, aziridine-2-yl, aziridine-2-ylmethyl, 1-cyanocyclopropyl, 2-cyanocyclopropyl or 2-cyanocyclobutyl; preferably, R₅ is hydrogen, —CN, —CH₂—CN, —CH(CH₃)CN, —CH₂—CH₂—CN, CH₂—CH₂—CH₂—CN, —CH(CH₃)—CH₂—CN, —CH₂—CH(CH₃)—CN, —CH(CH₂CH₃)—CN, prop-2-yn-1-yl, but-3-yn-1-yl or pent-3-yn-1-yl; more preferably, R₅ is —CN, —CH₂—CN, —CH₂—CH₂—CN.

10. The compound of any one of claims 1-9, wherein each of R₇ and R₉ is independently hydrogen, or alkyl, wherein said alkyl is unsubstituted or substituted with halogen, alkoxy, aminos, cycloalkyl, cycloalkoxy, heterocyclyl; preferably each of R₇ and R₉ is independently C₁₋₄alkyl; more preferably each of R₇ and R₉ is independently C₁₋₂alkyl.

11. The compound according to any one of claims 1-10, wherein R₇ and R₉ are each independently hydrogen, methyl, ethyl, isopropyl, n-propyl, methoxymethyl, 2-methoxyethyl, provided that at least one of R₇ and R₉ is not hydrogen.

12. The compound of any one of claims 1-11, wherein R₇ is methyl, and R₉ is methyl; or R₇ is hydrogen, and R₉ is methyl; or R₇ is methyl, and R₉ is hydrogen; or R₇ is hydrogen, and R₉ is ethyl; or R₇ is ethyl, and R₉ is hydrogen; or R₇ is ethyl, and R₉ is ethyl; or R₇ is methyl, and R₉ is ethyl.

13. The compound of any one of claims 1-12, wherein R₈ and R₁₀ are each hydrogen.

14. The compound of any one of claims 1-13, wherein the 5-position carbon on the piperazine ring is R-configuration, provided that R₉ is not hydrogen.

15. The compound of claim 14, wherein the 2-position carbon on the piperazine ring is a chiral carbon; and the 5-position carbon on the piperazine ring is R-configuration, provided that R₇ is hydrogen and R₉ is not hydrogen.

16. The compound of claim 14, wherein the 2-position carbon on the piperazine ring is S-configuration, and the 5-position carbon on the piperazine ring is R-configuration, provided that R₇ and R₉ are not both hydrogens.

17. The compound of claim 14, wherein the 2-position carbon and the 5-position carbon on the piperazine ring are both R-configuration, provided that R₇ is methoxymethyl and R₉ is not hydrogen.

18. The compound of any one of claims 1-13, wherein the 5-position carbon on the piperazine ring is S-configuration, provided that R₉ is not hydrogen; preferably the 5-position carbon on the piperazine ring is S-configuration, provided that R₉ is methoxymethyl.

19. The compound of claim 18, wherein the 2-position carbon on the piperazine ring is achiral carbon; and the 5-position carbon on the piperazine ring is S-configuration, provided that R₇ is hydrogen and R₉ is not hydrogen.

20. The compound of claim 18, wherein the 2-position carbon on the piperazine ring is S-configuration, provided that R₇ is methoxymethyl.

21. The compound of any one of claims 1-20, wherein L₁ is a direct bond, or —C(R_{L1})(R_{L2}), wherein said each of R_{L1}, or R_{L2} is independently hydrogen or C₁₋₄alkyl optionally substituted with halogen, deuterium, alkyl, alkylene, alkynyl, cyano; preferably L₁ is a direct bond, —CH₂—, —CH(CH₃)—, —CH(CD₃)-, —CH(CH₂CH₃)—, —CH(C₃H₇)—, —CH(CHF₂)—, or —C(CH₃)₂—; more preferably L₁ is —CH(CH₃)— or —CH(CD₃)-.

22. The compound of any one of claims 1-21, wherein the compound of formula (I) is a compound of formula (II): 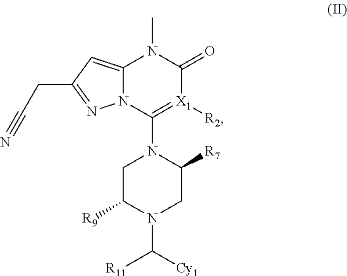
wherein each of R₇, R₉ and R₁₁ is, independently, methyl or ethyl, and the other variables are defined in claim 1.

23. The compound of any one of claims 1-22, wherein Cy₁ is aryl, heterocyclyl, heteroaryl, or cycloalkyl, each of which is unsubstituted or substituted with one, two or three substituents R₃ₐ, wherein each R₃ₐ is independently selected from hydroxy, alkoxy, alkyl, halogen, aminoalkyl, cycloalkyl, cycloalkyl, heterocyclyl or heterocyclyloxy, wherein each alkyl moiety of which is unsubstituted or substituted with halogen, alkoxy, hydroxy or heterocyclyl; and each of said cycloalkyl or heterocyclyl is unsubstituted or substituted with alkoxy, alkyl, halogen, or hydroxy; preferably R₃ₐ is F, Br, Cl, methyl, ethyl, isopropyl, trifluoromethyl, methoxy, ethoxy, isopropoxy, cyclopropyl, cyclobutane, difluoromethyl, 2-fluoro-2-methylethyl, oxetan-3-ylmethyloxy, difluoromethoxy, 2-methoxyethoxy, (2-methoxyethoxy)methyl, isopropoxy, or cyclopropoxy.

24. The compound of any one of claims 1-23, wherein Cy1 is heteroaryl optionally substituted with one, two or three substituents R₃ₐ, wherein each R₃ₐ is independently selected from hydroxy, alkoxy, alkyl, halogen, aminoalkyl, cycloalkyl, cycloalkyl, heterocyclyl or heterocyclyloxy, wherein each alkyl moiety of which is unsubstituted or substituted with halogen, alkoxy, hydroxy or heterocyclyl; and each of said cycloalkyl or heterocyclyl is unsubstituted or substituted with alkoxy, alkyl, halogen, or hydroxy; preferably R₃ₐ is F, Br, Cl, methyl, ethyl, isopropyl, trifluoromethyl, methoxy, ethoxy, isopropoxy, cyclopropyl, cyclobutane, difluoromethyl, 2-fluoro-2-methylethyl, oxetan-3-ylmethyloxy, difluoromethoxy, 2-methoxyethoxy, (2-methoxyethoxy)methyl, isopropoxy, or cyclopropoxy.

25. The compound of any one of claims 1-24, wherein Cy₁ is phenyl optionally substituted with one, two or three substituents R₃ₐ

26. The compound of claim 25, wherein Cy₁ is phenyl, which is substituted with one R₃ₐ at position 4 and optionally substituted with one or more R₃ₐ on the other position(s).

27. The compound of any one of claims 1-24, wherein Cy₁ is a monocyclic 5- to 9-membered heterocyclyl or heteroaryl, or a bicyclic 7- to 10-membered heterocyclyl or heteroaryl, each of which is unsubstituted or substituted with one, two or three R₃ₐ.

28. The compound of claim 27, wherein the monocyclic 5- to 9-membered heterocyclyl or heteroaryl, is 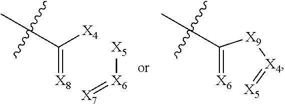
each of which is unsubstituted or substituted with one, two or three R₃ₐ, wherein each of X₄, X₅, X₆, X₇ and X₈ is independently selected from N or C, and X₉ is selected from C, N, S or O.

29. The compound of claim 28, wherein said monocyclic 5- to 9-membered heteroaryl is thiazole, isothiazole, triazole, pyridine, pyrazine, pyrimidine, each of which is unsubstituted or substituted with one, two or three R₃ₐ.

30. The compound of claim 28, wherein said monocyclic 5- to 9-membered heteroaryl is pyridin-2-yl, pyridin-3-yl, pyridin-4-yl, thiazole-2-yl, thiazole-4-yl, isothiazole-3-yl, isothiazole-4-yl, pyrazine-1-yl, pyrazine-2-yl, pyrimidin-2-yl, pyrimidin-4-yl, pyrimidin-5-yl, each of which is unsubstituted or substituted with one, two or three R₃ₐ.

31. The compound of any one of claims 1-24, wherein Cy¹ is

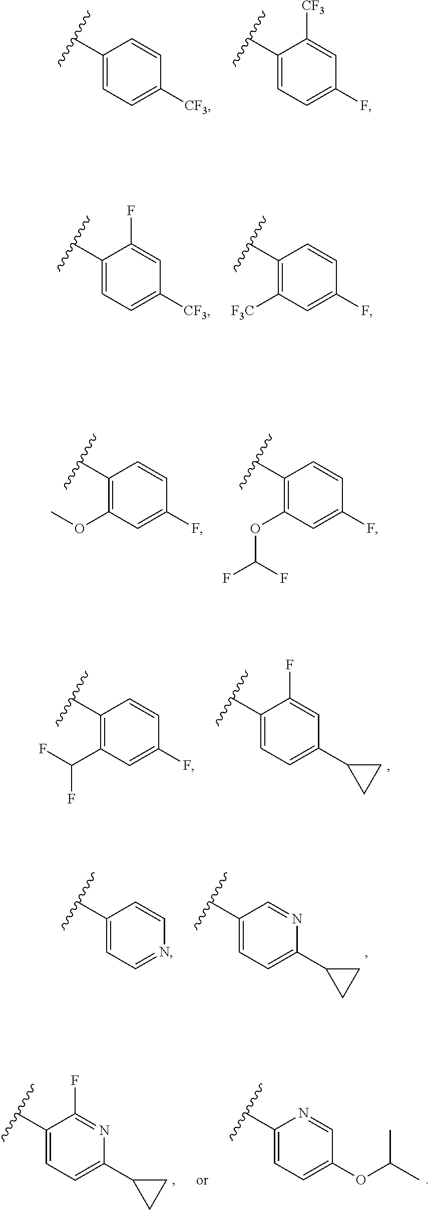

32. The compound of claim 27, wherein said bicyclic 7- to 10-membered heterocyclyl or heteroaryl, is 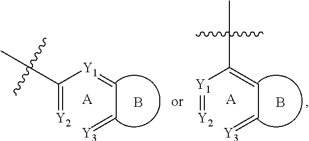
each of which is unsubstituted or substituted with one, two or three R₃ₐ, wherein A is a six-membered carbocycle or heterocycle; and B is selected from a 5- or 6-membered monocyclic carbocycle or monocyclic heterocycle fused to ring A to form an A-B bicyclic ring; and each of Y₁, Y₂, Y₃ is independently N or C.

33. The compound of claim 32, wherein said B is

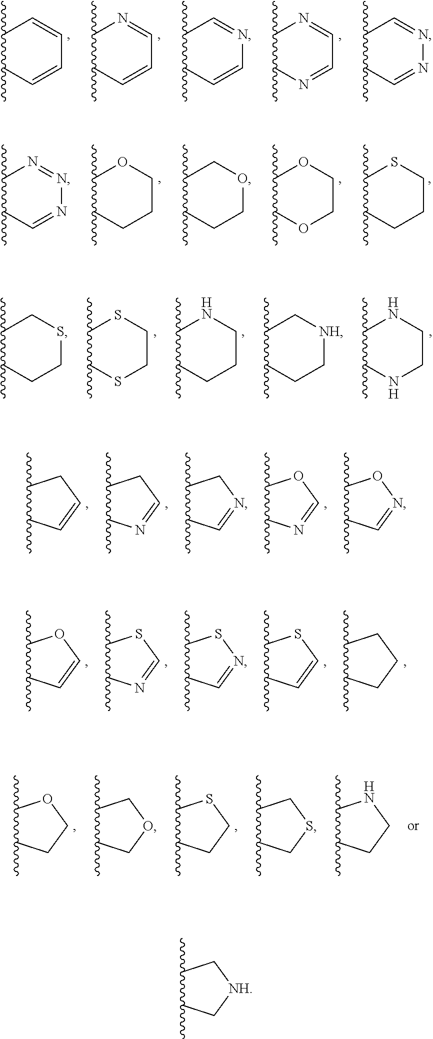

34. The compound of claim 33, wherein said bicyclic 7- to 10-membered is

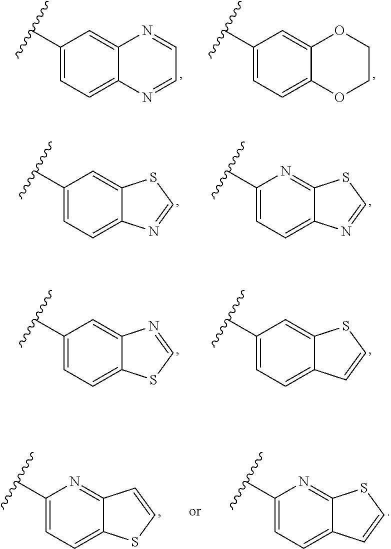

35. The compound of claim 27, wherein said bicyclic 7- to 10-membered heterocyclyl or heteroaryl, is 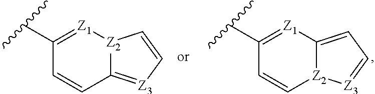
wherein each of Z₁, Z₂ and Z₃ is N or CH, provided at least two of Z₁, Z₂ and Z₃ are N.

36. The compound of claim 35, wherein said bicyclic 7- to 10-membered heterocyclyl or heteroaryl, is 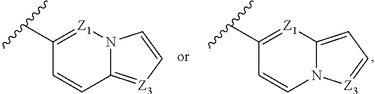
wherein each of Z₁ and Z₃ is N or CH, provided at least one of Z₁ and Z₂ is N; preferably

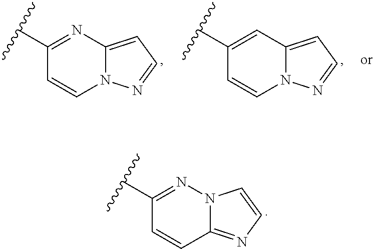 [SMILES: *c1ccn2nccc2n1 ; *c1ccn2nccc2c1 ; *c1ccc2nccn2n1]

37. The compound of claim 24, wherein said bicyclic 7- to 10-membered heterocyclyl or heteroaryl is 6,7-dihydro-5H-cyclopenta[b]pyridine, 6,7-dihydro-5H-cyclopenta[c]pyridine, chromane, isochromane, 2,3-dihydrobenzo[b][1,4]dioxine, thiochromane, isothiochromane, 2,3-dihydrobenzo[b][1,4]dithiine, quinoxalinyl, isoquinoline, quinoxaline, 2,3-dihydro-[1,4]dioxino[2,3-b]pyridine, 2,3-dihydro-[1,4]dioxino[2,3-c]pyridine, 3,4-dihydro-2H-pyrano[3,2-b]pyridine, 3,4-dihydro-2H-thiopyrano[2,3-b]pyridine, 2,3-dihydro-[1,4]dithiino[2,3-b]pyridine, 2,3-dihydro-[1,4]dithiino[2,3-c]pyridine, 1,2,3,4-tetrahydroquinoline, 1,2,3,4-tetrahydroisoquinoline, 5,6,7,8-tetrahydro-1,7-naphthyridine, 1,2,3,4-tetrahydro-2,7-naphthyridine, 1,2,3,4-tetrahydro-1,7-naphthyridine, 3H-indole, 1H-isoindole, benzofurane, benzo[b]thiophene, 3H-pyrrolo[3,2-b]pyridine, 7H-pyrrolo[3,4-b]pyridine, furo[2,3-b]pyridine, thieno[2,3-b]pyridine, benzo[d]thiazole, benzo[d]oxazole, oxazolo[5,4-b]pyridine, thiazolo[5,4-b]pyridine, oxazolo[4,5-b]pyridine, thiazolo[4,5-b]pyridine, 2,3-dihydro-1H-indene, 2,3-dihydrobenzofurane, 1,3-dihydroisobenzofurane, 1,3-dihydrobenzo[c]thiophene, 2,3-dihydrobenzo[b]thiophene, lbenzo[b]thiophene, thieno[3,2-b]pyridine, limidazo[1,2-b]pyridazine, pyrazolo[1,5-a]pyrimidine, lpyrazolo[1,5-a]pyridine, pyrrolo[1,2-b]pyridazine, imidazo[1,2-a]pyridine, or pyrrolo[1,2-a]pyrimidine, each of which is unsubstituted or substituted with one, two or three Ru.

38. The compound of claim 24, wherein said bicyclic 7- to 10-membered heterocyclyl or heteroaryl is 6,7-dihydro-5H-cyclopenta[b]pyridine-2-yl, 6,7-dihydro-5H-cyclopenta[b]pyridine-2-yl, 6,7-dihydro-5H-cyclopenta[c]pyridine-3-yl, 6,7-dihydro-5H-cyclopenta[c]pyridine-3-yl, chromane-7-yl, chromane-8-yl, isochromane-7-yl, isochromane-8-yl, 2,3-dihydrobenzo[b][1,4]dioxine-7-yl, 2,3-dihydrobenzo[b][1,4]dioxine-8-yl, thiochromane-7-yl, thiochromane-8-yl, 2,3-dihydrobenzo[b][1,4]dithiine-7-yl, 2,3-dihydrobenzo[b][1,4]dithiine-8-yl, quinoxalinyl-7-yl, quinoxalinyl-8-yl, isoquinoline-7-yl, isoquinoline-8-yl, quinoxaline-7-yl, quinoxaline-8-yl, 2,3-dihydro-[1,4]dioxino[2,3-b]pyridine-6-yl, 2,3-dihydro-[1,4]dioxino[2,3-b]pyridine-7-yl, 2,3-dihydro-[1,4]dioxino[2,3-c]pyridine-5-yl, 2,3-dihydro-[1,4]dioxino[2,3-c]pyridine-7-yl, 3,4-dihydro-2H-pyrano[3,2-b]pyridine-7-yl, 3,4-dihydro-2H-pyrano[3,2-b]pyridine-8-yl, 3,4-dihydro-2H-pyrano[3,2-b]pyridine-6-yl, 3,4-dihydro-2H-pyrano[2,3-b]pyridine-7-yl, 3,4-dihydro-2H-pyrano[2,3-b]pyridine-6-yl, 3,4-dihydro-2H-pyrano[2,3-b]pyridine-5-yl, 3,4-dihydro-2H-thiopyrano[3,2-b]pyridine-7-yl, 3,4-dihydro-2H-thiopyrano[3,2-b]pyridine-8-yl, 3,4-dihydro-2H-thiopyrano [3,2-b]pyridine-6-yl, 3,4-dihydro-2H-thiopyrano [2,3-b]pyridine-7-yl, 3,4-dihydro-2H-thiopyrano [2,3-b]pyridine-6-yl, 3,4-dihydro-2H-thiopyrano [2,3-b]pyridine-5-yl, 2,3-dihydro-[1,4]dithiino[2,3-b]pyridine-6-yl, 2,3-dihydro-[1,4]dithiino[2,3-b]pyridine-7-yl, 2,3-dihydro-[1,4]dithiino[2,3-c]pyridine-5-yl, 2,3-dihydro-[1,4]dithiino[2,3-c]pyridine-7-yl, 1,2,3,4-tetrahydroquinoline-7-yl, 1,2,3,4-tetrahydroquinoline-8-yl, 1,2,3,4-tetrahydroisoquinoline-7-yl, 1,2,3,4-tetrahydroisoquinoline-8-yl, 5,6,7,8-tetrahydro-1,7-naphthyridine-7-yl, 5,6,7,8-tetrahydro-1,7-naphthyridine-6-yl, 5,6,7,8-tetrahydro-1,7-naphthyridine-5-yl, 1,2,3,4-tetrahydro-2,7-naphthyridine-8-yl, 1,2,3,4-tetrahydro-2,7-naphthyridine-6-yl, 1,2,3,4-tetrahydro-2,7-naphthyridine-5-yl, 1,2,3,4-tetrahydro-1,7-naphthyridine-7-yl, 1,2,3,4-tetrahydro-1,7-naphthyridine-8-yl, 3H-indole-4-yl, 3H-indole-5-yl, 3H-indole-6-yl, 3H-indole-7-yl, 1H-isoindole-4-yl, 1H-isoindole-5-yl, 1H-isoindole-6-yl, 1H-isoindole-7-yl, benzofuran-4-yl, benzofuran-5-yl, benzofuran-6-yl, benzofuran-7-yl, benzo[b]thiophene-4-yl, benzo[b]thiophene-5-yl, benzo[b]thiophene-6-yl, benzo[b]thiophene-7-yl, 3H-pyrrolo[3,2-b]pyridine-5-yl, 3H-pyrrolo[3,2-b]pyridine-6-yl, 3H-pyrrolo[3,2-b]pyridine-7-yl, 7H-pyrrolo[3,4-b]pyridine-2-yl, 7H-pyrrolo[3,4-b]pyridine-3-yl, 7H-pyrrolo[3,4-b]pyridine-4-yl, furo[2,3-b]pyridine-4-yl, furo[2,3-b]pyridine-5-yl, furo[2,3-b]pyridine-6-yl, thieno[2,3-b]pyridine-4-yl, thieno[2,3-b]pyridine-5-yl, thieno[2,3-b]pyridine-6-yl, benzo[d]thiazole-4-yl, benzo[d]thiazole-5-yl, benzo[d]thiazole-6-yl, benzo[d]thiazole-7-yl, benzo[d]oxazole-4-yl, benzo[d]oxazole-5-yl, benzo[d]oxazole-6-yl, benzo[d]oxazole-7yl, oxazolo[5,4-b]pyridine-5-yl, oxazolo[5,4-b]pyridine-6-yl, oxazolo[5,4-b]pyridine-7-yl, thiazolo[5,4-b]pyridine-5-yl, thiazolo[5,4-b]pyridine-6-yl, thiazolo[5,4-b]pyridine-7-yl, oxazolo[4,5-b]pyridine-5-yl, oxazolo[4,5-b]pyridine-6-yl, oxazolo[4,5-b]pyridine-7-yl, thiazolo[4,5-b]pyridine-5-yl, thiazolo[4,5-b]pyridine-6-yl, thiazolo[4,5-b]pyridine-7-yl, 2,3-dihydro-1H-indene-4-yl, 2,3-dihydro-1H-indene-5-yl, 2,3-dihydrobenzofuran-4-yl, 2,3-dihydrobenzofuran-5-yl, 2,3-dihydrobenzofuran-6-yl, 2,3-dihydrobenzofuran-7-yl, 1,3-dihydroisobenzofuran-4-yl, 1,3-dihydroisobenzofuran-5-yl, 1,3-dihydrobenzo[c]thiophene-4-yl, 1,3-dihydrobenzo[c]thiophene-5-yl, 2,3-dihydrobenzo[b]thiophene-4-yl, 2,3-dihydrobenzo[b]thiophene-5-yl, 2,3-dihydrobenzo[b]thiophene-6-yl, 2,3-dihydrobenzo[b]thiophene-7-yl, benzo[b]thiophen-6-yl, benzo[b]thiophen-5-yl, benzo[b]thiophen-7-yl, thieno[3,2-b]pyridine-5-yl, thieno[3,2-b]pyridine-6-yl, thieno[3,2-b]pyridine-7-yl, imidazo[1,2-b]pyridazine-6-yl, imidazo[1,2-b]pyridazine-7-yl, imidazo[1,2-b]pyridazine-8-yl, pyrazolo[1,5-a]pyrimidin-5-yl, pyrazolo[1,5-a]pyrimidin-6-yl, pyrazolo[1,5-a]pyrimidin-7-yl, pyrazolo[1,5-a]pyridine-4-yl, pyrazolo[1,5-a]pyridine-5-yl, pyrazolo[1,5-a]pyridine-6-yl, pyrazolo[1,5-a]pyridine-7-yl, pyrrolo[1,2-b]pyridazine-2-yl, pyrrolo[1,2-b]pyridazine-3-yl, pyrrolo[1,2-b]pyridazine-4-yl, imidazo[1,2-a]pyridine-5-yl, imidazo[1,2-a]pyridine-6-yl, imidazo[1,2-a]pyridine-7-yl, imidazo[1,2-a]pyridine-8-yl, pyrrolo[1,2-a]pyrimidin-2-yl, pyrrolo[1,2-a]pyrimidin-3-yl, or pyrrolo[1,2-a]pyrimidin-4-yl, each of which is unsubstituted or substituted with one, two or three R₃a.

39. The compound of claim 24, wherein Cy₁ is quinoxalin-6-yl, 3-methylquinoxalin-6-yl, 3-(difluoromethyl)quinoxalin-6-yl, 3-methoxyquinoxalin-6-yl, 3-chloroquinoxalin-6-yl, 3,3-dimethyl-2,3-dihydro-[1,4]dioxino[2,3-b]pyridin-6-yl, 3,3-dimethyl-2,3-dihydrobenzo[b][1,4]dioxin-6-yl, 2,3-dihydrobenzo[b][1,4]dioxin-6-yl, 4-fluoro-2-(trifluoromethyl)phenyl, 4-fluoro-2-methoxyphenyl, 2-(difluoromethoxy)-4-fluorophenyl, 2-(difluoromethyl)-4-fluorophenyl, 4-cyclopropyl-2-fluorophenyl, 6-cyclopropylpyridin-3-yl, 5-isopropoxypyridin-2-yl, 6-cyclopropyl-2-fluoropyridin-3-yl, benzo[d]thiazol-6-yl, thiazolo[5,4-b]pyridin-5-yl, or 2-methylbenzo[d]thiazol-6-yl, benzo[d]thiazol-5-yl, 2-difluoromethyl-methylthieno[2,3-b]pyridine-6-yl, imidazo[1,2-b]pyridazine-6-yl, 2-methyl-imidazo[1,2-b]pyridazine-6-yl, 2-ethyl-imidazo[1,2-b]pyridazine-6-yl, pyrazolo[1,5-a]pyrimidin-5-yl, 2-methyl-pyrazolo[1,5-a]pyrimidin-5-yl, 2-fluoro-pyrazolo[1,5-a]pyrimidin-5-yl, 2-methyl-3-fluoro-pyrazolo[1,5-a]pyrimidin-5-yl, 3-fluoro-pyrazolo[1,5-a]pyrimidin-5-yl, 2-cyclopropyl-pyrazolo[1,5-a]pyrimidin-5-yl, 2-chloro-pyrazolo[1,5-a]pyrimidin-5-yl, 2,3-difluoro-pyrazolo[1,5-a]pyrimidin-5-yl, 2-chloro-3-fluoro-pyrazolo[1,5-a]pyrimidin-5-yl, pyrazolo[1,5-a]pyridine-5-yl, or 2-methyl-pyrazolo[1,5-a]pyridine-5-yl.

40. The compound of claim 24, wherein Cy₁ is

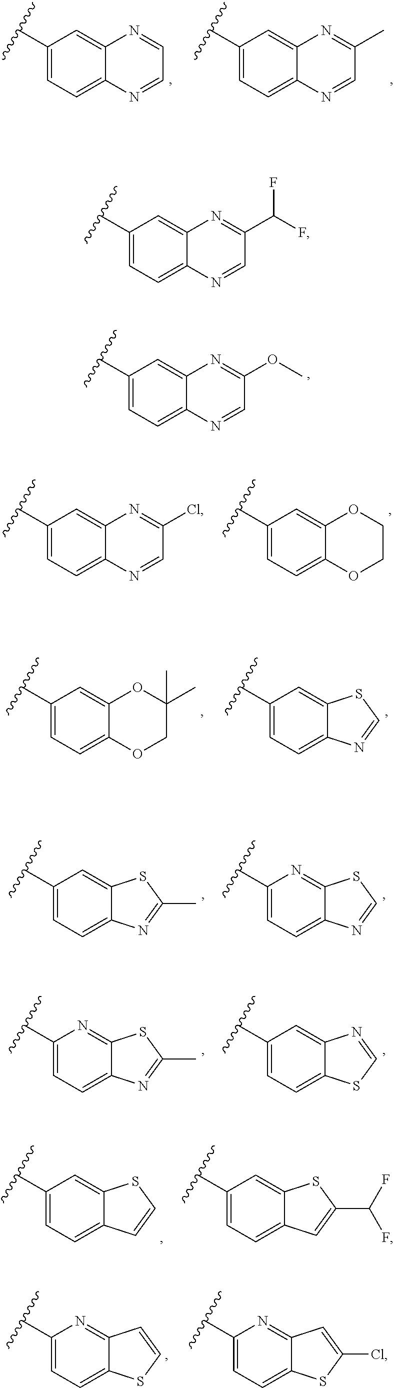

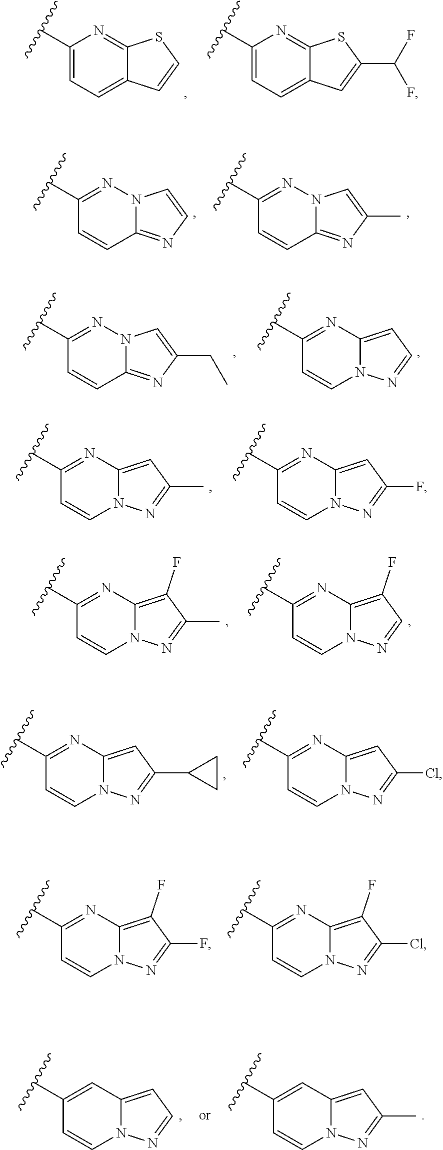

41. The compound of any one of claims 1-40, wherein the compound is selected from Table 1.

42. A compound, or a stereoisomer, or a pharmaceutically acceptable salt thereof, wherein said compounds is any one of the exemplified compounds.

43. A pharmaceutical composition comprising one or more compounds of any one of claims 1-41, or a stereoisomer or a pharmaceutically acceptable salt thereof, and a pharmaceutically acceptable excipient.

44. A method of treating cancer, comprising administering to a patient in need of such treatment a therapeutically effective amount of a compound of any one of claims 1-41 or a pharmaceutical composition of claim 43.
